SEQUENCE LISTING (276 sequences)

SEQ ID NO: 1 - protein, 116 aa, synthetic construct
QVQLQQSGAELMKPGASVKISCKATGYTFSTYWIEWVKQRPGHGLEWIGEILPGSGSTNYNEKFKGKATFTADASSNTAYMQLSSLTSEDSAVYYCARDGGAMDYWGQGTSVTVSS

SEQ ID NO: 2 - protein, 115 aa, synthetic construct
EIQLQQSGPELVKPGASVKMSCKASGYTFTDYVISWVKQRTGQGLEWIGEIYPGSHTYYNEKFKGKATLTADKSSNTAYMQLSSLTSEDSAVYFCALTAGAMDYWGQGTSVTVSS

SEQ ID NO: 3 - protein, 117 aa, synthetic construct
QVQLQQPGAELVKPGASVKLSCKASGYTFTNYWMHWVKQRPGQGLEWIGMIHPNSGSTNYNEKFKSKATLTVDKSSSTAYMQLSSLTSEDSAVYYCARPHGYPFGYWGQGTTLTVSS

SEQ ID NO: 4 - protein, 124 aa, synthetic construct
EVQLQQSGPELVKPGTSMKISCKASGYSFTDYTVNWVKQSHGKNLEWIGLINPYNGGTSYNQKFKGKATLTVDKSSSTAYMELLSLTSEDSAVYYCARWGYYYATSYYFAMDYWGQGTSVTVSS

SEQ ID NO: 5 - protein, 120 aa, synthetic construct
QVQLQQSGAELMKPGASVKLSCKATGYTFTGYWIEWVKQRPGHGLEWIGEILPGSGSTNYNEKFKGKATFTADTSSNTAYMQLSSLTTEDSAIYYCAYTVVATDWYFDVWGTGTTVTVSS

SEQ ID NO: 6 - protein, 116 aa, synthetic construct
EVKLMESGGGLVQPGASLRLSCAASGFTFTDYYMSWVRQPPGKAPEWLALIRNKANGYTTEYNASVKGRFTISRDNSQNILYLQMNTLRTEDSATYYCVRGGFAYWGQGTLVTVSA

SEQ ID NO: 7 - protein, 118 aa, synthetic construct
EVQLQQSGAELVRPGASVKLSCTASGFNIKDDYMHWVKQRPEQGLEWIGWIDPENGDTEYASKFQGKATITADTSSNTAYLQLSSLTSEDTAVYYCTTQYGPLYFDYWGQGTTLTVSS

SEQ ID NO: 8 - protein, 119 aa, synthetic construct
EVQLQQSGAELVRPGASVKLSCTASGFNIKDDYMHWVKQRPEQGLEWIGWIDPENGDTEYASKFQGKATITADTSSNTAYLQLSSLTSEDTAVYYCTTVAPYYYAMDYWGQGTSVTVSS

SEQ ID NO: 9 - protein, 118 aa, synthetic construct
EVQLQQSGAELVRPGASVKLSCTASGFNIKDDYMHWVKQRPEQGLEWIGWIDPENGDTEYASKFQGKATITADTSSNTAYLQLSSLTSEDTAVYYCTNYYGSSPFDYWGQGTTLTVSS

SEQ ID NO: 10 - protein, 116 aa, synthetic construct
QVQLKQSGPGLVQPSQSLSITCTVSGFSLTRYGVHWVRQSPGKGLEWLGVIWRGGSTDYNAAFMSRLSITKDNSKSQVFFKMNSLQADDTAIYYCAKEGTGILDSWGQGTTLTVSS

SEQ ID NO: 11 - protein, 119 aa, synthetic construct
EVQLQQSGAELVRPGASVKLSCTASGFNIKDDYMHWVKQRPEQGLEWIGWIDPENGDTEYASKFQGKATITADTSSNTAYLQLSSLTSEDTAVYYCTTVAPGYYAMDYWGQGTSVTVSS

SEQ ID NO: 12 - protein, 125 aa, synthetic construct
QVQLKQSGPGLVQPSQSLSITCTASGFSLTSYGVHWVRQSPGKGLEWLGVIWSGGSTDYSAAFISRLSISKDSSKSQVFFKMNSLQADDTAIYYCARNLPITSYDPSSFYAMDYWGQGTSVTVSS

SEQ ID NO: 13 - protein, 121 aa, synthetic construct
QVQLQQPGAELVKPGASVKLSCKASGYTFTSYWMQWVKQRPGQGLEWIGEIDPSDSYTNYNQKFKGKATLTVDTSSSTAYMQLSSLTSEDSAVYYCARGDYYGSSSWFAYWGQGTLVTVSA

SEQ ID NO: 14 - protein, 121 aa, synthetic construct
EVQLQQSGPELVKPGASVKMSCKASGYTFTDYNMHWVKQSHGKSLEWIGYINPNNGGTNYNQKFQGKATLTVNKSSRTAYMELRSLTSEDSAVYYCVRFYYGSRYYAMDYWGQGTSVTVSS

SEQ ID NO: 15 - protein, 119 aa, synthetic construct
QVQLQQSGPELVKPGASVKISCKASGYSFTSYYIHWVKQRPGQGLEWIGWIYPGSGNTKYNEKFKGKATLTADTSSSTAYMQLSSLTSEDSAVYYCARMGSSYWYFDVWGTGTTVTVSS

SEQ ID NO: 16 - protein, 118 aa, synthetic construct
QVQLQQSGPELVKPGASVKISCKASGYSFTSYYIHWVKQRPGQGLEWIGWIYPGSGNTKYNEKFKGKATLTADTSSSTAYMQLSSLTSEDSAVYYCARGNWHWYFDVWGTGTTVTVSS

SEQ ID NO: 17 - protein, 112 aa, synthetic construct
NIMMTQSPSSLAVSAGEKVTMSCKSSQRVLYSSNQKNYLAWYQQKPGQSPKLLIYWASTRESGVPDRFTGSGSGTDFTLTISTVQPEDLAVYYCHQYLSSRTFGGGTKLEIK

SEQ ID NO: 18 - protein, 112 aa, synthetic construct
NIMMTQSPSSLAVSAGEKVTMSCKSSQSVLYSSNQKNYLAWYQQKPGQSPKLLIYWASTRESGVPDRFTGSGSGTDFTLTISSVHAEDLAVYYCHQYLSSYTFGGGTKLEIK

SEQ ID NO: 19 - protein, 106 aa, synthetic construct
ENVLTQSPAIMSASLGEKVTMSCRASSSVNYMYWYQQKSDASPKLWIYYTSNLAPGVPARFSGSGSGNSYSLTISSMEGEDAATYYCQQFTSSPYTFGGGTKLEIK

SEQ ID NO: 20 - protein, 107 aa, synthetic construct
DIQMNQSPSSLSASLGDTITITCHASQNINVWLSWYQQKPGNIPKLLIYKASNLHTGVPSRFSGSGSGTGFTLTISSLQPEDIATYYCQQGQSYPWTFGGGTKLEIK

SEQ ID NO: 21 - protein, 106 aa, synthetic construct
ENVLTQSPAIMSASLGEKVTMSCRASSSVNYMYWYQQKSDASPKLWIYYTSNLAPGVPARFSGSGSGNSYSLTISSMEGEDAATYYCQQFTSSPWTFGGGTKLEIK

SEQ ID NO: 22 - protein, 109 aa, synthetic construct
QAVVIQESALTTSPGETVTLTCRSSTGAVTTSHYAIWVQEKPDHLFTGLIGGTNNRVPGVPARFSGSLIADKAALTITGAQTEDEAIYFCALWYTNQFIFGSGTKVTVL

SEQ ID NO: 23 - protein, 109 aa, synthetic construct
QAVVTQESALTTSPGETVTLTCRSSTGAVTTSKYANWVQEKPDHLFTGVIGGTNNRAPGVPARFSGSLIGDKAALTITGAQTEDEAIYFCALWYSNHWVFGGGTKLTVL

SEQ ID NO: 24 - protein, 105 aa, synthetic construct
QIVLTQSPAIMSASLGEEITLTCSASSSVSYMHWYQQKSGTSPKLLIYSTSNLASGVPSRFSGSGSGTFYSLTISSVEAEDAADYYCHQWSSYHTFGGGTKLEIK

SEQ ID NO: 25 - protein, 108 aa, synthetic construct
QIVLTQSPAIMSAFLGERVTMTCTASSSVSSRYLHWYQQKPGSSPKLWIYSTSNLASGVPARFSGSGSGTSYSLTISSMEAEDAATYYCHQYHRSPLTFGAGTKLELK

SEQ ID NO: 26 - protein, 111 aa, synthetic construct
DIVLTQSPASLAVSLGQRATISCRASKSVSTSGYSYMHWYQQKPGQPPTLLIYLASNLESGVPARFSGSGSGTDFTLNIHPVEEEDAATYYCQHSRELPYTFGGGTKLEIK

SEQ ID NO: 27 - protein, 106 aa, synthetic construct
ENVLTQSPAIMSASPGEKVTMTCRASSSVSSSYLHWYQQKSGASPKLWIYSTSNLASGVPARFSGSGSGTSYSLTISSVEAEDAATYYCQQYSGFTFGSGTKLEIK

SEQ ID NO: 28 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 29 - protein, 112 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGAGTKLELK

SEQ ID NO: 30 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIK

SEQ ID NO: 31 - protein, 108 aa, synthetic construct
ENVLTQSPAIMAASLGQKVTMTCSASSSVSSSYLHWYQQKSGASPKPLIHRTSNLASGVPARFSGSGSGTSYSLTISSVEAEDDATYYCQQWSGYPLTFGAGTKLELK

SEQ ID NO: 32 - protein, 108 aa, synthetic construct
EIVLTQSPALMAASPGEKVTITCSVSSSISSSNLHWYQQKSETSPKPWIYGTSNLASGVPVRFSGSGSGTSYSLTISSMEAEDAATYYCQQWSSYPLTFGAGTKLELK

SEQ ID NO: 33 - protein, 5 aa, synthetic construct
TYWIE

SEQ ID NO: 34 - protein, 17 aa, synthetic construct
EILPGSGSTNYNEKFKG

SEQ ID NO: 35 - protein, 7 aa, synthetic construct
DGGAMDY

SEQ ID NO: 36 - protein, 5 aa, synthetic construct
DYVIS

SEQ ID NO: 37 - protein, 16 aa, synthetic construct
EIYPGSHTYYNEKFKG

SEQ ID NO: 38 - protein, 7 aa, synthetic construct
TAGAMDY

SEQ ID NO: 39 - protein, 5 aa, synthetic construct
NYWMH

SEQ ID NO: 40 - protein, 17 aa, synthetic construct
MIHPNSGSTNYNEKFKS

SEQ ID NO: 41 - protein, 8 aa, synthetic construct
PHGYPFGY

SEQ ID NO: 42 - protein, 5 aa, synthetic construct
DYTVN

SEQ ID NO: 43 - protein, 17 aa, synthetic construct
LINPYNGGTSYNQKFKG

SEQ ID NO: 44 - protein, 15 aa, synthetic construct
WGYYYATSYYFAMDY

SEQ ID NO: 45 - protein, 5 aa, synthetic construct
GYWIE

SEQ ID NO: 46 - protein, 11 aa, synthetic construct
TVVATDWYFDV

SEQ ID NO: 47 - protein, 5 aa, synthetic construct
DYYMS

SEQ ID NO: 48 - protein, 19 aa, synthetic construct
LIRNKANGYTTEYNASVKG

SEQ ID NO: 49 - protein, 5 aa, synthetic construct
GGFAY

SEQ ID NO: 50 - protein, 5 aa, synthetic construct
DDYMH

SEQ ID NO: 51 - protein, 17 aa, synthetic construct
WIDPENGDTEYASKFQG

SEQ ID NO: 52 - protein, 9 aa, synthetic construct
QYGPLYFDY

SEQ ID NO: 53 - protein, 17 aa, synthetic construct
WIDPENGDTEYASKFQG

SEQ ID NO: 54 - protein, 10 aa, synthetic construct
VAPYYYAMDY

SEQ ID NO: 55 - protein, 9 aa, synthetic construct
YYGSSPFDY

SEQ ID NO: 56 - protein, 5 aa, synthetic construct
RYGVH

SEQ ID NO: 57 - protein, 16 aa, synthetic construct
VIWRGGSTDYNAAFMS

SEQ ID NO: 58 - protein, 8 aa, synthetic construct
EGTGILDS

SEQ ID NO: 59 - protein, 17 aa, synthetic construct
WIDPENGDTEYASKFQG

SEQ ID NO: 60 - protein, 10 aa, synthetic construct
VAPGYYAMDY

SEQ ID NO: 61 - protein, 5 aa, synthetic construct
SYGVH

SEQ ID NO: 62 - protein, 16 aa, synthetic construct
VIWSGGSTDYSAAFIS

SEQ ID NO: 63 - protein, 17 aa, synthetic construct
NLPITSYDPSSFYAMDY

SEQ ID NO: 64 - protein, 5 aa, synthetic construct
SYWMQ

SEQ ID NO: 65 - protein, 17 aa, synthetic construct
EIDPSDSYTNYNQKFKG

SEQ ID NO: 66 - protein, 12 aa, synthetic construct
GDYYGSSSWFAY

SEQ ID NO: 67 - protein, 5 aa, synthetic construct
DYNMH

SEQ ID NO: 68 - protein, 17 aa, synthetic construct
YINPNNGGTNYNQKFQG

SEQ ID NO: 69 - protein, 12 aa, synthetic construct
FYYGSRYYAMDY

SEQ ID NO: 70 - protein, 5 aa, synthetic construct
SYYIH

SEQ ID NO: 71 - protein, 17 aa, synthetic construct
WIYPGSGNTKYNEKFKG

SEQ ID NO: 72 - protein, 10 aa, synthetic construct
MGSSYWYFDV

SEQ ID NO: 73 - protein, 9 aa, synthetic construct
GNWHWYFDV

SEQ ID NO: 74 - protein, 17 aa, synthetic construct
KSSQRVLYSSNQKNYLA

SEQ ID NO: 75 - protein, 7 aa, synthetic construct
WASTRES

SEQ ID NO: 76 - protein, 8 aa, synthetic construct
HQYLSSRT

SEQ ID NO: 77 - protein, 17 aa, synthetic construct
KSSQSVLYSSNQKNYLA

SEQ ID NO: 78 - protein, 8 aa, synthetic construct
HQYLSSYT

SEQ ID NO: 79 - protein, 10 aa, synthetic construct
RASSSVNYMY

SEQ ID NO: 80 - protein, 7 aa, synthetic construct
YTSNLAP

SEQ ID NO: 81 - protein, 9 aa, synthetic construct
QQFTSSPYT

SEQ ID NO: 82 - protein, 11 aa, synthetic construct
HASQNINVWLS

SEQ ID NO: 83 - protein, 7 aa, synthetic construct
KASNLHT

SEQ ID NO: 84 - protein, 9 aa, synthetic construct
QQGQSYPWT

SEQ ID NO: 85 - protein, 9 aa, synthetic construct
QQFTSSPWT

SEQ ID NO: 86 - protein, 14 aa, synthetic construct
RSSTGAVTTSHYAI

SEQ ID NO: 87 - protein, 7 aa, synthetic construct
GTNNRVP

SEQ ID NO: 88 - protein, 9 aa, synthetic construct
ALWYTNQFI

SEQ ID NO: 89 - protein, 14 aa, synthetic construct
RSSTGAVTTSKYAN

SEQ ID NO: 90 - protein, 7 aa, synthetic construct
GTNNRAP

SEQ ID NO: 91 - protein, 9 aa, synthetic construct
ALWYSNHWV

SEQ ID NO: 92 - protein, 10 aa, synthetic construct
SASSSVSYMH

SEQ ID NO: 93 - protein, 7 aa, synthetic construct
STSNLAS

SEQ ID NO: 94 - protein, 8 aa, synthetic construct
HQWSSYHT

SEQ ID NO: 95 - protein, 12 aa, synthetic construct
TASSSVSSRYLH

SEQ ID NO: 96 - protein, 9 aa, synthetic construct
HQYHRSPLT

SEQ ID NO: 97 - protein, 15 aa, synthetic construct
RASKSVSTSGYSYMH

SEQ ID NO: 98 - protein, 7 aa, synthetic construct
LASNLES

SEQ ID NO: 99 - protein, 9 aa, synthetic construct
QHSRELPYT

SEQ ID NO: 100 - protein, 12 aa, synthetic construct
RASSSVSSSYLH

SEQ ID NO: 101 - protein, 7 aa, synthetic construct
QQYSGFT

SEQ ID NO: 102 - protein, 11 aa, synthetic construct
SASQGISNYLN

SEQ ID NO: 103 - protein, 7 aa, synthetic construct
YTSSLHS

SEQ ID NO: 104 - protein, 9 aa, synthetic construct
QQYSKLPFT

SEQ ID NO: 105 - protein, 16 aa, synthetic construct
RSSKSLLHSNGNTYLY

SEQ ID NO: 106 - protein, 7 aa, synthetic construct
RMSNLAS

SEQ ID NO: 107 - protein, 9 aa, synthetic construct
MQHLEYPFT

SEQ ID NO: 108 - protein, 11 aa, synthetic construct
RASQSISDYLH

SEQ ID NO: 109 - protein, 7 aa, synthetic construct
YASQSIS

SEQ ID NO: 110 - protein, 9 aa, synthetic construct
QNGHSFPYT

SEQ ID NO: 111 - protein, 12 aa, synthetic construct
SASSSVSSSYLH

SEQ ID NO: 112 - protein, 7 aa, synthetic construct
RTSNLAS

SEQ ID NO: 113 - protein, 9 aa, synthetic construct
QQWSGYPLT

SEQ ID NO: 114 - protein, 12 aa, synthetic construct
SVSSSISSSNLH

SEQ ID NO: 115 - protein, 7 aa, synthetic construct
GTSNLAS

SEQ ID NO: 116 - protein, 9 aa, synthetic construct
QQWSSYPLT

SEQ ID NO: 117 - protein, 121 aa, synthetic construct [sequence omitted]

SEQ ID NO: 118 - protein, 106 aa, synthetic construct [sequence omitted]

SEQ ID NO: 119 - protein, 5 aa, synthetic construct
SHWMH

SEQ ID NO: 120 - protein, 17 aa, synthetic construct
EFNPSNGRTNYNEKFKS

SEQ ID NO: 121 - protein, 12 aa, synthetic construct
RDYDYDGRYFDY

SEQ ID NO: 122 - protein, 10 aa, synthetic construct
SASSSVTYMY

SEQ ID NO: 123 - protein, 7 aa, synthetic construct
DTSNLAS

SEQ ID NO: 124 - protein, 9 aa, synthetic construct
QQWSSHIFT

SEQ ID NO: 125 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 126 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 127 - protein, 5 aa, synthetic construct
NYGVH

SEQ ID NO: 128 - protein, 16 aa, synthetic construct
VIWSGGNTDYNTPFTS

SEQ ID NO: 129 - protein, 11 aa, synthetic construct
ALTYYDYEFAY

SEQ ID NO: 130 - protein, 11 aa, synthetic construct
RASQSIGTNIH

SEQ ID NO: 131 - protein, 7 aa, synthetic construct
YASESIS

SEQ ID NO: 132 - protein, 9 aa, synthetic construct
QQNNNWPTT

SEQ ID NO: 133 - protein, 447 aa, synthetic construct
EVQLVESGGGLVQPGGSLRLSCAVSGYSITSGYSWNWIRQAPGKGLEWVASITYDGSTNYADSVKGRFTISRDDSKNTFYLQMNSLRAEDTAVYYCARGSHYFGHWHFAVWGQGTLVTVSSGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 215 aa, synthetic construct
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNR

SEQ ID NO: 135 - protein, 6 aa, synthetic construct
SGYSWN

SEQ ID NO: 136 - protein, 16 aa, synthetic construct
SITYDGSTNYADSVKG

SEQ ID NO: 137 - protein, 12 aa, synthetic construct
GSHYFGHWHFAV

SEQ ID NO: 138 - protein, 15 aa, synthetic construct
RASQSVDYDGDSYMN

SEQ ID NO: 139 - protein, 7 aa, synthetic construct
AASYLES

SEQ ID NO: 140 - protein, 9 aa, synthetic construct
QQSHEDPYT

SEQ ID NO: 141 - protein, 30 aa, synthetic construct
QVQLQQSGAELMKPGASVKISCKATGYTFS

SEQ ID NO: 142 - protein, 14 aa, synthetic construct
WVKQRPGHGLEWIG

SEQ ID NO: 143 - protein, 32 aa, synthetic construct
KATFTADASSNTAYMQLSSLTSEDSAVYYCAR

SEQ ID NO: 144 - protein, 11 aa, synthetic construct
WGQGTSVTVSS

SEQ ID NO: 145 - protein, 30 aa, synthetic construct
EIQLQQSGPELVKPGASVKMSCKASGYTFT

SEQ ID NO: 146 - protein, 14 aa, synthetic construct
WVKQRTGQGLEWIG

SEQ ID NO: 147 - protein, 32 aa, synthetic construct
KATLTADKSSNTAYMQLSSLTSEDSAVYFCAL

SEQ ID NO: 148 - protein, 11 aa, synthetic construct
WGQGTSVTVSS

SEQ ID NO: 149 - protein, 30 aa, synthetic construct
QVQLQQPGAELVKPGASVKLSCKASGYTFT

SEQ ID NO: 150 - protein, 14 aa, synthetic construct
WVKQRPGQGLEWIG

SEQ ID NO: 151 - protein, 31 aa, synthetic construct
ATLTVDKSSSTAYMQLSSLTSEDSAVYYCAR

SEQ ID NO: 152 - protein, 11 aa, synthetic construct
WGQGTTLTVSS

SEQ ID NO: 153 - protein, 30 aa, synthetic construct
EVQLQQSGPELVKPGTSMKISCKASGYSFT

SEQ ID NO: 154 - protein, 14 aa, synthetic construct
WVKQSHGKNLEWIG

SEQ ID NO: 155 - protein, 32 aa, synthetic construct
KATLTVDKSSSTAYMELLSLTSEDSAVYYCAR

SEQ ID NO: 156 - protein, 11 aa, synthetic construct
WGQGTSVTVSS

SEQ ID NO: 157 - protein, 30 aa, synthetic construct
QVQLQQSGAELMKPGASVKLSCKATGYTFT

SEQ ID NO: 158 - protein, 14 aa, synthetic construct
WVKQRPGHGLEWIG

SEQ ID NO: 159 - protein, 32 aa, synthetic construct
KATFTADTSSNTAYMQLSSLTTEDSAIYYCAY

SEQ ID NO: 160 - protein, 11 aa, synthetic construct
WGTGTTVTVSS

SEQ ID NO: 161 - protein, 30 aa, synthetic construct
EVKLMESGGGLVQPGASLRLSCAASGFTFT

SEQ ID NO: 162 - protein, 14 aa, synthetic construct
WVRQPPGKAPEWLA

SEQ ID NO: 163 - protein, 31 aa, synthetic construct
FTISRDNSQNILYLQMNTLRTEDSATYYCVR

SEQ ID NO: 164 - protein, 11 aa, synthetic construct
WGQGTLVTVSA

SEQ ID NO: 165 - protein, 30 aa, synthetic construct
EVQLQQSGAELVRPGASVKLSCTASGFNIK

SEQ ID NO: 166 - protein, 14 aa, synthetic construct
WVKQRPEQGLEWIG

SEQ ID NO: 167 - protein, 32 aa, synthetic construct
KATITADTSSNTAYLQLSSLTSEDTAVYYCTT

SEQ ID NO: 168 - protein, 11 aa, synthetic construct
WGQGTTLTVSS

SEQ ID NO: 169 - protein, 30 aa, synthetic construct; identical to SEQ ID NO: 165

SEQ ID NO: 170 - protein, 14 aa, synthetic construct
WVKQRPEQGLEWIG

SEQ ID NO: 171 - protein, 32 aa, synthetic construct; identical to SEQ ID NO: 167

SEQ ID NO: 172 - protein, 11 aa, synthetic construct
WGQGTSVTVSS

SEQ ID NO: 173 - protein, 30 aa, synthetic construct; identical to SEQ ID NO: 165

SEQ ID NO: 174 - protein, 14 aa, synthetic construct
WVKQRPEQGLEWIG

SEQ ID NO: 175 - protein, 32 aa, synthetic construct
KATITADTSSNTAYLQLSSLTSEDTAVYYCTN

SEQ ID NO: 176 - protein, 11 aa, synthetic construct
WGQGTTLTVSS

SEQ ID NO: 177 - protein, 30 aa, synthetic construct
QVQLKQSGPGLVQPSQSLSITCTVSGFSLT

SEQ ID NO: 178 - protein, 14 aa, synthetic construct
WVRQSPGKGLEWLG

SEQ ID NO: 179 - protein, 32 aa, synthetic construct
RLSITKDNSKSQVFFKMNSLQADDTAIYYCAK

SEQ ID NO: 180 - protein, 11 aa, synthetic construct
WGQGTTLTVSS

SEQ ID NO: 181 - protein, 30 aa, synthetic construct; identical to SEQ ID NO: 165

SEQ ID NO: 182 - protein, 14 aa, synthetic construct
WVRQSPGKGLEWLG

SEQ ID NO: 183 - protein, 32 aa, synthetic construct; identical to SEQ ID NO: 167

SEQ ID NO: 184 - protein, 11 aa, synthetic construct
WGQGTSVTVSS

SEQ ID NO: 185 - protein, 30 aa, synthetic construct
QVQLKQSGPGLVQPSQSLSITCTASGFSLT

SEQ ID NO: 186 - protein, 14 aa, synthetic construct
WVRQSPGKGLEWLG

SEQ ID NO: 187 - protein, 32 aa, synthetic construct
RLSISKDSSKSQVFFKMNSLQADDTAIYYCAR

SEQ ID NO: 188 - protein, 11 aa, synthetic construct
WGQGTSVTVSS

SEQ ID NO: 189 - protein, 30 aa, synthetic construct; identical to SEQ ID NO: 149

SEQ ID NO: 190 - protein, 14 aa, synthetic construct
WVKQRPGQGLEWIG

SEQ ID NO: 191 - protein, 32 aa, synthetic construct
KATLTVDTSSSTAYMQLSSLTSEDSAVYYCAR

SEQ ID NO: 192 - protein, 11 aa, synthetic construct
WGQGTLVTVSA

SEQ ID NO: 193 - protein, 30 aa, synthetic construct
EVQLQQSGPELVKPGASVKMSCKASGYTFT

SEQ ID NO: 194 - protein, 14 aa, synthetic construct
WVKQSHGKSLEWIG

SEQ ID NO: 195 - protein, 32 aa, synthetic construct
KATLTVNKSSRTAYMELRSLTSEDSAVYYCVR

SEQ ID NO: 196 - protein, 11 aa, synthetic construct
WGQGTSVTVSS

SEQ ID NO: 197 - protein, 30 aa, synthetic construct
QVQLQQSGPELVKPGASVKISCKASGYSFT

SEQ ID NO: 198 - protein, 14 aa, synthetic construct
WVKQRPGQGLEWIG

SEQ ID NO: 199 - protein, 32 aa, synthetic construct
KATLTADTSSSTAYMQLSSLTSEDSAVYYCAR

SEQ ID NO: 200 - protein, 11 aa, synthetic construct
WGTGTTVTVSS

SEQ ID NO: 201 - protein, 30 aa, synthetic construct; identical to SEQ ID NO: 197

SEQ ID NO: 202 - protein, 14 aa, synthetic construct
WVKQRPGQGLEWIG

SEQ ID NO: 203 - protein, 32 aa, synthetic construct; identical to SEQ ID NO: 199

SEQ ID NO: 204 - protein, 11 aa, synthetic construct
WGTGTTVTVSS

SEQ ID NO: 205 - protein, 23 aa, synthetic construct
NIMMTQSPSSLAVSAGEKVTMSC

SEQ ID NO: 206 - protein, 15 aa, synthetic construct
WYQQKPGQSPKLLIY

SEQ ID NO: 207 - protein, 32 aa, synthetic construct
GVPDRFTGSGSGTDFTLTISTVQPEDLAVYYC

SEQ ID NO: 208 - protein, 10 aa, synthetic construct
FGGGTKLEIK

SEQ ID NO: 209 - protein, 23 aa, synthetic construct
NIMMTQSPSSLAVSAGEKVTMSC

SEQ ID NO: 210 - protein, 15 aa, synthetic construct
WYQQKPGQSPKLLIY

SEQ ID NO: 211 - protein, 32 aa, synthetic construct
GVPDRFTGSGSGTDFTLTISSVHAEDLAVYYC

SEQ ID NO: 212 - protein, 10 aa, synthetic construct
FGGGTKLEIK

SEQ ID NO: 213 - protein, 23 aa, synthetic construct
ENVLTQSPAIMSASLGEKVTMSC

SEQ ID NO: 214 - protein, 15 aa, synthetic construct
WYQQKSDASPKLWIY

SEQ ID NO: 215 - protein, 32 aa, synthetic construct
GVPARFSGSGSGNSYSLTISSMEGEDAATYYC

SEQ ID NO: 216 - protein, 10 aa, synthetic construct
FGGGTKLEIK

SEQ ID NO: 217 - protein, 23 aa, synthetic construct
DIQMNQSPSSLSASLGDTITITC

SEQ ID NO: 218 - protein, 15 aa, synthetic construct
WYQQKPGNIPKLLIY

SEQ ID NO: 219 - protein, 32 aa, synthetic construct
GVPSRFSGSGSGTGFTLTISSLQPEDIATYYC

SEQ ID NO: 220 - protein, 10 aa, synthetic construct
FGGGTKLEIK

SEQ ID NO: 221 - protein, 23 aa, synthetic construct
ENVLTQSPAIMSASLGEKVTMSC

SEQ ID NO: 222 - protein, 15 aa, synthetic construct
WYQQKSDASPKLWIY

SEQ ID NO: 223 - protein, 32 aa, synthetic construct; identical to SEQ ID NO: 215

SEQ ID NO: 224 - protein, 10 aa, synthetic construct
FGGGTKLEIK

SEQ ID NO: 225 - protein, 22 aa, synthetic construct
QAVVIQESALTTSPGETVTLTC

SEQ ID NO: 226 - protein, 15 aa, synthetic construct
WVQEKPDHLFTGLIG

SEQ ID NO: 227 - protein, 32 aa, synthetic construct
GVPARFSGSLIADKAALTITGAQTEDEAIYFC

SEQ ID NO: 228 - protein, 9 aa, synthetic construct
GSGTKVTVL

SEQ ID NO: 229 - protein, 22 aa, synthetic construct
QAVVTQESALTTSPGETVTLTC

SEQ ID NO: 230 - protein, 15 aa, synthetic construct
WVQEKPDHLFTGVIG

SEQ ID NO: 231 - protein, 32 aa, synthetic construct
GVPARFSGSLIGDKAALTITGAQTEDEAIYFC

SEQ ID NO: 232 - protein, 10 aa, synthetic construct
FGGGTKLTVL

SEQ ID NO: 233 - protein, 23 aa, synthetic construct
QIVLTQSPAIMSASLGEEITLTC

SEQ ID NO: 234 - protein, 15 aa, synthetic construct
WYQQKSGTSPKLLIY

SEQ ID NO: 235 - protein, 32 aa, synthetic construct
GVPSRFSGSGSGTFYSLTISSVEAEDAADYYC

SEQ ID NO: 236 - protein, 10 aa, synthetic construct
FGGGTKLEIK

SEQ ID NO: 237 - protein, 23 aa, synthetic construct
QIVLTQSPAIMSAFLGERVTMTC

SEQ ID NO: 238 - protein, 15 aa, synthetic construct
WYQQKPGSSPKLWIY

SEQ ID NO: 239 - protein, 32 aa, synthetic construct
GVPARFSGSGSGTSYSLTISSMEAEDAATYYC

SEQ ID NO: 240 - protein, 10 aa, synthetic construct
FGAGTKLELK

SEQ ID NO: 241 - protein, 23 aa, synthetic construct
DIVLTQSPASLAVSLGQRATISC

SEQ ID NO: 242 - protein, 15 aa, synthetic construct
WYQQKPGQPPTLLIY

SEQ ID NO: 243 - protein, 32 aa, synthetic construct
GVPARFSGSGSGTDFTLNIHPVEEEDAATYYC

SEQ ID NO: 244 - protein, 10 aa, synthetic construct
FGGGTKLEIK

SEQ ID NO: 245 - protein, 23 aa, synthetic construct
ENVLTQSPAIMSASPGEKVTMTC

SEQ ID NO: 246 - protein, 15 aa, synthetic construct
WYQQKSGASPKLWIY

SEQ ID NO: 247 - protein, 32 aa, synthetic construct
GVPARFSGSGSGTSYSLTISSVEAEDAATYYC

SEQ ID NO: 248 - protein, 10 aa, synthetic construct
FGSGTKLEIK

SEQ ID NO: 249 - protein, 23 aa, synthetic construct
DIQMTQTTSSLSASLGDRVTISC

SEQ ID NO: 250 - protein, 15 aa, synthetic construct
WYQQKPDGTVKLLIY

SEQ ID NO: 251 - protein, 32 aa, synthetic construct
GVPSRFSGSGSGTDYSLTISNLEPEDIATYYC

SEQ ID NO: 252 - protein, 10 aa, synthetic construct
FGSGTKLEIK

SEQ ID NO: 253 - protein, 23 aa, synthetic construct
DIVMTQAAPSVPVTPGESVSISC

SEQ ID NO: 254 - protein, 15 aa, synthetic construct
WFLQRPGQSPQLLIY

SEQ ID NO: 255 - protein, 32 aa, synthetic construct
GVPDRFSGSGSGTAFTLRISRVEAEDVGVYYC

SEQ ID NO: 256 - protein, 10 aa, synthetic construct
FGAGTKLELK

SEQ ID NO: 257 - protein, 23 aa, synthetic construct
DIVMTQSPATLSVTPGDRVSLSC

SEQ ID NO: 258 - protein, 15 aa, synthetic construct
WYQQKSHESPRLLIK

SEQ ID NO: 259 - protein, 32 aa, synthetic construct
GIPSRFSGSGSGSDFTLSINSVEPEDVGVYYC

SEQ ID NO: 260 - protein, 10 aa, synthetic construct
FGGGTKLEIK

SEQ ID NO: 261 - protein, 23 aa, synthetic construct
ENVLTQSPAIMAASLGQKVTMTC

SEQ ID NO: 262 - protein, 15 aa, synthetic construct
WYQQKSGASPKPLIH

SEQ ID NO: 263 - protein, 32 aa, synthetic construct
GVPARFSGSGSGTSYSLTISSVEAEDDATYYC

SEQ ID NO: 264 - protein, 10 aa, synthetic construct
FGAGTKLELK

SEQ ID NO: 265 - protein, 23 aa, synthetic construct
EIVLTQSPALMAASPGEKVTITC

SEQ ID NO: 266 - protein, 15 aa, synthetic construct
WYQQKSETSPKPWIY

SEQ ID NO: 267 - protein, 32 aa, synthetic construct
GVPVRFSGSGSGTSYSLTISSMEAEDAATYYC

SEQ ID NO: 268 - protein, 10 aa, synthetic construct
FGAGTKLELK

SEQ ID NO: 269 - protein, 123 aa, synthetic construct
QVQLVQSGAEVMKPGSSVKVSCKASGYTFSWYWLEWVRQAPGHGLEWMGEIDPGTFTTNYNEKFKARVTFTADTSTSTAYMELSSLRSEDTAVYYCARFSHFSGSNYDYFDYWGQGTLVTVSS

SEQ ID NO: 270 - protein, 107 aa, synthetic construct
EIVMTQSPATLSVSPGERATLSCRASQSIGTNIHWYQQKPGQAPRLLIYYASESISGIPARFSGSGSGTEFTLTISSLQSEDFAVYYCQQSWSWPTTFGGGTKVEIK

SEQ ID NO: 271 - protein, 5 aa, synthetic construct
WYWLE

SEQ ID NO: 272 - protein, 17 aa, synthetic construct
EIDPGTFTTNYNEKFKA

SEQ ID NO: 273 - protein, 14 aa, synthetic construct
FSHFSGSNYDYFDY

SEQ ID NO: 274 - protein, 11 aa, synthetic construct
RASQSIGTNIH

SEQ ID NO: 275 - protein, 7 aa, synthetic construct
YASESIS

SEQ ID NO: 276 - protein, 9 aa, synthetic construct
QQSWSWPTT

(19) United States
(12) Patent Application Publication
(10) Pub. No.: US 2026/0055194 A1
(43) Pub. Date: Feb. 26, 2026

(54) BISPECIFIC ANTIBODIES AND CONSTRUCTS FOR LYSOSOMAL TARGETING DEGRADATION AND METHODS OF USE THEREOF

(71) Applicant: LYCIA THERAPEUTICS, INC., South San Francisco, CA (US)

(72) Inventors: Darrin Anthony LINDHOUT, Mountain View, CA (US); Matthew James SHURTLEFF, Redwood City, CA (US); Richard Gar Wai Yau, San Francsico, CA (US)

(21) Appl. No.: 19/103,204

(22) PCT Filed: Aug. 12, 2023
(86) PCT No.: PCT/US2023/030120

§ 371 Date: Feb. 11, 2025

Related U.S. Application Data
(60) Provisional application No. 63/397,751, filed on Aug. 12, 2022.

(51) Int. Cl. C07K 16/28; C07K 16/18; C07K 16/42

(52) U.S. Cl. CPC C07K 16/2863; C07K 16/18; C07K 16/4291; C07K 2317/24; C07K 2317/31; C07K 2317/34; C07K 2317/52; C07K 2317/622; C07K 2317/77; C07K 2317/92

(57) ABSTRACT

Provided herein are antigen-binding proteins (ABPs) that selectively bind to M6PR and its isoforms and homologs, and compositions comprising the ABPs. Also provided herein are bispecific antigen-binding proteins (ABPs) that selectively bind to internalizing receptors, and a soluble target molecule or cell surface target molecule and its isoforms and homologs, and compositions comprising the ABPs.

### 1. CROSS-REFERENCE TO RELATED APPLICATIONS

This application claims the benefit of U.S. Provisional Application No. 63/397,751, filed Aug. 12, 2022, which is hereby incorporated in its entirety by reference.

### 2. SEQUENCE LISTING

The instant application contains a Sequence Listing which has been submitted via Patent Center and is hereby incorporated by reference in its entirety. Said XML copy, created on XXXX, is named XXXX, and is XXX bytes in size.

### 3. BACKGROUND

Internalizing receptors are receptors that move from the plasma membrane to the inside of the cell when cell surface receptors are monoubiquitinated following ligand-induced activation. These receptors are subsequently taken up into endocytic vesicles from where they are either targeted to the lysosome or vacuole for degradation or recycled back to the plasma membrane.

One example of an internalizing receptor includes Mannose-6-Phosphate Receptor (M6PR), a transmembrane glycoprotein that target enzymes to lysosomes. These receptors play an important role in transporting proteins from the Golgi complex and cell surface to the lysosome. Cation independent (CI) M6PRs are multifunctional in that these receptors bind at the cell surface to two distinct classes of ligands, M6P-bearing proteins, and insulin-like growth factor 2 (IGF2). Therefore, CI-M6PRs are synonymously called “M6PR” and “IGF2-R”.

IGF2 plays key roles in metabolic regulation through three types of receptors, two tyrosine kinase receptors (IGF1 receptor and insulin receptor isoform A) and CI-M6PR. The stimulation of the type 1 receptor tyrosine kinase induces a protein phosphorylation cascade leading to biological effects, particularly the insulin-mediated growth and increase in CI-M6PR expression, which mediates endocytosis and clearance of IGF2. The lysosomal degradation of IGF2 is critical because elevated levels of IGF2 induce overgrowth. When CI-M6PR binds to IGF2, it blocks IGF2-stimulated DNA synthesis in hepatocytes and fibroblasts, and at physiological concentrations this receptor can block tumor growth mediated by IGF2.

Thus, there is a need for developing internalizing receptor antigen-binding proteins (ABPs) that can be used for treatment, diagnosis, and research of various diseases, including cancer, lysosomal, and autoimmune disease.

Additionally, targeting soluble target molecules or cell surface target molecules for internalization can be useful for the treatment of additional diseases.

For example, IgE, a soluble molecule, is known to interact with two major receptors, FcεRI and CD23/FcεRII4, which are involved in different immunological processes. Binding of allergen-specific IgE to FcεRI expressed on immunological effector cells including basophils and mast cells occurs with high affinity. This interaction occurs via two asymmetric binding sites on the receptor and is stabilized through the induction of a conformational change in IgE. Exposure to allergens induces cross-linking of IgE-bound FcεRI resulting in immediate activation of allergic effector cells, which culminates in cellular degranulation and the release of vasoactive and pro-inflammatory mediators. Omalizumab, an anti-IgE antibody, binds to free IgE and prevents the binding of IgE to FcεRI on mast cells and basophils. Omalizumab has been approved for severe persistent allergic asthma and chronic spontaneous urticaria. The therapeutic efficacy of anti-IgE has also been reported in allergic rhinitis, allergic bronchopulmonary aspergillosis, latex allergy, atopic dermatitis, allergic urticaria, anaphylaxis, and others.

In another example, a cell surface target molecule, epidermal growth factor receptor (EGFR), regulates normal growth and differentiation, but dysregulation of the receptor or one of the EGFR ligands is involved in the pathogenesis of many cancers. EGFR and insulin-like growth factor receptor (IFGR)play key roles in the tumorigenesis of several types of human cancer.

Inhibition of receptor tyrosine kinases such as EGFR has emerged as an effective treatment strategy for certain human malignancies (for a review see Roussidis A E, In Vivo. 2002 16 (6): 459-69). While targeted monotherapy may initially be effective in treating cancer, therapeutic resistance often follows, possibly as a result of upregulation of other signaling cascades (see e.g., Nahta R et al., Breast Cancer Res. 2006 8 (6): 215 and Horn L et al., Clin Lung Cancer. 2007 8: S68-73). Matuzumab, a humanized immunoglobulin G (1) (IgG (1)) anti-EGFR monoclonal antibody, blocks activation of EGFR. Accordingly, there exists a need for developing improved therapeutics related to targeting of cell surface molecules such as EGFR.

### 4. SUMMARY

Provided herein are novel antibody binding proteins (ABPs) comprising a first antigen binding moiety with binding specificity for an internalizing domain of cation-independent mannose 6 phosphate receptor (CI-M6PR) and methods of using such ABPs. In some embodiments, the internalizing domain is a fragment of the human CI-M6PR.

Also provided herein are novel ABPs comprising a first binding moiety with binding specificity for human CI-M6PR and a target binding moiety with specificity for a soluble extracellular target molecule or a cell surface target molecule, and methods of using such ABPs. In some embodiments, the target binding moiety is a second antigen binding moiety. In some embodiments, the target binding moiety is conjugated to the first antigen binding moiety, optionally via a linker.

Also provided herein are ABPs comprising a cargo moiety. In some embodiments, the cargo moiety is a polypeptide that is fused to either the first or second antigen binding moiety. In some embodiments, the cargo moiety is conjugated to either the first or second antigen binding moiety, optionally via a linker.

In some embodiments, the first antigen binding moiety specifically binds to a domain of human CI-M6PR selected from: domain 1, domain 4, domain 5, domain 6, domain 7, and domain 8.

In some embodiments, the first antigen binding moiety binds human CI-M6PR with high affinity. In some embodiments, the first antigen binding moiety binds human CI-M6PR with a dissociation equilibrium constant (K_{D}) of about 10 nM or less. In some embodiments, the first antigen binding moiety binds CI-M6PR with a K_{D} between about 1 nM and about 500 nM. In some embodiments, the first antigen binding moiety binds CI-M6PR with a K_{D} between about 10 nM and about 100 nM. In some embodiments, the first antigen binding moiety binds CI-M6PR with a K_{D} between about 100 nM and about 200 nM. In some embodiments, the first antigen binding moiety binds CI-M6PR with a K_{D} between about 200 nM and about 300 nM. In some embodiments, the first antigen binding moiety binds CI-M6PR with a K_{D} between about 300 nM and about 400 nM. In some embodiments, the first antigen binding moiety binds CI-M6PR with a K_{D} between about 400 nM and about 500 nM.

In some embodiments, the off rate (K_{off}) of the first antigen binding moiety for CI-M6PR is between 1×10⁻⁸ s⁻¹ and 0.1 s⁻¹. In some embodiments, K_{off} of the first antigen binding moiety for CI-M6PR is between 1×10⁻⁶ s⁻¹ and 1×10⁻² s⁻¹. In some embodiments, K_{off} of the first antigen binding moiety for CI-M6PR is about 1×10⁻⁵ s⁻¹ or slower.

In some embodiments, the binding of the first antigen binding moiety to human CI-M6PR is pH dependent. In some embodiments, the first antigen binding moiety is released from CI-M6PR at a pH of 7.4 or lower. In some embodiments, the first antigen binding moiety is released from CI-M6PR at a pH of 6.0 or lower. In some embodiments, the first antigen binding moiety is released from CI-M6PR at a pH of 5.5 or lower. In some embodiments, the first antigen binding moiety is released from CI-M6PR at a pH of 5.0 or lower.

In some embodiments, the first antigen binding moiety comprises (a) a light chain CDR3 (LCDR3) having the sequence of SEQ ID NO: 76 and a heavy chain CDR3 (HCDR3) having the sequence of SEQ ID NO: 35; or (b) a light chain CDR3 having the sequence of SEQ ID NO: 78 and a heavy chain CDR3 having the amino acid sequence of SEQ ID NO: 38; or (c) a light chain CDR3 having the sequence of SEQ ID NO: 81 and a heavy chain CDR3 having the sequence of SEQ ID NO: 41; or (d) a light chain CDR3 having the sequence of SEQ ID NO: 84 and a heavy chain CDR3 having the sequence of SEQ ID NO: 44; or (e) a light chain CDR3 having the sequence of SEQ ID NO: 85 and a heavy chain CDR3 having the sequence of SEQ ID NO: 46; or (f) a light chain CDR3 having the sequence of SEQ ID NO: 88 and a heavy chain CDR3 having the sequence of SEQ ID NO: 49; or (g) a light chain CDR3 having the sequence of SEQ ID NO: 91 and a heavy chain CDR3 having the sequence of SEQ ID NO: 52; or (h) a light chain CDR3 having the sequence of SEQ ID NO: 94 and a heavy chain CDR3 having the sequence of SEQ ID NO: 54; or (i) a light chain CDR3 having the sequence of SEQ ID NO: 96 and a heavy chain CDR3 having the sequence of SEQ ID NO: 55; or (j) a light chain CDR3 having the sequence of SEQ ID NO: 99 and a heavy chain CDR3 having the sequence of SEQ ID NO: 58; or (k) a light chain CDR3 having the sequence of SEQ ID NO: 101 and a heavy chain CDR3 having the sequence of SEQ ID NO: 60; or (1) a light chain CDR3 having the sequence of SEQ ID NO: 104 and a heavy chain CDR3 having the sequence of SEQ ID NO: 63; or (m) a light chain CDR3 having the sequence of SEQ ID NO: 107 and a heavy chain CDR3 having the sequence of SEQ ID NO: 66; or (n) a light chain CDR3 having the sequence of SEQ ID NO: 110 and a heavy chain CDR3 having the sequence of SEQ ID NO: 69; or (o) a light chain CDR3 having the sequence of SEQ ID NO: 113 and a heavy chain CDR3 having the sequence of SEQ ID NO: 72; or (p) a light chain CDR3 having the sequence of SEQ ID NO: 116 and a heavy chain CDR3 having the sequence of SEQ ID NO: 73.

In some embodiments, the first antigen binding moiety comprises (a) a light chain CDR1 (LCDR1) having the sequence of SEQ ID NO: 74 and a heavy chain CDR1 (HCDR1) having the sequence of SEQ ID NO: 33; and a light chain CDR2 (LCDR2) having the sequence of SEQ ID NO: 75 and a heavy chain CDR2 (HCDR2) having the sequence of SEQ ID NO: 34; or (b) an LCDR1 having the sequence of SEQ ID NO: 77 and a heavy chain HCDR1 having the sequence of SEQ ID NO: 36; and an LCDR2 having the sequence of SEQ ID NO: 75 and a HCDR2 having the sequence of SEQ ID NO: 37; or (c) an LCDR1 having the sequence of SEQ ID NO: 79 and a heavy chain HCDR1 having the sequence of SEQ ID NO: 39; and an LCDR2 having the sequence of SEQ ID NO: 80 and a HCDR2 having the sequence of SEQ ID NO: 40; or (d) an LCDR1 having the sequence of SEQ ID NO: 82 and a heavy chain HCDR 1 having the sequence of SEQ ID NO: 42; and an LCDR2 having the sequence of SEQ ID NO: 83 and a HCDR2 having the sequence of SEQ ID NO: 43; or (e) an LCDR1 having the sequence of SEQ ID NO: 79 and a heavy chain HCDR1 having the sequence of SEQ ID NO: 45; and an LCDR2 having the sequence of SEQ ID NO: 80 and a HCDR2 having the sequence of SEQ ID NO: 34; or (f) an LCDR1 having the sequence of SEQ ID NO: 86 and a heavy chain HCDR1 having the sequence of SEQ ID NO: 47; and an LCDR2 having the sequence of SEQ ID NO: 87 and a HCDR2 having the sequence of SEQ ID NO: 48; or (g) an LCDR1 having the sequence of SEQ ID NO: 89 and a heavy chain HCDR1 having the sequence of SEQ ID NO: 50; and an LCDR2 having the sequence of SEQ ID NO: 90 and a HCDR2 having the sequence of SEQ ID NO: 53; or (h) an LCDR1 having the sequence of SEQ ID NO: 92 and a heavy chain HCDR1 having the sequence of SEQ ID NO: 50; and an LCDR2 having the sequence of SEQ ID NO: 93 and a HCDR2 having the sequence of SEQ ID NO: 53; or (i) an LCDR1 having the sequence of SEQ ID NO: 95 and a heavy chain HCDR1 having the sequence of SEQ ID NO: 50; and an LCDR2 having the sequence of SEQ ID NO: 93 and a HCDR2 having the sequence of SEQ ID NO: 51; or (j) an LCDR1 having the sequence of SEQ ID NO: 97 and a heavy chain HCDR1 having the sequence of SEQ ID NO: 56; and an LCDR2 having the sequence of SEQ ID NO: 98 and a HCDR2 having the sequence of SEQ ID NO: 57; or (k) an LCDR1 having the sequence of SEQ ID NO: 100 and a heavy chain HCDR1 having the sequence of SEQ ID NO: 50; and an LCDR2 having the sequence of SEQ ID NO: 93 and a HCDR2 having the sequence of SEQ ID NO: 59; or (1) an LCDR1 having the sequence of SEQ ID NO: 102 and a heavy chain HCDR1 having the sequence of SEQ ID NO: 61; and an LCDR2 having the sequence of SEQ ID NO: 103 and a HCDR2 having the sequence of SEQ ID NO: 62; or (m) an LCDR1 having the sequence of SEQ ID NO: 105 and a heavy chain HCDR1 having the sequence of SEQ ID NO: 64; and an LCDR2 having the sequence of SEQ ID NO: 106 and a HCDR2 having the sequence of SEQ ID NO: 65; or (n) an LCDR1 having the sequence of SEQ ID NO: 108 and a heavy chain HCDR1 having the sequence of SEQ ID NO: 67; and an LCDR2 having the sequence of SEQ ID NO: 109 and a HCDR2 having the sequence of SEQ ID NO: 68; or (0) an LCDR1 having the sequence of SEQ ID NO: 111 and a heavy chain HCDR1 having the sequence of SEQ ID NO: 70; and an LCDR2 having the sequence of SEQ ID NO: 112 and a HCDR2 having the sequence of SEQ ID NO: 71; or (p) an LCDR1 having the sequence of SEQ ID NO: 114 and a heavy chain HCDR1 having the sequence of SEQ ID NO: 70; and an LCDR2 having the sequence of SEQ ID NO: 115 and a HCDR2 having the sequence of SEQ ID NO: 71.

In some embodiments, the first antigen binding moiety comprises a variable light chain (VL) having an amino acid sequence that is at least 95% identical to an amino acid sequence selected from SEQ ID NOs: 17-32.

In some embodiments, the first antigen binding moiety comprises a variable heavy chain (VH) having an amino acid sequence that is at least 95% identical to an amino acid sequence selected from SEQ ID NOs.: 1-16.

In some embodiments, the first antigen binding moiety comprises a variable light chain (VL) having an amino acid sequence that is at least 90% identical to an amino acid sequence selected from SEQ ID NOs: 17-32.

In some embodiments, the first antigen binding moiety comprises a variable heavy chain (VH) having an amino acid sequence that is at least 90% identical to an amino acid sequence selected from SEQ ID NOs.: 1-16.

In some embodiments, the first antigen binding moiety is an antibody fragment (e.g., single chain variable fragment (scFv) or an antigen-binding fragment (Fab)).

In some embodiments, the ABP disclosed herein is transported to the lysosome. In some embodiments, the ABP is transported back to the cell surface following internalization. In some embodiments, the ABP is degraded in the cell.

In some embodiments, the ABP comprises a multi-specific (e.g., bispecific) antibody (see, e.g., FIG. 1). In some embodiments, the multi-specific antibody comprises a first antigen binding moiety and a second antigen binding moiety. In certain embodiments, the first antigen-binding moiety is an antigen-binding fragment selected from Fab, F(ab′)₂, single chain antibodies (e.g. scFv), diabodies, triabodies, tetrabodies, and domain antibodies. In some embodiments, the antibody is selected from a human antibody, a humanized antibody, or a chimeric antibody. In certain embodiments, the second antigen-binding moiety is an antigen-binding fragment selected from Fab, F(ab′)₂, single chain antibodies (e.g. scFv), diabodies, triabodies, tetrabodies, and domain antibodies. In some embodiments, the antibody is selected from a human antibody, a humanized antibody, or a chimeric antibody.

Also provided herein are methods of degrading a soluble target or a cell surface target molecule comprising (a) contacting the with a multi-specific antigen binding protein (ABP), wherein the ABP comprises a first antigen binding moiety that specifically binds to a cation-independent mannose 6 phosphate receptor (CI-M6PR) on the surface of a cell; and (b) transporting the ABP, the target molecule, and the CI-M6PR to a lysosome within a cell, wherein the target molecule is degraded in the lysosome. In some embodiments, the method further comprises (c) transporting the ABP to the cell surface.

Also provided herein are methods of internalizing a target molecule comprising (a) contacting the target molecule with an antigen binding protein (ABP) as disclosed herein and (b) internalizing the ABP within a cell.

### 5. BRIEF DESCRIPTION OF THE DRAWINGS

FIG. 1 provides schematics of bispecific antigen binding proteins (ABPs).

FIGS. 2A-B provide non-limiting examples of a multi-specific antigen binding protein with a first antigen binding moiety with a full-length antibody that targets M6PR and a second antigen binding moiety with an scFv that binds a target molecule such as IgE (FIG. 2A) or EGFR (FIG. 2B).

FIGS. 3A-3B provide non-limiting examples of a multi-specific antigen binding protein with a first antigen binding moiety with an scFv that targets M6PR and a second antigen binding moiety comprising a full-length antibody that binds a target molecule such as IgE (FIG. 3A) or EGFR (FIG. 3B).

FIG. 4 shows cell surface and intracellular staining of human M6PR in WT and M6PR-KO K562 cells.

FIG. 5 shows titer confirmation data of mice using recombinant antigens and confirmed via flow-based assay using human cell lines (K562 WT, K562 M6PR-KO, Hela WT and Hela M6PR-KO) and cross-screened against two murine cell lines (Dipak MC38 and 4T1).

FIG. 6 provides a schematic of the biosensor preparation and binding events for the affinity assessment of the top 192 clones via Octet for on/off rates and K_{D} for human M6PR.

FIG. 7 provides a baseline measure for each biosensor loaded with a representative mIgG clone from supernatant (as well as poly mlgG control). Additional biosensors were regenerated for measurement of additional analyte interactions (mouse M6PR-His6 and off-target binding to His). Binding curve data for a representative clone is shown in the top panel.

FIG. 8 provides an outline of how M6PR targeted monoclonal antibodies are screened for uptake activity.

FIGS. 9A-G show response curves from binding assays of various anti-M6PR antibodies.

FIG. 10 shows a table of affinities (K_{D}) and kinetics (Kₒₙ, K_{off}) for 15 anti-M6PR antibodies.

FIG. 11 is a schematic showing domains D1 to D9 of human CI-M6PR (left panel) and an antibody binding to D2; the antibody does not bind to the chimeric CI-M6PR in which D2 of human CI-M6PR has been replaced with the corresponding domain from mouse CI-M6PR Ligands of CI-M6PR are listed adjacent to the domain in which they bind.

FIG. 12A shows an illustration of the assay used for epitope binning. A coupled antibody (Coupled Ab) is an anti-M6PR antibody that is labeled with a probe.

FIG. 12B shows binding curves with each line indicating a different bin based on the kinetics of the curve.

FIG. 13 shows a network analysis of epitope binning. Left panel shows bin assignments for each of the top 12 anti-M6PR antibodies. Right panel shows the locations of the Bin 7, Bin 17, and Bin 3 within the entire network analysis.

FIG. 14 shows an expression gel for each of the HIS-tagged D1-D9 CI-M6PR domains.

FIGS. 15A-15H show binding curves for different anti-M6PR antibodies with binding to mouse CI-M6PR D1 to D9 domains (LT011-muD1-LT011muD9). Human wild type CI-M6PR was used as a positive control (huLT011).

FIG. 16 is a schematic summarizing results of domain mapping from FIGS. 15A-15H.

FIG. 17A shows experimental workflow for evaluating pH association and dissociation of anti-M6PR antibodies from CI-M6PR.

FIGS. 17B-17D shows binding curves for association at pH 7.4 and dissociation at pH 5.0, pH 6.0, and pH 7.4 for clones 17F11 (FIG. 17B), 18G4 (FIG. 17C), and 21D5 (FIG. 17D).

FIG. 18 is a histogram quantifying pH-dependent dissociation for 9 different anti-M6PR antibody clones. pH-dependent dissociation was determined by dividing the K_{off} of each antibody at pH 5.0 (pH5.0/7.4) or pH 6.0 (pH6.0/pH7.4) by that at pH 7.4. Dissociation is quantified as fold change of binding shift following 300 seconds at the specified pH.

FIG. 19A is a schematic illustrating steps and biology underlying a pH rodo uptake assay.

FIG. 19B shows a histogram of mean fluorescence intensity for a pH rodo uptake assay for 30 anti-M6PR antibodies. Dotted line represents the uptake for the KLH-IGF2 positive control. * indicates antibodies used at less than 25 nM due to low expression during production.

FIGS. 20A-20F shows uptake curves for anti-M6PR-omalizumab bispecific antibodies (FIG. 20A and FIG. 20B) and anti-M6PR antibodies (FIGS. 20C-20F). Uptake of IgE was measured by mean fluorescence intensity (MFI) of the Alexa Fluor 488 conjugated to IgE. Uptake was measured at various concentrations of bispecific antibodies or antibodies, as indicated on the x-axis.

FIG. 21 provides a schematic of the surface HiBit assay and total HiBit assay and example readouts for each assay.

FIG. 22A is a histogram showing span (i.e., surface EGFR depletion) for 16 matuzumab-antiM6PR bispecific antibodies.

FIG. 22B is a histogram showing span (i.e., total EGFR depletion) for 16 matuzumab-antiM6PR bispecific antibodies.

FIG. 22C shows percentage of surface EGFR activity of exemplary matuzumab-antiM6PR bispecific antibodies (3C7, 53B11).

FIG. 22D shows percentage of total EGFR activity of exemplary matuzumab-antiM6PR bispecific antibodies (3C7, 53B11).

FIG. 23 is a schematic of experimental workflow for a hIgE uptake assay using omalizumab-antiM6PR (MPR6-Oma)bispecific antibodies.

FIG. 24A shows internalization and recycling of M6PR6-Oma bispecific antibodies by HepG2 cells (as indicated by mean fluorescence intensity (MFI))

FIG. 24B shows quantification of internalization and recycling of M6PR-Oma bispecific antibody shown in FIG. 24A. 0′ condition was used as internal baseline and set at 100%. Quantification was measured as hlgE surface labelling/uptake.

FIG. 25A shows internalization and recycling of MPR6-Oma bispecific antibodies by K562 cells (as indicated by mean fluorescence intensity (MFI)).

FIG. 25B shows quantification of internalization and recycling of M6PR-Oma bispecific antibody shown in FIG. 25A. 0′ condition was used as internal baseline and set at 100%. Quantification was measured as hlgE surface labeling/uptake.

FIGS. 26A-26B show C5 uptake by anti-M6PR/anti-C5 bispecific antibodies using C5 fluorescently labeled with pH rodo (FIG. 26A) or AF647 (FIG. 26B).

FIGS. 27A-27B LUM-antiM6PR bispecific antibody mediated HER3 internalization and impaired cell viability of trastuzumab-resistant cells. FIG. 27A shows surface depletion (i.e., span) and total depletion (i.e., span) of HER3 for a subset of LUM-antiM6PR bispecific antibodies. Lumretuzumab (LUM) humanized antibuman epidermal growth factor receptor 3 (HER3) monoclonal antibody and a NRGI (Neuregulin 1) antibody were used as a positive control. A non-binding antibody (KLH) fused to LUM was used as a negative control for uptake activity. FIG. 27B shows cell viability after treatment of the subset of LUM-antiM6PR bispecific antibodies.

### 6. DETAILED DESCRIPTION

### 6.1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

The following terms, unless otherwise indicated, shall be understood to have the following meanings:

The terms “M6PR,” “M6PR protein,” and “M6PR antigen” are used interchangeably herein to refer to human M6PR, human CI-M6PR, or any variants (e.g., splice variants and allelic variants), isoforms, and species homologs of human M6PR that are naturally expressed by cells, or that are expressed by cells transfected with an M6PR gene. In some aspects, the M6PR protein is a M6PR protein naturally expressed by a primate (e.g., a monkey or a human), a rodent (e.g., a mouse or a rat), a dog, a camel, a cat, a cow, a goat, a horse, or a sheep. In some aspects, the M6PR protein is human cation-independent M6PR (NCBI Accession NP_000867; SEQ ID NO: 276).

The term “internalizing receptor” refers to a receptor that can move from a region outside a cell (e.g. including a cell surface) to the inside of a cell. Internalizing receptors include, but are not limited to, endocytic receptors and receptors that transport to lysosomes. Non-limiting examples of internalizing receptors include mannose-6-phosphate receptor (CD-M6PR), cation-independent mannose-6-phosphate receptor (CI-M6PR).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (VL) and a light chain constant region. The light chain constant region typically comprises one domain, abbreviated CL.

The term “antigen-binding protein” (ABP) refers to a protein comprising one or more antigen-binding domains that specifically bind to an antigen or epitope. In some embodiments, the antigen-binding domain binds the antigen or epitope with specificity and affinity similar to that of naturally occurring antibodies. In some embodiments, the ABP comprises an antibody. In some embodiments, the ABP consists of an antibody. In some embodiments, the ABP consists essentially of an antibody. In some embodiments, the ABP comprises an alternative scaffold. In some embodiments, the ABP consists of an alternative scaffold. In some embodiments, the ABP consists essentially of an alternative scaffold. In some embodiments, the ABP comprises an antibody fragment. In some embodiments, the ABP consists of an antibody fragment. In some embodiments, the ABP consists essentially of an antibody fragment. A “internalizing receptor ABP,” “anti-internalizing receptor ABP,” or “internalizing receptor-specific ABP” is an ABP, as provided herein, which specifically binds to the antigen of an internalizing receptor. In some embodiments, the ABP binds the extracellular domain of an internalizing receptor. In certain embodiments, a internalizing receptor ABP provided herein binds to an epitope of an internalizing receptor that is conserved between or among internalizing receptor proteins from different species. A “soluble target molecule ABP,” “anti-soluble target molecule ABP,” or “soluble target molecule-specific ABP” is an ABP, as provided herein, which specifically binds to the antigen of a soluble target molecule. In some embodiments, the ABP binds the extracellular domain of a soluble target molecule. In certain embodiments, a soluble target molecule ABP provided herein binds to an epitope of an soluble target molecule that is conserved between or among soluble target molecule proteins from different species. A “cell surface target molecule ABP,” “anti-cell surface target molecule ABP,” or “cell surface target molecule-specific ABP” is an ABP, as provided herein, which specifically binds to the antigen of a cell surface target molecule. In some embodiments, the ABP binds the extracellular domain of a cell surface target molecule. In certain embodiments, a cell surface target molecule ABP provided herein binds to an epitope of a cell surface target molecule that is conserved between or among cell surface target molecule proteins from different species.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies. One example of an antigen-binding domain is an antigen-binding domain formed by a VH-VL dimer. An antibody is one type of ABP.

The term “bispecific ABP” or “multi-specific” refers to an ABP, e.g., an antibody, with at least two different antigen binding moieties (e.g., antigen binding domains) that each specifically bind a different antigen, e.g., an internalization receptor and a soluble target molecule or a cell surface target molecule. A bispecific ABP can be of any number of valencies, e.g., bivalent, trivalent, tetravalent, and the like.

The term “multi-specific ABP” refers to an ABP, e.g., an antibody, with at least two (e.g., at least three, at least four, at least five, at least six, etc.) different antigen binding moieties (e.g., antigen binding domains) that each specifically bind a different antigen, e.g., an internalization receptor, and a soluble target molecule or a cell surface target molecule. A bispecific ABP can be of any number of valencies, e.g., bivalent, trivalent, tetravalent, and the like. The term “antigen-binding moiety” or “antigen-binding domain” means the portion of an ABP that is capable of specifically binding to an antigen or epitope.

The terms “full length antibody,” “intact antibody,” and “whole antibody” are used herein interchangeably to refer to an antibody having a structure substantially similar to a naturally occurring antibody structure and having heavy chains that comprise an Fc region.

The term “Fc region” means the C-terminal region of an immunoglobulin heavy chain that, in naturally occurring antibodies, interacts with Fc receptors and certain proteins of the complement system. The structures of the Fc regions of various immunoglobulins, and the glycosylation sites contained therein, are known in the art. See Schroeder and Cavacini, J. Allergy Clin. Immunol., 2010, 125: S41-52, incorporated by reference in its entirety. The Fc region may be a naturally occurring Fc region, or an Fc region modified as described elsewhere in this disclosure.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and VL generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

Table 1 provides the positions of CDR1-L (CDR1 of V_{L}), CDR2-L (CDR2 of V_{L}), CDR3-L (CDR3 of V_{L}), CDR1-H (CDR1 of V_{H}), CDR2-H (CDR2 of V_{H}), and CDR3-H (CDR3 of V_{H}), as identified by the Kabat and Chothia schemes. For CDR1-H, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 1
Residues in CDRs according to Kabat
and Chothia numbering schemes.

| CDR | Kabat | Chothia |
|---|---|---|
| CDR1-L | 24-34 | 24-34 |
| CDR2-L | 50-56 | 50-56 |
| CDR3-L | 89-97 | 89-97 |
| CDR1-H (Kabat | 31-35B | 26-32 or 34* |
| Numbering) | | |
| CDR1-H (Chothia | 31-35 | 26-32 |
| Numbering) | | |
| CDR2-H | 50-65 | 52-56 |
| CDR3-H | 95-102 | 95-102 |

*The C-terminus of CDR1-H, when numbered using the Kabat numbering convention, varies between 32 and 34, depending on the length of the CDR.

The “EU numbering scheme” is generally used when referring to a residue in an antibody heavy chain constant region (e.g., as reported in Kabat et al., supra).

An “antibody fragment” comprises a portion of an intact antibody, such as the antigen-binding or variable region of an intact antibody. Antibody fragments include, for example, Fv fragments, Fab fragments, F(ab′)₂ fragments, Fab′ fragments, scFv (sFv) fragments, and scFv-Fc fragments.

“Fv” fragments comprise a non-covalently linked dimer of one heavy chain variable domain and one light chain variable domain.

“Fab” fragments comprise, in addition to the heavy and light chain variable domains, the constant domain of the light chain and the first constant domain (CH1) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with β-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is a (GGGGS) n. In some embodiments, n=1, 2, 3, 4, 5, or 6. See Antibodies from Escherichia coli. In Rosenberg M. & Moore G.P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminal of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG4 Fc domain.

The term “single domain antibody” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety.

A “monospecific ABP” is an ABP that comprises a binding site that specifically binds to a single epitope. An example of a monospecific ABP is a naturally occurring IgG molecule which, while divalent, recognizes the same epitope at each antigen-binding domain. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated ABP” or “isolated nucleic acid” is an ABP or nucleic acid that has been separated and/or recovered from a component of its natural environment. Components of the natural environment may include enzymes, hormones, and other proteinaceous or nonproteinaceous materials. In some embodiments, an isolated ABP is purified to a degree sufficient to obtain at least 15 residues of N-terminal or internal amino acid sequence, for example by use of a spinning cup sequenator. In some embodiments, an isolated ABP is purified to homogeneity by gel electrophoresis (e.g., SDS-PAGE) under reducing or nonreducing conditions, with detection by Coomassie blue or silver stain. An isolated ABP includes an ABP in situ within recombinant cells, since at least one component of the ABP's natural environment is not present. In some embodiments, an isolated ABP or isolated nucleic acid is prepared by at least one purification step. In some embodiments, an isolated ABP or isolated nucleic acid is purified to at least 80%, 85%, 90%, 95%, or 99% by weight. In some embodiments, an isolated ABP or isolated nucleic acid is purified to at least 80%, 85%, 90%, 95%, or 99% by volume. In some embodiments, an isolated ABP or isolated nucleic acid is provided as a solution comprising at least 85%, 90%, 95%, 98%, 99% to 100% ABP or nucleic acid by weight. In some embodiments, an isolated ABP or isolated nucleic acid is provided as a solution comprising at least 85%, 90%, 95%, 98%, 99% to 100% ABP or nucleic acid by volume.

“Affinity” refers to the strength of the sum total of non-covalent interactions between a single binding site of a molecule (e.g., an ABP) and its binding partner (e.g., an antigen or epitope). Unless indicated otherwise, as used herein, “affinity” refers to intrinsic binding affinity, which reflects a 1:1 interaction between members of a binding pair (e.g., ABP and antigen or epitope). The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORER) or biolayer interferometry (e.g., FORTEBIOR).

With regard to the binding of an ABP to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the ABP to the target molecule is competitively inhibited by the control molecule. For example, in some embodiments, the affinity of an internalizing receptor ABP for a non-target molecule is less than about 50% of the affinity for internalizing receptor. In some embodiments, the affinity of a internalizing receptor ABP for a non-target molecule is less than about 40% of the affinity for internalizing receptor. In some embodiments, the affinity of a internalizing receptor ABP for a non-target molecule is less than about 30% of the affinity for internalizing receptor. In some embodiments, the affinity of a internalizing receptor ABP for a non-target molecule is less than about 20% of the affinity for internalizing receptor. In some embodiments, the affinity of a internalizing receptor ABP for a non-target molecule is less than about 10% of the affinity for internalizing receptor. In some embodiments, the affinity of a internalizing receptor ABP for a non-target molecule is less than about 1% of the affinity for internalizing receptor. In some embodiments, the affinity of a internalizing receptor ABP for a non-target molecule is less than about 0.1% of the affinity for internalizing receptor.

The term “k_{d}” (sec⁻¹), as used herein, refers to the dissociation rate constant of a particular ABP-antigen interaction. This value is also referred to as the K_{off} value.

The term “kₐ” (M⁻¹×sec⁻¹), as used herein, refers to the association rate constant of a particular ABP-antigen interaction. This value is also referred to as the Kₒₙ value.

The term “K_{D}” (M), as used herein, refers to the dissociation equilibrium constant of a particular ABP-antigen interaction. K_{D}=kₐ/kₐ.

The term “K_{A}” (M⁻¹), as used herein, refers to the association equilibrium constant of a particular ABP-antigen interaction. K_{A}=kₐ/k_{d}.

An “affinity matured” ABP is one with one or more alterations (e.g., in one or more CDRs or FRs) that result in an improvement in the affinity of the ABP for its antigen, compared to a parent ABP which does not possess the alteration(s). In one embodiment, an affinity matured ABP has nanomolar or picomolar affinity for the target antigen. Affinity matured ABPs may be produced using a variety of methods known in the art. For example, Marks et al. (Bio Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310⁻³³¹⁹⁹; and Hawkins et al, J. Mol. Biol., 1992, 226:889-896; each of which is incorporated by reference in its entirety.

An “immunoconjugate” is an ABP conjugated to one or more heterologous molecule(s).

“Effector functions” refer to those biological activities mediated by the Fc region of an antibody, which activities may vary depending on the antibody isotype. Examples of antibody effector functions include Clq binding to activate complement dependent cytotoxicity (CDC), Fc receptor binding to activate antibody-dependent cellular cytotoxicity (ADCC), and antibody dependent cellular phagocytosis (ADCP).

When used herein in the context of two or more ABPs, the term “competes with” or “cross-competes with” indicates that the two or more ABPs compete for binding to an antigen (e.g., internalizing receptor). In one exemplary assay, internalizing receptor is coated on a surface and contacted with a first internalizing receptor ABP, after which a second internalizing receptor ABP is added. In another exemplary assay, a first internalizing receptor ABP is coated on a surface and contacted with internalizing receptor, and then a second internalizing receptor ABP is added. If the presence of the first internalizing receptor ABP reduces binding of the second internalizing receptor ABP, in either assay, then the ABPs compete. The term “competes with” also includes combinations of ABPs where one ABP reduces binding of another ABP, but where no competition is observed when the ABPs are added in the reverse order. However, in some embodiments, the first and second ABPs inhibit binding of each other, regardless of the order in which they are added. In some embodiments, one ABP reduces binding of another ABP to its antigen by at least 25%, at least 50%, at least 60%, at least 70%, at least 80%, at least 85%, at least 90%, or at least 95%. A skilled artisan can select the concentrations of the antibodies used in the competition assays based on the affinities of the ABPs for internalizing receptor and the valency of the ABPs. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (www.ncbi.nlm.nih.gov/books/NBK92434/; accessed Sep. 29, 2015); Silman et al., Cytometry, 2001, 44:30-37; and Finco et al., J. Pharm. Biomed. Anal., 2011, 54:351-358; each of which is incorporated by reference in its entirety.

The term “epitope” means a portion of an antigen the specifically binds to an ABP. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an ABP binds can be determined using known techniques for epitope determination such as, for example, testing for ABP binding to internalizing receptor, a soluble target molecule, and/or a cell surface target molecule variants with different point-mutations, or to chimeric internalizing receptor, soluble target molecule, and/or cell surface target molecule variants.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. By way of example, the groups of amino acids provided in TABLES 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, NY. An ABP generated by making one or more conservative substitutions of amino acid residues in a parent ABP is referred to as a “conservatively modified variant.”

The term “treating” (and variations thereof such as “treat” or “treatment”) refers to clinical intervention in an attempt to alter the natural course of a disease or condition in a subject in need thereof. Treatment can be performed both for prophylaxis and during the course of clinical pathology. Desirable effects of treatment include preventing occurrence or recurrence of disease, alleviation of symptoms, diminish of any direct or indirect pathological consequences of the disease, preventing metastasis, decreasing the rate of disease progression, amelioration or palliation of the disease state, and remission or improved prognosis.

As used herein, the term “therapeutically effective amount” or “effective amount” refers to an amount of an ABP or pharmaceutical composition provided herein that, when administered to a subject, is effective to treat a disease or disorder.

As used herein, the term “subject” means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an ABP provided herein. In some aspects, the disease or condition is a cancer. In some aspects, the disease or condition is a viral infection.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “cytotoxic agent,” as used herein, refers to a substance that inhibits or prevents a cellular function and/or causes cell death or destruction.

A “chemotherapeutic agent” refers to a chemical compound useful in the treatment of cancer. Chemotherapeutic agents include “anti-hormonal agents” or “endocrine therapeutics” which act to regulate, reduce, block, or inhibit the effects of hormones that can promote the growth of cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein. The terms “cell proliferative disorder” and “proliferative disorder” refer to disorders that are associated with some degree of abnormal cell proliferation. In some embodiments, the cell proliferative disorder is a cancer.

The term “pharmaceutical composition” refers to a preparation which is in such form as to permit the biological activity of an active ingredient contained therein to be effective in treating a subject, and which contains no additional components which are unacceptably toxic to the subject.

The terms “modulate”, and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

A “variant” of a polypeptide (e.g., an antibody) comprises an amino acid sequence wherein one or more amino acid residues are inserted into, deleted from and/or substituted into the amino acid sequence relative to the native polypeptide sequence, and retains essentially the same biological activity as the native polypeptide. The biological activity of the polypeptide can be measured using standard techniques in the art (for example, if the variant is an antibody, its activity may be tested by binding assays, as described herein). Variants of the invention include fragments, analogs, recombinant polypeptides, synthetic polypeptides, and/or fusion proteins.

A “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, CA and Baron et al., 1995, Nucleic Acids Res. 23:3605-06.

A “host cell” is a cell that can be used to express a nucleic acid, e.g., a nucleic acid of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include CS-9 cells, the COS-7 line of monkey kidney cells (ATCC CRL 1651) (see Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31), Hela cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Typically, a host cell is a cultured cell that can be transformed or transfected with a polypeptide-encoding nucleic acid, which can then be expressed in the host cell.

The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### 6.3. Nucleic Acids

In one aspect, the present invention provides isolated nucleic acid molecules. The nucleic acids comprise, for example, polynucleotides that encode all or part of an antigen binding protein, for example, one or both chains of an antibody of the invention, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing. The nucleic acids can be any length. They can be, for example, 5, 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length, and/or can comprise one or more additional sequences, for example, regulatory sequences, and/or be part of a larger nucleic acid, for example, a vector. The nucleic acids can be single-stranded or double-stranded and can comprise RNA and/or DNA nucleotides, and artificial variants thereof (e.g., peptide nucleic acids).

Nucleic acids encoding antibody polypeptides (e.g., heavy or light chain, variable domain only, or full length) can be isolated from cells of mice that have been immunized with an internalizing receptor, soluble target molecule, and/or a cell surface target molecule. The nucleic acid can be isolated by conventional procedures such as polymerase chain reaction (PCR).

Nucleic acid sequences encoding the variable regions of the heavy and light chain variable regions are shown herein. The skilled artisan will appreciate that, due to the degeneracy of the genetic code, each of the polypeptide sequences disclosed herein is encoded by a large number of other nucleic acid sequences. The present invention provides each degenerate nucleotide sequence encoding each antigen binding protein of the invention.

The invention further provides nucleic acids that hybridize to other nucleic acids (e.g., nucleic acids comprising a nucleotide sequence of any of an internalizing receptor, soluble target molecule, and/or a cell surface target molecule gene) under particular hybridization conditions. Methods for hybridizing nucleic acids are well-known in the art. See, e.g., Curr. Prot. in Mol. Biol., John Wiley & Sons, N.Y. (1989), 6.3.1-6.3.6. As defined herein, a moderately stringent hybridization condition uses a prewashing solution containing 5×sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C., in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequences that are at least 65, 70, 75, 80, 85, 90, 95, 98, or 99% identical to each other typically remain hybridized to each other. The basic parameters affecting the choice of hybridization conditions and guidance for devising suitable conditions are set forth by, for example, Sambrook, Fritsch, and Maniatis (1989, Molecular Cloning: A Laboratory Manual, Cold Spring Harbor Laboratory Press, Cold Spring Harbor, N.Y., chapters 9 and 11; and Curr. Prot. in Mol. Biol. 1995, Ausubel et al., eds., John Wiley & Sons, Inc., sections 2.10 and 6.3-6.4), and can be readily determined by those having ordinary skill in the art based on, for example, the length and/or base composition of the DNA.

Changes can be introduced by mutation into a nucleic acid, thereby leading to changes in the amino acid sequence of a polypeptide (e.g., an antigen binding protein) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property (e.g., binding to an internalizing receptor, soluble target molecule, and/or a cell surface target molecule).

### 6.4. Antibody

Internalizing receptor antibodies can be purified from host cells that have been transfected by a gene encoding the antibodies by elution of filtered supernatant of host cell culture fluid using a Heparin HP column, using a salt gradient.

A Fab fragment is a monovalent fragment having the V_{L}, V_{H}, C_{L} and C_{H1} domains; a F(ab′)₂ fragment is a bivalent fragment having two Fab fragments linked by a disulfide bridge at the hinge region; a Fd fragment has the V_{H} and CH1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546, 1989).

Polynucleotide and polypeptide sequences of particular light and heavy chain variable domains are described below. Antibodies comprising a light chain and heavy chain are designated by combining the name of the light chain and the name of the heavy chain variable domains. In some embodiments, light chain variable sequences are provided in SEQ ID Nos: 32-63, and heavy chain variable sequences are provided in SEQ ID Nos: 1-31.

In other embodiments, an antibody may comprise a specific heavy or light chain, while the complementary light or heavy chain variable domain remains unspecified. In particular, certain embodiments herein include antibodies that bind a specific antigen (such as M6PR) by way of a specific light or heavy chain, such that the complementary heavy or light chain may be promiscuous, or even irrelevant, but may be determined by, for example, screening combinatorial libraries. Portolano et al., J. Immunol. V. 150 (3), pp. 880-887 (1993); Clackson et al., Nature v. 352 pp. 624-628 (1991); Adler et al., A natively paired antibody library yields drug leads with higher sensitivity and specificity than a randomly paired antibody library, MAbs (2018)); Adler et al., Rare, high-affinity mouse anti-PD-1 antibodies that function in checkpoint blockade, discovered using microfluidics and molecular genomics, MAbs (2017).

Naturally occurring immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4. The assignment of amino acids to each domain is in accordance with the definitions of Kabat et al. in Sequences of Proteins of Immunological Interest, 5th Ed., US Dept. of Health and Human Services, PHS, NIH, NIH Publication no. 91-3242, 1991.

The term “human antibody,” also referred to as “fully human antibody,” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. In one embodiment, one or more of the CDRs are derived from a human anti-intemalizing receptor antibody. In another embodiment, all of the CDRs are derived from a human anti-internalizing receptor antibody. In another embodiment, the CDRs from more than one human anti-internalizing receptor antibodies are mixed and matched in a chimeric antibody. For instance, a chimeric antibody may comprise a CDR1 from the light chain of a first human anti-internalizing receptor antibody, a CDR2 and a CDR3 from the light chain of a second human anti-internalizing receptor antibody, and the CDRs from the heavy chain from a third anti-internalizing receptor antibody. Further, the framework regions may be derived from one of the same anti-internalizing receptor antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind an internalizing receptor).

Fragments or analogs of antibodies can be readily prepared by those of ordinary skill in the art following the teachings of this specification and using techniques well-known in the art. Preferred amino- and carboxy-termini of fragments or analogs occur near boundaries of functional domains. Structural and functional domains can be identified by comparison of the nucleotide and/or amino acid sequence data to public or proprietary sequence databases. Computerized comparison methods can be used to identify sequence motifs or predicted protein conformation domains that occur in other proteins of known structure and/or function. Methods to identify protein sequences that fold into a known three-dimensional structure are known. See, e.g., Bowie et al., 1991, Science 253:164.

Antigen binding fragments derived from an antibody can be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 89:230, 1960; Porter, Biochem. J. 73:119, 1959; Edelman et al., in Methods in Enzymology 1:422 (Academic Press 1967); and by Andrews, S. M. and Titus, J. A. in Current Protocols in Immunology (Coligan J.E., et al., eds), John Wiley & Sons, New York (2003), pages 2.8.1 2.8.10 and 2.10A.1 2.10A.5. Other methods for cleaving antibodies, such as separating heavy chains to form monovalent light heavy chain fragments (Fd), further cleaving of fragments, or other enzymatic, chemical, or genetic techniques may also be used, so long as the fragments bind to the antigen that is recognized by the intact antibody.

An antibody fragment may also be any synthetic or genetically engineered protein. For example, antibody fragments include isolated fragments consisting of the light chain variable region, “Fv” fragments consisting of the variable regions of the heavy and light chains, recombinant single chain polypeptide molecules in which light and heavy variable regions are connected by a peptide linker (scFv proteins).

Another form of an antibody fragment is a peptide comprising one or more complementarity determining regions (CDRs) of an antibody. CDRs (also termed “minimal recognition units”, or “hypervariable region”) can be incorporated into a molecule either covalently or noncovalently to make it an antigen binding protein. CDRs can be obtained by constructing polynucleotides that encode the CDR of interest. Such polynucleotides are prepared, for example, by using the polymerase chain reaction to synthesize the variable region using mRNA of antibody producing cells as a template (see, for example, Larrick et al., Methods: A Companion to Methods in Enzymology 2:106, 1991; Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley Liss, Inc. 1995).

Thus, in one embodiment, the binding agent comprises at least one CDR as described herein. The binding agent may comprise at least two, three, four, five or six CDR's as described herein. The binding agent may further comprise at least one variable region domain of an antibody described herein. The variable region domain may be of any size or amino acid composition and will generally comprise at least one CDR sequence responsible for binding to human M6PR, for example HCDR1, HCDR2, HCDR3, LCDR1, LCDR2, and LCDR3, specifically described herein and which is adjacent to or in frame with one or more framework sequences. In general terms, the variable (V) region domain may be any suitable arrangement of immunoglobulin heavy (V_{H}) and/or light (V_{L}) chain variable domains. Thus, for example, the V region domain may be monomeric and be a V_{H} or V_{L} domain, which is capable of independently binding human M6PR with an affinity ranging from 1 nM to 1 pM. Alternatively, the V region domain may be dimeric and contain V_{H} V_{H}, V_{H} V_{L}, or V_{L} V_{L}, dimers. The V region dimer comprises at least one V_{H} and at least one V_{L} chain that may be non covalently associated (hereinafter referred to as Fv). If desired, the chains may be covalently coupled either directly, for example via a disulfide bond between the two variable domains, or through a linker, for example a peptide linker, to form a single chain Fv (scFV).

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C terminal amino acid to at least one other antibody domain or a fragment thereof. Thus, for example, a V_{H} domain that is present in the variable region domain may be linked to an immunoglobulin C_{H1} domain, or a fragment thereof. Similarly, a V_{L} domain may be linked to a CK domain or a fragment thereof. In this way, for example, the antibody may be a Fab fragment wherein the antigen binding domain contains associated V_{H} and V_{L} domains covalently linked at their C termini to a C_{H1} and CK domain, respectively. The C_{H1} domain may be extended with further amino acids, for example to provide a hinge region or a portion of a hinge region domain as found in a Fab′ fragment, or to provide further domains, such as antibody CH2 and CH3 domains.

As described herein, antibodies comprise at least one of these CDRs. For example, one or more CDR may be incorporated into known antibody framework regions (IgG1, IgG2, etc.), or conjugated to a suitable vehicle to enhance the half-life thereof. Suitable vehicles include, but are not limited to Fc, polyethylene glycol (PEG), albumin, transferrin, and the like. These and other suitable vehicles are known in the art. Such conjugated CDR peptides may be in monomeric, dimeric, tetrameric, or other form. In one embodiment, one or more water-soluble polymer is bonded at one or more specific position, for example at the amino terminus, of a binding agent.

### 6.5. Antigen Binding Protein

In one aspect, the present invention provides antigen binding proteins (e.g., antibodies, antibody fragments, antibody derivatives, antibody muteins, and antibody variants), that bind to an internalizing receptor, a soluble target molecule, and/or a cell surface target molecule.

In some embodiments, the ABP comprises an antigen-binding moiety (e.g., at least one antigen binding moiety) that specifically binds to an antigen of an internalizing receptor. In certain embodiments, binding of the antigen facilitates intracellular transport of the ABP by the internalizing receptor.

In certain embodiments, a first antigen binding moiety specifically binds to an epitope on the antigen that facilitates intracellular transport of the ABP by the internalizing receptor. In certain embodiments, the internalizing receptor is a cell surface internalizing receptor. In certain embodiments, the internalizing receptor is an endocytic receptor. In certain embodiments, the internalizing receptor is capable of targeting a lysosome. In certain embodiments, the internalizing receptor is an oncogenic receptor.

In certain embodiments, the internalizing receptor is CI-M6PR.

In some embodiments, the antigen binding moiety comprises an antigen-binding fragment selected from the group consisting of: Fab, F(ab′)₂, single chain antibodies (scFv), (scFv) 2, diabodies, triabodies, tetrabodies, and domain antibodies.

In some embodiments, the antigen binding moiety comprises a Fab fragment and an Fc domain. In some embodiments, “Fab-Fc” fragments comprise a Fab fragment attached to an Fc domain. For example, an Fc domain may be attached to the C-terminal of the Fab fragment. Any suitable Fc domain known in the art or described herein may be used. In some cases, the Fc domain comprises an IgG4 Fc domain.

In another aspect of the present disclosure, the ABP comprises a multi-specific ABP. In some embodiments, the multi-specific ABP of the present disclosure comprises a first antigen-binding moiety and a second antigen binding moiety.

In some embodiments, the multi-specific ABP comprises a first antigen binding moiety that specifically binds to an antigen of an internalizing receptor. In certain embodiments, binding of the antigen facilitates intracellular transport of the multi-specific ABP by the internalizing receptor.

In certain embodiments, a first antigen binding moiety specifically binds to an epitope on the antigen that facilitates intracellular transport of the multi-specific ABP by the internalizing receptor. In certain embodiments, the particular epitope provides an advantageous function for internalization. In certain embodiments, the internalizing receptor is a cell surface internalizing receptor. In certain embodiments, the internalizing receptor is an endocytic receptor. In certain embodiments, the internalizing receptor is capable of targeting a lysosome. In certain embodiments, the internalizing receptor is an oncogenic receptor.

In some embodiments, the multi-specific ABP binds to antigen of an internalizing receptor where the internalizing receptor is cation-dependent mannose-6-phosphate receptor (CD-M6PR).

In certain embodiments, the internalizing receptor is CD-M6PR. In certain embodiments, the internalizing receptor is CI-M6PR.

In particular embodiments the internalizing receptor is CI-M6PR. In some embodiments, binding of the antigen-binding moiety that binds to CI-M6PR is an allosteric binder and/or direct confirmational agonist that rapidly promotes internalization of M6PR. In some embodiments, binding of the antigen binding moiety to CI-M6PR facilitates binding of M6PR to M6P (e.g., competing and/or non-competing binding).

In some embodiments, the first antigen binding moiety comprises an antigen-binding fragment selected from the group consisting of: Fab, F(ab′)₂, single chain antibodies (scFv), (scFv) 2, diabodies, triabodies, tetrabodies, and domain antibodies.

In some embodiments, the antigen binding moiety comprises a Fab fragment and an Fc domain. In some embodiments, “Fab-Fc” fragments comprise a Fab fragment attached to an Fc domain. For example, an Fc domain may be attached to the C-terminal of the Fab fragment. Any suitable Fc domain known in the art or described herein may be used. In some cases, the Fc domain comprises an IgG4 Fc domain.

In certain embodiments, the multi-specific ABP comprises a second antigen binding moiety that specifically binds to an antigen of a soluble target molecule or a cell surface target molecule. In some embodiments, internalization of the soluble target molecule or the cell surface target molecule provides a therapeutic benefit.

In certain embodiments, the second antigen binding moiety specifically binds to the soluble target moiety. In some embodiments, the soluble target molecule is Immunoglobulin E (IgE). Non-limiting examples of an antigen-binding moiety that binds to IgE can be found in U.S. Pat. Nos. 8,071,097 and 7,867,494, and PCT Patent Publication No.: WO2017211928A1, which are hereby incorporated by reference in their entireties.

In some embodiments, the second antigen binding moiety comprises: a second variable light chain region (V_{L}) CDR1 having an amino acid sequence that is at least 70%, at least 75%, at least 80%, at least 85%, at least 90%, at least 95%, at least 96%, at least 97%, at least 98%, at least 99%, or 100% identical to an amino acid sequence of SEQ ID NO.: 138. In some embodiments the second antigen binding moiety comprises: a second variable light chain region (V_{L}) CDR2 having an amino acid sequence that is at least 70%, at least 75%, at least 80%, at least 85%, at least 90%, at least 95%, at least 96%, at least 97%, at least 98%, at least 99%, or 100% identical to an amino acid sequence of SEQ ID NO.: 139. In certain embodiments, the second antigen binding moiety comprises: a second variable light chain region (V_{L}) CDR3 having an amino acid sequence that is at least 70%, at least 75%, at least 80%, at least 85%, at least 90%, at least 95%, at least 96%, at least 97%, at least 98%, at least 99%, or 100% identical to an amino acid sequence of SEQ ID NO.: 140.

In some embodiments, the second antigen binding moiety comprises: a second variable heavy chain region (V_{H}) CDR1 having an amino acid sequence that is at least 70%, at least 75%, at least 80%, at least 85%, at least 90%, at least 95%, at least 96%, at least 97%, at least 98%, at least 99%, or 100% identical to an amino acid sequence of: SEQ ID NO.: 135. In some embodiments, the second antigen binding moiety comprises: a second variable heavy chain region (V_{H}) CDR2 having an amino acid sequence that is at least 70%, at least 75%, at least 80%, at least 85%, at least 90%, at least 95%, at least 96%, at least 97%, at least 98%, at least 99%, or 100% identical to an amino acid sequence of SEQ ID NO.: 136. In some embodiments, the second antigen binding moiety comprises: a second variable heavy chain region (V_{H}) CDR3 having an amino acid sequence that is at least 70%, at least 75%, at least 80%, at least 85%, at least 90%, at least 95%, at least 96%, at least 97%, at least 98%, at least 99%, or 100% identical to an amino acid sequence of SEQ ID NO.: 137.

In some embodiments, the second antigen binding moiety comprises a variable light chain (V_{L}) having an amino acid sequence that is at least 70%, at least 75%, at least 80%, at least 85%, at least 90%, at least 95%, at least 96%, at least 97%, at least 98%, at least 99%, or 100% identical to an amino acid sequence of SEQ ID NO: 134. In some embodiments, the second antigen binding moiety comprises a variable heavy chain (V_{H}) having an amino acid sequence that is at least 70%, at least 75%, at least 80%, at least 85%, at least 90%, at least 95%, at least 96%, at least 97%, at least 98%, at least 99%, or 100% identical to an amino acid sequence of SEQ ID NO.: 133.

In certain embodiments, the second antigen binding moiety specifically binds to the cell surface target molecule. In particular embodiments, the cell surface target molecule is selected from: ERBB receptors (e.g., EGFR, ERBB2, ERBB3, ERBB4), erythropoietin-producing hepatocellular (EPH) receptors, fibroblast growth factor (FGF) receptors (e.g., FGFR1, FGFR2, FGFR3, FGFR4, FGFR5), platelet-derived growth factor (PDGF) receptors (e.g., PDGFR-A, PDGFR-B), vascular endothelial growth factor (VEGF) receptors (e.g., VEGFR1/FLT1, VEGFR2/FLK1, VEGF3), tyrosine kinase with immunoglobulin-like and EGF-like domains (TIE) receptors, insulin-like growth factor (IGF) receptors (e.g., INS-R, IGFIR, IR-R), Discoidin Domain (DD) receptors, receptor for c-Met (MET), recepteur d′origine nantais (RON); also known as macrophage stimulating 1 receptor, Flt3 fins-related tyrosine kinase 3 (Flt3), colony stimulating factor 1 (CSF1) receptor, adhesion related kinase receptor (e.g., Axl), receptor for c-kit (KIT) and insulin receptor related (IRR) receptors.

In certain embodiments, the cell surface target molecule is epithelial growth factor receptor (EGFR). Non-limiting examples of antigen binding moieties that bind to EGFR are found in U.S. Pat. Nos. 10,954,286, 10,844,127, and 11,040, 111, which are hereby incorporated by reference in their entireties.

In some embodiments, the second antigen binding moiety comprises: a second variable light chain region (V_{L}) CDR1 having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 122 and SEQ ID NO: 130. In some embodiments, the second antigen binding moiety comprises a second V_{L} CDR2 having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 123 and SEQ ID NO: 131. In certain embodiments, the second antigen binding moiety comprises a second V_{L} CDR3 having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 124 and SEQ ID NO: 132. In certain embodiments, the second antigen binding moiety comprises a second variable heavy chain region (V_{H}) CDR1 having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 119 and SEQ ID NO: 127.

In certain embodiments, the second antigen binding moiety comprises a second V_{H} CDR2 having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 120 and SEQ ID NO: 128. In certain embodiments, the second antigen binding moiety comprises a second V_{H} CDR3 having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 121 and SEQ ID NO: 129.

In some embodiments, the second antigen binding moiety comprises a variable light chain (V_{L}) having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 118 and SEQ ID NO: 126. In some embodiments, the second antigen binding moiety comprises a second variable heavy chain (V_{H}) having an amino acid sequence that is at least 70%, at least 75%, at least 80%, at least 85%, at least 90%, at least 95%, at least 96%, at least 97%, at least 98%, at least 99%, or 100% identical to an amino acid sequence selected from SEQ ID NO.: 117 and SEQ ID NO: 125.

In some embodiments, the second antigen binding moiety comprises an antigen-binding fragment selected from the group consisting of: Fab, F(ab′)₂, single chain antibodies (scFv), (scFv) 2, diabodies, triabodies, tetrabodies, and domain antibodies.

An antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two binding sites.

Antigen binding proteins in accordance with the present invention include antigen binding proteins that facilitate intracellular transport of the ABP by the internalizing receptor. Antigen binding proteins in accordance with the present invention also include multi-specific ABPs that include a second antigen binding protein that internalize a soluble target molecule or the cell surface target molecule to provide a therapeutic benefit.

Different antigen binding proteins may bind to different domains of an internalizing receptor, a soluble target molecule, or a cell surface target molecule by different mechanisms of action. As indicated herein inter alia, the domain region are designated such as to be inclusive of the group, unless otherwise indicated. For example, amino acids 4-12 refers to nine amino acids: amino acids at positions 4, and 12, as well as the seven intervening amino acids in the sequence.

Derivatives of ABPs within the scope of this invention include covalent or aggregative conjugates of ABPs, or fragments thereof, with other proteins or polypeptides, such as by expression of recombinant fusion proteins comprising heterologous polypeptides fused to the N-terminus or C-terminus of an ABP polypeptide. For example, the conjugated peptide may be a heterologous signal (or leader)polypeptide, e.g., the yeast alpha-factor leader, or a peptide such as an epitope tag. Antigen binding protein-containing fusion proteins can comprise peptides added to facilitate purification or identification of antigen binding protein (e.g., poly-His). An antigen binding protein also can be linked to the FLAG peptide Asp-Tyr-Lys-Asp-Asp-Asp-Asp-Lys (DYKDDDDK) as described in Hopp et al., Bio Technology 6:1204, 1988, and U.S. Pat. No. 5,011,912. The FLAG peptide is highly antigenic and provides an epitope reversibly bound by a specific monoclonal antibody (mAb), enabling rapid assay and facile purification of expressed recombinant protein. Reagents useful for preparing fusion proteins in which the FLAG peptide is fused to a given polypeptide are commercially available (Sigma, St. Louis, MO).

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., 1994, EMBO J. 13:3992-4001. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

In some embodiments, the Fc region comprises a human IgG1, a human IgG2, a human IgG4 or a variant thereof. In some embodiments, the Fc region comprises a modification that affects an effector function. In some embodiments, the Fc region comprises a LALAPG a N297A, a DANA, a LALA, or a N297Q mutation. The LALAPG mutation can refer to L234A, L235A, and P329G mutations in the Fc region. The DANA mutation can refer to D265A and N297A mutations in the Fc region. The LALA mutation can refer to L234A and L235A mutation in the Fc region.

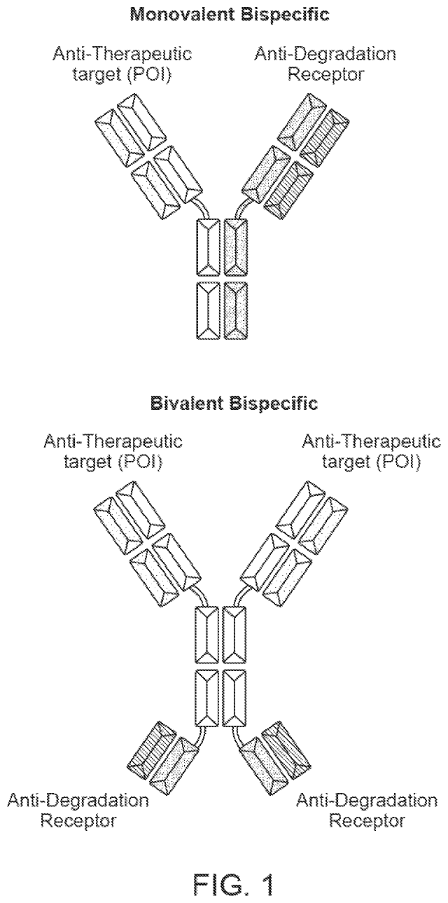

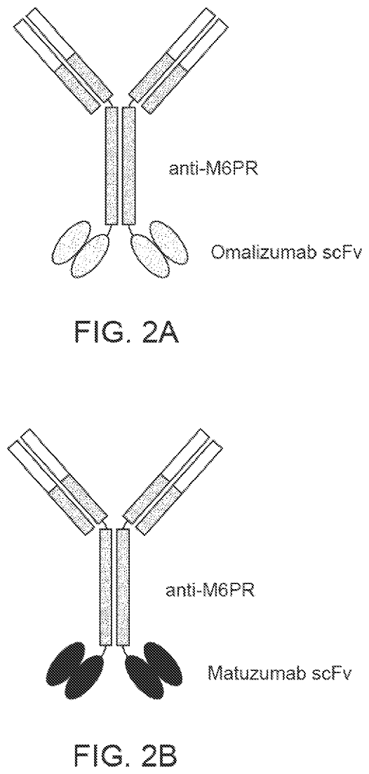

In some embodiments, the first antigen binding moiety of a multi-specific ABP is a Fab format and the second antigen binding moiety of a multi-specific ABP is an scFv format or the first antigen binding moiety is an scFv format and the second antigen binding moiety is a Fab format (See, e.g., FIG. 1 and FIGS. 2A-B and3A-3B showing non-limiting examples of anti-M6PR/IgE and anti-M6PR/EGFR multi-specific antibodies). In some embodiments, the first antigen binding moiety of a multi-specific ABP is a Fab format and the second antigen binding moiety of a multi-specific ABP is a Fab format. In some embodiments, the first antigen binding moiety of a multi-specific ABP is a scFv format and the second antigen binding moiety of a multi-specific ABP is a scFv format.

In some embodiments, the multi-specific ABP comprises a full length antibody sequence that binds to an internalizing receptor, comprising a variable light chain amino acid sequence an internalizing receptor antibody, a variable heavy chain amino acid sequence of an internalizing receptor antibody, an FC region of an internalizing receptor antibody; and an antigen binding moiety that binds to a soluble target molecule or a cell surface target molecule, such as an scFv or Fab region of an antibody that binds to a soluble target molecule or a cell surface target molecule, as shown in FIGS. 2A-2B.

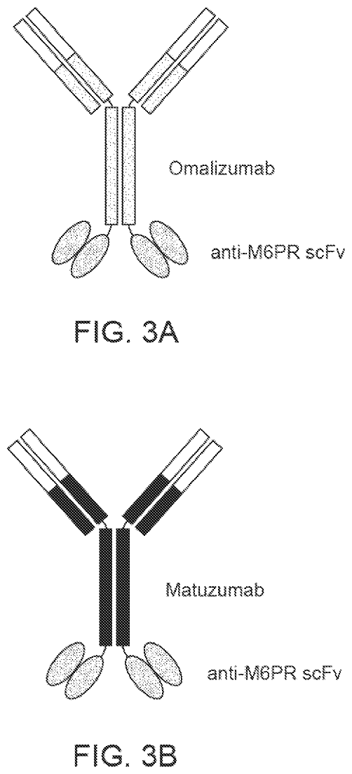

In some embodiments, the multi-specific ABP comprises a full length antibody sequence that targets a soluble target molecule or cell surface target molecule, comprising a variable light chain amino acid sequence of a soluble target molecule or a cell surface target molecule antibody, a variable heavy chain amino acid sequence of an a soluble target molecule or a cell surface target molecule antibody, an FC region of a soluble target molecule or a cell surface target molecule antibody; and an antigen binding moiety that binds to an internalizing receptor, such as an scFv or Fab region of an antibody that binds to an internalizing receptor, as shown in FIGS. 3A-3B.

Antigen binding proteins (e.g., antibodies, antibody fragments, multi-specific antibodies, and antibody derivatives) of the invention can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In one embodiment, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lantto et al., 2002, Methods Mol. Biol. 178:303-16.

In one aspect, the present disclosure provides antigen binding moieties that comprise antigen-binding fragments of an internalizing receptor, a soluble target molecule, and/or a cell surface target molecule of the invention. Such fragments can consist entirely of antibody-derived sequences or can comprise additional sequences. Examples of antigen-binding fragments include Fab, F(ab′)₂, single chain antibodies (scFv), (scFv) 2, diabodies, triabodies, tetrabodies, and domain antibodies. Other examples are provided in Lunde et al., 2002, Biochem. Soc. Trans. 30:500-06.

In some embodiments, the present disclosure provides a first antigen binding moiety that is a first antigen binding fragment, and a second antigen binding moiety that is a second antigen binding fragment.

Single chain antibodies (scFv) may be formed by linking heavy and light chain variable domain (Fv region) fragments via an amino acid bridge (short peptide linker, e.g., a synthetic sequence of amino acid residues), resulting in a single polypeptide chain. Such single-chain Fvs (scFvs) have been prepared by fusing DNA encoding a peptide linker between DNAs encoding the two variable domain polypeptides (V_{L} and V_{H}). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108, Bird et al., 1988, Science 242:423-26 and Huston et al., 1988, Proc. Natl. Acad. Sci. USA 85:5879-83). By combining different V_{L} and V_{H}-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, de Graaf et al., 2002, Methods Mol Biol. 178:379-87. ScFvs comprising the variable domain combinations 45A7, 45A12, 43C8, 12D5, 53B11, 35E2, 06D12, 03C12, 18G4, 02C7, 14A3, 10H5, 06G11, 06H10, 01E5, 21E12, 06F1, 20E12, 21D5, 12A1, 06D1, 07B2, 07E11, 56A2, 01A7, 07A4, 16H4, 08D8, 06C1, 35B2, and 17F11 are encompassed by the present invention.

Antigen Binding Moiety that Binds to M6PR

In one aspect of the present disclosure, an ABP comprises an antigen binding protein comprising at least a first antigen binding moiety that binds to M6PR. In some embodiments, the present disclosure provides antigen binding moieties that comprise a light chain variable region selected from SEQ ID NOs: 17-32 of Table 6 or a heavy chain variable region selected from SEQ ID NOs: 1-16 of Table 5, and fragments, derivatives, muteins, and variants thereof.

Also shown below are the locations of the CDRs (underlined) that create part of the antigen-binding site, while the Framework Regions (FRs) are the intervening segments of these variable domain sequences. In both light chain variable regions and heavy chain variable regions there are three CDRs (CDR1-3) and four FRs (FR 1-4). The CDR regions of each light and heavy chain also are grouped by antibody type. Antigen binding proteins of the invention include, for example, antigen binding proteins having a combination of light chain and heavy chain variable domains selected from the group of combinations of antibody clones consisting of 3C7, 6D1, 6D12, 6F1, 6H10, 8D8, 10H5-2, 11B12, 12D5, 16H4, 17F11, 18G4, 21D5, 43C8, 45A12, and 53B11 of Tables 5-8.

In some embodiments, a first antigen binding moiety comprises all six CDR sequences (three CDRs of light chain “LCDR1-LCDR3” and three CDRs of heavy chain “HCDR1-HCDR3”) identical to one of the clones in the library of internal receptor binding clones, of tables 5-8. In some embodiments, a first antigen binding moiety comprises three out of six CDR sequences (three CDRs of light chain or three CDRs of heavy chain) identical to one of the clones in the library of M6PR binding clones of Tables 5-8. In some embodiments, a first antigen binding moiety comprises one, two, three, four, or five out of six CDR sequences identical to one of the clones in the library of M6PR binding clones of Tables 5-8.

In one embodiment, the present invention provides a first antigen binding moiety comprising a light chain variable domain comprising a sequence of amino acids that differs from the sequence of a light chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 6 by one or more residues, wherein each such sequence difference is independently either a deletion, insertion, or substitution of one amino acid residue. In another embodiment, the light-chain variable domain comprises a sequence of amino acids that is at least 70%, 75%, 80%, 85%, 90%, 95%, 97%, or 99% identical to the sequence of a light chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 6. In another embodiment, the light chain variable domain comprises a sequence of amino acids that is encoded by a nucleotide sequence that is at least 70%, 75%, 80%, 85%, 90%, 95%, 97%, or 99% identical to a nucleotide sequence that encodes a light chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 6. In another embodiment, the light chain variable domain comprises a sequence of amino acids that is encoded by a polynucleotide that hybridizes under moderately stringent conditions to the complement of a polynucleotide that encodes a light chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 6. In another embodiment, the light chain variable domain comprises a sequence of amino acids that is encoded by a polynucleotide that hybridizes under moderately stringent conditions to the complement of a polynucleotide that encodes a light chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 6. In another embodiment, the light chain variable domain comprises a sequence of amino acids that is encoded by a polynucleotide that hybridizes under moderately stringent conditions to a complement of a light chain polynucleotide of 3C7, 6D1, 6D12, 6F1, 6H10, 8D8, 10H5-2, 11B12, 12D5, 16H4, 17F11, 18G4, 21D5, 43C8, 45A12, and 53B11 of Table 6.

In another embodiment, the present invention provides a first antigen binding moiety comprising a heavy chain variable domain comprising a sequence of amino acids that differs from the sequence of a heavy chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 5 by one or more residues, wherein each such sequence difference is independently either a deletion, insertion, or substitution of one amino acid residue. In another embodiment, the heavy chain variable domain comprises a sequence of amino acids that is at least 70%, 75%, 80%, 85%, 90%, 95%, 96%, 97%, 98%, or 99% identical to the sequence of a heavy chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 5. In another embodiment, the heavy chain variable domain comprises a sequence of amino acids that is encoded by a nucleotide sequence that is at least 70%, 75%, 80%, 85%, 90%, 95%, 96%, 97%, 98%, or 99% identical to a nucleotide sequence that encodes a heavy chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 5. In another embodiment, the heavy chain variable domain comprises a sequence of amino acids that is encoded by a polynucleotide that hybridizes under moderately stringent conditions to the complement of a polynucleotide that encodes a heavy chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 5. In another embodiment, the heavy chain variable domain comprises a sequence of amino acids that is encoded by a polynucleotide that hybridizes under moderately stringent conditions to the complement of a polynucleotide that encodes a heavy chain variable domain selected from the group consisting of 3C7, 6D1, 6D12, 6F1, 6H10, 8D8, 10H5-2, 11B12, 12D5, 16H4, 17F11, 18G4, 21D5, 43C8, 45A12, and 53B11 of Table 5. In another embodiment, the heavy chain variable domain comprises a sequence of amino acids that is encoded by a polynucleotide that hybridizes under moderately stringent conditions to a complement of a heavy chain polynucleotide disclosed herein.

In some embodiments, the heavy chain variable domain comprises a sequence of amino acids that is at least 70%, 75%, 80%, 85%, 90%, 95%, 96%, 97%, 98%, or 99% identical to an amino acid sequence selected from SEQ ID NOs.: 1-16. In some embodiments, the light chain variable domain comprises a sequence of amino acids that is at least 70%, 75%, 80%, 85%, 90%, 95%, 96%, 97%, 98%, or 99% identical to an amino acid sequence selected from SEQ ID NOs: 17-32.

Particular embodiments of antigen binding moieties of the present disclosure comprise one or more amino acid sequences that are identical to the amino acid sequences of one or more of the CDRs and/or FRs referenced herein. In one embodiment, the antigen binding moiety comprises a light chain CDR1 (LCDR1) sequence illustrated above. In another embodiment, the antigen binding moiety comprises a light chain CDR2 (LCDR2) sequence illustrated above. In another embodiment, the antigen binding moiety comprises a light chain CDR3 (LCDR3) sequence illustrated above. In another embodiment, the antigen binding moiety comprises a heavy chain CDR1 (HCDR1) sequence illustrated above. In another embodiment, the antigen binding moiety comprises a heavy chain CDR2 (HCDR2) sequence illustrated above. In another embodiment, the antigen binding moiety comprises a heavy chain CDR3 (HCDR3) sequence illustrated above.

In one embodiment, the present invention provides an antigen binding moiety that comprises one or more CDR sequences that differ from a CDR sequence shown above by no more than 5, 4, 3, 2, or 1 amino acid residues.

In some embodiments, at least one of the antigen binding moiety's HCDR1 sequences is a HCDR1 sequence from 3C7, 6D1, 6D12, 6F1, 6H10, 8D8, 10H5-2, 11B12, 12D5, 16H4, 17F11, 18G4, 21D5, 43C8, 45A12, and 53B11 of Table 7. In some embodiments, at least one of the antigen binding moiety's HCDR2 sequences is a HCDR2 sequence from 3C7, 6D1, 6D12, 6F1, 6H10, 8D8, 10H5-2, 11B12, 12D5, 16H4, 17F11, 18G4, 21D5, 43C8, 45A12, and 53B11 of Table 7. In some embodiments, at least one of the antigen binding moiety's HCDR3 sequences is a HCDR3 sequence from 3C7, 6D1, 6D12, 6F1, 6H10, 8D8, 10H5-2, 11B12, 12D5, 16H4, 17F11, 18G4, 21D5, 43C8, 45A12, and 53B11 of Table 7.

In some embodiments, the antigen binding moiety comprises a variable heavy chain region (V_{H}) CDR 1 having an amino acid sequence that is at least 70%, at least 80%, at least 85%, at least 90%, at least 95%, at least 96%, at least 96%, at least 98%, at least 99%, or at least 100% identical to an amino acid sequence selected from SEQ ID NOs.: 33, 36, 39, 42, 45, 47, 50, 56, 61, 64, 67, and 70. In some embodiments, the antigen binding moiety comprises a V_{H} CDR2 having an amino acid sequence that is at least 70%, at least 80%, at least 85%, at least 90%, at least 95%, at least 96%, at least 96%, at least 98%, at least 99%, or at least 100% identical to an amino acid sequence selected from SEQ ID NOs.: 34, 37, 40, 43, 48, 51, 53, 57, 59, 62, 65, 68, and 71. In some embodiments, the antigen binding moiety comprises a V_{H} CDR3 having an amino acid sequence that is at least 70%, at least 80%, at least 85%, at least 90%, at least 95%, at least 96%, at least 96%, at least 98%, at least 99%, or at least 100% identical to an amino acid sequence selected from SEQ ID NOs.: 35, 38, 41, 44, 46, 49, 52, 54, 55, 58, 60, 63, 66, 69, 72, and 73.

In some embodiments, the antigen binding moiety comprises a variable heavy chain region (V_{H}) CDR 1 having an amino acid sequence that is at least 95% identical to an amino acid sequence selected from SEQ ID NOs.: 33, 36, 39, 42, 45, 47, 50, 56, 61, 64, 67, and 70. In some embodiments, the antigen binding moiety comprises a V_{H} CDR2 having an amino acid sequence that is at least 95% identical to an amino acid sequence selected from SEQ ID NOs.: 34, 37, 40, 43, 48, 51, 53, 57, 59, 62, 65, 68, and 71. In some embodiments, the antigen binding moiety comprises a V_{H} CDR3 having an amino acid sequence that is at least 95% identical to an amino acid sequence selected from SEQ ID NOs.: 35, 38, 41, 44, 46, 49, 52, 54, 55, 58, 60, 63, 66, 69, 72, and 73.

In another embodiment, the antigen binding moiety's light chain LCDR1 sequence is a light chain LCDR1 sequence from 3C7, 6D1, 6D12, 6F1, 6H10, 8D8, 10H5-2, 11B12, 12D5, 16H4, 17F11, 18G4, 21D5, 43C8, 45A12, and 53B11 of Table 8. In some embodiments, the antigen binding moiety's light chain LCDR2 sequence is a light chain LCDR2 sequence from 3C7, 6D1, 6D12, 6F1, 6H10, 8D8, 10H5-2, 11B12, 12D5, 16H4, 17F11, 18G4, 21D5, 43C8, 45A12, and 53B11 of Table 8. In some embodiments, the antigen binding moiety's light chain LCDR3 sequence is a light chain LCDR3 sequence from 3C7, 6D1, 6D12, 6F1, 6H10, 8D8, 10H5-2, 11B12, 12D5, 16H4, 17F11, 18G4, 21D5, 43C8, 45A12, and 53B11 of Table 8.

In some embodiments, the antigen binding moiety comprises a variable light chain region (V_{L}) CDR1 having an amino acid sequence that is at least 70%, at least 80%, at least 85%, at least 90%, at least 95%, at least 96%, at least 96%, at least 98%, at least 99%, or at least 100% identical to an amino acid sequence selected from SEQ ID NOs.: 74, 77, 79, 82, 86, 89, 92, 95, 97, 100, 102, 105, 108, 111, and 114. In some embodiments, the antigen binding moiety comprises a V_{L} CDR2 having an amino acid sequence that is at least 70%, at least 80%, at least 85%, at least 90%, at least 95%, at least 96%, at least 96%, at least 98%, at least 99%, or at least 100% identical to an amino acid sequence selected from SEQ ID NOs. 75, 80, 83, 87, 90, 93, 98, 103, 106, 109, 112, and 115. In some embodiments, the antigen binding moiety comprises a V_{L} CDR3 having an amino acid sequence that is at least 70%, at least 80%, at least 85%, at least 90%, at least 95%, at least 96%, at least 96%, at least 98%, at least 99%, or at least 100% identical to an amino acid sequence selected from SEQ ID NOs.: 76, 78, 81, 84, 85, 88, 91, 94, 96, 99, 101, 104, 107, 110, 113, and 116.

In some embodiments, the antigen binding moiety comprises a variable light chain region (V_{L}) CDR1 having an amino acid sequence that is at least 95% identical to an amino acid sequence selected from SEQ ID NOs.: 74, 77, 79, 82, 86, 89, 92, 95, 97, 100, 102, 105, 108, 111, and 114. In some embodiments, the antigen binding moiety comprises a V_{L} CDR2 having an amino acid sequence that is at least 95% identical to an amino acid sequence selected from SEQ ID NOs. 75, 80, 83, 87, 90, 93, 98, 103, 106, 109, 112, and 115. In some embodiments, the antigen binding moiety comprises a V_{L} CDR3 having an amino acid sequence that is at least 95% identical to an amino acid sequence selected from SEQ ID NOs.: 76, 78, 81, 84, 85, 88, 91, 94, 96, 99, 101, 104, 107, 110, 113, and 116.

In another embodiment, the antigen binding moiety's comprises 1, 2, 3, 4, or 5 CDR sequence(s) that each independently differs by 6, 5, 4, 3, 2, 1, or 0 single amino acid additions, substitutions, and/or deletions from a CDR sequence of 3C7, 6D1, 6D12, 6F1, 6H10, 8D8, 10H5-2, 11B12, 12D5, 16H4, 17F11, 18G4, 21D5, 43C8, 45A12, and 53B11, and the antigen binding protein further comprises 1, 2, 3, 4, or 5 CDR sequence(s) that each independently differs by 6, 5, 4, 3, 2, 1, or 0 single amino acid additions, substitutions, and/or deletions from a CDR sequence. In some embodiments, the antigen binding moiety's comprises 1, 2, 3, 4, or 5 CDR sequence(s) that each has at least 50%, 55%, 60%, 65%, 70%, 75%, 80%, 85%, 90%, 95%, 96%, 97%, 98%, or 99% sequence identity to a CDR sequence of 3C7, 6D1, 6D12, 6F1, 6H10, 8D8, 10H5-2, 11B12, 12D5, 16H4, 17F11, 18G4, 21D5, 43C8, 45A12, and 53B11.

The nucleotide sequences of 3C7, 6D1, 6D12, 6F1, 6H10, 8D8, 10H5-2, 11B12, 12D5, 16H4, 17F11, 18G4, 21D5, 43C8, 45A12, and 53B11, or the amino acid sequences of 3C7, 6D1, 6D12, 6F1, 6H10, 8D8, 10H5-2, 11B12, 12D5, 16H4, 17F11, 18G4, 21D5, 43C8, 45A12, and 53B11, can be altered, for example, by random mutagenesis or by site-directed mutagenesis (e.g., oligonucleotide-directed site-specific mutagenesis) to create an altered polynucleotide comprising one or more particular nucleotide substitutions, deletions, or insertions as compared to the non-mutated polynucleotide. Examples of techniques for making such alterations are described in Walder et al., 1986, Gene 42:133; Bauer et al. 1985, Gene 37:73; Craik, BioTechniques, January 1985, 12-19; Smith et al., 1981, Genetic Engineering: Principles and Methods, Plenum Press; and U.S. Pat. Nos. 4,518,584 and 4,737,462. These and other methods can be used to make, for example, derivatives of anti-internalizing receptor antibodies that have a desired property, for example, increased affinity, avidity, or specificity for an internalizing receptor, increased activity or stability in vivo or in vitro, or reduced in vivo side-effects as compared to the underivatized antibody.

In one embodiment, an antigen binding moiety of the invention comprises the IgG1 heavy chain domain of any of 3C7, 6D1, 6D12, 6F1, 6H10, 8D8, 10H5-2, 11B12, 12D5, 16H4, 17F11, 18G4, 21D5, 43C8, 45A12, and 53B1lor a fragment of the IgG1 heavy chain domain of any of 3C7, 6D1, 6D12, 6F1, 6H10, 8D8, 10H5-2, 11B12, 12D5, 16H4, 17F11, 18G4, 21D5, 43C8, 45A12, and 53B11. In another embodiment, an antigen binding moiety of the invention comprises the kappa light chain constant chain region of 3C7, 6D1, 6D12, 6F1, 6H10, 8D8, 10H5-2, 11B12, 12D5, 16H4, 17F11, 18G4, 21D5, 43C8, 45A12, and 53B11, or a fragment of the kappa light chain constant region of 3C7, 6D1, 6D12, 6F1, 6H10, 8D8, 10H5-2, 11B12, 12D5, 16H4, 17F11, 18G4, 21D5, 43C8, 45A12, and 53B11. In another embodiment, an antigen binding moiety of the invention comprises both the IgG1 heavy chain domain, or a fragment thereof, of 3C7, 6D1, 6D12, 6F1, 6H10, 8D8, 10H5-2, 11B12, 12D5, 16H4, 17F11, 18G4, 21D5, 43C8, 45A12, and 53B11 and the kappa light chain domain, or a fragment thereof, of 3C7, 6D1, 6D12, 6F1, 6H10, 8D8, 10H5-2, 11B12, 12D5, 16H4, 17F11, 18G4, 21D5, 43C8, 45A12, and 53B11.

In some embodiments, at least one antigen binding moiety of the present invention include those comprising, for example, the variable domain combinations 3C7, 6D1, 6D12, 6F1, 6H10, 8D8, 10H5-2, 11B12, 12D5, 16H4, 17F11, 18G4, 21D5, 43C8, 45A12, and 53B11, having a desired isotype (for example, IgA, IgG1, IgG2, IgG3, IgG4, IgM, IgE, and IgD) as well as Fab or F(ab′)₂ fragments thereof. Moreover, if an IgG4 is desired, it may also be desired to introduce a point mutation (CPSCP->CPPCP) in the hinge region as described in Bloom et al., 1997, Protein Science 6:407, incorporated by reference herein) to alleviate a tendency to form intra-H chain disulfide bonds that can lead to heterogeneity in the IgG4 antibodies.

In one embodiment, the antigen binding moiety of an antigen binding protein has a K_{off} of 1×10⁻⁴ s⁻¹ or lower. In another embodiment, the K_{off} is 5×10⁻⁵ s⁻¹ or lower. In another embodiment, the K_{off} is substantially the same as an antibody having a combination of light chain and heavy chain variable domain sequences selected from the group of combinations consisting of 3C7, 6D1, 6D12, 6F1, 6H10, 8D8, 10H5-2, 11B12, 12D5, 16H4, 17F11, 18G4, 21D5, 43C8, 45A12, and 53B11 of Tables 5-6. In another embodiment, the antigen binding moiety binds to M6PR with substantially the same K_{off} as an antibody that comprises one or more CDRs from an antibody having a combination of light chain and heavy chain variable domain sequences selected from the group of combinations consisting of 3C7, 6D1, 6D12, 6F1, 6H10, 8D8, 10H5-2, 11B12, 12D5, 16H4, 17F11, 18G4, 21D5, 43C8, 45A12, and 53B11 of Tables 7-8. In another embodiment, the antigen binding moiety binds to M6PR with substantially the same K_{off} as an antibody that comprises one of the amino acid sequences illustrated above. In another embodiment, the antigen binding moiety binds to M6PR with substantially the same K_{off} as an antibody that comprises one or more CDRs from an antibody that comprises one of the amino acid sequences illustrated above.

Antigen binding proteins of the present disclosure that comprise a first antigen binding moiety may have a binding affinity for an internalizing receptor of less than or equal to 5×10⁻⁷M, less than or equal to 1×10⁻⁷M, less than or equal to 0.5×10⁻⁷M, less than or equal to 1×10⁻⁸ M, less than or equal to 1×10⁻⁹M, less than or equal to 1×10⁻¹⁰ M, less than or equal to 1×10⁻¹¹ M, or less than or equal to 1×10⁻¹² M. In some embodiments, the first antigen binding moiety binds the internalizing receptor with a K_{D} ranging from 1 nM to 1 pM.

In some embodiments, a first antigen binding moiety binds the internalizing receptor with a K_{D} of less than 500 nM. In some embodiments, a first antigen binding moiety binds the internalizing receptor with a K_{D} of less than 200 nM. In some embodiments, a first antigen binding moiety binds the internalizing receptor with a K_{D} of less than 25 nM. In some embodiments, a first antigen binding moiety binds the internalizing receptor with a K_{D} of less than 5 nM. In some embodiments, a first antigen binding moiety binds the internalizing receptor with a K_{D} of less than 2 nM. In some embodiments, the first antigen binding moiety binds the internalizing receptor with a K_{D} ranging from 1×10⁻⁷ M to 5×10⁻¹² M. In some embodiments, the first antigen binding moiety binds the internalizing receptor with a K_{D} ranging from 2×10⁻⁷ to 9.5×10⁻⁸ M.

The affinity of an antibody or antigen binding protein for its binding partner can be determined by one of ordinary skill in the art using conventional techniques, for example those described by Scatchard et al. (Ann. N.Y. Acad. Sci. 51:660-672 (1949)) or by surface plasmon resonance (SPR; BIAcore, Biosensor, Piscataway, NJ). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the SPR signal can be analyzed to yield apparent rate constants for the association and dissociation phases of the binding reaction. The ratio of these values gives the apparent equilibrium constant (affinity) (see, e.g., Wolff et al., Cancer Res. 53:2560-65 (1993)).

An antibody or antigen binding protein according to the present disclosure may belong to any immunoglobin class, for example IgG, IgE, IgM, IgD, or IgA. It may be obtained from or derived from an animal, for example, fowl (e.g., chicken) and mammals, which includes but is not limited to a mouse, rat, hamster, rabbit, or other rodent, cow, horse, sheep, goat, camel, human, or other primate.

### 6.6. Monoclonal Antibody

In another aspect, the present invention provides monoclonal antibodies that bind to an internalizing receptor (e.g., M6PR). Monoclonal antibodies of the invention may be generated using a variety of known techniques. In general, monoclonal antibodies that bind to specific antigens may be obtained by methods known to those skilled in the art (see, for example, Kohler et al., Nature 256:495, 1975; Coligan et al. (eds.), Current Protocols in Immunology, 1:2.5.12.6.7 (John Wiley & Sons 1991); U.S. Pat. Nos. RE 32,011, 4,902,614, 4,543,439, and 4,411,993; Monoclonal Antibodies, Hybridomas: A New Dimension in Biological Analyses, Plenum Press, Kennett, McKearn, and Bechtol (eds.) (1980); and Antibodies: A Laboratory Manual, Harlow and Lane (eds.), Cold Spring Harbor Laboratory Press (1988); Picksley et al., “Production of monoclonal antibodies against proteins expressed in E. coli,” in DNA Cloning 2: Expression Systems, 2nd Edition, Glover et al. (eds.), page 93 (Oxford University Press 1995)). Antibody fragments may be derived therefrom using any suitable standard technique such as proteolytic digestion, or optionally, by proteolytic digestion (for example, using papain or pepsin) followed by mild reduction of disulfide bonds and alkylation. Alternatively, such fragments may also be generated by recombinant genetic engineering techniques as described herein.

Monoclonal antibodies can be obtained by injecting an animal, for example, a rat, hamster, a rabbit, or preferably a mouse, including for example a transgenic or a knock-out, as known in the art, with an immunogen comprising human M6PR or a fragment thereof, according to methods known in the art and described herein. The presence of specific antibody production may be monitored after the initial injection and/or after a booster injection by obtaining a serum sample and detecting the presence of an antibody that binds to human M6PR or peptide using any one of several immunodetection methods known in the art and described herein. From animals producing the desired antibodies, lymphoid cells, most commonly cells from the spleen or lymph node, are removed to obtain B-lymphocytes. The B lymphocytes are then fused with a drug-sensitized myeloma cell fusion partner, preferably one that is syngeneic with the immunized animal and that optionally has other desirable properties (e.g., inability to express endogenous Ig gene products, e.g., P3X63-Ag 8.653 (ATCC No. CRL 1580); NSO, SP20) to produce hybridomas, which are immortal eukaryotic cell lines.

The lymphoid (e.g., spleen) cells and the myeloma cells may be combined for a few minutes with a membrane fusion-promoting agent, such as polyethylene glycol or a nonionic detergent, and then plated at low density on a selective medium that supports the growth of hybridoma cells but not unfused myeloma cells. A preferred selection media is HAT (hypoxanthine, aminopterin, thymidine). After a sufficient time, usually about one to two weeks, colonies of cells are observed. Single colonies are isolated, and antibodies produced by the cells may be tested for binding activity to human M6PR, using any one of a variety of immunoassays known in the art and described herein. The hybridomas are cloned (e.g., by limited dilution cloning or by soft agar plaque isolation) and positive clones that produce an antibody specific to M6PR are selected and cultured. The monoclonal antibodies from the hybridoma cultures may be isolated from the supernatants of hybridoma cultures.

An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Monoclonal antibodies can be isolated and purified by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)). Monoclonal antibodies may be purified by affinity chromatography using an appropriate ligand selected based on particular properties of the antibody (e.g., heavy or light chain isotype, binding specificity, etc.). Examples of a suitable ligand, immobilized on a solid support, include Protein A, Protein G, a constant region (light chain or heavy chain) antibody, an anti-idiotype antibody, and a M6PR binding protein, or fragment or variant thereof.

An antibody of the present disclosure may also be a humanized or fully human monoclonal antibody.

### 6.7. pH Dependent Antigen Binding

In one aspect, the present invention provides an antigen binding protein (ABP) comprising a first antigen binding moiety that specifically binds to an internalizing domain of cation-independent mannose 6 phosphate receptor (CI-M6PR) that facilitates intracellular transport of the ABP, where the binding of the ABP to the CI-M6PR is pH dependent. In such cases, the first antigen binding moiety preferentially disassociated from the antigen in intracellular compartments having a lower pH.

In some embodiments, the first antigen binding moiety binds to CI-M6PR with higher affinity at pH 7.4 than at pH 6.0. In some embodiments, the first antigen binding moiety binds to CI-M6PR with higher affinity at pH 7.4 than at pH 5.5. In some embodiments, the first antigen binding moiety binds to CI-M6PR with higher affinity at pH 7.4 than at pH 5.0.

In some embodiments, the antigen binding moiety is released from CI-M6PR at a pH of 7.4 or lower. In some embodiments, the antigen binding moiety is released from CI-M6PR at a pH of 6.0 or lower. In some embodiments, the antigen binding moiety is released from CI-M6PR at a pH of 5.5 or lower. In some embodiments, the antigen binding moiety is released from CI-M6PR at a pH of 5.0 or lower.

In some embodiments, the first antigen binding moiety has a K_{D} ratio or K_{off} ratio at pH 6.0/pH 7.4 that ranges between 1, 2, 5, 10, 20, 25, 50, 75 or 100 or more.

In some embodiments, the first antigen binding moiety has a K_{D} ratio or K_{off} ratio at pH 5.5/pH 7.4 that ranges between 1, 2, 5, 10, 20, 25, 50, 75 or 100 or more.

In some embodiments, the first antigen binding moiety has a K_{D} ratio or K_{off} ratio at pH 5.0/pH 7.4 that ranges between 1, 2, 5, 10, 20, 25, 50, 75 or 100 or more.

### 6.8. Internalization Domain

In some embodiments, the antigen binding domain specifically binds to an internalizing domain of a target molecule. An internalizing domain of a target molecule facilitates, participates in, or otherwise contributes to intracellular transport of the ABP. In some embodiments, the internalizing domain of a target molecule facilitates, participates in, or otherwise contributes to transportation of the ABP to an endo-lysosomal compartment. In some embodiments, where the target molecule is present on the surface of a cell, after facilitating, participating in, or otherwise contributing to the internalization of the ABP, the internalizing domain of a target molecule facilitates, participates in, or otherwise contributes to recycling of the target molecule to the surface of the cell.

In some embodiments, where the antigen binding domain specifically binds to an internalizing domain of a target molecule, binding activates the target molecule, whereby the activated target molecule facilitates, participates in, or otherwise contributes to intracellular transport of the ABP.

### 6.9. Target Binding Moiety

In one aspect, the present invention provides an antigen binding protein (ABP) comprising a first antigen binding moiety that specifically binds to an internalizing domain of cation-independent mannose 6 phosphate receptor (CI-M6PR) that facilitates intracellular transport of the ABP and a target binding moiety that specifically binds to a target molecule.

In some embodiments, the ABP is an aptamer that specifically binds to a target molecule, such as a target protein. In some embodiments, ABP is a peptide or protein (e.g., peptidic binding motif, protein domain, engineered polypeptide, or glycoprotein) that specifically binds to a target molecule, such as a target protein. In some embodiments, ABP is an antibody or antibody fragment that specifically binds to a target molecule, such as a target protein. In some embodiments, Y is a polynucleotide or oligonucleotide that specifically binds to a target molecule, such as a target protein or a target nucleic acid.

In some embodiments, a peptide or protein that specifically binds to a target molecule includes, without limitation, a peptidic binding motif, a protein domain, an engineered polypeptide, or a glycoprotein.

In some embodiments, the target binding moiety is an aptamer that specifically binds to a target molecule. Aptamers are nucleic acid molecules having specific binding affinity to molecule through interactions other than classic Watson-Crick base pairing. Aptamers are capable of specifically binding to selected targets and modulating the target's activity (e.g., binding aptamers may block the target molecule's ability to function). A typical aptamer is 10⁻¹⁵ kDa in size (30-45 nucleotides), binds its target with sub-nanomolar affinity, and discriminates against closely related targets (e.g., aptamers will typically not bind other proteins from the same gene family). An aptamer bind to target molecules via interactions (e.g., hydrogen bonding, electrostatic complementarity, hydrophobic contacts, steric exclusion) that drive affinity and specificity.

In some embodiments, the targeting binding moiety is a ligand that specifically binds to a target molecule. In some embodiments, the ligand is a molecule capable of modulating signaling. In some embodiments, the ligand is a cytokine (e.g., a proinflammatory cytokine or an anti-inflammatory cytokine). Non-limiting examples of cytokines include IL-1 family cytokines (non-limiting examples of IL-1A, IL-1B, IL-18, and IL-33), TNF-family cytokines (non-limiting examples include: TNFalpha, TNFbeta, and 4-1BBL), Interferon family cytokines (non-limiting examples include: IFNalpha (IFNA), IFNgamma (IFNG), and IFNbeta (IFNB)), IL-6 family cytokines (non-limiting examples include: IL-6, IL-11 and LIF), IL-10 family cytokines (non-limiting examples include: IL-10, IL-19, IL-20, IL-22), and TGF beta family cytokines (e.g., TGFbeta1, TGFbeta2, and TGFbeta3).

In some embodiments, the target binding moiety is a second antigen binding moiety that specifically binds to a different target molecule than the first antigen binding moiety.

In some embodiments, the target binding moiety is a second antigen binding moiety that specifically binds to the same target molecule as the first antigen binding moiety. In such cases, the second antigen binding domain can be any of the second antigen binding domains described herein. In some embodiments, the target binding moiety is a second antigen binding moiety that specifically binds to the same target molecule but a different epitope as the first antigen binding moiety.

In some embodiments, the target binding moiety is a small molecule.

In some embodiments, the target binding moiety is conjugated to the first antigen binding moiety, optionally via a linker (e.g., any of the linkers described herein). In some embodiments, the target binding moiety is conjugated to the first antigen binding moiety using a linker (e.g., any of the linkers described herein). In some embodiments, the linker is selected based on the type of target binding moiety.

In some embodiments, target binding moiety is conjugated to the first antigen binding moiety using a linker selected as described in U.S. Pat. Nos. 7,837,980B2; 8,906,376B2; 10,201,615B2; 10,933, 112B2; U.S. Patent Publication No. 2019/0290775A1; 2022/0111066A1; and PCT Publication No. WO 2021/259506A1.

In some embodiments where the target binding moiety is a small molecule, the small molecule can be coupled to that antigen binding protein at an activatable site. Suitable activatable sites include conjugation points such as thiol groups, amino groups (e.g., the epsilon amino group of lysine residues or at the N-terminus of proteins), vicinal hydroxyl groups (1,2-diols) (e.g., oxidized carbohydrates) and carboxyl groups (e.g., the C-terminus of proteins, aspartic acid and glutamic acid residues, and carbohydrate, such as sialic acid residues).

In some embodiments where the target binding moiety is a small molecule, the small molecule can be coupled directly to a conjugation point on an antigen binding protein. For example, a drug can be attached by alkylation of the ε-amino group of antibody lysines, reductive amination of oxidized carbohydrate or reaction with a hydrazide, transesterification between hydroxyl and carboxyl groups, amidation at amino groups or carboxyl groups, and conjugation to thiols (e.g., interchain thiols) or introduced thiols by, for example, alkylating lysines with 2-iminothiolane. Suitable methods conjugating drugs to conjugation points are disclosed in, for example, Current Protocols in Protein Science (John Wiley & Sons, Inc.), Chapter 15: Chemical Modifications of Proteins, which is incorporated by reference herein in its entirety.

In some embodiments where the target binding moiety is a small molecule, the small molecule can be coupled indirectly to the antigen binding protein via another molecule, such as a linker. For example, a drug also can be conjugated via a maleimide group coupled to a sulfhydryl group in, for example, the hinge region of an antibody. In some embodiments, antigen binding protein (e.g., antibody) conjugates can be made by reacting a maleimide-derivatized form of the drug with the antigen binding domain (e.g., the antibody). In some embodiments, an antigen binding protein (e.g., an antibody) conjugate can be made by reducing an antigen binding domain (e.g., an antibody) to produce the reduced antibody, producing an amine drug, derivatizing the amine drug with maleimide to produce a maleimide-derivatized drug, and reacting the maleimide-derivatized drug with the antigen binding domain (e.g., the antibody).

In some embodiments where the target binding moiety is a small molecule, two or more small molecules can be coupled (e.g., directly or indirectly) to the antigen binding protein.

### 6.9.1. Target Molecules

In some embodiments where the ABP includes a target binding moiety that specifically binds to a target molecule, the target molecule is selected from: a soluble extracellular target molecule, a cell surface target molecule, a toxin, a pathogen, and a therapeutic agent.

In some embodiments, the target molecule is an immunoglobulin. In some embodiments, the target molecule is Immunoglobulin E (IgE).

In some embodiments, the target molecule is a receptor in the epidermal growth factor receptor (EGFR) family.

### 6.10. Cargo Moiety

In one aspect, the present invention provides an antigen binding protein (ABP) comprising a first antigen binding moiety that specifically binds to an internalizing domain of cation-independent mannose 6 phosphate receptor (CI-M6PR) that facilitates intracellular transport of the ABP and a cargo moiety. In some embodiments, the cargo moiety is fused to the antigen binding protein. In some embodiments, the cargo moiety is fused to the N-terminus of the antigen binding protein. For example, where the ABP comprises a Fab fragment and a cargo moiety, the cargo moiety is fused to the N-terminus of the ABP. In some embodiments, the cargo moiety is fused to the C-terminus of the antigen binding protein. For example, where the ABP comprises a Fab-Fc fragment and the Fc domain is attached to the C-terminus of the Fab fragment, the cargo moiety is fused to the C-terminus of the Fc domain (e.g., the domains are ordered from N-terminus to C-terminus: Fab-Fc-cargo moiety)

In another aspect, the present invention provides an antigen binding protein (ABP) comprising a first antigen binding moiety that specifically binds to an internalizing domain of cation-independent mannose 6 phosphate receptor (CI-M6PR) that facilitates intracellular transport of the ABP, a cargo moiety fused to the antigen binding protein, and a target binding moiety that specifically binds to a target molecule (e.g., any of the target molecules described herein).

In some embodiments, the cargo moiety is selected from a small molecule, a drug, a dye, a fluorophore, a monosaccharide, a disaccharide, a trisaccharide, a biomolecule, a nanoparticle, and a viral composition.

In some embodiments, the cargo moiety is a biomolecule selected from a polypeptide, polynucleotide, aptamer, polysaccharide, glycoprotein, lipid, enzyme, antibody, and antibody fragment.

In some embodiments, the cargo moiety is conjugated to the first antigen binding moiety, optionally via a linker (e.g., any of the linkers described herein). In some embodiments, the cargo moiety is conjugated to the first antigen binding moiety using a linker (e.g., any of the linkers described herein). In some embodiments, the linker is selected based on the type of target binding moiety.

### 6.11. Linkers

The terms “linker”, “linking moiety” and “linking group” are used interchangeably and refer to a linking moiety that covalently connects two or more moieties or compounds, such as ligands and other moieties of interest. In some cases, the linker is divalent and connects two moieties. In certain cases, the linker is a branched linking group that is trivalent or of a higher multivalency. In some cases, the linker that connects the two or more moieties has a linear or branched backbone of 500 atoms or less (such as 400 atoms or less, 300 atoms or less, 200 atoms or less, 100 atoms or less, 80 atoms or less, 60 atoms or less, 50 atoms or less, 40 atoms or less, 30 atoms or less, or even 20 atoms or less) in length, e.g., as measured between the two or more moieties. A linking moiety may be a covalent bond that connects two groups or a linear or branched chain of between 1 and 500 atoms in length, for example of about 1, 2, 3, 4, 5, 6, 8, 10, 12, 14, 16, 18, 20, 30, 40, 50, 100, 150, 200, 300, 400 or 500 carbon atoms in length, where the linker may be linear, branched, cyclic or a single atom. In certain cases, one, two, three, four, five or more, ten or more, or even more carbon atoms of a linker backbone may be optionally substituted with heteroatoms, e.g., sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, one or more of the following: oligo (ethylene glycol), ether, thioether, disulfide, amide, carbonate, carbamate, tertiary amine, alkyl which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle, a cycloalkyl group or a heterocycle group, where 2 or more atoms, e.g., 2, 3 or 4 atoms, of the cyclic group are included in the backbone.

In some embodiments, a “linker” or linking moiety is derived from a molecule with two reactive termini, one for conjugation to a moiety of interest, e.g., a biomolecule (e.g., an antibody) and the other for conjugation to a moiety that binds to a cell surface receptor M6PR. When the moiety of interest is a polypeptide, the polypeptide conjugation reactive terminus of the linker is in some cases a site that is capable of conjugation to the polypeptide through a cysteine thiol or lysine amine group on the polypeptide, and so is can be a thiol-reactive group such as a maleimide or a dibromomaleimide, or as defined herein, or an amine-reactive group such as an active ester (e.g., perfluorophenyl ester or tetrafluorophenyl ester), or as defined herein.

In certain embodiments of the formula described herein, the linker includes one or more straight or branched-chain carbon moieties and/or polyether (e.g., ethylene glycol) moieties (e.g., repeating units of —CH2CH2O—), and combinations thereof. In certain embodiments, these linkers optionally have amide linkages, urea or thiourea linkages, carbamate linkages, ester linkages, amino linkages, ether linkages, thioether linkages, sulfhydryl linkages, or other hetero functional linkages. In certain embodiments, the linker comprises one or more of carbon atoms, nitrogen atoms, sulfur atoms, oxygen atoms, and combinations thereof. In certain embodiments, the linker comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker comprises a linear structure. In certain embodiments, the linker comprises a branched structure. In certain embodiments, the linker comprises a cyclic structure.

In certain embodiments, the linker includes one or more linking moieties independently selected from —C1-20-alkylene-, —NHCO—C1-6-alkylene-, —CONH—C1-6-alkylene-, —NH C1-6-alkylene-, —NHCONH—C1-6-alkylene-, —NHCSNH—C1-6-alkylene-, —C1-6-alkylene-NHCO—, —C1-6-alkylene-CONH—, —C1-6-alkylene-NH—, —C1-6-alkylene-NHCONH—, —C1-6-alkylene-NHCSNH—, O(CH2)p-, —(OCH2CH2)p-, —NHCO—, —CONH—, —NHSO2-, —SO2NH—, —CO—, —SO2-, —O—, —S—, monocyclic heteroaryl (e.g., 1,2,3-triazole), monocyclic aryl (e.g., phenyl, e.g., 1,4-linked phenyl or 1,3-linked phenyl), monocyclic heterocycle (e.g., pyrrolidine-2,5-dione, piperazine or piperidine ring as described herein), amino acid residue (naturally or non-naturally occurring amino acid residue), —NH—, and —NMe-, wherein each p is independently 1 to 50.

### 6.11.1. Chemoselective Ligation Groups

A chemoselective ligation group is a group having a reactive functionality or function group capable of conjugation to a compatible group of a second moiety. For example, chemoselective ligation groups (or a precursor thereof) may be one of a pair of groups associated with a conjugation chemistry such as azido-alkyne click chemistry, copper free click chemistry, Staudinger ligation, tetrazine ligation, hydrazine-iso-Pictet-Spengler (HIPS) ligation, cysteine-reactive ligation chemistry (e.g., thiol-maleimide, thiol-haloacetamide or alkyne hydrothiolation), amine-active ester coupling, reductive amination, dialkyl squarate chemistry, etc.,

Chemoselective ligation groups that may be utilized in linking two moieties, include, but are not limited to, amino (e.g., a N-terminal amino or a lysine sidechain group of a polypeptide), azido, aryl azide, alkynyl (e.g., ethynyl or cyclooctyne or derivative), active ester (e.g., N-hydroxysuccinimide (NHS) ester, sulfo-NHS ester or PFP ester or thioester), haloacetamide (e.g., iodoacetamide or bromoacetamide), chloroacetyl, bromoacetyl, hydrazide, maleimide, vinyl sulfone, 2-sulfonyl pyridine, cyano-alkyne, thiol (e.g., a cysteine residue), disulfide or protected thiol, isocyanate, isothiocyanate, aldehyde, ketone, alkoxyamine, hydrazide, aminooxy, phosphine, HIPS hydrazinyl-indolyl group, or aza-HIPS hydrazinyl-pyrrolo-pyridinyl group, tetrazine, cyclooctene, squarate, and the like.

Conjugates of this disclosure may be made using a variety of linkers and/or bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that the conjugates described herein may be prepared using any suitable methods as disclosed in the art (see, e.g., Bioconjugate Techniques (Hermanson ed., 2d ed. 2008)).

In some embodiments, a chemoselective ligation group is capable of spontaneous conjugation to a compatible chemical group when the two groups come into contact under suitable conditions (e.g., copper free Click chemistry conditions). In some instances, the chemoselective ligation group is capable of conjugation to a compatible chemical group when the two groups come into contact in the presence of a catalyst or other reagent (e.g., copper catalyzed Click chemistry conditions). In some embodiments, the chemoselective ligation group is a photoactive ligation group. For example, upon irradiation with ultraviolet light, a diazirine group can form reactive carbenes, which can insert into C—H, N—H, and O—H bonds of a second moiety.

### 6.12. Sequences

Antibodies 3C7, 6D1, 6D12, 6F1, 6H10, 8D8, 10H5-2, 11B12, 12D5, 16H4, 17F11, 18G4, 21D5, 43C8, 45A12, and 53B11 comprise heavy and light chain V (J) D polynucleotides (e.g. of antibody clones 3C7, 6D1, 6D12, 6F1, 6H10, 8D8, 10H5-2, 11B12, 12D5, 16H4, 17F11, 18G4, 21D5, 43C8, 45A12, and 53B11). Antibodies 3C7, 6D1, 6D12, 6F1, 6H10, 8D8, 10H5-2, 11B12, 12D5, 16H4, 17F11, 18G4, 21D5, 43C8, 45A12, and 53B11 comprise the variable light chain and variable heavy chain sequences listed in Tables 5-6. CDR sequences in the light chain and heavy chain are also provided with a specific SEQ ID NO.

TABLE 5
Full length M6PR ABP variable heavy chain amino acid sequences

| Antibody | | SEQ ID |
|---|---|---|
| Clone | Variable Heavy Chain | NO: |
| 45A12 | QVQLQQSGAELMKPGASVKISCKATGYTFSTYWIEWVK | 1 |
| | QRPGHGLEWIGEILPGSGSTNYNEKFKGKATFTADASSN | |
| | TAYMQLSSLTSEDSAVYYCARDGGAMDYWGQGTSVTV | |
| | SS | |
| 43C8 | EIQLQQSGPELVKPGASVKMSCKASGYTFTDYVISWVK | 2 |
| | QRTGQGLEWIGEIYPGSHTYYNEKFKGKATLTADKSSN | |
| | TAYMQLSSLTSEDSAVYFCALTAGAMDYWGQGTSVTV | |
| | SS | |
| 12D5 | QVQLQQPGAELVKPGASVKLSCKASGYTFTNYWMHW | 3 |
| | VKQRPGQGLEWIGMIHPNSGSTNYNEKFKSKATLTVDK | |
| | SSSTAYMQLSSLTSEDSAVYYCARPHGYPFGYWGQGTT | |
| | LTVSS | |
| 53B11 | EVQLQQSGPELVKPGTSMKISCKASGYSFTDYTVNWVK | 4 |
| | QSHGKNLEWIGLINPYNGGTSYNQKFKGKATLTVDKSS | |
| | STAYMELLSLTSEDSAVYYCARWGYYYATSYYFAMDY | |
| | WGQGTSVTVSS | |
| 06D12 | QVQLQQSGAELMKPGASVKLSCKATGYTFTGYWIEWV | 5 |
| | KQRPGHGLEWIGEILPGSGSTNYNEKFKGKATFTADTSS | |
| | NTAYMQLSSLTTEDSAIYYCAYTVVATDWYFDVWGTG | |
| | TTVTVSS | |
| 18G4 | EVKLMESGGGLVQPGASLRLSCAASGFTFTDYYMSWV | 6 |
| | RQPPGKAPEWLALIRNKANGYTTEYNASVKGRFTISRD | |
| | NSQNILYLQMNTLRTEDSATYYCVRGGFAYWGQGTLV | |
| | TVSA | |
| 10H5-2 | EVQLQQSGAELVRPGASVKLSCTASGFNIKDDYMHWV | 7 |
| | KQRPEQGLEWIGWIDPENGDTEYASKFQGKATITADTSS | |
| | NTAYLQLSSLTSEDTAVYYCTTQYGPLYFDYWGQGTTL | |
| | TVSS | |
| 06H10 | EVQLQQSGAELVRPGASVKLSCTASGFNIKDDYMHWV | 8 |
| | KQRPEQGLEWIGWIDPENGDTEYASKFQGKATITADTSS | |
| | NTAYLQLSSLTSEDTAVYYCTTVAPYYYAMDYWGQGT | |
| | SVTVSS | |
| 06F1 | EVQLQQSGAELVRPGASVKLSCTASGFNIKDDYMHWV | 9 |
| | KQRPEQGLEWIGWIDPENGDTEYASKFQGKATITADTSS | |
| | NTAYLQLSSLTSEDTAVYYCTNYYGSSPFDYWGQGTTL | |
| | TVSS | |
| 21D5 | QVQLKQSGPGLVQPSQSLSITCTVSGFSLTRYGVHWVRQ | 10 |
| | SPGKGLEWLGVIWRGGSTDYNAAFMSRLSITKDNSKSQ | |
| | VFFKMNSLQADDTAIYYCAKEGTGILDSWGQGTTLTVS | |
| | S | |
| 06D1 | EVQLQQSGAELVRPGASVKLSCTASGFNIKDDYMHWV | 11 |
| | KQRPEQGLEWIGWIDPENGDTEYASKFQGKATITADTSS | |
| | NTAYLQLSSLTSEDTAVYYCTTVAPGYYAMDYWGQGT | |
| | SVTVSS | |
| 16H4 | QVQLKQSGPGLVQPSQSLSITCTASGFSLTSYGVHWVRQ | 12 |
| | SPGKGLEWLGVIWSGGSTDYSAAFISRLSISKDSSKSQVF | |
| | FKMNSLQADDTAIYYCARNLPITSYDPSSFYAMDYWGQ | |
| | GTSVTVSS | |
| 08D8 | QVQLQQPGAELVKPGASVKLSCKASGYTFTSYWMQWV | 13 |
| | KQRPGQGLEWIGEIDPSDSYTNYNQKFKGKATLTVDTSS | |
| | STAYMQLSSLTSEDSAVYYCARGDYYGSSSWFAYWGQ | |
| | GTLVTVSA | |
| 17F11 | EVQLQQSGPELVKPGASVKMSCKASGYTFTDYNMHWV | 14 |
| | KQSHGKSLEWIGYINPNNGGTNYNQKFQGKATLTVNKS | |
| | SRTAYMELRSLTSEDSAVYYCVRFYYGSRYYAMDYWG | |
| | QGTSVTVSS | |
| 3C7 | QVQLQQSGPELVKPGASVKISCKASGYSFTSYYIHWVK | 15 |
| | QRPGQGLEWIGWIYPGSGNTKYNEKFKGKATLTADTSS | |
| | STAYMQLSSLTSEDSAVYYCARMGSSYWYFDVWGTGT | |
| | TVTVSS | |
| 11B12 | QVQLQQSGPELVKPGASVKISCKASGYSFTSYYIHWVK | 16 |
| | QRPGQGLEWIGWIYPGSGNTKYNEKFKGKATLTADTSS | |
| | STAYMQLSSLTSEDSAVYYCARGNWHWYFDVWGTGTT | |
| | VTVSS | |

TABLE 6
Full length M6PR ABP variable light chain amino acid sequences

| Antibody | | SEQ ID |
|---|---|---|
| Clone | Variable Light Chain | NO: |
| 45A12 | NIMMTQSPSSLAVSAGEKVTMSCKSSQRVLYSSNQKNYL | 17 |
| | AWYQQKPGQSPKLLIYWASTRESGVPDRFTGSGSGTDFT | |
| | LTISTVQPEDLAVYYCHQYLSSRTFGGGTKLEIK | |
| 43C8 | NIMMTQSPSSLAVSAGEKVTMSCKSSQSVLYSSNQKNYL | 18 |
| | AWYQQKPGQSPKLLIYWASTRESGVPDRFTGSGSGTDFT | |
| | LTISSVHAEDLAVYYCHQYLSSYTFGGGTKLEIK | |
| 12D5 | ENVLTQSPAIMSASLGEKVTMSCRASSSVNYMYWYQQK | 19 |
| | SDASPKLWIYYTSNLAPGVPARFSGSGSGNSYSLTISSME | |
| | GEDAATYYCQQFTSSPYTFGGGTKLEIK | |
| 53B11 | DIQMNQSPSSLSASLGDTITITCHASQNINVWLSWYQQKP | 20 |
| | GNIPKLLIYKASNLHTGVPSRFSGSGSGTGFTLTISSLQPED | |
| | IATYYCQQGQSYPWTFGGGTKLEIK | |
| 06D12 | ENVLTQSPAIMSASLGEKVTMSCRASSSVNYMYWYQQK | 21 |
| | SDASPKLWIYYTSNLAPGVPARFSGSGSGNSYSLTISSME | |
| | GEDAATYYCQQFTSSPWTFGGGTKLEIK | |
| 18G4 | QAVVIQESALTTSPGETVTLTCRSSTGAVTTSHYAIWVQE | 22 |
| | KPDHLFTGLIGGTNNRVPGVPARFSGSLIADKAALTITGA | |
| | QTEDEAIYFCALWYTNQFIFGSGTKVTVL | |
| 10H5-2 | QAVVTQESALTTSPGETVTLTCRSSTGAVTTSKYANWVQ | 23 |
| | EKPDHLFTGVIGGTNNRAPGVPARFSGSLIGDKAALTITG | |
| | AQTEDEAIYFCALWYSNHWVFGGGTKLTVL | |
| 06H10 | QIVLTQSPAIMSASLGEEITLTCSASSSVSYMHWYQQKSG | 24 |
| | TSPKLLIYSTSNLASGVPSRFSGSGSGTFYSLTISSVEAEDA | |
| | ADYYCHQWSSYHTFGGGTKLEIK | |
| 06F1 | QIVLTQSPAIMSAFLGERVTMTCTASSSVSSRYLHWYQQ | 25 |
| | KPGSSPKLWIYSTSNLASGVPARFSGSGSGTSYSLTISSME | |
| | AEDAATYYCHQYHRSPLTFGAGTKLELK | |
| 21D5 | DIVLTQSPASLAVSLGQRATISCRASKSVSTSGYSYMHWY | 26 |
| | QQKPGQPPTLLIYLASNLESGVPARFSGSGSGTDFTLNIHP | |
| | VEEEDAATYYCQHSRELPYTFGGGTKLEIK | |
| 06D1 | ENVLTQSPAIMSASPGEKVTMTCRASSSVSSSYLHWYQQ | 27 |
| | KSGASPKLWIYSTSNLASGVPARFSGSGSGTSYSLTISSVE | |
| | AEDAATYYCQQYSGFTFGSGTKLEIK | |
| 16H4 | DIQMTQTTSSLSASLGDRVTISCSASQGISNYLNWYQQKP | 28 |
| | DGTVKLLIYYTSSLHSGVPSRFSGSGSGTDYSLTISNLEPE | |
| | DIATYYCQQYSKLPFTFGSGTKLEIK | |
| 08D8 | DIVMTQAAPSVPVTPGESVSISCRSSKSLLHSNGNTYLYW | 29 |
| | FLQRPGQSPQLLIYRMSNLASGVPDRFSGSGSGTAFTLRIS | |
| | RVEAEDVGVYYCMQHLEYPFTFGAGTKLELK | |
| 17F11 | DIVMTQSPATLSVTPGDRVSLSCRASQSISDYLHWYQQKS | 30 |
| | HESPRLLIKYASQSISGIPSRFSGSGSGSDFTLSINSVEPEDV | |
| | GVYYCQNGHSFPYTFGGGTKLEIK | |
| 3C7 | ENVLTQSPAIMAASLGQKVTMTCSASSSVSSSYLHWYQQ | 31 |
| | KSGASPKPLIHRTSNLASGVPARFSGSGSGTSYSLTISSVE | |
| | AEDDATYYCQQWSGYPLTFGAGTKLELK | |
| 11B12 | EIVLTQSPALMAASPGEKVTITCSVSSSISSSNLHWYQQKS | 32 |
| | ETSPKPWIYGTSNLASGVPVRFSGSGSGTSYSLTISSMEAE | |
| | DAATYYCQQWSSYPLTFGAGTKLELK | |

TABLE 7
M6PR variable heavy chain ABP CDRs (HCDRs)

| Antibody | | SEQ ID |
|---|---|---|
| Clone | Heavy Chain CDRs | NO: |
| 45A12 | HCDR1: TYWIE | 33 |
| | HCDR2: EILPGSGSTNYNEKFKG | 34 |
| | HCDR3: DGGAMDY | 35 |
| 43C8 | HCDR1: DYVIS | 36 |
| | HCDR2: EIYPGSHTYYNEKFKG | 37 |
| | HCDR3: TAGAMDY | 38 |
| 12D5 | HCDR1: NYWMH | 39 |
| | HCDR2: MIHPNSGSTNYNEKFKS | 40 |
| | HCDR3: PHGYPFGY | 41 |
| 53B11 | HCDR1: DYTVN | 42 |
| | HCDR2: LINPYNGGTSYNQKFKG | 43 |
| | HCDR3: WGYYYATSYYFAMDY | 44 |
| 06D12 | HCDR1: GYWIE | 45 |
| | HCDR2: EILPGSGSTNYNEKFKG | 34 |
| | HCDR3: TVVATDWYFDV | 46 |
| 18G4 | HCDR1: DYYMS | 47 |
| | HCDR2: LIRNKANGYTTEYNASVKG | 48 |
| | HCDR3: GGFAY | 49 |
| 10H5-2 | HCDR1: DDYMH | 50 |
| | HCDR2: WIDPENGDTEYASKFQG | 51 |
| | HCDR3: QYGPLYFDY | 52 |
| 06H10 | HCDR1: DDYMH | 50 |
| | HCDR2: WIDPENGDTEYASKFQG | 53 |
| | HCDR3: VAPYYYAMDY | 54 |
| 06F1 | HCDR1: DDYMH | 50 |
| | HCDR2: WIDPENGDTEYASKFQG | 51 |
| | HCDR3: YYGSSPFDY | 55 |
| 21D5 | HCDR1: RYGVH | 56 |
| | HCDR2: VIWRGGSTDYNAAFMS | 57 |
| | HCDR3: EGTGILDS | 58 |
| 06D1 | HCDR1: DDYMH | 50 |
| | HCDR2: WIDPENGDTEYASKFQG | 59 |
| | HCDR3: VAPGYYAMDY | 60 |
| 16H4 | HCDR1: SYGVH | 61 |
| | HCDR2: VIWSGGSTDYSAAFIS | 62 |
| | HCDR3: NLPITSYDPSSFYAMDY | 63 |
| 08D8 | HCDR1: SYWMQ | 64 |
| | HCDR2: EIDPSDSYTNYNQKFKG | 65 |
| | HCDR3: GDYYGSSSWFAY | 66 |
| 17F11 | HCDR1: DYNMH | 67 |
| | HCDR2: YINPNNGGTNYNQKFQG | 68 |
| | HCDR3: FYYGSRYYAMDY | 69 |
| 3C7 | HCDR1: SYYIH | 70 |
| | HCDR2: WIYPGSGNTKYNEKFKG | 71 |
| | HCDR3: MGSSYWYFDV | 72 |
| 11B12 | HCDR1: SYYIH | 70 |
| | HCDR2: WIYPGSGNTKYNEKFKG | 71 |
| | HCDR3: GNWHWYFDV | 73 |

TABLE 8
M6PR variable light chain ABP CDRs (LCDRs)

| Antibody | | SEQ ID |
|---|---|---|
| Clone | Light Chain CDRs | NO: |
| 45A12 | LCDR1: KSSQRVLYSSNQKNYLA | 74 |
| | LCDR2: WASTRES | 75 |
| | LCDR3: HQYLSSRT | 76 |
| 43C8 | LCDR1: KSSQSVLYSSNQKNYLA | 77 |
| | LCDR2: WASTRES | 75 |
| | LCDR3: HQYLSSYT | 78 |
| 12D5 | LCDR1: RASSSVNYMY | 79 |
| | LCDR2: YTSNLAP | 80 |
| | LCDR3: QQFTSSPYT | 81 |
| 53B11 | LCDR1: HASQNINVWLS | 82 |
| | LCDR2: KASNLHT | 83 |
| | LCDR3: QQGQSYPWT | 84 |
| 06D12 | LCDR1: RASSSVNYMY | 79 |
| | LCDR2: YTSNLAP | 80 |
| | LCDR3: QQFTSSPWT | 85 |
| 18G4 | LCDR1: RSSTGAVTTSHYAI | 86 |
| | LCDR2: GTNNRVP | 87 |
| | LCDR3: ALWYTNQFI | 88 |
| 10H5-2 | LCDR1: RSSTGAVTTSKYAN | 89 |
| | LCDR2: GTNNRAP | 90 |
| | LCDR3: ALWYSNHWV | 91 |
| 06H10 | LCDR1: SASSSVSYMH | 92 |
| | LCDR2: STSNLAS | 93 |
| | LCDR3: HQWSSYHT | 94 |
| 06F1 | LCDR1: TASSSVSSRYLH | 95 |
| | LCDR2: STSNLAS | 93 |
| | LCDR3: HQYHRSPLT | 96 |
| 21D5 | LCDR1: RASKSVSTSGYSYMH | 97 |
| | LCDR2: LASNLES | 98 |
| | LCDR3: QHSRELPYT | 99 |
| 06D1 | LCDR1: RASSSVSSSYLH | 100 |
| | LCDR2: STSNLAS | 93 |
| | LCDR3: QQYSGFT | 101 |
| 16H4 | LCDR1: SASQGISNYLN | 102 |
| | LCDR2: YTSSLHS | 103 |
| | LCDR3: QQYSKLPFT | 104 |
| 08D8 | LCDR1: RSSKSLLHSNGNTYLY | 105 |
| | LCDR2: RMSNLAS | 106 |
| | LCDR3: MQHLEYPFT | 107 |
| 17F11 | LCDR1: RASQSISDYLH | 108 |
| | LCDR2: YASQSIS | 109 |
| | LCDR3: QNGHSFPYT | 110 |
| 3C7 | LCDR1: SASSSVSSSYLH | 111 |
| | LCDR2: RTSNLAS | 112 |
| | LCDR3: QQWSGYPLT | 113 |
| 11B12 | LCDR1: SVSSSISSSNLH | 114 |
| | LCDR2: GTSNLAS | 115 |
| | LCDR3: QQWSSYPLT | 116 |

TABLE 9
Exemplary anti-EGFR antibody sequences

| Name | Heavy Chain | Light Chain | HCDRs 1-3 | LCDRs 1-3 |
|---|---|---|---|---|
| Matuzumab | QVQLVQSGAE | DIQMTQSPSS | HCDR1: | LCDR1: |
| | VKKPGASVKV | LSASVGDRV | SHWMH | SASSSVTYMY |
| | SCKASGYTFT | TITCSASSSVT | (SEQ ID NO: | (SEQ ID NO: 122) |
| | SHWMHWVRQ | YMYWYQQK | 119) | LCDR2: |
| | APGQGLEWIG | PGKAPKLLIY | HCDR2: | DTSNLAS |
| | EFNPSNGRTN | DTSNLASGVP | EFNPSNGRTN | (SEQ ID NO: 123) |
| | YNEKFKSKAT | SRFSGSGSGT | YNEKFKS | LCDR3: |
| | MTVDTSTNTA | DYTFTISSLQP | (SEQ ID NO: | QQWSSHIFT |
| | YMELSSLRSE | EDIATYYCQQ | 120) | (SEQ ID NO: 124) |
| | DTAVYYCASR | WSSHIFTFGQ | HCDR3: | |
| | DYDYDGRYF | GTKVEIK | RDYDYDGRY | |
| | DYWGQGTLV | (SEQ ID NO: | FDY | |
| | TVSS | 118) | (SEQ ID NO: | |
| | (SEQ ID NO: | | 121) | |
| | 117) | | | |
| Cetuximab | QVQLKQSGPG | DILLTQSPVIL | HCDR1: | LCDR1: |
| | LVQPSQSLSIT | SVSPGERVSF | NYGVH | RASQSIGTNIH |
| | CTVSGFSLTN | SCRASQSIGT | (SEQ ID NO: | (SEQ ID NO: 130) |
| | YGVHWVRQS | NIHWYQQRT | 127) | LCDR2: |
| | PGKGLEWLGV | NGSPRLLIKY | HCDR2: | YASESIS |
| | IWSGGNTDYN | ASESISGIPSR | VIWSGGNTD | (SEQ ID NO: 131) |
| | TPFTSRLSINK | FSGSGSGTDF | YNTPFTS | LCDR3: |
| | DNSKSQVFFK | TLSINSVESE | (SEQ ID NO: | QQNNNWPTT |
| | MNSLQSNDTA | DIADYYCQQ | 128) | (SEQ ID NO: 132) |
| | IYYCARALTY | NNNWPTTFG | HCDR3: | |
| | YDYEFAYWG | AGTKLELK | ALTYYDYEF | |
| | QGTLVTVSA | (SEQ ID NO: | AY | |
| | (SEQ ID NO: | 126) | (SEQ ID NO: | |
| | 125) | | 129) | |

TABLE 10
Exemplary anti-IgE antibody sequences

| Name | Heavy Chain | Light Chain | HCDRs 1-3 | LCDRs 1-3 |
|---|---|---|---|---|
| omalizumab | EVOL VESGGG | DIQLTQSPSSL | HCDR1: | LCDR1: |
| | LVQPGGSLRL | SASVGDRVTIT | SGYSWN | RASQSVDYDGD |
| | SCAVSGYSITS | CRASQSVDYD | (SEQ ID NO.: | SYMN |
| | GYSWNWIRQ | GDSYMNWYQ | 135) | (SEQ ID NO.: 138), |
| | APGKGLEWV | QKPGKAPKLL | HCDR2: | LCDR2: |
| | ASITYDGSTN | IYAASYLESG | SITYDGSTN | AASYLES |
| | YADSVKGRFT | VPSRFSGSGSG | YADSVKG | (SEQ ID NO.: 139), |
| | ISRDDSKNTFY | TDFTLTISSLQ | (SEQ ID NO.: | LCDR3: |
| | LQMNSLRAED | PEDFATYYCQ | 136) | QQSHEDPYT |
| | TAVYYCARGS | QSHEDPYTFG | HCDR3: | (SEQ ID NO.: 140) |
| | HYFGHWHFA | QGTKVEIKRT | GSHYFGHW | |
| | VWGQGTLVT | VAAPSVF | HFAV (SEQ | |
| | VSSGPSVFPLA | IFPPSDEQLKS | ID NO.: 137) | |
| | PSSKSTSGGTA | GTASVVCLLN | | |
| | ALGCLVKDYF | NFYPREAKVQ | | |
| | PEPVTVSWNS | WKVDNALQS | | |
| | GALTSGVHTF | GNSQESVTEQ | | |
| | PAVLQSSGLY | DSKDSTYSLSS | | |
| | SLSSVVTVPSS | TLTLSKADYE | | |
| | SLGTQTYICN | KHKVYACEVT | | |
| | VNHKPSNTKV | HQGLSSPVTK | | |
| | DKKAEPKSCD | SFNR (SEQ ID | | |
| | KTHTCPPCPAP | NO: 134) | | |
| | ELLGGPSVFLF | | | |
| | PPKPKDTLMIS | | | |
| | RTPEVTCVVV | | | |
| | DVSHEDPEVK | | | |
| | FNWYVDGVE | | | |
| | VHNAKTKPRE | | | |
| | EQYNSTYRVV | | | |
| | SVLTVLHQDW | | | |
| | LNGKEYKCKV | | | |
| | SNKALPAPIEK | | | |
| | TISKAKGQPRE | | | |
| | PQVYTLPPSR | | | |
| | DELTKNQVSL | | | |
| | TCLVKGFYPS | | | |
| | DIAVEWESNG | | | |
| | QPENNYKTTP | | | |
| | PVLDSDGSFFL | | | |
| | YSKLTVDKSR | | | |
| | WQQGNVFSCS | | | |
| | VMHEALHNH | | | |
| | YTQKSLSLSPG | | | |
| | K | | | |
| | (SEQ ID NO: | | | |
| | 133) | | | |
| ligelizumab | QVQLVQSGAE | EIVMTQSPATL | HCDR1: | LCDR1: |
| | VMKPGSSVKV | SVSPGERATLS | WYWLE | RASQSIGTNIH |
| | SCKASGYTFS | CRASQSIGTNI | (SEQ ID NO: | (SEQ ID NO: 274) |
| | WYWLEWVRQ | HWYQQKPGQ | 271) | LCDR2: |
| | APGHGLEWM | APRLLIYYASE | HCDR2: | YASESIS |
| | GEIDPGTFTTN | SISGIPA | EIDPGTFTTN | (SEQ ID NO: 275) |
| | YNEKFKARVT | RFSGSGSGTEF | YNEKFKA | LCDR3: |
| | FTADTSTSTA | TLTISSLQSED | (SEQ ID NO: | QQSWSWPTT |
| | YMELSSLRSE | FAVYYCQQS | 272) | (SEQ ID NO: 276) |
| | DTAVYYCARF | WSWPTTFGGG | HCDR3: | |
| | SHFSGSNYDY | TKVEIK | FSHFSGSNY | |
| | FDYWGQGTL | (SEQ ID NO: | DYFDY | |
| | VTVSS | 270) | (SEQ ID NO: | |
| | (SEQ ID NO: | | 273) | |
| | 269) | | | |

TABLE 11
M6PR variable heavy chain ABP framework region (FR) sequences

| Antibody | | SEQ ID |
|---|---|---|
| Clone | Heavy Chain FRs | NO: |
| 45A12 | HFR1: QVQLQQSGAELMKPGASVKISCKATGYTFS | 141 |
| | HFR2: WVKQRPGHGLEWIG | 142 |
| | HFR3: KATFTADASSNTAYMQLSSLTSEDSAVYYCAR | 143 |
| | HFR4: WGQGTSVTVSS | 144 |
| 43C8 | HFR1: EIQLQQSGPELVKPGASVKMSCKASGYTFT | 145 |
| | HFR2: WVKQRTGQGLEWIG | 146 |
| | HFR3: KATLTADKSSNTAYMQLSSLTSEDSAVYFCAL | 147 |
| | HFR4: WGQGTSVTVSS | 148 |
| 12D5 | HFR1: QVQLQQPGAELVKPGASVKLSCKASGYTFT | 149 |
| | HFR2: WVKQRPGQGLEWIG | 150 |
| | HFR3: ATLTVDKSSSTAYMQLSSLTSEDSAVYYCAR | 151 |
| | HFR4: WGQGTTLTVSS | 152 |
| 53B11 | HFR1: EVQLQQSGPELVKPGTSMKISCKASGYSFT | 153 |
| | HFR2: WVKQSHGKNLEWIG | 154 |
| | HFR3: KATLTVDKSSSTAYMELLSLTSEDSAVYYCAR | 155 |
| | HFR4: WGQGTSVTVSS | 156 |
| 06D12 | HFR1: QVQLQQSGAELMKPGASVKLSCKATGYTFT | 157 |
| | HFR2: WVKQRPGHGLEWIG | 158 |
| | HFR3: KATFTADTSSNTAYMQLSSLTTEDSAIYYCAY | 159 |
| | HFR4: WGTGTTVTVSS | 160 |
| 18G4 | HFR1: EVKLMESGGGLVQPGASLRLSCAASGFTFT | 161 |
| | HFR2: WVRQPPGKAPEWLA | 162 |
| | HFR3: FTISRDNSQNILYLQMNTLRTEDSATYYCVR | 163 |
| | HFR4: WGQGTLVTVSA | 164 |
| 10H5-2 | HFR1: EVQLQQSGAELVRPGASVKLSCTASGFNIK | 165 |
| | HFR2: WVKQRPEQGLEWIG | 166 |
| | HFR3: KATITADTSSNTAYLQLSSLTSEDTAVYYCTT | 167 |
| | HFR4: WGQGTTLTVSS | 168 |
| 06H10 | HFR1: EVQLQQSGAELVRPGASVKLSCTASGFNIK | 169 |
| | HFR2: WVKQRPEQGLEWIG | 170 |
| | HFR3: KATITADTSSNTAYLQLSSLTSEDTAVYYCTT | 171 |
| | HFR4: WGQGTSVTVSS | 172 |
| 06F1 | HFR1: EVQLQQSGAELVRPGASVKLSCTASGFNIK | 173 |
| | HFR2: WVKQRPEQGLEWIG | 174 |
| | HFR3: KATITADTSSNTAYLQLSSLTSEDTAVYYCTN | 175 |
| | HFR4: WGQGTTLTVSS | 176 |
| 21D5 | HFR1: QVQLKQSGPGLVQPSQSLSITCTVSGFSLT | 177 |
| | HFR2: WVRQSPGKGLEWLG | 178 |
| | HFR3: RLSITKDNSKSQVFFKMNSLQADDTAIYYCAK | 179 |
| | HFR4: WGQGTTLTVSS | 180 |
| 06D1 | HFR1: EVQLQQSGAELVRPGASVKLSCTASGFNIK | 181 |
| | HFR2: WVRQSPGKGLEWLG | 182 |
| | HFR3: KATITADTSSNTAYLQLSSLTSEDTAVYYCTT | 183 |
| | HFR4: WGQGTSVTVSS | 184 |
| 16H4 | HFR1: QVQLKQSGPGLVQPSQSLSITCTASGFSLT | 185 |
| | HFR2: WVRQSPGKGLEWLG | 186 |
| | HFR3: RLSISKDSSKSQVFFKMNSLQADDTAIYYCAR | 187 |
| | HFR4: WGQGTSVTVSS | 188 |
| 08D8 | HFR1: QVQLQQPGAELVKPGASVKLSCKASGYTFT | 189 |
| | HFR2: WVKQRPGQGLEWIG | 190 |
| | HFR3: KATLTVDTSSSTAYMQLSSLTSEDSAVYYCAR | 191 |
| | HFR4: WGQGTLVTVSA | 192 |
| 17F11 | HFR1: EVQLQQSGPELVKPGASVKMSCKASGYTFT | 193 |
| | HFR2: WVKQSHGKSLEWIG | 194 |
| | HFR3: KATLTVNKSSRTAYMELRSLTSEDSAVYYCVR | 195 |
| | HFR4: WGQGTSVTVSS | 196 |
| 3C7 | HFR1: QVQLQQSGPELVKPGASVKISCKASGYSFT | 197 |
| | HFR2: WVKQRPGQGLEWIG | 198 |
| | HFR3: KATLTADTSSSTAYMQLSSLTSEDSAVYYCAR | 199 |
| | HFR4: WGTGTTVTVSS | 200 |
| 11B12 | HFR1: QVQLQQSGPELVKPGASVKISCKASGYSFT | 201 |
| | HFR2: WVKQRPGQGLEWIG | 202 |
| | HFR3: KATLTADTSSSTAYMQLSSLTSEDSAVYYCAR | 203 |
| | HFR4: WGTGTTVTVSS | 204 |

TABLE 12
M6PR variable light chain ABP framework region (FR) sequences

| Antibody | | SEQ ID |
|---|---|---|
| Clone | Light Chain FRs | NO: |
| 45A12 | LFR1: NIMMTQSPSSLAVSAGEKVTMSC | 205 |
| | LFR2: WYQQKPGQSPKLLIY | 206 |
| | LFR3: GVPDRFTGSGSGTDFTLTISTVQPEDLAVYYC | 207 |
| | LFR4: FGGGTKLEIK | 208 |
| 43C8 | LFR1: NIMMTQSPSSLAVSAGEKVTMSC | 209 |
| | LFR2: WYQQKPGQSPKLLIY | 210 |
| | LFR3: GVPDRFTGSGSGTDFTLTISSVHAEDLAVYYC | 211 |
| | LFR4: FGGGTKLEIK | 212 |
| 12D5 | LFR1: ENVLTQSPAIMSASLGEKVTMSC | 213 |
| | LFR2: WYQQKSDASPKLWIY | 214 |
| | LFR3: GVPARFSGSGSGNSYSLTISSMEGEDAATYYC | 215 |
| | LFR4: FGGGTKLEIK | 216 |
| 53B11 | LFR1: DIQMNQSPSSLSASLGDTITITC | 217 |
| | LFR2: WYQQKPGNIPKLLIY | 218 |
| | LFR3: GVPSRFSGSGSGTGFTLTISSLQPEDIATYYC | 219 |
| | LFR4: FGGGTKLEIK | 220 |
| 06D12 | LFR1: ENVLTQSPAIMSASLGEKVTMSC | 221 |
| | LFR2: WYQQKSDASPKLWIY | 222 |
| | LFR3: GVPARFSGSGSGNSYSLTISSMEGEDAATYYC | 223 |
| | LFR4: FGGGTKLEIK | 224 |
| 18G4 | LFR1: QAVVIQESALTTSPGETVTLTC | 225 |
| | LFR2: WVQEKPDHLFTGLIG | 226 |
| | LFR3: GVPARFSGSLIADKAALTITGAQTEDEAIYFC | 227 |
| | LFR4: GSGTKVTVL | 228 |
| 10H5-2 | LFR1: QAVVTQESALTTSPGETVTLTC | 229 |
| | LFR2: WVQEKPDHLFTGVIG | 230 |
| | LFR3: GVPARFSGSLIGDKAALTITGAQTEDEAIYFC | 231 |
| | LFR4: FGGGTKLTVL | 232 |
| 06H10 | LFR1: QIVLTQSPAIMSASLGEEITLTC | 233 |
| | LFR2: WYQQKSGTSPKLLIY | 234 |
| | LFR3: GVPSRFSGSGSGTFYSLTISSVEAEDAADYYC | 235 |
| | LFR4: FGGGTKLEIK | 236 |
| 06F1 | LFR1: QIVLTQSPAIMSAFLGERVTMTC | 237 |
| | LFR2: WYQQKPGSSPKLWIY | 238 |
| | LFR3: GVPARFSGSGSGTSYSLTISSMEAEDAATYYC | 239 |
| | LFR4: FGAGTKLELK | 240 |
| 21D5 | LFR1: DIVLTQSPASLAVSLGQRATISC | 241 |
| | LFR2: WYQQKPGQPPTLLIY | 242 |
| | LFR3: GVPARFSGSGSGTDFTLNIHPVEEEDAATYYC | 243 |
| | LFR4: FGGGTKLEIK | 244 |
| 06D1 | LFR1: ENVLTQSPAIMSASPGEKVTMTC | 245 |
| | LFR2: WYQQKSGASPKLWIY | 246 |
| | LFR3: GVPARFSGSGSGTSYSLTISSVEAEDAATYYC | 247 |
| | LFR4: FGSGTKLEIK | 248 |
| 16H4 | LFR1: DIQMTQTTSSLSASLGDRVTISC | 249 |
| | LFR2: WYQQKPDGTVKLLIY | 250 |
| | LFR3: GVPSRFSGSGSGTDYSLTISNLEPEDIATYYC | 251 |
| | LFR4: FGSGTKLEIK | 252 |
| 08D8 | LFR1: DIVMTQAAPSVPVTPGESVSISC | 253 |
| | LFR2: WFLQRPGQSPQLLIY | 254 |
| | LFR3: GVPDRFSGSGSGTAFTLRISRVEAEDVGVYYC | 255 |
| | LFR4: FGAGTKLELK | 256 |
| 17F11 | LFR1: DIVMTQSPATLSVTPGDRVSLSC | 257 |
| | LFR2: WYQQKSHESPRLLIK | 258 |
| | LFR3: GIPSRFSGSGSGSDFTLSINSVEPEDVGVYYC | 259 |
| | LFR4: FGGGTKLEIK | 260 |
| 3C7 | LFR1: ENVLTQSPAIMAASLGQKVTMTC | 261 |
| | LFR2: WYQQKSGASPKPLIH | 262 |
| | LFR3: GVPARFSGSGSGTSYSLTISSVEAEDDATYYC | 263 |
| | LFR4: FGAGTKLELK | 264 |
| 11B12 | LFR1: EIVLTQSPALMAASPGEKVTITC | 265 |
| | LFR2: WYQQKSETSPKPWIY | 266 |
| | LFR3: GVPVRFSGSGSGTSYSLTISSMEAEDAATYYC | 267 |
| | LFR4: FGAGTKLELK | 268 |

### 6.13. Pharmaceutical Compositions

Pharmaceutical compositions containing the proteins and polypeptides of the present invention are also provided. Such compositions comprise a therapeutically or prophylactically effective amount of the polypeptide or protein in a mixture with pharmaceutically acceptable materials, and physiologically acceptable formulation materials.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition.

Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed. (1980) and 20^{th} Ed. (2000), Mack Publishing Company, Easton, PA.

Optionally, the composition additionally comprises one or more physiologically active agents, for example, an anti-angiogenic substance, a chemotherapeutic substance (such as capecitabine, 5-fluorouracil, or doxorubicin), an analgesic substance, etc., non-exclusive examples of which are provided herein. In various particular embodiments, the composition comprises one, two, three, four, five, or six physiologically active agents in addition to an internalizing receptor-binding protein, a cell surface target molecule, and/or a soluble target molecule.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the polypeptide. For example, suitable compositions may be water for injection, physiological saline solution for parenteral administration.

### 6.14. Methods of Use

In some embodiments, antigen binding proteins of the present disclosure may be used to treat a subject with a disease or disorder associated with the presence of an excess amount of a cell surface target molecule and/or a soluble target molecule.

In some embodiments, antigen binding proteins of the present disclosure may be used to treat a subject with an allergic condition including, but not limited to allergic asthma, nasal polyps, and urticaria.

Therapeutic antibodies may be used that specifically bind to an internalizing receptor. Additionally, therapeutic antibodies may be used that specifically bind to an internalizing receptor and either a soluble target molecule or a cell surface target molecule.

In vivo and/or in vitro assays may optionally be employed to help identify optimal dosage ranges. Effective doses may be extrapolated from dose-response curves derived from in vitro or animal model test systems.

Aspects of the present disclosure include a method of producing an isolated multi-specific antigen binding protein (ABP) comprising an antigen binding moiety that specifically binds to an internalizing receptor and facilitates intracellular transport of the ABP by the internalizing receptor. In some embodiments, the antigen binding moiety specifically binds to an epitope on the antigen that facilitates intracellular transport of the ABP by the internalizing receptor.

Aspects of the present disclosure provide a method of treating a disease comprising the step of administering to a subject in need thereof an effective amount of the multi-specific ABP of the present disclosure or the pharmaceutical composition of the present disclosure.

A composition can be administered alone or in combination with other treatments, either simultaneously or sequentially dependent upon the condition to be treated. For example, the pharmaceutical composition can be administered in combination with one or more drugs targeting a different soluble or cell surface target molecule.

### 6.15. EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18^{th} Edition (Easton, Pennsylvania: Mack Publishing Company, 1990); Carey and Sundberg Advanced Organic Chemistry 3rd Ed. (Plenum Press) Vols A and B (1992). Furthermore, methods of generating and selecting antibodies explained in Adler et al., A natively paired antibody library yields drug leads with higher sensitivity and specificity than a randomly paired antibody library, MAbs (2018), and Adler et al., Rare, high-affinity mouse anti-PD-1 antibodies that function in checkpoint blockade, discovered using microfluidics and molecular genomics, MAbs (2017), which are incorporated by reference in its entirety herein, can be employed.

### 6.15.1. Example 1: Generating Anti-M6PR Antibodies

Antibody generation for monoclonal antibodies that specifically bind MP6R receptor utilized a hybridoma-based antibody discovery approach using a recombinant antigen of human M6PR and various cell lines that support mouse immunization and antibody screening. Mouse immunizations were performed using proprietary hyperimmune DiversimAb/DiverGimAb mice (Abveris). Hybridomas were characterized by flow cytometry, ELISA, and Octet Biolayer Interferometry (BLI) as part of cryopreservation of hybridomas, supernatant samples and antibody sequencing.

### Materials and Methods

Each of the following protocols were used for the respective assays described herein.

### Titer Test (ELISA) Method: Indirect ELISA Protocol (in Brief)

1 Coat high-binding ELISA plate with antigen overnight at 4° C.,
2 Aspirate, block wells with 2% BSA in 1×PBS for 1 hour.
3 Aspirate, add anti-sera dilutions in blocking solution, incubate at RT for 1 hour.
4 Wash wells 4× in 1×PBST.
5 Add HRP conjugated secondary antibody in blocking solution, incubate at RT for 45 minutes.
6 Wash wells 5× in 1×PBST.
7 Add TMB substrate, followed by stop solution. Read at 450 nm.

Note: (−) ctrl NMS=Normal Mouse Serum

### Titer Test (FLOW) Cohort 1, 2 Method: FACS Staining Protocol (Overview of Key Steps)

1 Seed 100,000 cells per well
2 Resuspend in 50 μL titrated serum (starting at 1:100, 3×dilutions for 11 points).
3 Incubate for 1 hr on ice.
4 Resuspend in 50 μL secondary antibody (1:500 anti-muFc-A488 and 1:500 Live/Dead Near IR Viability).
5 Incubate for 30 min on ice.
6 Read at least 5000 events on iQue.
   Primary Screen (Flow) Method: Flow Cytometry by iQue (Key Steps)
1 Seed 100 k cells per well.
2 Resuspend in 100 μL culture medium.
3 Incubate for 1 hr on ice.
4 Resuspend in 50 μL secondary antibody.
5 Incubate for 30 min on ice.
6 Fix with 1% PFA.
7 Read at least 5000 events on iQue.
   Secondary Screen (Flow) Method: Flow Cytometry by iQue (Key Steps).
1 Seed 100 k cells per well.
2 Resuspend in 100 μL culture medium.
3 Incubate for 1 hr on ice.
4 Resuspend in 50 μL secondary antibody.
5 Incubate for 30 min on ice.
6 Fix with 1% PFA.
7 Read at least 5000 events on iQue.
   Secondary Screen (ELISA) Method: Indirect ELISA protocol
1 Coat high-binding ELISA plate with antigen overnight at 4° C.
2 Aspirate, block wells with 2% BSA in 1×PBS for 1 hour.
3 Aspirate, add supes 1:1 in blocking solution, incubate at RT for 1 hour.
4 Wash wells 4× in 1×PBST.
5 Add HRP conjugated secondary antibody in blocking solution, incubate at RT for 45 minutes.
6 Wash wells 5× in 1×PBST.
7 Add TMB substrate, followed by stop solution. Read at 450 nm.
(−) ctrl H10=media negative control.
(+) ctrl H11=Cohort 1 anti-sera positive control.
(+) ctrl H12=Cohort 2 anti-sera positive control.

### Characterization of K562 Cells for Antibody Screening

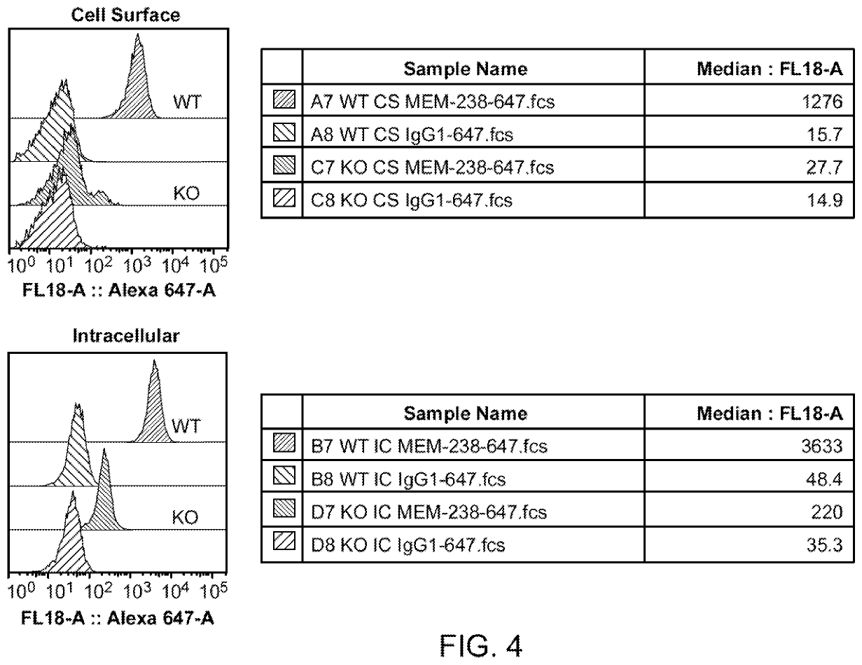

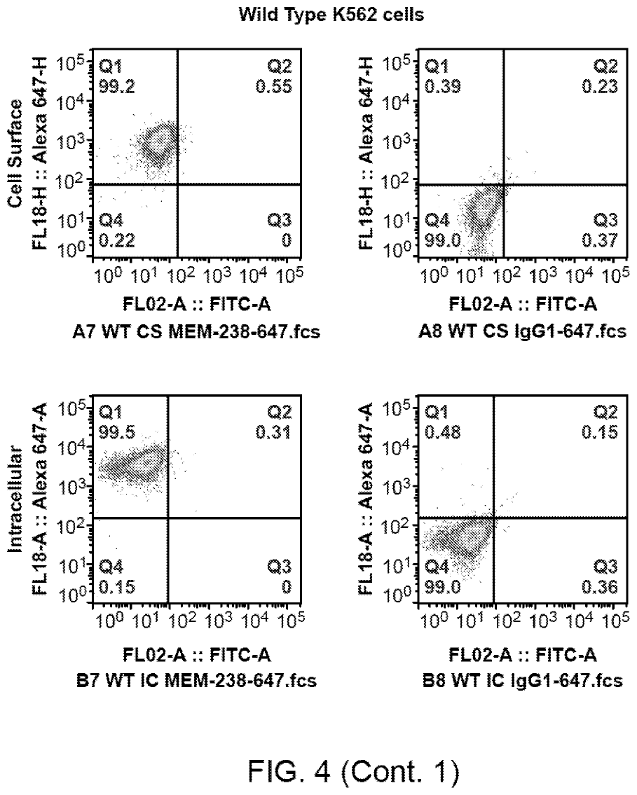

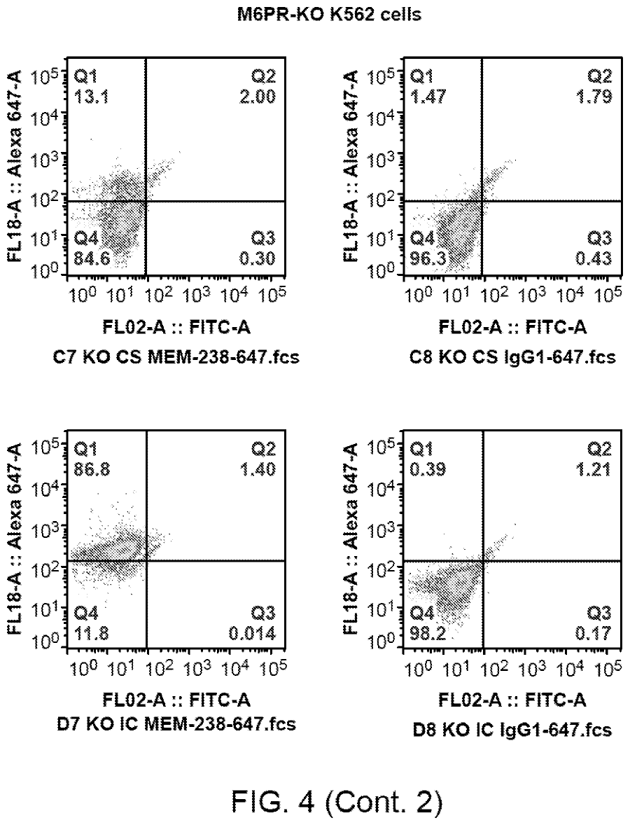

Wild type K562 cells that express M6PR and M6PR knock out (KO) K562 cells that do not contain the M6PR gene, were used for antibody screening. Cell surface and intracellular staining of human M6PR in WT and M6PR-KO K562 cells is shown in FIG. 4.

Antibodies and fluorescent stains used for antibody screening assays include anti-M6PR (clone MEM238)-Alexafluor647 (Abcam).

Cells were iced, followed by staining and fixing for the cell surface stain. For the intracellular stain, cells were fixed, permeabilized, and stained.

TABLE 13
Reagents used for immunization and screenings:

| Reagent | Use |
|---|---|
| Recombinant human M6PR ECD-His | Immunization & screening (+) |
| Recombinant mouse M6PR ECD-His | Screening (+/−) |
| K562 WT cells (human) | screening (+) |
| K562 M6PR KO cells (human) | screening (−) |
| Hela WT cells (human) | screening (+) |
| Hela M6PR KO cells (human) | screening (−) |
| Dipak MC38 cells (mouse) | screening (+/−) |
| 4T1 cells (mouse) | screening (+/−) |
| Alexa Fluor ® 647 Anti-M6PR Antibody | screening (+) |

### Mouse Immunization and Titer Testing

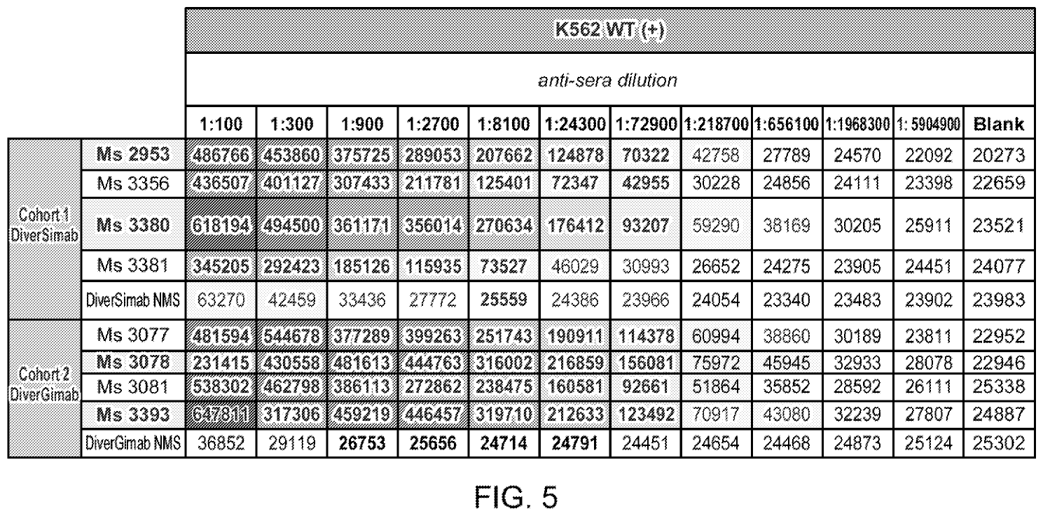

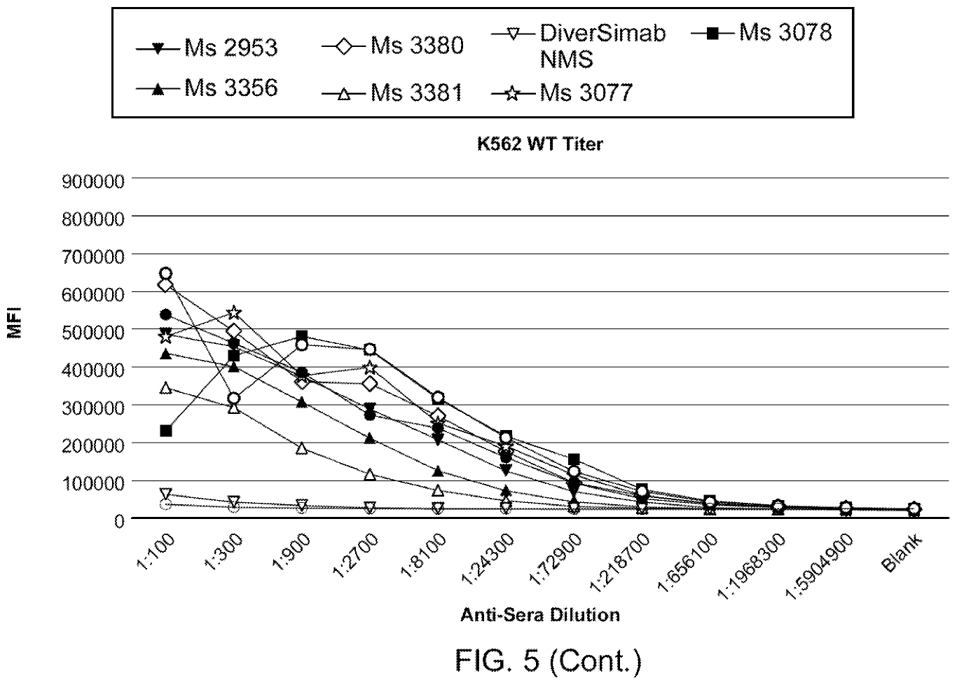

Cohorts of mice were immunized according to the following method and schedule and with the reagents described in Table 15. Daily health monitoring of mice was performed. Two cohorts of mice (n=4/cohort) of DiverSimab and DiverGimAb each received a priming dosing of 100 ug/animal of recombinant human M6PR protein and were boosted each week with 20 ug/animal of recombinant human M6PR protein for 4 weeks. Following completion of the immunization schedule, titer from each mouse was assessed using recombinant antigens and confirmed via a flow-based assay using human cell lines (K562 WT, K562 M6PR-KO, Hela WT and Hela M6PR-KO) and cross-screened against two murine cell lines (Dipak MC38 and 4T1) (FIG. 5). Mice possessing high titers were progressed to fusion.

### Hybridoma Fusion and Antibody Screening

Tissues were harvested including lymph nodes and/or spleens. A high-efficiency electrofusion was employed and hybridomas were seeded and allowed to grow in culture prior to high-throughput primary screening (flow cytometry) of clones. Primary screening (flow cytometry) was performed using K562 cells (WT and M6PR-KO) for binding confirmation. Clones were evaluated by ELISA to assess binding of each clone to human and mouse M6PR antigen. Clones that were positive for binding with human M6PR antigen were progressed to secondary flow-based screening using human cell lines (K562 WT, K562 M6PR-KO, Hela WT and Hela M6PR-KO) and murine cell lines (Dipak MC38 and 4T1) to confirm specific antibody binding to human M6PR. No mouse cross-reactive monoclonal antibodies were identified; all monoclonal antibodies specifically bound human M6PR.

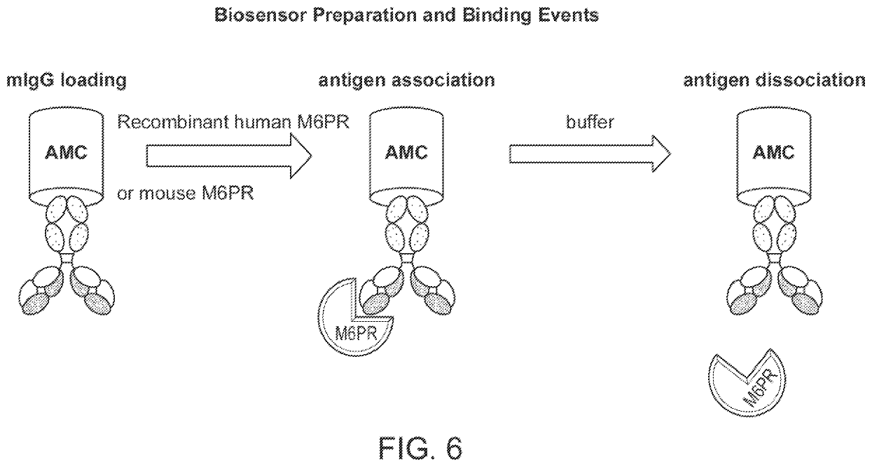

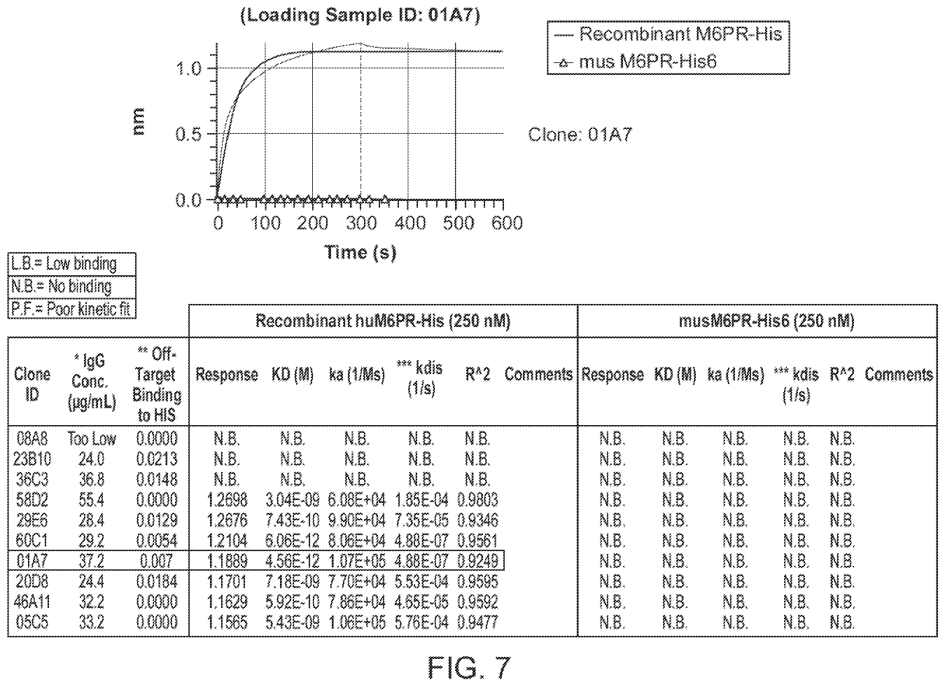

The top 192 clones were progressed to affinity measurements via Octet for measuring on/off rates and K_{D} against the human antigen. All 192 clones were sequenced using a Sanger sequencing process to generate paired V_{H}/V_{L} variable region sequences, antibody isotype data, and germline family. Individual anti-mouse biosensors were loaded with mIgGs from supernatants (as well as poly mlgG control) (FIG. 6). A baseline was measured for each biosensor. Loaded biosensors were then allowed to bind recombinant His-tagged human M6PR and the association rate was measured. Biosensors were moved to a buffer well to measure the dissociation rate. Results for a representative clone are shown in FIG. 7. Biosensors were regenerated for measurement of additional analyte interactions (mouse M6PR-His6 and off-target binding to His). Clones that possessed affinity to human M6PR and displayed no binding to His control and/or mouse M6PR were progressed to binning for determination of epitope diversity.

### 6.15.2. Example 2: Evaluating Anti-CI-M6PR Monoclonal Antibodies Generated by Assaying Hybridoma Supernatant

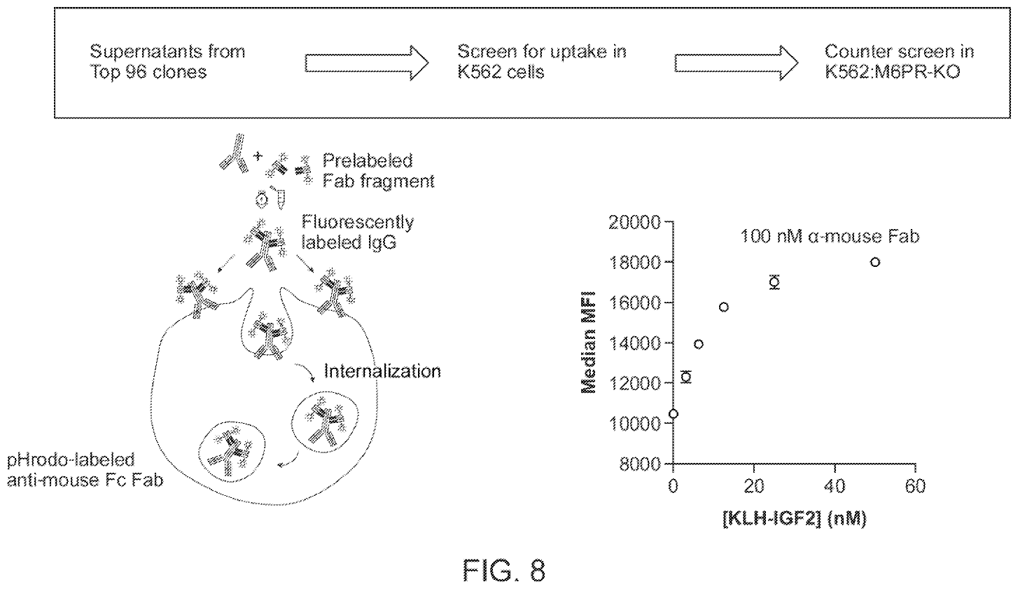

CI-M6PR binding antibodies were screened for uptake activity as shown in FIG. 8. Since CI-M6PR binds and internalizes IGF2, a non-binding antibody (KLH) fused to IGF2 was used as a positive control for uptake activity. K562 cells were seeded at 30,000 cells per well of a 96 well tissue culture plate in 100 ul RPMI (Gibco #61870143) with 10% v/v FBS (VWR #89510⁻¹⁸⁸), 2 mM L-alanyl-L-glutamine dipeptide, 100 units/ml penicillin and 100 μg/ml streptomycin (Gibco #15140148). Separately, 50 ul of CI-M6PR clonal hybridoma supernatant was mixed with 50 ul of Zenon pHrodo-iFL IgG labeling reagent (Thermo Fisher #Z25609) and incubated for 15 min at room temperature. The hybridoma supernatant/pHrodo mixture was layered on top of the K562 cells and incubated 18 hrs to allow uptake and lysosomal accumulation. Uptake was measured using a Novocyte Advanteon Flow Cytometer (Agilent Technologies) with the 488 nM laser and FITC detection configuration.

Table 14 below shows binding affinity and cellular internalization measured for various anti-CI-M6PR monoclonal antibodies according to the method described above.

TABLE 14
CI-M6PR monoclonal antibody characteristics

| | | Affinity | Uptake |
|---|---|---|---|
| Clone # | Clone ID | KD (M) | pHrodo (MFI) |
| 1 | 58D2 | 3.04E−09 | 12449 |
| 2 | 01A7 | 4.56E−12 | 11925 |
| 3 | 20D8 | 7.18E−09 | 9002 |
| 4 | 05C5 | 5.43E−09 | 77032 |
| 5 | 59G3 | 6.97E−09 | 10607 |
| 6 | 59F5 | 1.14E−09 | 10349 |
| 7 | 23F8 | 3.21E−10 | 18274 |
| 8 | 43E7 | 1.20E−08 | 16462 |
| 9 | 40D5 | 4.43E−09 | 15151 |
| 10 | 06D12 | 9.56E−09 | 46954 |
| 11 | 06D1 | 1.71E−08 | 22374 |
| 12 | 01E5 | 1.04E−08 | 53356 |
| 13 | 48C7 | 2.40E−08 | 11463 |
| 14 | 16H10 | 5.68E−09 | 12560 |
| 15 | 30H6 | 9.42E−09 | 14365 |
| 16 | 56F11 | 5.03E−09 | 18654 |
| 17 | 43A4 | 5.15E−09 | 11597 |
| 18 | 06H10 | 3.83E−08 | 65811 |
| 19 | 49D10 | 8.49E−10 | 11797 |
| 20 | 14A3 | 2.54E−08 | 42482 |
| 21 | 06C1 | 1.08E−08 | 16858 |
| 22 | 35E2 | 3.99E−09 | 27113 |
| 23 | 45A7 | 5.56E−09 | 12819 |
| 24 | 55B3 | 7.84E−09 | 11358 |
| 25 | 22D3 | 1.12E−09 | 18374 |
| 26 | 12D5 | 1.22E−08 | 35805 |
| 27 | 43D5 | 3.04E−08 | 13006 |
| 28 | 43C8 | 5.27E−09 | 30810 |
| 29 | 01B10 | 1.39E−08 | 12091 |
| 30 | 54D8 | 2.09E−08 | 8541 |
| 31 | 27D12 | 3.89E−10 | 13238 |
| 32 | 48G12 | 6.25E−10 | 10270 |
| 33 | 53B11 | 2.59E−09 | 9695 |
| 34 | 33H6 | 4.88E−09 | 9701 |
| 35 | 35B2 | 1.08E−08 | 17211 |
| 36 | 45A12 | 3.00E−08 | 35990 |
| 37 | 29B3 | 5.19E−09 | 13363 |
| 38 | 48D3 | 3.32E−09 | 17808 |
| 39 | 38E12 | 5.72E−09 | 10617 |
| 40 | 07H3 | 3.25E−08 | 11087 |
| 41 | 54G12 | 2.18E−09 | 9918 |
| 42 | 44F2 | 5.66E−09 | 11045 |
| 43 | 34F9 | 3.35E−08 | 10057 |
| 44 | 13H3 | 3.47E−08 | 12070 |
| 45 | 18G4 | 8.74E−09 | 10412 |
| 46 | 06G11 | 2.42E−08 | 29022 |
| 47 | 44A8 | 4.79E−08 | 14462 |
| 48 | 37A10 | 5.22E−08 | 17130 |
| 49 | 08G8 | 1.78E−08 | 15178 |
| 50 | 22D11 | 5.64E−09 | 9636 |
| 51 | 56E1 | 3.04E−08 | 18705 |
| 52 | 04F7 | 4.05E−08 | 14719 |
| 53 | 33G7 | 9.12E−09 | 10506 |
| 54 | 13E6 | 2.72E−08 | 13533 |
| 55 | 17F11 | 1.05E−08 | 12508 |
| 56 | 22D8 | 1.01E−08 | 12438 |
| 57 | 09A2 | 9.20E−09 | 10968 |
| 58 | 11H7 | 9.04E−08 | 15842 |
| 59 | 10H5 | 2.15E−08 | 15809 |
| 60 | 03D6 | 7.31E−08 | 12025 |
| 61 | 38H4 | 4.72E−08 | 9631 |
| 62 | 26H1 | 7.66E−08 | 9778 |
| 63 | 06A7 | 1.04E−07 | 14151 |
| 64 | 10C8 | 3.61E−08 | 18946 |
| 65 | 03C10 | 8.11E−08 | 11435 |
| 66 | 03C12 | 2.37E−09 | 250356 |
| 67 | 06F1 | 1.73E−08 | 34189 |
| 68 | 06A8 | 2.17E−07 | 9969 |
| 69 | 23D1 | 1.02E−08 | 24870 |
| 70 | 08A11 | 5.45E−08 | 14672 |
| 71 | 20E12 | 7.61E−08 | 14766 |
| 72 | 09F4 | 4.98E−08 | 9637 |
| 73 | 15F3 | 8.18E−08 | 14037 |
| 74 | 05E7 | 1.08E−07 | 11730 |
| 75 | 11B12 | 9.06E−08 | 42985 |
| 76 | 42G9 | 3.11E−09 | 23624 |
| 77 | 14H5 | 1.63E−07 | 11351 |
| 78 | 21D5 | 4.31E−08 | 9985 |
| 79 | 07G8 | 3.86E−08 | 12945 |
| 80 | 29A3 | 8.00E−08 | 11595 |
| 81 | 09H1 | 2.33E−09 | 17499 |
| 82 | 02E3 | 3.09E−08 | 10142 |
| 83 | 04G9 | 6.48E−08 | 13773 |
| 84 | 08D8 | 2.59E−07 | 10949 |
| 85 | 03C7 | 1.75E−08 | 41682 |
| 86 | 56A2 | 4.12E−12 | 12672 |
| 87 | 22A1 | 1.36E−07 | 9877 |
| 88 | 02C7 | 5.50E−09 | 15479 |
| 89 | 12A5 | 8.06E−09 | 9664 |
| 90 | 07A4 | 1.40E−06 | 10106 |
| 91 | 21E12 | 1.26E−11 | 10442 |
| 92 | 09E5 | 9.40E−08 | 9735 |
| 93 | 14D2 | 2.02E−06 | 11324 |
| 94 | 28B8 | 6.39E−09 | 17584 |
| 95 | 16H4 | 6.15E−07 | 13427 |
| 96 | 15F2 | 5.09E−09 | 12871 |
| Negative | No | N/A | 13019 |
| | Antibody | | |
| Positive | KLH- | N/A | 209856 |
| | IGF2 | | |

### 6.15.3. Example 3: Anti-MP6PR Antibodies Bind Human M6PR

31 of the 96 anti-M6PR antibodies identified in Example 2 were isolated, cloned, and recombinantly expressed as monoclonal antibodies.

### Binding Affinity and Kinetics

Each of the 31 antibodies was assessed for its ability to bind CI-M6PR. Each of the anti-M6PR antibodies that bound CI-M6PR was also assessed for binding affinity and kinetics.

Anti-M6PR antibodies binding to human CI-M6PR were measured by biolayer interferometry (BLI) using a GatorPrime instrument. Anti-M6PR antibodies were prepared in PBS buffer supplemented with 0.002% Tween-20, 0.02% BSA, and 0.005% NaN3 and immobilized on sensors coated with Anti-Mouse IgG Fc antibodies. Immobilized anti-M6PR antibodies were then incubated with varying concentrations of purified recombinant CI-M6PR (domains 1-9)protein prepared in the same buffer. During the experiment, the reaction plate was maintained at 30° C. and shaken at 1,000 rpm. Data were fit using the GatorOne Software v2.10, where response curves were aligned at the association step. Processed association and dissociation curves were fit globally using a 1:1 binding model to obtain kinetic constants for each antibody.

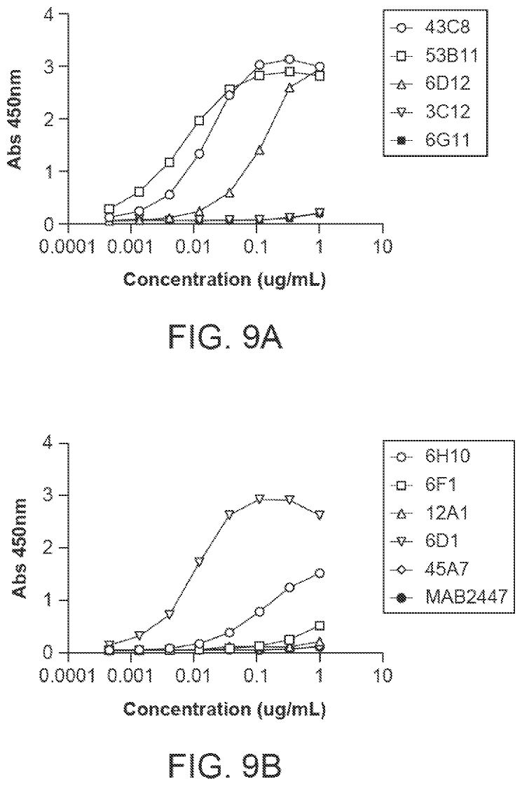

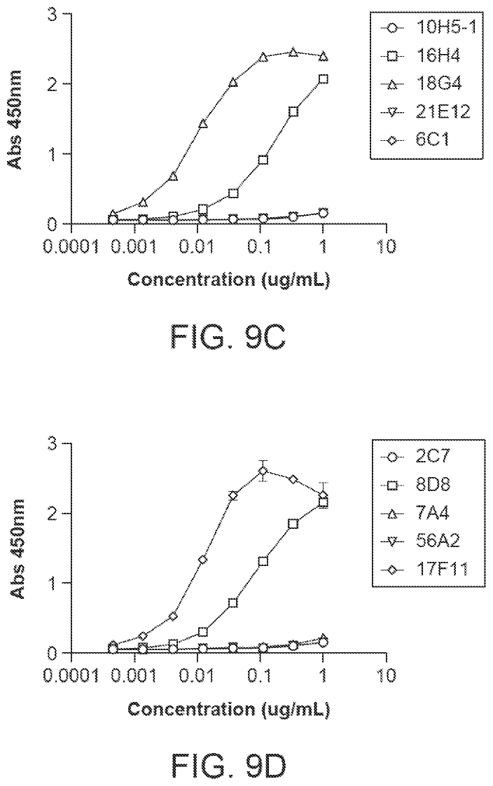

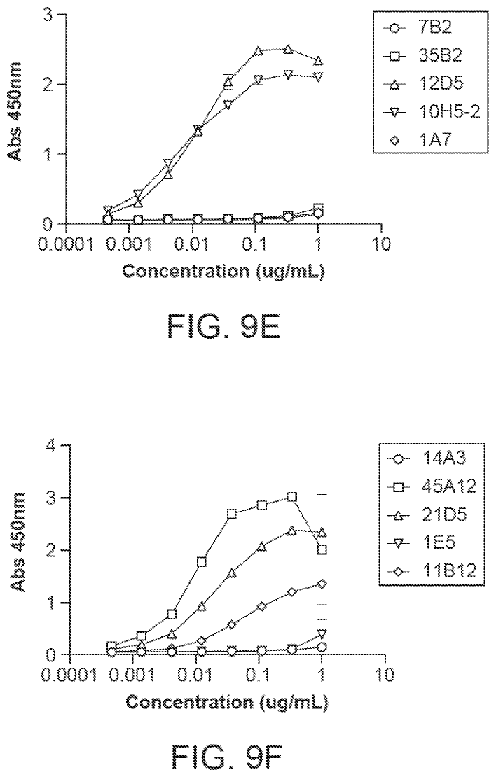

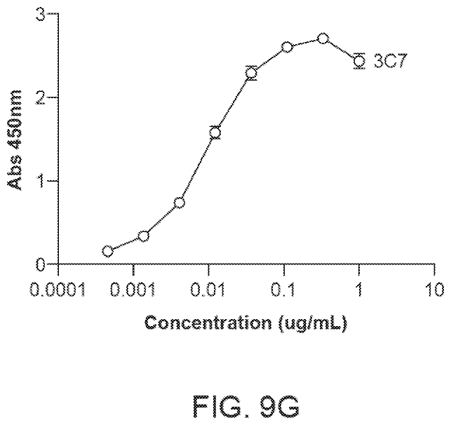

Results of ELISAs in FIGS. 9A-G show that 16 of the 31 anti-M6PR antibodies bound to human M6PR.

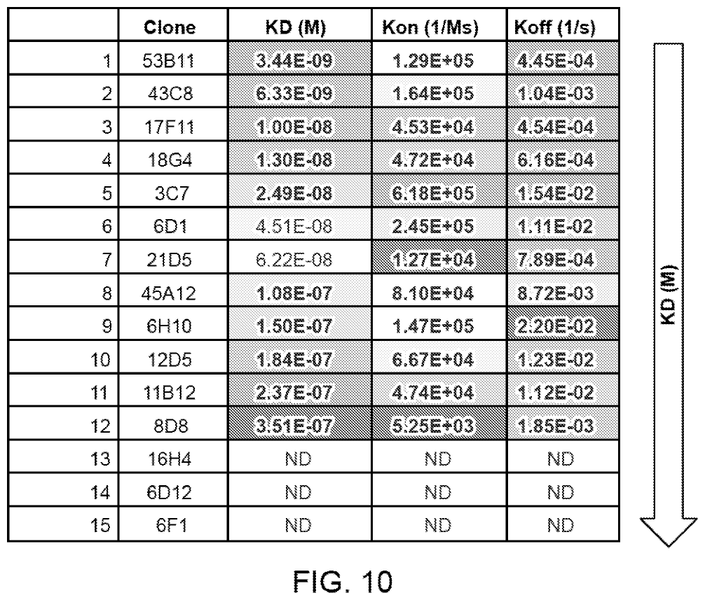

Results shown in FIG. 10 demonstrate that the top 15 anti-M6PR antibodies exhibit a range of affinities (K_{D}) and kinetics (Kₒₙ, K_{off}).

### Epitope Binning and Domain Mapping

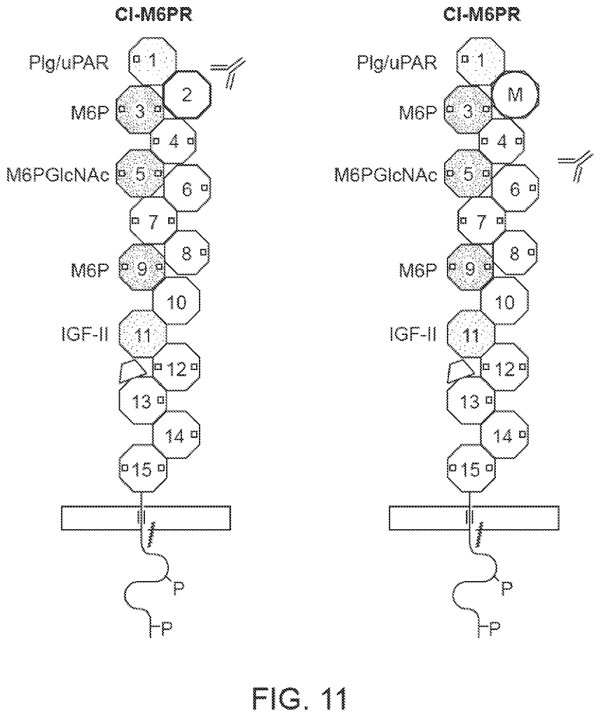

Each of the 16 anti-M6PR antibodies identified as binding to human CI-M6PR (FIGS. 9A-9G) were assessed by epitope binning and domain mapping to determine where each antibody binds on CI-M6PR (FIG. 11).

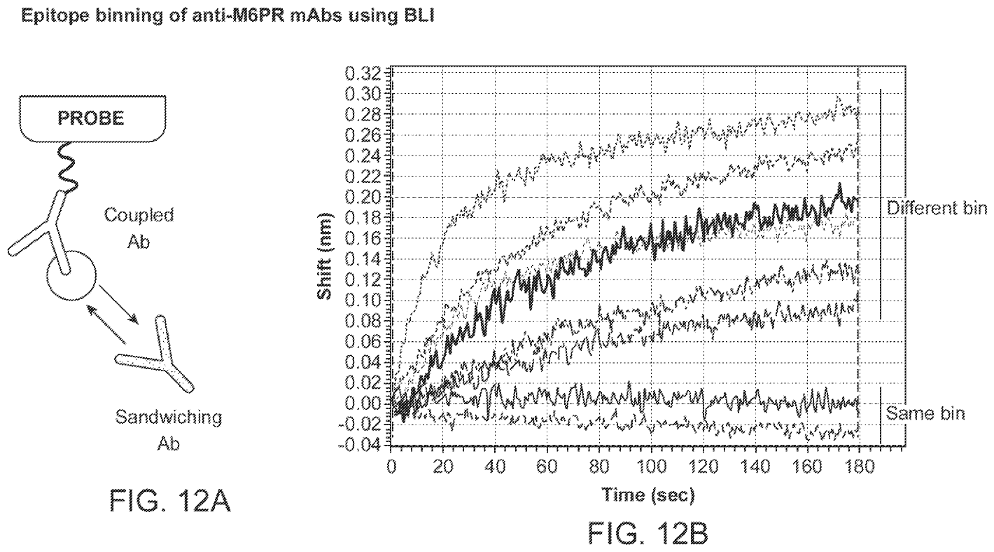

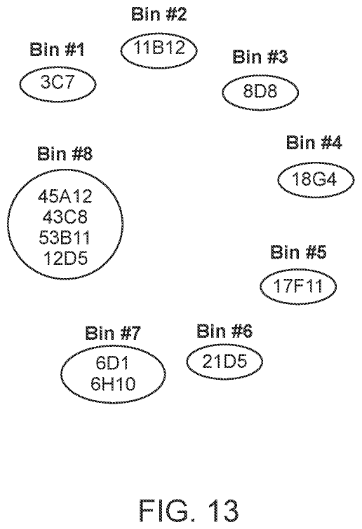

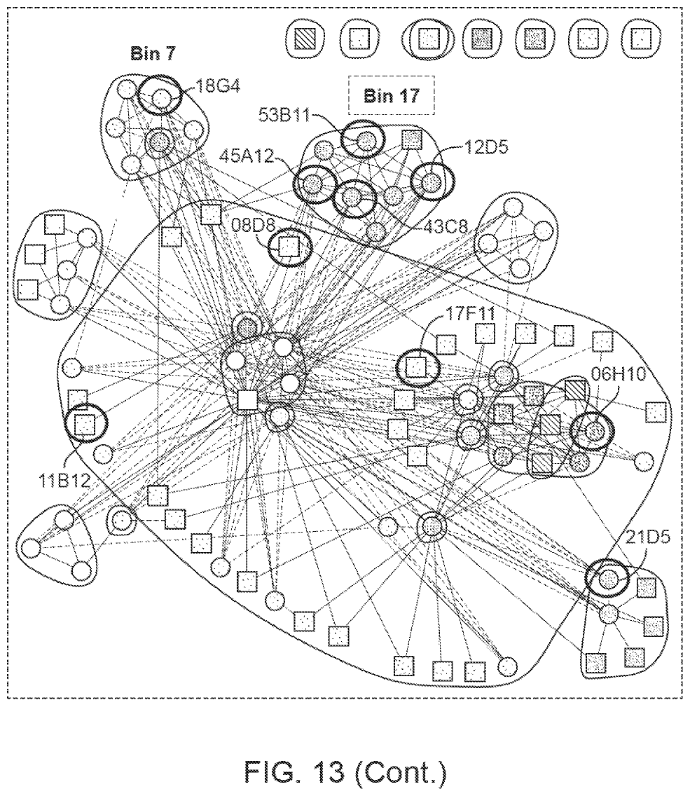

To determine epitope bins, anti-M6PR antibodies and CI-M6PR protein were immobilized onto anti-Mouse IgG Fc sensors according to the method described above. These probes were then incubated with a second anti-M6PR antibody. Antibodies that led to any further increases in signal as detected by the sensors were determined to be in a separate epitope bin from the antibody already immobilized on the sensor. Antibodies that led to a decrease in signal were determined to be in the same epitope bin as the antibody already immobilized on the sensor. See FIG. 12A for a non-limiting illustration of the assay and FIG. 12B for an example plot showing signal shift for different epitope bins. As summarized in FIG. 13, 12 of the preferred anti-M6PR antibodies represent 8 separate epitope bins.

In order to perform domain mapping of the anti-M6PR antibodies, a series of chimeric human/mouse CI-M6PR constructs were cloned and purified. Each of the 9 domains (D1-D9) of human CI-M6PR was individually replaced with the equivalent domain from mouse CI-M6PR for all 9 domains. Each chimeric construct was N-terminally tagged with a HIS-tag.

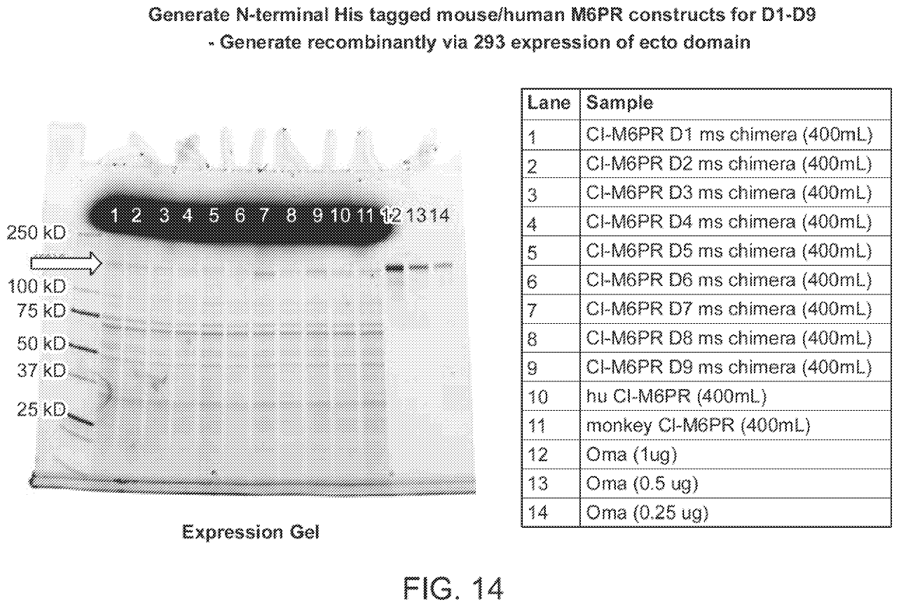

Each of the 9 HIS-tagged CI-M6PR domains were then generated recombinantly via HEK293 expression of the ecto domain. FIG. 14 shows expression of each of the HIS-tagged D1-D9 CI-M6PR domains. Recombinantly expressed HIS-tagged D1-D9 CI-M6PR domains were purified by harvesting the HEK293 media and using HisTrap affinity columns. All domains were purified as non-aggregated monovalent entities of approximately 200 kDa. Recovery was around 1-2 mg/mL transient.

BLI experiments were performed to test the binding of each anti-M6PR antibody to each chimeric CI-M6PR protein according to the method described above. Wild-type human CI-M6PR was used as a positive control. If the replacement of a specific domain of human CI-M6PR with that of mouse CI-M6PR resulted in a loss of binding, that domain was identified as the domain of human CI-M6PR to which the given antibody binds.

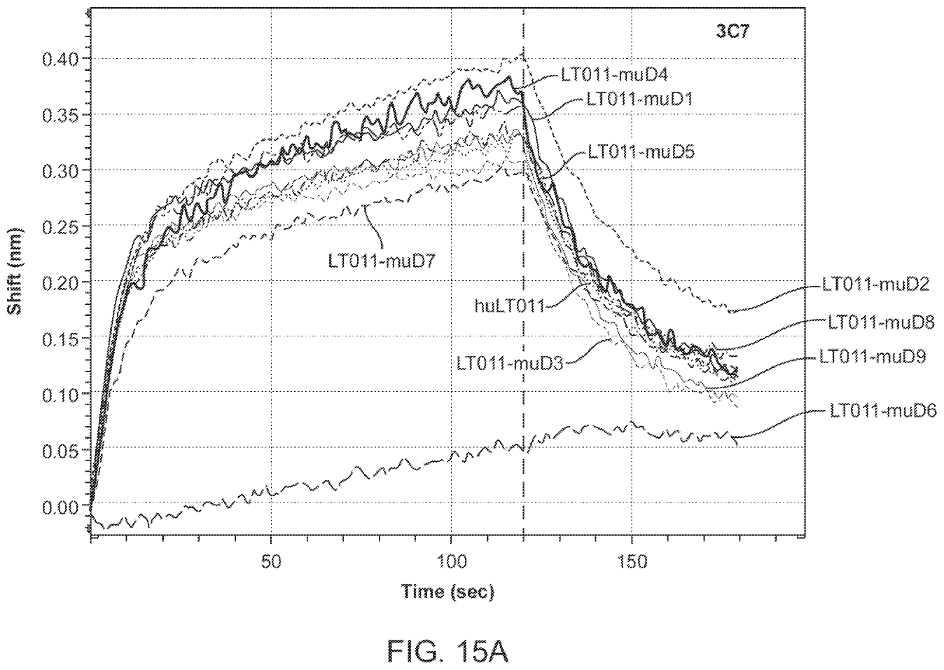

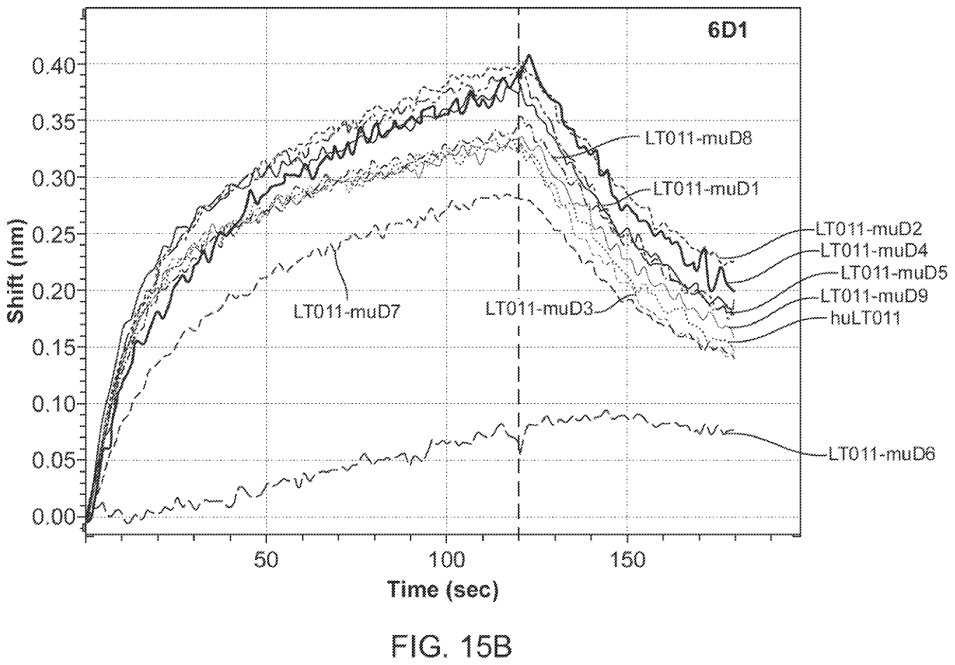

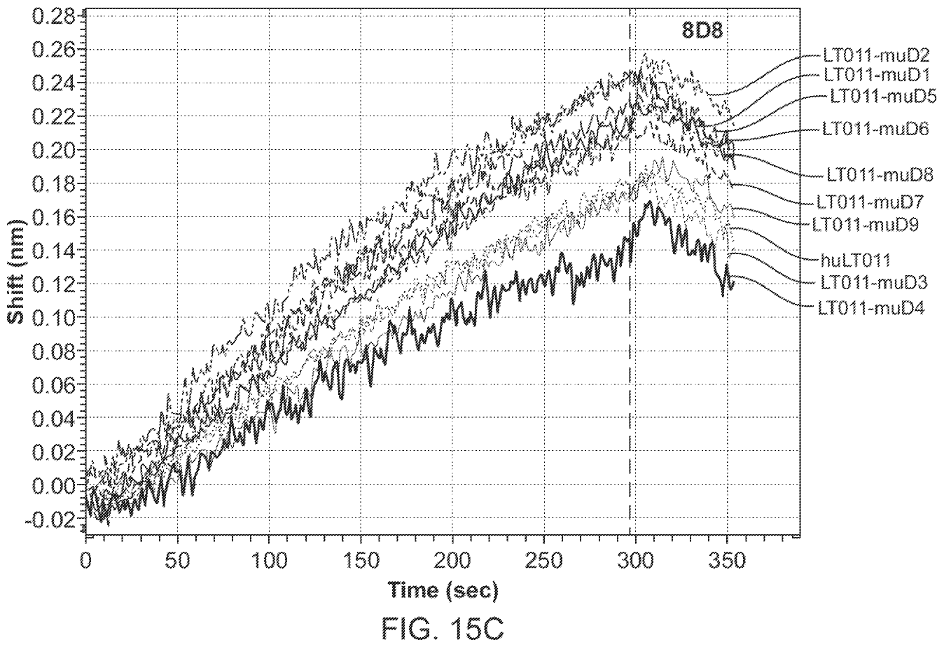

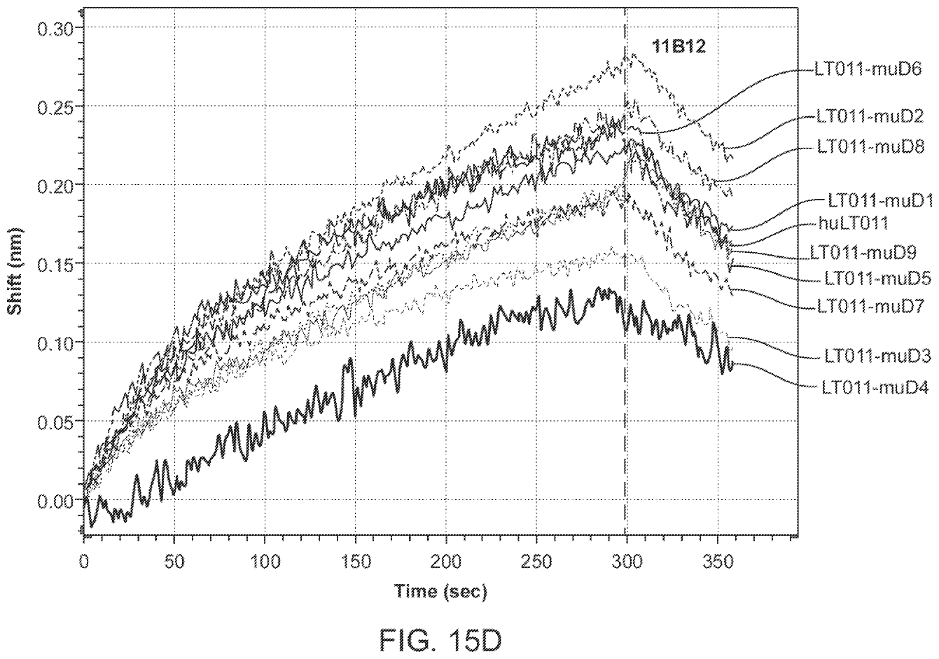

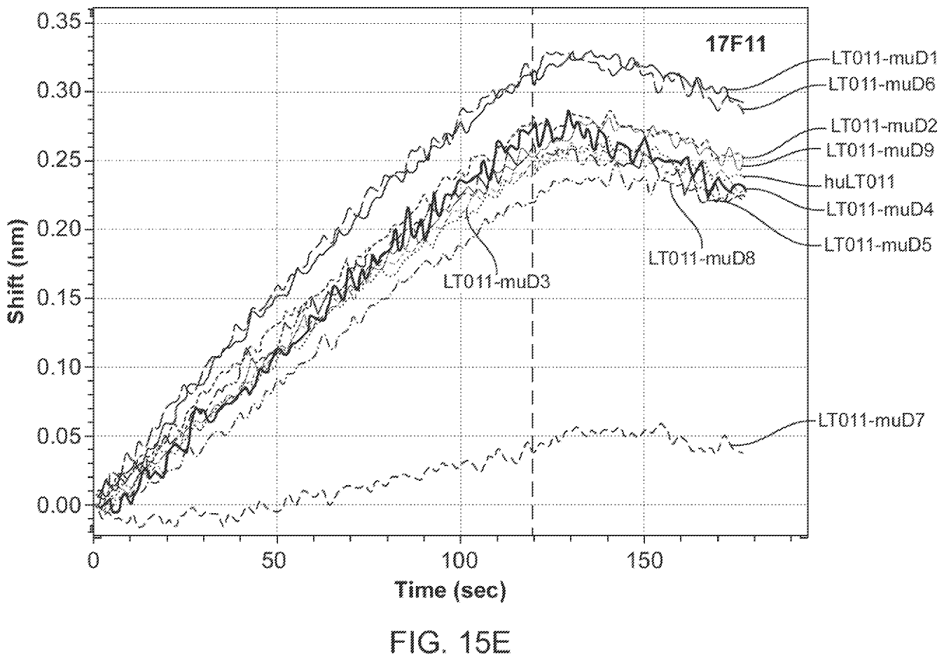

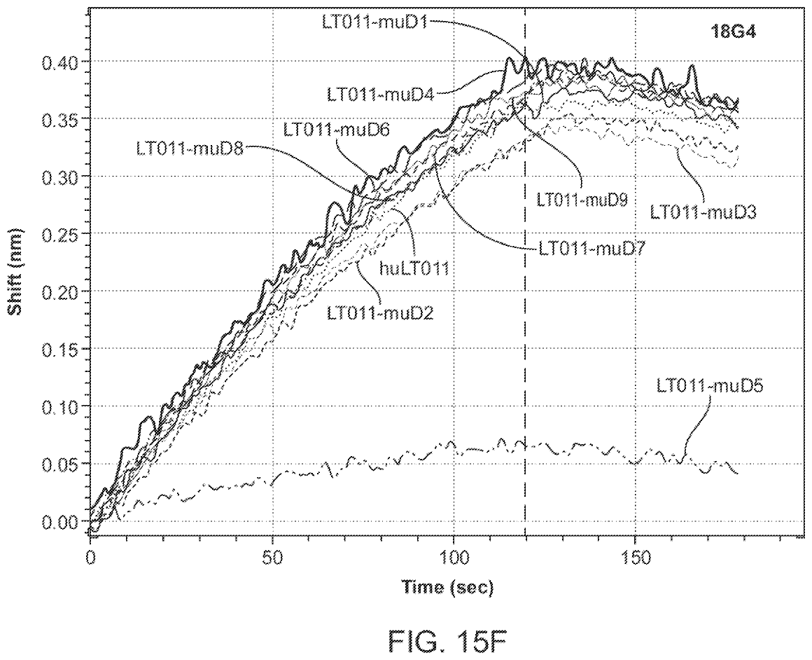

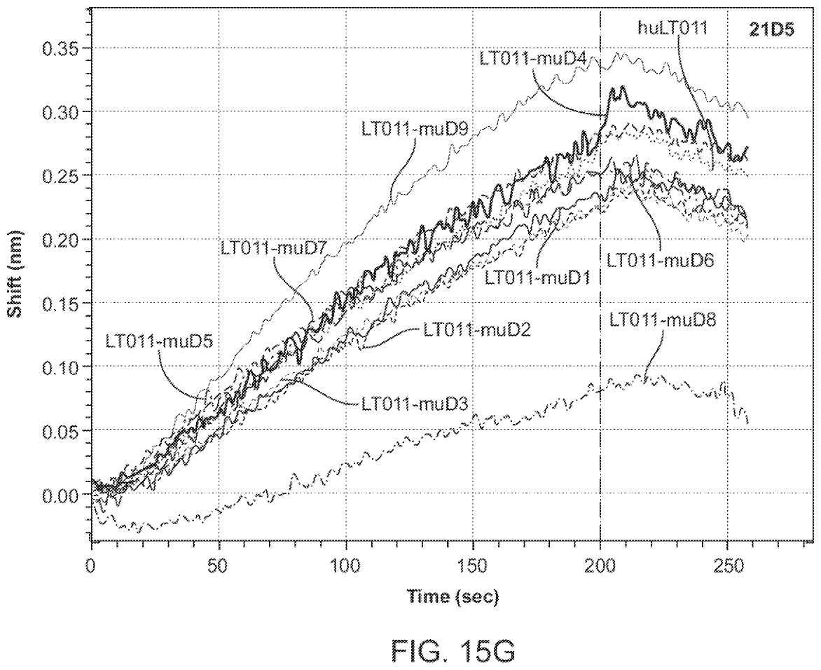

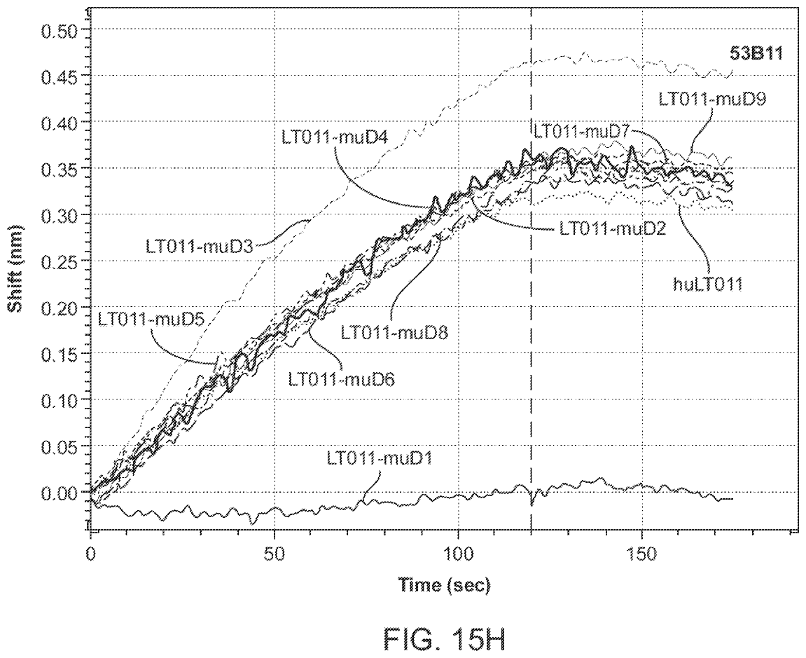

As shown in FIGS. 15A-B and FIGS. 15D-H, anti-M6PR antibodies domains were mapped for clones 3C7, 6D1, 11B12, 17F11, 18G4, 21D5, and 53B11. Clone 8D8 (FIG. 15C) showed similar binding to each of the chimeric CI-M6PR proteins, which meant the domain of human CI-M6PR to which the clone 8D8 antibody binds could not be determined using this assay.

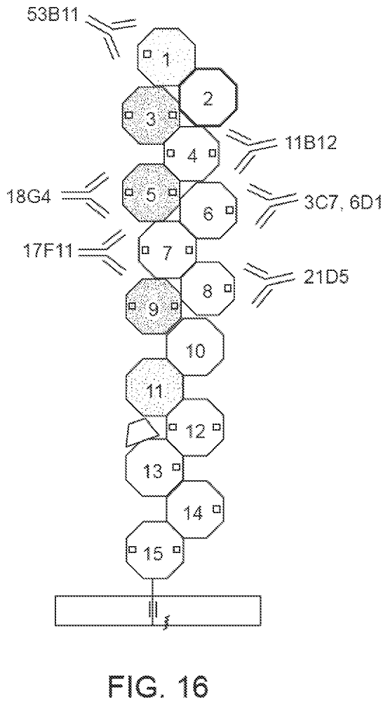

A schematic summarizing the domain mapping results for anti-M6PR antibody clones 3C7, 6D1, 11B12, 17F11, 18G4, 21D5, and 53B11 is shown in FIG. 16. For example, 53B11 binds to domain 1 on CI-M6PR, 11B12 binds to domain 4 on CI-M6PR, 18G4 binds to domain 5 on CI-M6PR, 3C7 and 6D1 each bind to domain 6 on CI-M6PR, 17F11 binds to domain 7 on CI-M6PR, and 21D5 binds to domain 8 on CI-M6PR.

### 6.15.4. Example 4: Evaluating pH Dependent Dissociation of Anti-M6PR Ab from CI-M6PR

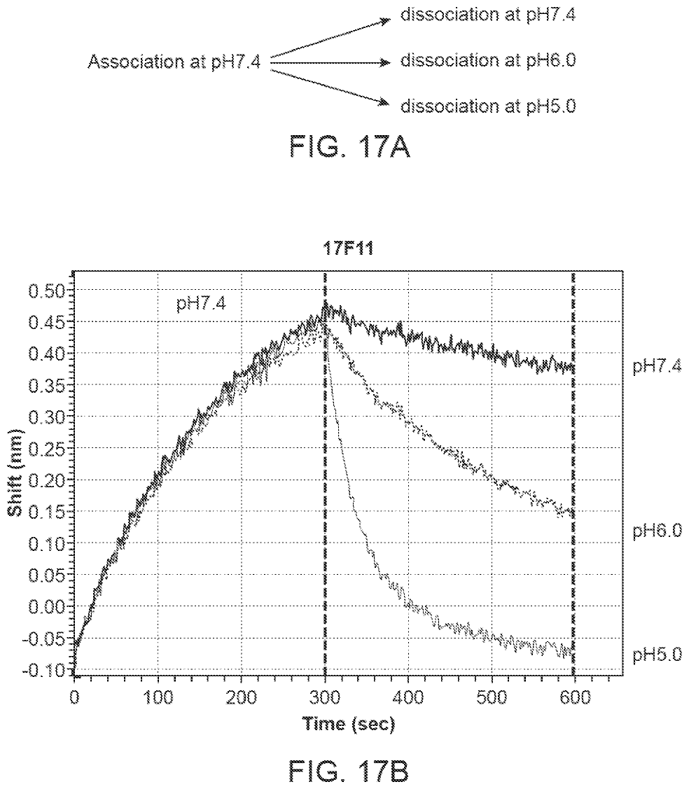

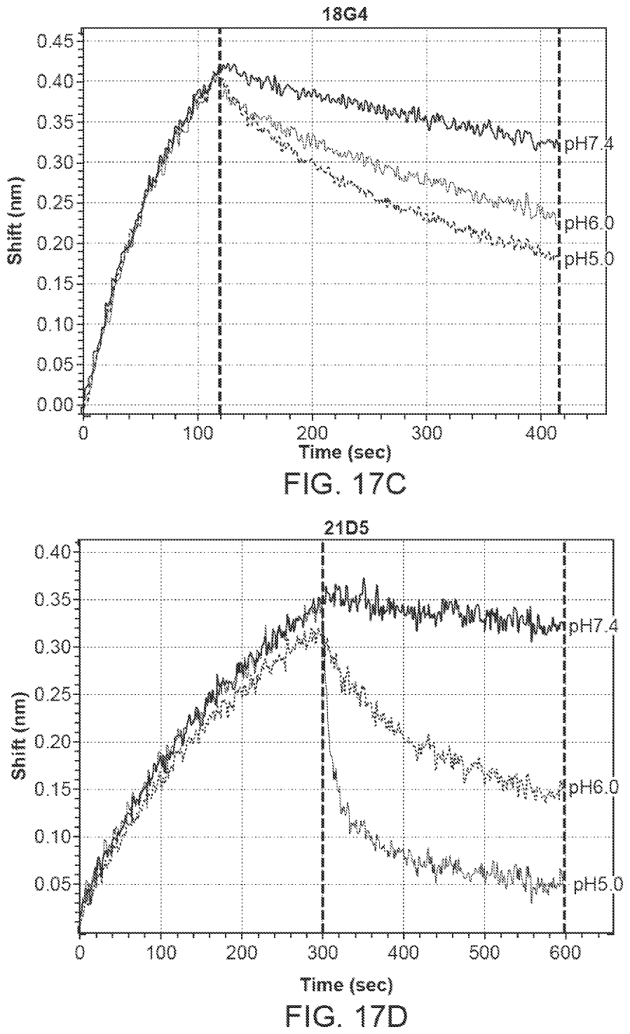

Anti-M6PR antibodies were immobilized onto anti-Mouse IgG1 Fc coated sensor according to the method described above and incubated with human CI-M6PR proteins prepared in citric acid buffer pH 7.4. For dissociation, the sensors were transferred to wells containing citric acid buffer at pH 7.4, pH 6.0, or pH 5.0 (see FIG. 17A). FIGS. 17B-17D show binding curves for clones 17F11 (FIG. 17B), 18G4 (FIG. 17C), and 21D5 (FIG. 17D) with clones 17F11 and 21D5 showing stronger pH dependent disassociation than for clone 18G4.

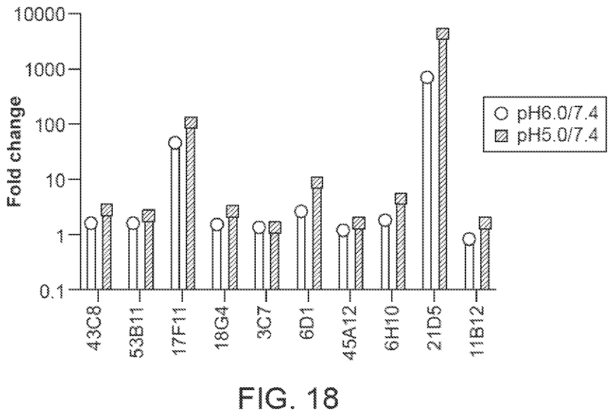

To quantify pH-dependent dissociation the calculated K_{off} of each antibody at pH 5.0 and pH 6.0 was divided by that at pH 7.4 to determine the fold increase of the off-rate for each antibody at the indicated pH. FIG. 18 shows that disassociation rates of antibody clones 17F11 and 21D5 are increased at low pH (pH 5.0).

### 6.15.5. Example 5: Cellular Uptake of Anti-M6PR Antibodies

### pH Rodo Uptake Assay for 31 Anti-M6PR Antibodies

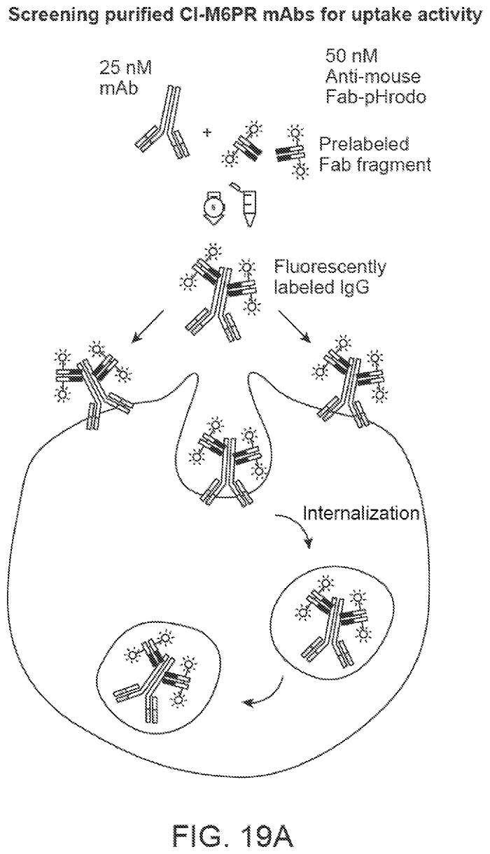

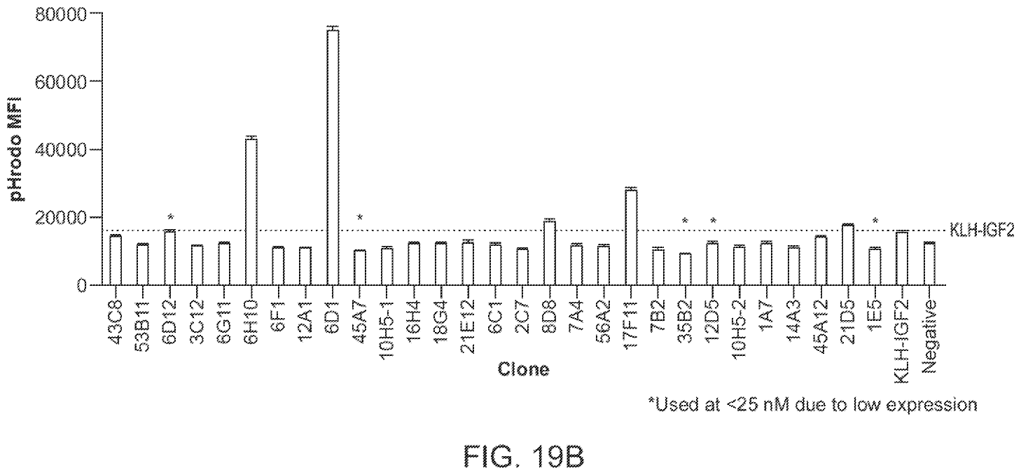

Recombinantly expressed anti-M6PR antibodies were assessed for uptake activity using a pH rodo uptake assay (FIG. 19A). In particular, mAb and Zenon pHrodo green Fab were precomplexed at 50 nM mAb and 100 nM Zenono pHrodo green iFL (2×final concentrations) in 100 ul RPMI+10% Fetal Bovine Serum (FBS) and 1×penicillin/streptomycin (pen/strep) for 30 minutes at room temperature. 100 ul of each mAb: Zenon pHrodo Fab was added to 100 ul RPMI+10% FBS and 1×pen/strep containing 100,000 K562 cells. Plates were incubated for 24 hours at 37° C. with 5% CO2. Cells were pelleted and washed 1× with FACS wash buffer (PBS+1% Bovine Serum Albumin (BSA)). Anti-M6PR antibody uptake was assayed by flow cytometry measuring the pHrodo green signal in the FITC channel.

As CI-M6PR binds and internalizes IGF2, a non-binding antibody (KLH) fused to IGF2 was used as a positive control for uptake activity. Antibodies were identified that demonstrated uptake activity greater than the KLH-IGF2 positive control. As shown in FIG. 19B, antibody clones 6D12, 6H10, 6D1, 17F11, and 21D5 show greater uptake then KLH-IGF2.

### Assessing Anti-M6PR Anti-IgE Bispecific Antibodies for Uptake

A subset of anti-M6PR antibodies were assessed for uptake activity using an IgE-pH rodo uptake assay. In particular, M6PR-Oma bispecific antibodies (BsAb) (FIG. 2A) and IgE fluorescently labeled with Alexa Fluor 488 or Alexa Fluor 647 (IgE-AF488 or IgE-AF647) were precomplexed at 200 nM (2× final) each in RPMI+10% FBS and 1× pen/strep for 30 mins at room temperature. A 6× log 3 serial dilution was performed and 100 ul of each BsAb: IgE dilution was added to 100 ul RPMI+10% FBS and 1×pen/strep containing 100,000 K562 cells. Plates were incubated for 24 hours at 37° C. with 5% CO2. Cells were pelleted and washed 1× with FACS wash buffer (PBS+1% BSA). Anti-M6PR antibody uptake was assayed by flow cytometry measuring signal from AF488 and/or AF647.

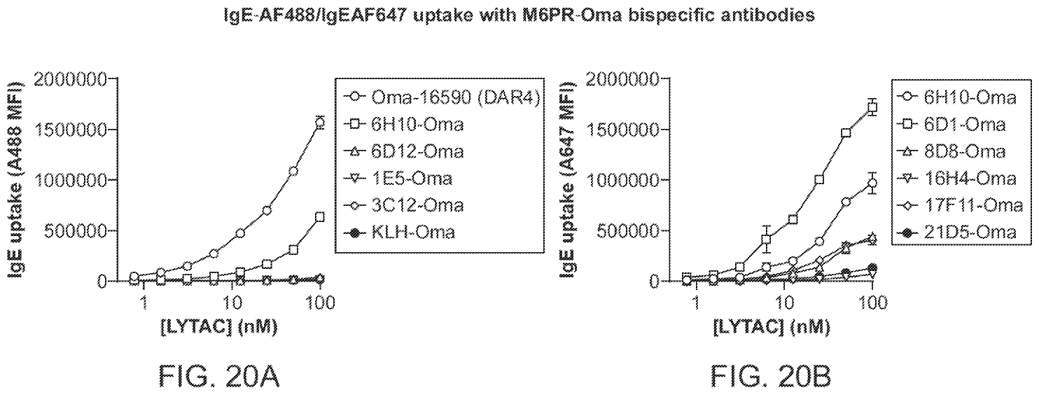

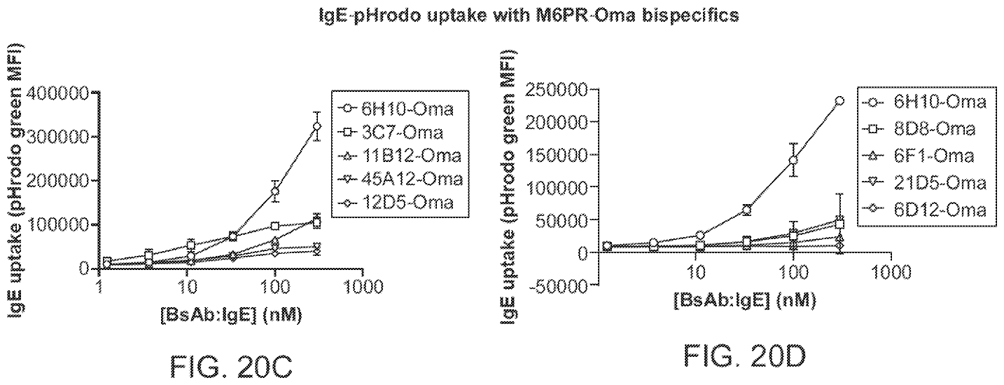

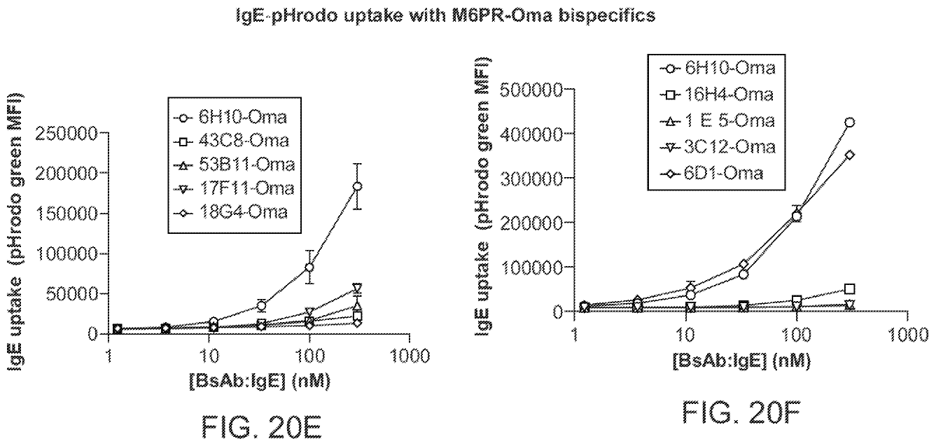

A non-binding antibody (KLH) fused to Omalizumab (Oma) was used as a negative control for uptake activity. An Oma-16590 (Dar4)bispecific antibody was used as a positive control for uptake activity. FIGS. 20A-20F show uptake for anti-M6PR-Oma bispecific antibodies. Results shown in FIG. 20A and FIG. 20B demonstrate that 6H10-Oma, 6D1-Oma, 8D8-Oma, and 17F11-Oma transport fluorescently labeled IgE (IgE-AF488 or IgE-AF647) into cells. Results shown in FIGS. 20C-20F demonstrate that 6H10-Oma and 6D1-Oma internalize pHrodo labeled IgE (IgE-phrodo) into acidic compartments while other M6PR-Oma bispecifics do not. In particular, bispecific antibodies including 6D1-oma, 8D8-oma, 17F11-oma, and 6H10-oma showed uptake of IgE.

### Assessing Anti-M6PR Anti-(′5 Bispecific Antibodies for (′5 Internalization and Uptake

To assess whether the anti-M6PR antibodies were capable of internalizing C5 as a target, bispecific antibodies with one anti-M6PR binding arm and one anti-C5 binding arm (Eculizumab “ECU”) were generated and assayed for uptake activity using C5 labeled with pHrodo green.

HeLa cells were plated at 15,000 cells per well in a 96 well plate and incubated overnight. Anti-M6PR/anti-C5 bispecific antibodies were added at 100 nM in 100 μL complete growth medium (DMEM+10% FBS). In a separate 96 well plate, C5 labeled with pHrodo green was added to growth media at 500 nM in 100 μL complete growth medium and a 2 fold serial dilution was performed. Serially diluted C5 was then combined with the anti-M6PR/anti-C5 bispecific (53B11-ECU, 43C8-ECU, 12D5-ECU, 18G4-ECU) in 200 μL final volume, added to HeLa cells, and incubated for 18 hours at 37° C. Internalization of C5 to the lysosome was measured by flow cytometry. Data was collected in the APC channel and mean fluorescent intensities were calculated.

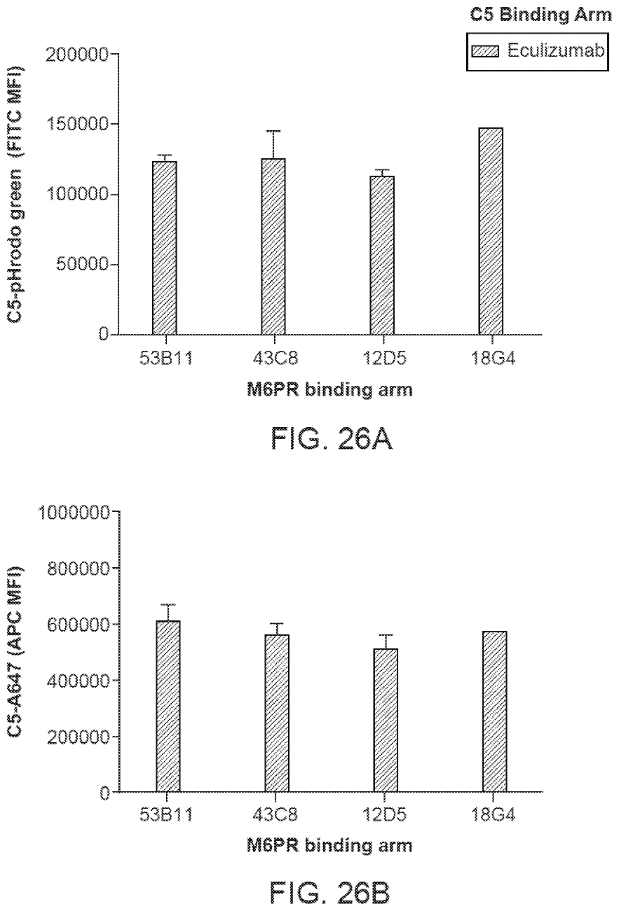

Results shown in FIG. 26A (pH rodo) and FIG. 26B (AF647) demonstrate that anti-M6PR/antiC5 bispecific antibodies (53B11-ECU, 43C8-ECU, 12D5-ECU, 18G4-ECU) transport fluorescently labeled C5 (C5-AF488 or C5-AF647) into cells.

### 6.15.6. Example 6: Assessing Internalization and Degradation of Anti-M6PR Antibodies

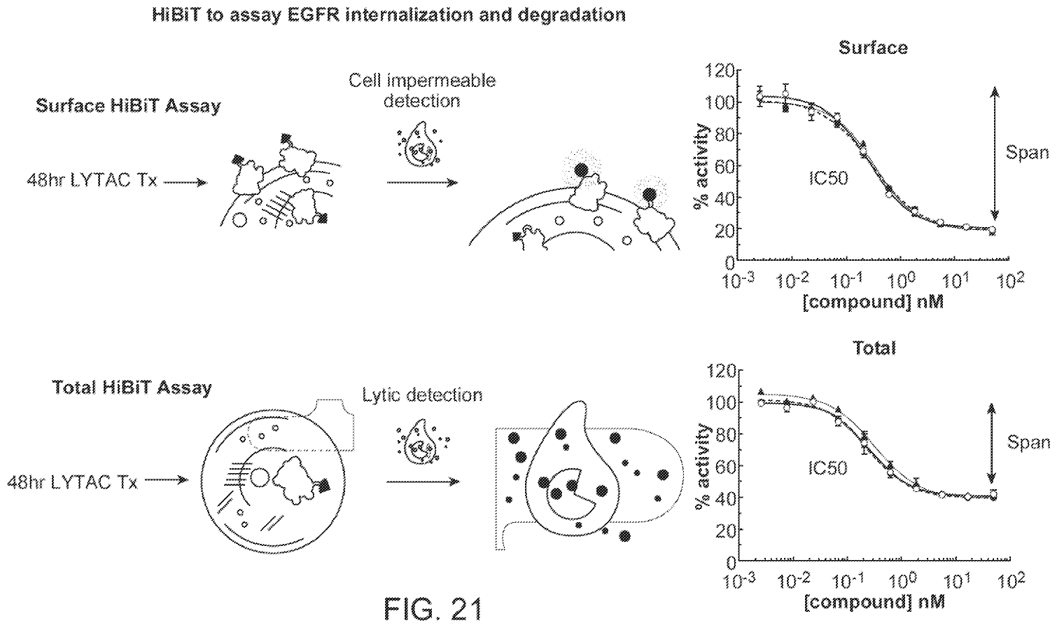

Cell surface depletion and total depletion of HiBiT-tagged protein of interest were measured using Nano-Glo HiBiT Extracellular Detection System (Promega, Cat. No. N2422) and Nano-Glo HiBiT Lytic Detection System (Promega, Cat. No. N3050), respectively. FIG. 21 provides a schematic of the surface HiBit assay and total HiBit assay where Span indicates the degree of depletion of the target (e.g., EGFR).

### HiBIT Assay Protocol

Briefly, HCT116 cells expressing HiBiT-tagged EGFR were plated in white, tissue culture-treated 96 well plates at 5,000 cells per well in 100 μL and grown for 24 hours at 37° C. Matuzumab-antiM6PR bispecific antibodies were diluted in media to prepare 3-fold dilution series from 40-0.002 nM (2× final concentrations) and added to cells in 100 μL per well, along with media-only control.

An EGFR ligand, EGF, was used as a positive control, assayed in 3-fold dilution series from 100-0.005 nM (2× final concentrations). Following antibody or control treatment, cells were grown for 48 hours at 37° C. and media was replaced with 50 μL fresh media and 50 μL either extracellular or lytic HiBiT detection reagent prepared according to manufacturer's instructions. Briefly, LgBiT protein and Nano-Glo HiBiT substrate were diluted at 1:100 and 1:50 ratios respectively, in corresponding Nano-Glo HiBiT buffer. Following three minutes shaking and 10 minutes incubation at ambient temperature, HiBiT signal was detected by measuring luminescence on an Envision plate reader (Perkin Elmer). Dose responses were conducted in duplicate and normalized to media-only control. IC50 values were determined by fitting to 4-parameter curves in GraphPad Prism, and span was calculated from the difference between the first and last points on the curve.

### Results

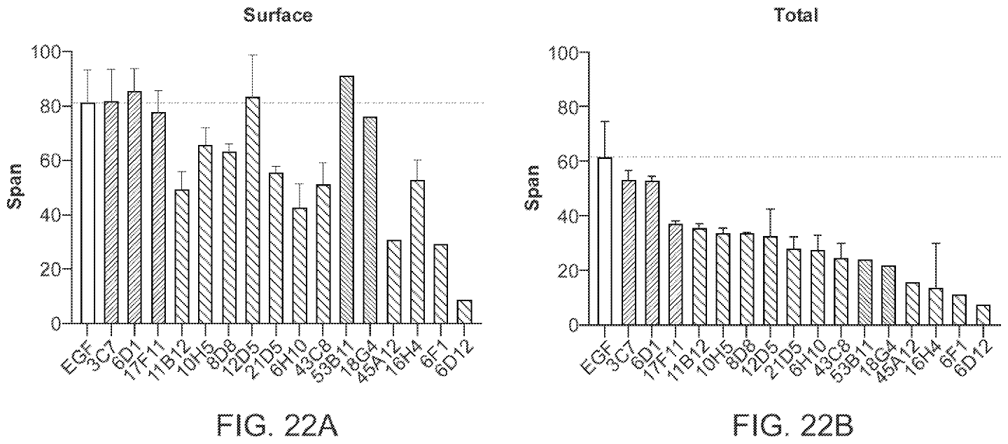

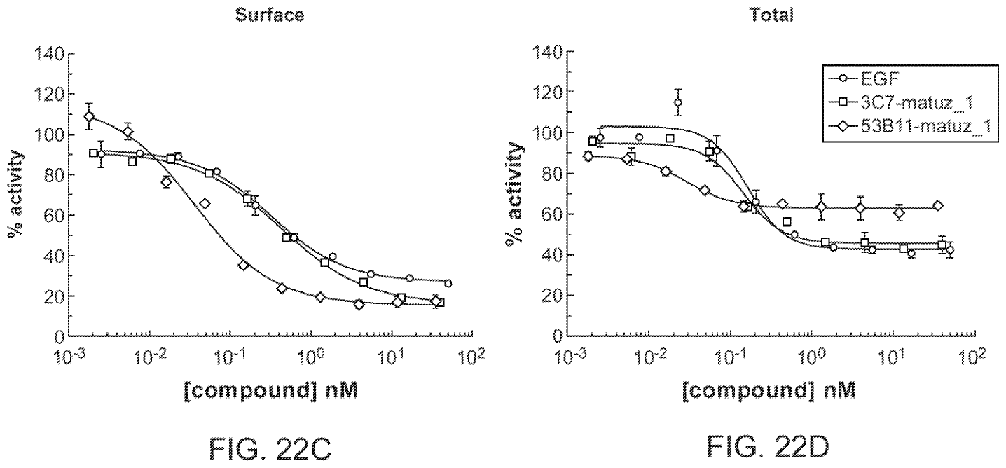

FIG. 22A shows surface depletion (i.e., span) of EGFR for 16 matuzumab-antiM6PR bispecific antibodies. FIG. 22B shows total depletion (i.e., span) of EGFR for 16 matuzumab-antiM6PR bispecific antibodies. FIG. 22C shows percentage of surface EGFR activity of exemplary matuzumab-antiM6PR bispecific antibodies (3C7-matuz, 53B11-matuz). FIG. 22D shows percentage of total EGFR activity of exemplary matuzumab-antiM6PR bispecific antibodies (3C7-matuz, 53B11-matuz). Taken together, this data showed that anti-M6PR antibody clones 3C7, 6D1, and 17F11 are similar to EGF (positive control) in both EGFR internalization and degradation, with sub nanomolar potency.

### 6.15.7. Example 7: Assessing Recycling of Anti-M6PR Antibodies

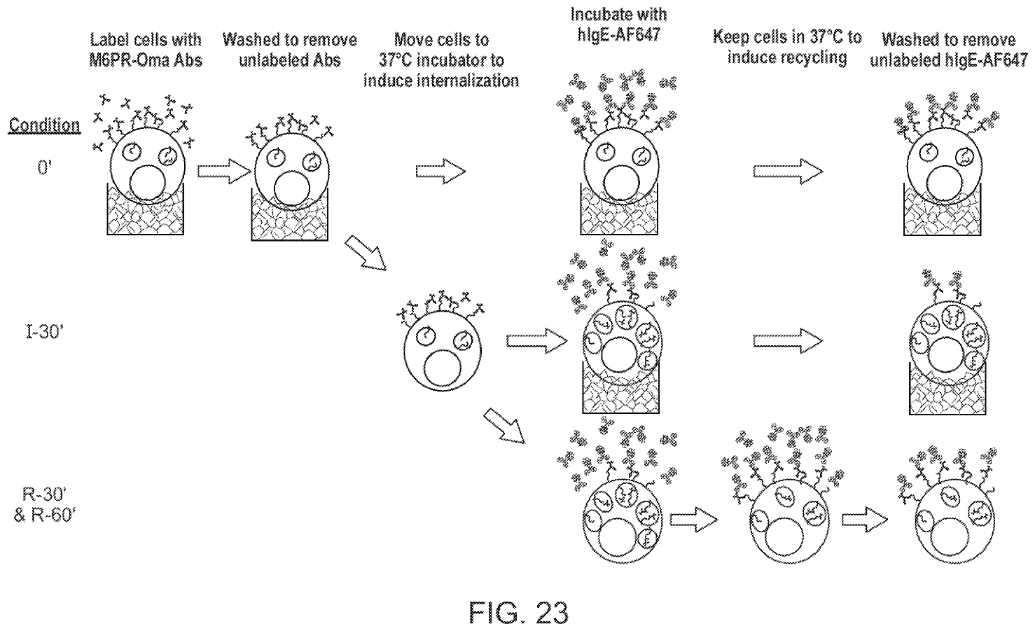

Recycling of anti-M6PR-omalizumab (M6PR-Oma)bispecific antibodies was assessed following internalization using a hlgE uptake assay (see, e.g., FIG. 23). In particular, M6PR-Oma bispecific antibodies were diluted in RPMI+10% FBS to a 2× final concentration. 100 ul of M6PR-Oma bispecific antibodies were added to 100 ul RPMI+10% FBS and 1×pen/strep containing 100,000 K562 cells or 100,000 HepG2 cells. Cells were contacted with M6PR-Oma bispecific antibodies, thereby labeling the cells with the bispecific antibodies. After labeling, cells were washed to remove unbound (unlabeled) M6PR-Oma bispecific antibodies. Cells were then split into the four conditions as indicated in FIG. 23: 0′; I-30′, R-30′, and R-60′.

For 0′ condition, cells were incubated with hlgE-Alexa Fluor (AF) 647 (“hIgE-AF647”) without first allowing the M6PR-Oma bispecific antibodies to internalize. The 0′ control cells were then washed and assessed for the presence of M6PR-Oma bispecific antibodies at the surface of the cells using flow cytometry to measure AF647.

For conditions I-30′, R-30′, and R-60′, cells were incubated at 37° C. for 30 minutes to allow internalization of the M6PR-Oma bispecific antibodies. Following the 30 minute incubation, cells were exposed to hIgE-AF647. Cells in the I-30′ condition were washed to remove unlabeled hlgE-AF647 and assessed for the presence of M6PR-Oma bispecific antibodies at the surface of the cells using flow cytometry to measure AF647. Cells in the R-30′ and R-60′ were maintained at 37° C. for the remainder of the designated incubation period to induce recycling. Cells in the R-30′ and R-60′ were then washed to remove unlabeled hIgE-AF647 and assessed for presence of M6PR-Oma bispecific antibodies at the surface of the cells using flow cytometry to measure AF647.

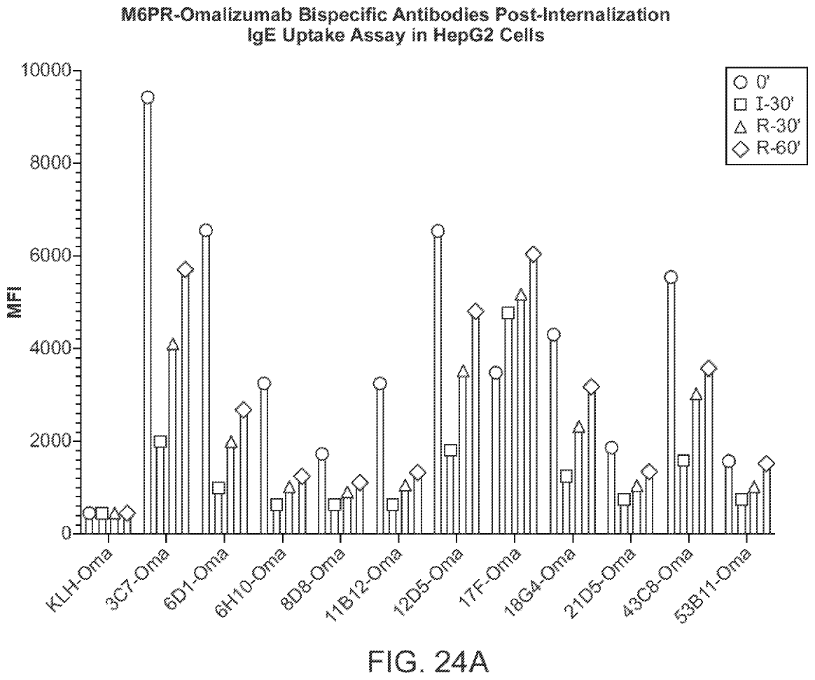

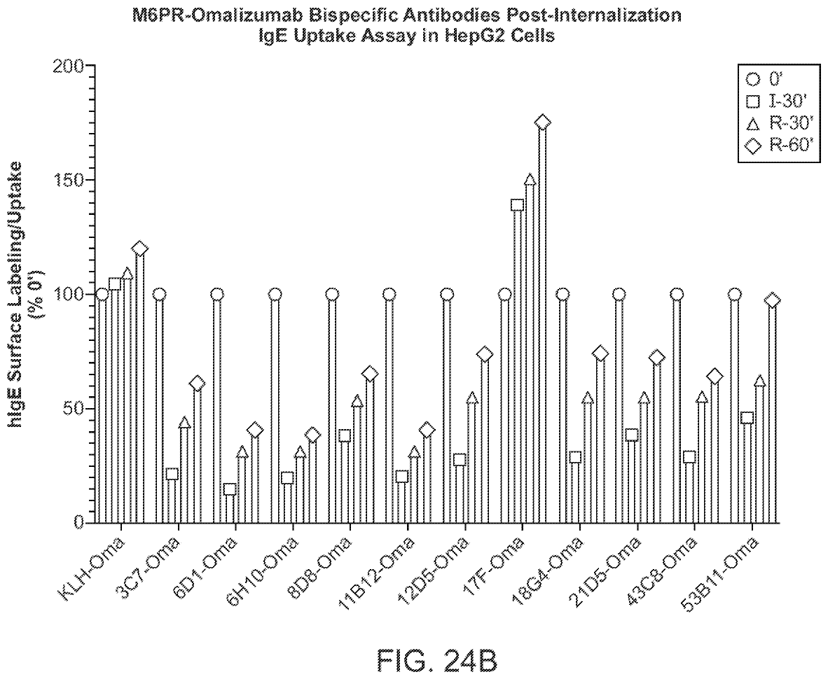

FIG. 24A shows internalization and recycling of M6PR-Oma bispecific antibodies by HepG2 cells as measured by mean fluorescence intensity (MFI). Each antibody was assessed for recycling following each of the indicated incubation protocols as described above and illustrated in FIG. 23. The ability of a bispecific antibody to internalize and recycle to the surface was evaluated by comparing MFI at the I-30′, R-30′, and R-60′ conditions to the 0′ condition (FIG. 24B). Based on this analysis, clones 12D5, 21D5, 18G4, and 53B11 showed recycling of M6PR-Oma bispecific antibodies most similar to the 0′ baseline condition, indicating that these M6PR-Oma bispecific antibodies have characteristics the enable uptake and recycling in HepG2 cells.

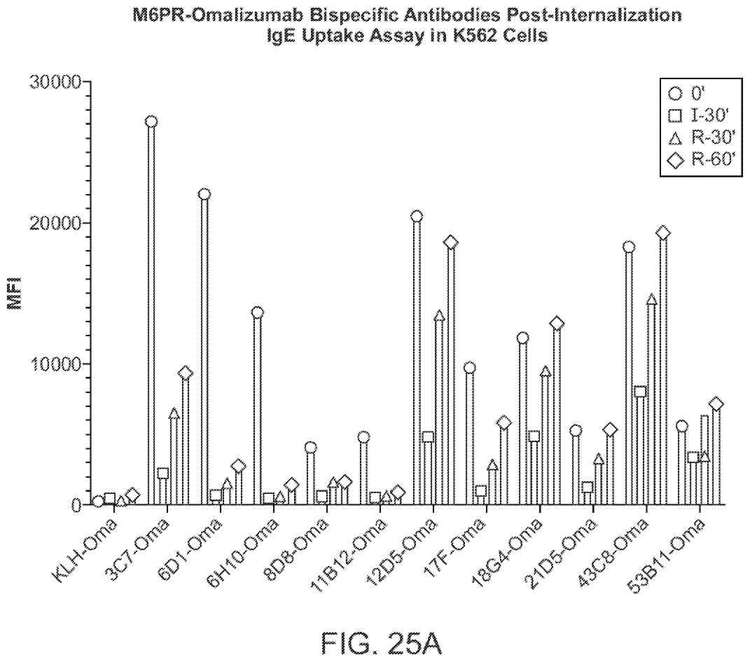

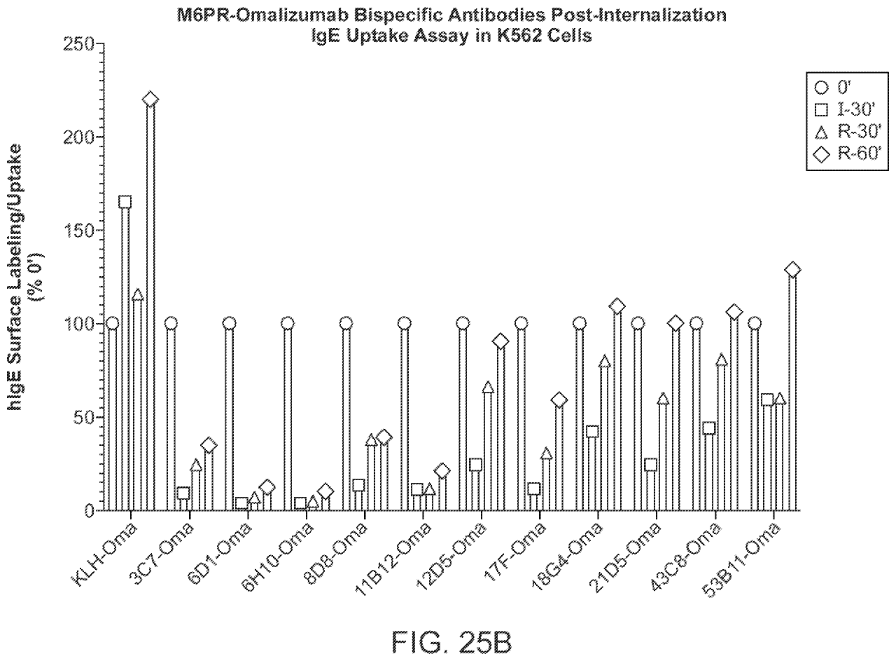

FIG. 25A shows internalization and recycling of MPR6-Oma bispecific antibodies by K562 cells as measured by mean fluorescence intensity (MFI). Each antibody was assessed for recycling following each of the indicated incubation protocols as described above and illustrated in FIG. 23. The ability of a bispecific antibody to internalize and recycle to the surface was evaluated by comparing MFI the I-30′, R-30′, and R-60′ conditions to the 0′ condition (FIG. 25B). Based on this analysis, clones 12D5, 18G4, 21D5, 43C8, and 53B11 showed recycling of M6PR-Oma bispecific antibodies most similar to the 0′ baseline condition, indicating that these matuzumab-anti-M6PR bispecific antibodies facilitate uptake and recycling in K562 cells.

### 6.15.8. Example 8: Assessing Internalization and Degradation of HER3

Cell surface depletion and total depletion of HiBiT-tagged HER3 (human epidermal growth receptor 3) were measured using Nano-Glo HiBiT Extracellular Detection System (Promega, Cat. No. N2422) and Nano-Glo HiBiT Lytic Detection System (Promega, Cat. No. N3050), respectively.

### HiBIT Assay Protocol

Briefly, HCC1419 cells (trastuzumab-resistant) expressing HiBiT-tagged HER3 were plated in tissue culture-treated 96 well plates in 100 μL and grown for 24 hours at 37° C. LUM-antiM6PR bispecific antibodies were diluted in media to prepare 3-fold dilution series from 20-0.001 nM (2× final concentrations) and added to cells in 100 μL per well, along with media-only control. Lumretuzumab (LUM), a humanized antihuman epidermal growth factor receptor 3 (HER3) monoclonal antibody and a NRGI (Neuregulin 1) andbody were used as a positive control A non-binding antibody (KLH) fused to LUM was used as a negative control for uptake activity.

Following LUM-antiM6PR bispecific antibodies or control treatment, cells were grown for 48 hours at 37° C. and media was replaced with 50 μL fresh media and 50 μL either extracellular or lytic HiBiT detection reagent prepared according to manufacturer's instructions. Briefly, LgBiT protein and Nano-Glo HiBiT substrate were diluted at 1:100 and 1:50 ratios respectively, in corresponding Nano-Glo HiBiT buffer. Following three minutes shaking and 10 minutes incubation at ambient temperature, HiBiT signal was detected by measuring luminescence on an Envision plate reader (Perkin Elmer). Dose responses were conducted in duplicate and normalized to media-only control. IC50 values were determined by fitting to 4-parameter curves in GraphPad Prism, and span was calculated from the difference between the first and last points on the curve.

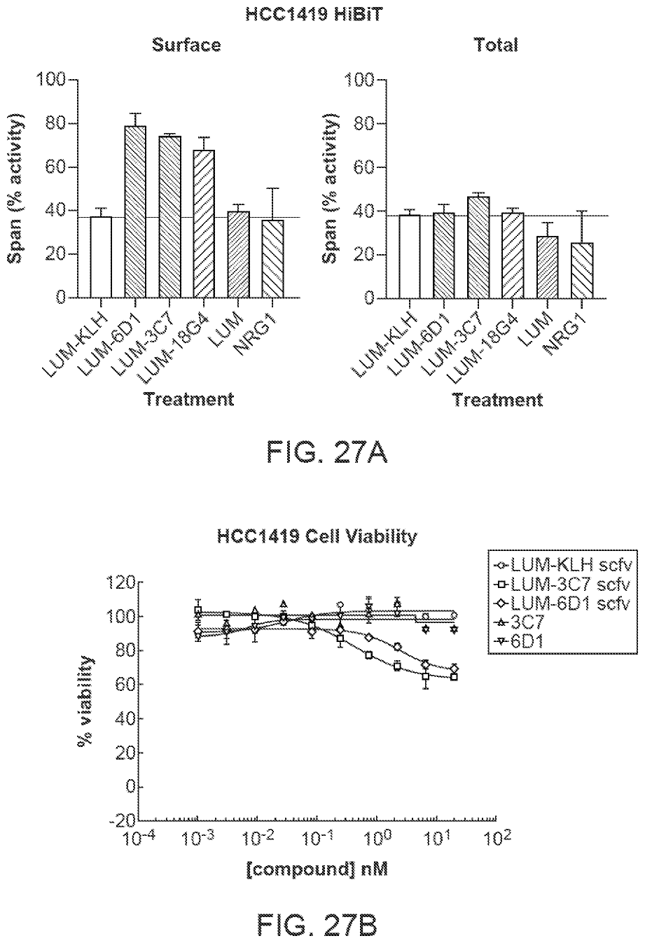

LUM-antiM6PR mediated HER3 internalization or degradation was evaluated at 48 hours. Cell viability after LUM-antiM6PR treatment was evaluated at 72 hours. The results are shown in FIGS. 27A-27B.

### Results

FIG. 27A shows surface depletion (i.e., span) and total depletion (i.e., span) of HER3 for a subset of LUM-antiM6PR bispecific antibodies. FIG. 27B shows cell viability after treatment of the subset of LUM-antiM6PR bispecific antibodies. FIG. 27A left panel shows percentage of surface HER3 activity of exemplary LUM-antiM6PR bispecific antibodies (6D1, 3C7, 18G4). FIG. 27A right panel shows percentage of total HER3 activity of the exemplary LUM-antiM6PR bispecific antibodies (6D1, 3C7, 18G4). FIG. 27B shows cell viability after treatment of the exemplary LUM-antiM6PR bispecific antibodies (6D1, 3C7,18G4). Taken together, this data showed that scfv LUM-antiM6PR bispecific antibodies increase HER3 internalization compared to the parental antibody (LUM). Additionally, the data demonstrate that scfv LUM-antiM6PR bispecific antibodies are efficient in impairing cell viability in trastuzumab-resistant cells.

### 7. INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57 (b) (1), to relate to each and every individual publication, database entry (e.g. Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### 8. CLAUSES

Clause 1. An antigen binding protein (ABP) comprising a first antigen binding moiety that specifically binds to an internalizing domain of cation-independent mannose 6 phosphate receptor (CI-M6PR) that facilitates intracellular transport of the ABP.

Clause 2. The ABP of clause 1, further comprising a target binding moiety that specifically binds to a target molecule.

Clause 3. The ABP of clause 2, wherein the target binding moiety is a second antigen binding moiety that specifically binds to the target molecule.

Clause 4. The ABP of clause 2, wherein the target binding moiety is conjugated to the first antigen binding moiety, optionally via a linker.

Clause 5. The ABP of any one of clauses 2-4, wherein the target molecule is a soluble extracellular target molecule or a cell surface target molecule.

Clause 6. The ABP of any one of the preceding clauses, further comprising a linked cargo moiety.

Clause 7. The ABP of clause 6, wherein the cargo moiety is a polypeptide that is fused to the first or second antigen binding moiety.

Clause 8. The ABP of clause 6, wherein the cargo moiety is conjugated to the first or second antigen binding moiety, optionally via a linker.

Clause 9. The ABP of clause 1 or 2, wherein the first antigen binding moiety binds to a domain of CI-M6PR selected from: domain 1, domain 4, domain 5, domain 6, domain 7, and domain 8.

Clause 10. The ABP of clause 1 or 2, wherein the first antigen binding moiety binds to domain 1 of CI-M6PR.

Clause 11. The ABP of clause 1 or 2, wherein the first antigen binding moiety binds to domain 4 of CI-M6PR.

Clause 12. The ABP of clause 1 or 2, wherein the first antigen binding moiety binds to domain 5 of CI-M6PR.

Clause 13. The ABP of clause 1 or 2, wherein the first antigen binding moiety binds to domain 6 of CI-M6PR.

Clause 14. The ABP of clause 1 or 2, wherein the first antigen binding moiety binds to domain 7 of CI-M6PR.

Clause 15. The ABP of clause 1 or 2, wherein the first antigen binding moiety binds to domain 8 of CI-M6PR.

Clause 16. The ABP of any one of the preceding clauses, wherein the first antigen binding moiety binds the CI-M6PR with high affinity.

Clause 17. The ABP of clause 16, wherein the first antigen binding moiety binds CI-M6PR with a dissociation equilibrium constant (K_{D}) of about 10 nM or less.

Clause 18. The ABP of any one of the preceding clauses, wherein the first antigen binding moiety binds CI-M6PR with a K_{D} between about 1 nM and about 500 nM.

Clause 19. The ABP of clause 18, wherein the first antigen binding moiety binds CI-M6PR with a K_{D} between about 10 nM and about 100 nM.

Clause 20. The ABP of clause 18, wherein the first antigen binding moiety binds CI-M6PR with a K_{D} between about 100 nM and about 200 nM.

Clause 21. The ABP of clause 18, wherein the first antigen binding moiety binds CI-M6PR with a K_{D} between about 200 nM and about 300 nM.

Clause 22. The ABP of clause 18, wherein the first antigen binding moiety binds CI-M6PR with a K_{D} between about 300 nM and about 400 nM.

Clause 23. The ABP of clause 18, wherein the first antigen binding moiety binds CI-M6PR with a K_{D} between about 400 nM and about 500 nM.

Clause 24. The ABP of any one of the preceding clauses, wherein the off rate (K_{off}) of the first antigen binding moiety for CI-M6PR is between 1×10⁻⁸ s⁻¹ and 0.1 s⁻¹.

Clause 25. The ABP of any one of the preceding clauses, wherein K_{off} of the first antigen binding moiety for CI-M6PR is between 1×10⁻⁶ s⁻¹ and 1×10⁻² s⁻¹.

Clause 26. The ABP of any one of the preceding clauses, wherein K_{off} of the first antigen binding moiety for CI-M6PR is about 1×10⁻⁵ s⁻¹ or slower.

Clause 27. The ABP of any one of the preceding clauses, wherein the binding of the first antigen binding moiety to CI-M6PR is pH dependent.

Clause 28. The ABP of any one of the preceding clauses, wherein the first antigen binding moiety is released from CI-M6PR at a pH of 7.4 or lower.

Clause 29. The ABP of any one of the preceding clauses, wherein the first antigen binding moiety is released from CI-M6PR at a pH of 6.5 or lower.

Clause 30. The ABP of any one of the preceding clauses, wherein the first antigen binding moiety is released from CI-M6PR at a pH of 6.0 or lower.

Clause 31. The ABP of any one of the preceding clauses, wherein the first antigen binding moiety is released from CI-M6PR at a pH of 5.5 or lower.

Clause 32. The ABP of any one of the preceding clauses, wherein the first antigen binding moiety is released from CI-M6PR at a pH of 5.0 or lower.

Clause 33. The ABP of any one of the preceding clauses, wherein the first antigen binding moiety comprises a light chain CDR3 (LCDR3) having the sequence of SEQ ID NO: 76 and a heavy chain CDR3 (HCDR3) having the sequence of SEQ ID NO: 35.

Clause 34. The ABP of any one of clauses 1-32, wherein the first antigen binding moiety comprises a light chain CDR3 having the sequence of SEQ ID NO: 78 and a heavy chain CDR3 having the amino acid sequence of SEQ ID NO: 38.

Clause 35. The ABP of any one of clauses 1-32, wherein the first antigen binding moiety comprises a light chain CDR3 having the sequence of SEQ ID NO: 81 and a heavy chain CDR3 having the sequence of SEQ ID NO: 41.

Clause 36. The ABP of any one of clauses 1-32, wherein the first antigen binding moiety comprises a light chain CDR3 having the sequence of SEQ ID NO: 84 and a heavy chain CDR3 having the sequence of SEQ ID NO: 44.

Clause 37. The ABP of any one of clauses 1-32, wherein the first antigen binding moiety comprises a light chain CDR3 having the sequence of SEQ ID NO: 85 and a heavy chain CDR3 having the sequence of SEQ ID NO: 46.

Clause 38. The ABP of any one of clauses 1-32, wherein the first antigen binding moiety comprises a light chain CDR3 having the sequence of SEQ ID NO: 88 and a heavy chain CDR3 having the sequence of SEQ ID NO: 49.

Clause 39. The ABP of any one of clauses 1-32, wherein the first antigen binding moiety comprises a light chain CDR3 having the sequence of SEQ ID NO: 91 and a heavy chain CDR3 having the sequence of SEQ ID NO: 52.

Clause 40. The ABP of any one of clauses 1-32, wherein the first antigen binding moiety comprises a light chain CDR3 having the sequence of SEQ ID NO: 94 and a heavy chain CDR3 having the sequence of SEQ ID NO: 54.

Clause 41. The ABP of any one of clauses 1-32, wherein the first antigen binding moiety comprises a light chain CDR3 having the sequence of SEQ ID NO: 96 and a heavy chain CDR3 having the sequence of SEQ ID NO: 55.

Clause 42. The ABP of any one of clauses 1-32, wherein the first antigen binding moiety comprises a light chain CDR3 having the sequence of SEQ ID NO: 99 and a heavy chain CDR3 having the sequence of SEQ ID NO: 58.

Clause 43. The ABP of any one of clauses 1-32, wherein the first antigen binding moiety comprises a light chain CDR3 having the sequence of SEQ ID NO: 101 and a heavy chain CDR3 having the sequence of SEQ ID NO: 60.

Clause 44. The ABP of any one of clauses 1-32, wherein the first antigen binding moiety comprises a light chain CDR3 having the sequence of SEQ ID NO: 104 and a heavy chain CDR3 having the sequence of SEQ ID NO: 63.

Clause 45. The ABP of any one of clauses 1-32, wherein the first antigen binding moiety comprises a light chain CDR3 having the sequence of SEQ ID NO: 107 and a heavy chain CDR3 having the sequence of SEQ ID NO: 66.

Clause 46. The ABP of any one of clauses 1-32, wherein the first antigen binding moiety comprises a light chain CDR3 having the sequence of SEQ ID NO: 110 and a heavy chain CDR3 having the sequence of SEQ ID NO: 69.

Clause 47. The ABP of any one of clauses 1-32, wherein the first antigen binding moiety comprises a light chain CDR3 having the sequence of SEQ ID NO: 113 and a heavy chain CDR3 having the sequence of SEQ ID NO: 72.

Clause 48. The ABP of any one of clauses 1-32, wherein the first antigen binding moiety comprises a light chain CDR3 having the sequence of SEQ ID NO: 116 and a heavy chain CDR3 having the sequence of SEQ ID NO: 73.

Clause 49. The ABP of clause 33, wherein the first antigen binding moiety further comprises a light chain CDR1 (LCDR1) having the sequence of SEQ ID NO: 74 and a heavy chain CDR1 (HCDR1) having the sequence of SEQ ID NO: 33; and a light chain CDR2 (LCDR2) having the sequence of SEQ ID NO: 75 and a heavy chain CDR2 (HCDR2) having the sequence of SEQ ID NO: 34.

Clause 50. The ABP of clause 34, wherein the first antigen binding moiety further comprises an LCDR1 having the sequence of SEQ ID NO: 77 and a heavy chain HCDR1 having the sequence of SEQ ID NO: 36; and an LCDR2 having the sequence of SEQ ID NO: 75 and a HCDR2 having the sequence of SEQ ID NO: 37.

Clause 51. The ABP of clause 35, wherein the first antigen binding moiety further comprises an LCDR1 having the sequence of SEQ ID NO: 79 and a heavy chain HCDR1 having the sequence of SEQ ID NO: 39; and an LCDR2 having the sequence of SEQ ID NO: 80 and a HCDR2 having the sequence of SEQ ID NO: 40.

Clause 52. The ABP of clause 36, wherein the first antigen binding moiety further comprises an LCDR1 having the sequence of SEQ ID NO: 82 and a heavy chain HCDR1 having the sequence of SEQ ID NO: 42; and an LCDR2 having the sequence of SEQ ID NO: 83 and a HCDR2 having the sequence of SEQ ID NO: 43.

Clause 53. The ABP of clause 37, wherein the first antigen binding moiety further comprises an LCDR1 having the sequence of SEQ ID NO: 79 and a heavy chain HCDR1 having the sequence of SEQ ID NO: 45; and an LCDR2 having the sequence of SEQ ID NO: 80 and a HCDR2 having the sequence of SEQ ID NO: 34.

Clause 54. The ABP of clause 38, wherein the first antigen binding moiety further comprises an LCDR1 having the sequence of SEQ ID NO: 86 and a heavy chain HCDR1 having the sequence of SEQ ID NO: 47; and an LCDR2 having the sequence of SEQ ID NO: 87 and a HCDR2 having the sequence of SEQ ID NO: 48.

Clause 55. The ABP of clause 39, wherein the first antigen binding moiety further comprises an LCDR1 having the sequence of SEQ ID NO: 89 and a heavy chain HCDR1 having the sequence of SEQ ID NO: 50; and an LCDR2 having the sequence of SEQ ID NO: 90 and a HCDR2 having the sequence of SEQ ID NO: 53.

Clause 56. The ABP of clause 40, wherein the first antigen binding moiety further comprises an LCDR1 having the sequence of SEQ ID NO: 92 and a heavy chain HCDR1 having the sequence of SEQ ID NO: 50; and an LCDR2 having the sequence of SEQ ID NO: 93 and a HCDR2 having the sequence of SEQ ID NO: 53.

Clause 57. The ABP of clause 41, wherein the first antigen binding moiety further comprises an LCDR1 having the sequence of SEQ ID NO: 95 and a heavy chain HCDR1 having the sequence of SEQ ID NO: 50; and an LCDR2 having the sequence of SEQ ID NO: 93 and a HCDR2 having the sequence of SEQ ID NO: 51.

Clause 58. The ABP of clause 42, wherein the first antigen binding moiety further comprises an LCDR1 having the sequence of SEQ ID NO: 97 and a heavy chain HCDR1 having the sequence of SEQ ID NO: 56; and an LCDR2 having the sequence of SEQ ID NO: 98 and a HCDR2 having the sequence of SEQ ID NO: 57.

Clause 59. The ABP of clause 43, wherein the first antigen binding moiety further comprises an LCDR1 having the sequence of SEQ ID NO: 100 and a heavy chain HCDR1 having the sequence of SEQ ID NO: 50; and an LCDR2 having the sequence of SEQ ID NO: 93 and a HCDR2 having the sequence of SEQ ID NO: 59.

Clause 60. The ABP of clause 44, wherein the first antigen binding moiety further comprises an LCDR1 having the sequence of SEQ ID NO: 102 and a heavy chain HCDR1 having the sequence of SEQ ID NO: 61; and an LCDR2 having the sequence of SEQ ID NO: 103 and a HCDR2 having the sequence of SEQ ID NO: 62.

Clause 61. The ABP of clause 45, wherein the first antigen binding moiety further comprises an LCDR1 having the sequence of SEQ ID NO: 105 and a heavy chain HCDR1 having the sequence of SEQ ID NO: 64; and an LCDR2 having the sequence of SEQ ID NO: 106 and a HCDR2 having the sequence of SEQ ID NO: 65.

Clause 62. The ABP of clause 46, wherein the first antigen binding moiety further comprises an LCDR1 having the sequence of SEQ ID NO: 108 and a heavy chain HCDR1 having the sequence of SEQ ID NO: 67; and an LCDR2 having the sequence of SEQ ID NO: 109 and a HCDR2 having the sequence of SEQ ID NO: 68.

Clause 63. The ABP of clause 47, wherein the first antigen binding moiety further comprises an LCDR1 having the sequence of SEQ ID NO: 111 and a heavy chain HCDR1 having the sequence of SEQ ID NO: 70; and an LCDR2 having the sequence of SEQ ID NO: 112 and a HCDR2 having the sequence of SEQ ID NO: 71.

Clause 64. The ABP of clause 48, wherein the first antigen binding moiety further comprises an LCDR1 having the sequence of SEQ ID NO: 114 and a heavy chain HCDR1 having the sequence of SEQ ID NO: 70; and an LCDR2 having the sequence of SEQ ID NO: 115 and a HCDR2 having the sequence of SEQ ID NO: 71.

Clause 65. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 205, LFR2 is at least 95% identical to the sequence of SEQ ID NO: 206, LFR3 is at least 95% identical to the sequence of SEQ ID NO: 207; and LFR4 is at least 95% identical to the sequence of SEQ ID NO: 208.

Clause 66. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 209, LFR2 is at least 95% identical to the sequence of SEQ ID NO: 210, LFR3 is at least 95% identical to the sequence of SEQ ID NO: 211; and LFR4 is at least 95% identical to the sequence of SEQ ID NO: 212.

Clause 67. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 213, LFR2 is at least 95% identical to the sequence of SEQ ID NO: 214, LFR3 is at least 95% identical to the sequence of SEQ ID NO: 215; and LFR4 is at least 95% identical to the sequence of SEQ ID NO: 216.

Clause 68. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 217, LFR2 is at least 95% identical to the sequence of SEQ ID NO: 218, LFR3 is at least 95% identical to the sequence of SEQ ID NO: 219; and LFR4 is at least 95% identical to the sequence of SEQ ID NO: 220.

Clause 69. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 221, LFR2 is at least 95% identical to the sequence of SEQ ID NO: 222, LFR3 is at least 95% identical to the sequence of SEQ ID NO: 223; and LFR4 is at least 95% identical to the sequence of SEQ ID NO: 224.

Clause 70. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 225, LFR2 is at least 95% identical to the sequence of SEQ ID NO: 226, LFR3 is at least 95% identical to the sequence of SEQ ID NO: 227; and LFR4 is at least 95% identical to the sequence of SEQ ID NO: 228.

Clause 71. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 229, LFR2 is at least 95% identical to the sequence of SEQ ID NO: 230, LFR3 is at least 95% identical to the sequence of SEQ ID NO: 231; and LFR4 is at least 95% identical to the sequence of SEQ ID NO: 232.

Clause 72. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 233, LFR2 is at least 95% identical to the sequence of SEQ ID NO: 234, LFR3 is at least 95% identical to the sequence of SEQ ID NO: 235; and LFR4 is at least 95% identical to the sequence of SEQ ID NO: 236.

Clause 73. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 237, LFR2 is at least 95% identical to the sequence of SEQ ID NO: 238, LFR3 is at least 95% identical to the sequence of SEQ ID NO: 239; and LFR4 is at least 95% identical to the sequence of SEQ ID NO: 240.

Clause 74. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 241, LFR2 is at least 95% identical to the sequence of SEQ ID NO: 242, LFR3 is at least 95% identical to the sequence of SEQ ID NO: 243; and LFR4 is at least 95% identical to the sequence of SEQ ID NO: 244.

Clause 75. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 245, LFR2 is at least 95% identical to the sequence of SEQ ID NO: 246, LFR3 is at least 95% identical to the sequence of SEQ ID NO: 247; and LFR4 is at least 95% identical to the sequence of SEQ ID NO: 248.

Clause 76. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 249, LFR2 is at least 95% identical to the sequence of SEQ ID NO: 250, LFR3 is at least 95% identical to the sequence of SEQ ID NO: 251; and LFR4 is at least 95% identical to the sequence of SEQ ID NO: 252.

Clause 77. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 253, LFR2 is at least 95% identical to the sequence of SEQ ID NO: 254, LFR3 is at least 95% identical to the sequence of SEQ ID NO: 255; and LFR4 is at least 95% identical to the sequence of SEQ ID NO: 256.

Clause 78. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 257, LFR2 is at least 95% identical to the sequence of SEQ ID NO: 258, LFR3 is at least 95% identical to the sequence of SEQ ID NO: 259; and LFR4 is at least 95% identical to the sequence of SEQ ID NO: 260.

Clause 79. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 261, LFR2 is at least 95% identical to the sequence of SEQ ID NO: 262, LFR3 is at least 95% identical to the sequence of SEQ ID NO: 263; and LFR4 is at least 95% identical to the sequence of SEQ ID NO: 264.

Clause 80. The ABP of any one of the preceding clauses, wherein the first antigen binding moiety comprises a variable light chain (VL) comprising framework regions LFR1, LFR2, LFR3, and LFR4 wherein LFR1 is at least 95% identical to the sequence of SEQ ID NO: 265, LFR2 is at least 95% identical to the sequence of SEQ ID NO: 266, LFR3 is at least 95% identical to the sequence of SEQ ID NO: 267; and LFR4 is at least 95% identical to the sequence of SEQ ID NO: 268.

Clause 81. The ABP of clause 65, wherein the first antigen binding moiety comprises a variable heavy chain (VH) comprising framework regions HFR1, HFR2, HFR3, and HFR4 wherein HFR1 is at least 95% identical to the sequence of SEQ ID NO: 141, HFR2 is at least 95% identical to the sequence of SEQ ID NO: 142, HFR3 is at least 95% identical to the sequence of SEQ ID NO: 143; and HFR4 is at least 95% identical to the sequence of SEQ ID NO: 144.

Clause 82. The ABP of clause 66, wherein the first antigen binding moiety comprises a variable heavy chain (VH) comprising framework regions HFR1, HFR2, HFR3, and HFR4 wherein HFR1 is at least 95% identical to the sequence of SEQ ID NO: 145, HFR2 is at least 95% identical to the sequence of SEQ ID NO: 146, HFR3 is at least 95% identical to the sequence of SEQ ID NO: 147; and HFR4 is at least 95% identical to the sequence of SEQ ID NO: 148; or

Clause 83. The ABP of clause 67, wherein the first antigen binding moiety comprises a variable heavy chain (VH) comprising framework regions HFR1, HFR2, HFR3, and HFR4 wherein HFR1 is at least 95% identical to the sequence of SEQ ID NO: 149, HFR2 is at least 95% identical to the sequence of SEQ ID NO: 150, HFR3 is at least 95% identical to the sequence of SEQ ID NO: 151; and HFR4 is at least 95% identical to the sequence of SEQ ID NO: 152.

Clause 84. The ABP of clause 68, wherein the first antigen binding moiety comprises a variable heavy chain (VH) comprising framework regions HFR1, HFR2, HFR3, and HFR4 wherein HFR1 is at least 95% identical to the sequence of SEQ ID NO: 153, HFR2 is at least 95% identical to the sequence of SEQ ID NO: 154, HFR3 is at least 95% identical to the sequence of SEQ ID NO: 155; and HFR4 is at least 95% identical to the sequence of SEQ ID NO: 156.

Clause 85. The ABP of clause 69, wherein the first antigen binding moiety comprises a variable heavy chain (VH) comprising framework regions HFR1, HFR2, HFR3, and HFR4 wherein HFR1 is at least 95% identical to the sequence of SEQ ID NO: 157, HFR2 is at least 95% identical to the sequence of SEQ ID NO: 158, HFR3 is at least 95% identical to the sequence of SEQ ID NO: 159; and HFR4 is at least 95% identical to the sequence of SEQ ID NO: 160.

Clause 86. The ABP of clause 70, wherein the first antigen binding moiety comprises a variable heavy chain (VH) comprising framework regions HFR1, HFR2, HFR3, and HFR4 wherein HFR1 is at least 95% identical to the sequence of SEQ ID NO: 161, HFR2 is at least 95% identical to the sequence of SEQ ID NO: 162, HFR3 is at least 95% identical to the sequence of SEQ ID NO: 163; and HFR4 is at least 95% identical to the sequence of SEQ ID NO: 164.

Clause 87. The ABP of clause 71, wherein the first antigen binding moiety comprises a variable heavy chain (VH) comprising framework regions HFR1, HFR2, HFR3, and HFR4 wherein HFR1 is at least 95% identical to the sequence of SEQ ID NO: 165, HFR2 is at least 95% identical to the sequence of SEQ ID NO: 166, HFR3 is at least 95% identical to the sequence of SEQ ID NO: 167; and HFR4 is at least 95% identical to the sequence of SEQ ID NO: 168.

Clause 88. The ABP of clause 72, wherein the first antigen binding moiety comprises a variable heavy chain (VH) comprising framework regions HFR1, HFR2, HFR3, and HFR4 wherein HFR1 is at least 95% identical to the sequence of SEQ ID NO: 169, HFR2 is at least 95% identical to the sequence of SEQ ID NO: 170, HFR3 is at least 95% identical to the sequence of SEQ ID NO: 171; and HFR4 is at least 95% identical to the sequence of SEQ ID NO: 172.

Clause 89. The ABP of clause 73, wherein the first antigen binding moiety comprises a variable heavy chain (VH) comprising framework regions HFR1, HFR2, HFR3, and HFR4 wherein HFR1 is at least 95% identical to the sequence of SEQ ID NO: 173, HFR2 is at least 95% identical to the sequence of SEQ ID NO: 174, HFR3 is at least 95% identical to the sequence of SEQ ID NO: 175; and HFR4 is at least 95% identical to the sequence of SEQ ID NO: 176.

Clause 90. The ABP of clause 74, wherein the first antigen binding moiety comprises a variable heavy chain (VH) comprising framework regions HFR1, HFR2, HFR3, and HFR4 wherein HFR1 is at least 95% identical to the sequence of SEQ ID NO: 177, HFR2 is at least 95% identical to the sequence of SEQ ID NO: 178, HFR3 is at least 95% identical to the sequence of SEQ ID NO: 179; and HFR4 is at least 95% identical to the sequence of SEQ ID NO: 180.

Clause 91. The ABP of clause 75, wherein the first antigen binding moiety comprises a variable heavy chain (VH) comprising framework regions HFR1, HFR2, HFR3, and HFR4 wherein HFR1 is at least 95% identical to the sequence of SEQ ID NO: 181, HFR2 is at least 95% identical to the sequence of SEQ ID NO: 182, HFR3 is at least 95% identical to the sequence of SEQ ID NO: 183; and HFR4 is at least 95% identical to the sequence of SEQ ID NO: 184.

Clause 92. The ABP of clause 76, wherein the first antigen binding moiety comprises a variable heavy chain (VH) comprising framework regions HFR1, HFR2, HFR3, and HFR4 wherein HFR1 is at least 95% identical to the sequence of SEQ ID NO: 185, HFR2 is at least 95% identical to the sequence of SEQ ID NO: 186, HFR3 is at least 95% identical to the sequence of SEQ ID NO: 187; and HFR4 is at least 95% identical to the sequence of SEQ ID NO: 188.

Clause 93. The ABP of clause 77, wherein the first antigen binding moiety comprises a variable heavy chain (VH) comprising framework regions HFR1, HFR2, HFR3, and HFR4 wherein HFR1 is at least 95% identical to the sequence of SEQ ID NO: 189, HFR2 is at least 95% identical to the sequence of SEQ ID NO: 190, HFR3 is at least 95% identical to the sequence of SEQ ID NO: 191; and HFR4 is at least 95% identical to the sequence of SEQ ID NO: 192; or

Clause 94. The ABP of clause 78, wherein the first antigen binding moiety comprises a variable heavy chain (VH) comprising framework regions HFR1, HFR2, HFR3, and HFR4 wherein HFR1 is at least 95% identical to the sequence of SEQ ID NO: 193, HFR2 is at least 95% identical to the sequence of SEQ ID NO: 194, HFR3 is at least 95% identical to the sequence of SEQ ID NO: 195; and HFR4 is at least 95% identical to the sequence of SEQ ID NO: 196; or

Clause 95. The ABP of clause 79, wherein the first antigen binding moiety comprises a variable heavy chain (VH) comprising framework regions HFR1, HFR2, HFR3, and HFR4 wherein HFR1 is at least 95% identical to the sequence of SEQ ID NO: 197, HFR2 is at least 95% identical to the sequence of SEQ ID NO: 198, HFR3 is at least 95% identical to the sequence of SEQ ID NO: 199; and HFR4 is at least 95% identical to the sequence of SEQ ID NO: 200; or

Clause 96. The ABP of clause 80, wherein the first antigen binding moiety comprises a variable heavy chain (VH) comprising framework regions HFR1, HFR2, HFR3, and HFR4 wherein HFR1 is at least 95% identical to the sequence of SEQ ID NO: 201, HFR2 is at least 95% identical to the sequence of SEQ ID NO: 202, HFR3 is at least 95% identical to the sequence of SEQ ID NO: 203; and HFR4 is at least 95% identical to the sequence of SEQ ID NO: 204.

Clause 97. The ABP of any of the preceding clauses, wherein the first antigen binding moiety comprises a variable light chain (VL) having an amino acid sequence that is at least 90% identical to an amino acid sequence selected from SEQ ID NOs: 17-32.

Clause 98. The ABP of any of the preceding clauses, wherein the first antigen binding moiety comprises a variable heavy chain (VH) having an amino acid sequence that is at least 90% identical to an amino acid sequence selected from SEQ ID NOs.: 1-16.

Clause 99. The ABP of any one of the preceding clauses, wherein the first antigen binding moiety is an antibody fragment (e.g., single chain variable fragment (scFv) or antigen-binding fragment).

Clause 100. The ABP of any one of the preceding clauses, wherein the ABP is transported to the lysosome.

Clause 101. The ABP of any one of the preceding clauses, wherein the ABP is transported back to the cell surface following internalization.

Clause 102. The ABP of any one of clauses 1-100, wherein the ABP is degraded in the cell.

Clause 103. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 82,
the LCDR2 has the sequence of SEQ ID NO: 83,
the LCDR3 has the sequence of SEQ ID NO: 84,
the HCDR 1 has the sequence of SEQ ID NO: 42,
the HCDR2 has the sequence of SEQ ID NO: 43, and
the HCDR3 has the sequence of SEQ ID NO: 44.

Clause 104. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 92,
the LCDR2 has the sequence of SEQ ID NO: 93,
the LCDR3 has the sequence of SEQ ID NO: 94,
the HCDR 1 has the sequence of SEQ ID NO: 50,
the HCDR2 has the sequence of SEQ ID NO: 53, and
the HCDR3 has the sequence of SEQ ID NO: 54.

Clause 105. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 97,
the LCDR2 has the sequence of SEQ ID NO: 98,
the LCDR3 has the sequence of SEQ ID NO: 99,
the HCDR 1 has the sequence of SEQ ID NO: 56,
the HCDR2 has the sequence of SEQ ID NO: 57, and
the HCDR3 has the sequence of SEQ ID NO: 58.

Clause 106. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 100,
the LCDR2 has the sequence of SEQ ID NO: 93,
the LCDR3 has the sequence of SEQ ID NO: 101,
the HCDR 1 has the sequence of SEQ ID NO: 50,
the HCDR2 has the sequence of SEQ ID NO: 59, and
the HCDR3 has the sequence of SEQ ID NO: 60.

Clause 107. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 108,
the LCDR2 has the sequence of SEQ ID NO: 109,
the LCDR3 has the sequence of SEQ ID NO: 110,
the HCDR 1 has the sequence of SEQ ID NO: 67,
the HCDR2 has the sequence of SEQ ID NO: 68, and
the HCDR3 has the sequence of SEQ ID NO: 69.

Clause 108. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 111,
the LCDR2 has the sequence of SEQ ID NO: 112,
the LCDR3 has the sequence of SEQ ID NO: 113,
the HCDR 1 has the sequence of SEQ ID NO: 70,
the HCDR2 has the sequence of SEQ ID NO: 71, and
the HCDR3 has the sequence of SEQ ID NO: 72.

Clause 109. A bifunctional molecule comprising:
a first moiety that specifically binds to cation-independent mannose 6 phosphate receptor (CI-M6PR), wherein the first moiety is an antibody or antibody fragment; and
a second moiety that specifically binds a cell surface target molecule or extracellular target molecule, wherein the second moiety is selected from an antibody, an antigen-binding fragment, a ligand, and a small molecule.

Clause 110. The bifunctional molecule of clause 109, wherein the bifunctional molecule is a polypeptide.

Clause 111. The bifunctional molecule of clause 109, wherein the first moiety and the second moiety are covalently attached via a linker.

Clause 112. The bifunctional molecule of clause 111, wherein the second moiety is a small molecule.

Clause 113. The bifunctional molecule of any one of clauses 109-112, wherein the first binding moiety comprises a light chain CDR3 (LCDR3) having the sequence of SEQ ID NO: 76 and a heavy chain CDR3 (HCDR3) having the sequence of SEQ ID NO: 35.

Clause 114. The bifunctional molecule of any one of clauses 109-112, wherein the first binding moiety comprises a light chain CDR3 having the sequence of SEQ ID NO: 78 and a heavy chain CDR3 having the amino acid sequence of SEQ ID NO: 38.

Clause 115. The bifunctional molecule of any one of clauses 109-112, wherein the first binding moiety comprises a light chain CDR3 having the sequence of SEQ ID NO: 81 and a heavy chain CDR3 having the sequence of SEQ ID NO: 41.

Clause 116. The bifunctional molecule of any one of clauses 109-112, wherein the first binding moiety comprises a light chain CDR3 having the sequence of SEQ ID NO: 84 and a heavy chain CDR3 having the sequence of SEQ ID NO: 44.

Clause 117. The bifunctional molecule of any one of clauses 109-112, wherein the first binding moiety comprises a light chain CDR3 having the sequence of SEQ ID NO: 85 and a heavy chain CDR3 having the sequence of SEQ ID NO: 46.

Clause 118. The bifunctional molecule of any one of clauses 109-112, wherein the first binding moiety comprises a light chain CDR3 having the sequence of SEQ ID NO: 88 and a heavy chain CDR3 having the sequence of SEQ ID NO: 49.

Clause 119. The bifunctional molecule of any one of clauses 109-112, wherein the first binding moiety comprises a light chain CDR3 having the sequence of SEQ ID NO: 91 and a heavy chain CDR3 having the sequence of SEQ ID NO: 52.

Clause 120. The bifunctional molecule of any one of clauses 109-112, wherein the first binding moiety comprises a light chain CDR3 having the sequence of SEQ ID NO: 94 and a heavy chain CDR3 having the sequence of SEQ ID NO: 54.

Clause 121. The bifunctional molecule of any one of clauses 109-112, wherein the first binding moiety comprises a light chain CDR3 having the sequence of SEQ ID NO: 96 and a heavy chain CDR3 having the sequence of SEQ ID NO: 55.

Clause 122. The bifunctional molecule of any one of clauses 109-112, wherein the first binding moiety comprises a light chain CDR3 having the sequence of SEQ ID NO: 99 and a heavy chain CDR3 having the sequence of SEQ ID NO: 58.

Clause 123. The bifunctional molecule of any one of clauses 109-112, wherein the first binding moiety comprises a light chain CDR3 having the sequence of SEQ ID NO: 101 and a heavy chain CDR3 having the sequence of SEQ ID NO: 60.

Clause 124. The bifunctional molecule of any one of clauses 109-112, wherein the first binding moiety comprises a light chain CDR3 having the sequence of SEQ ID NO: 104 and a heavy chain CDR3 having the sequence of SEQ ID NO: 63.

Clause 125. The bifunctional molecule of any one of clauses 109-112, wherein the first binding moiety comprises a light chain CDR3 having the sequence of SEQ ID NO: 107 and a heavy chain CDR3 having the sequence of SEQ ID NO: 66.

Clause 126. The bifunctional molecule of any one of clauses 109-112, wherein the first binding moiety comprises a light chain CDR3 having the sequence of SEQ ID NO: 110 and a heavy chain CDR3 having the sequence of SEQ ID NO: 69.

Clause 127. The bifunctional molecule of any one of clauses 109-112, wherein the first binding moiety comprises a light chain CDR3 having the sequence of SEQ ID NO: 113 and a heavy chain CDR3 having the sequence of SEQ ID NO: 72.

Clause 128. The bifunctional molecule of any one of clauses 109-112, wherein the first binding moiety comprises a light chain CDR3 having the sequence of SEQ ID NO: 116 and a heavy chain CDR3 having the sequence of SEQ ID NO: 73.

Clause 129. The bifunctional molecule of clause 113, wherein the first moiety further comprises a light chain CDR1 (LCDR1) having the sequence of SEQ ID NO: 74 and a heavy chain CDR1 (HCDR1) having the sequence of SEQ ID NO: 33; and a light chain CDR2 (LCDR2) having the sequence of SEQ ID NO: 75 and a heavy chain CDR2 (HCDR2) having the sequence of SEQ ID NO: 34.

Clause 130. The bifunctional molecule of clause 114, wherein the first moiety further comprises an LCDR1 having the sequence of SEQ ID NO: 77 and a heavy chain HCDR1 having the sequence of SEQ ID NO: 36; and an LCDR2 having the sequence of SEQ ID NO: 75 and a HCDR2 having the sequence of SEQ ID NO: 37.

Clause 131. The bifunctional molecule of clause 115, wherein the first moiety further comprises an LCDR1 having the sequence of SEQ ID NO: 79 and a heavy chain HCDR1 having the sequence of SEQ ID NO: 39; and an LCDR2 having the sequence of SEQ ID NO: 80 and a HCDR2 having the sequence of SEQ ID NO: 40.

Clause 132. The bifunctional molecule of clause 116, wherein the first moiety further comprises an LCDR1 having the sequence of SEQ ID NO: 82 and a heavy chain HCDR1 having the sequence of SEQ ID NO: 42; and an LCDR2 having the sequence of SEQ ID NO: 83 and a HCDR2 having the sequence of SEQ ID NO: 43.

Clause 133. The bifunctional molecule of clause 117, wherein the first moiety further comprises an LCDR1 having the sequence of SEQ ID NO: 79 and a heavy chain HCDR1 having the sequence of SEQ ID NO: 45; and an LCDR2 having the sequence of SEQ ID NO: 80 and a HCDR2 having the sequence of SEQ ID NO: 34.

Clause 134. The bifunctional molecule of clause 118, wherein the first moiety further comprises an LCDR1 having the sequence of SEQ ID NO: 86 and a heavy chain HCDR1 having the sequence of SEQ ID NO: 47; and an LCDR2 having the sequence of SEQ ID NO: 87 and a HCDR2 having the sequence of SEQ ID NO: 48.

Clause 135. The bifunctional molecule of clause 119, wherein the first moiety further comprises an LCDR1 having the sequence of SEQ ID NO: 89 and a heavy chain HCDR1 having the sequence of SEQ ID NO: 50; and an LCDR2 having the sequence of SEQ ID NO: 90 and a HCDR2 having the sequence of SEQ ID NO: 53.

Clause 136. The bifunctional molecule of clause 120, wherein the first moiety further comprises an LCDR1 having the sequence of SEQ ID NO: 92 and a heavy chain HCDR1 having the sequence of SEQ ID NO: 50; and an LCDR2 having the sequence of SEQ ID NO: 93 and a HCDR2 having the sequence of SEQ ID NO: 53.

Clause 137. The bifunctional molecule of clause 121, wherein the first moiety further comprises an LCDR1 having the sequence of SEQ ID NO: 95 and a heavy chain HCDR1 having the sequence of SEQ ID NO: 50; and an LCDR2 having the sequence of SEQ ID NO: 93 and a HCDR2 having the sequence of SEQ ID NO: 51.

Clause 138. The bifunctional molecule of clause 122, wherein the first moiety further comprises an LCDR1 having the sequence of SEQ ID NO: 97 and a heavy chain HCDR1 having the sequence of SEQ ID NO: 56; and an LCDR2 having the sequence of SEQ ID NO: 98 and a HCDR2 having the sequence of SEQ ID NO: 57.

Clause 139. The bifunctional molecule of clause 123, wherein the first moiety further comprises an LCDR1 having the sequence of SEQ ID NO: 100 and a heavy chain HCDR1 having the sequence of SEQ ID NO: 50; and an LCDR2 having the sequence of SEQ ID NO: 93 and a HCDR2 having the sequence of SEQ ID NO: 59.

Clause 140. The bifunctional molecule of clause 124, wherein the first moiety further comprises an LCDR1 having the sequence of SEQ ID NO: 102 and a heavy chain HCDR1 having the sequence of SEQ ID NO: 61; and an LCDR2 having the sequence of SEQ ID NO: 103 and a HCDR2 having the sequence of SEQ ID NO: 62.

Clause 141. The bifunctional molecule of clause 125, wherein the first moiety further comprises an LCDR1 having the sequence of SEQ ID NO: 105 and a heavy chain HCDR 1 having the sequence of SEQ ID NO: 64; and an LCDR2 having the sequence of SEQ ID NO: 106 and a HCDR2 having the sequence of SEQ ID NO: 65.

Clause 142. The bifunctional molecule of clause 126, wherein the first moiety further comprises an LCDR1 having the sequence of SEQ ID NO: 108 and a heavy chain HCDR1 having the sequence of SEQ ID NO: 67; and an LCDR2 having the sequence of SEQ ID NO: 109 and a HCDR2 having the sequence of SEQ ID NO: 68.

Clause 143 The bifunctional molecule of clause 127, wherein the first moiety further comprises an LCDR1 having the sequence of SEQ ID NO: 111 and a heavy chain HCDR1 having the sequence of SEQ ID NO: 70; and an LCDR2 having the sequence of SEQ ID NO: 112 and a HCDR2 having the sequence of SEQ ID NO: 71.

Clause 144. The bifunctional molecule of clause 128, wherein the first moiety further comprises an LCDR1 having the sequence of SEQ ID NO: 114 and a heavy chain HCDR1 having the sequence of SEQ ID NO: 70; and an LCDR2 having the sequence of SEQ ID NO: 115 and a HCDR2 having the sequence of SEQ ID NO: 71.

Clause 145. The bifunctional molecule of any one of clauses 109-144, wherein the first moiety comprises a variable light chain (V_{L}) comprising framework regions LFR1, LFR2, LFR3, and LFR4 wherein LFR1 is at least 95% identical to the sequence of SEQ ID NO: 205, LFR2 is at least 95% identical to the sequence of SEQ ID NO: 206, LFR3 is at least 95% identical to the sequence of SEQ ID NO: 207; and LFR4 is at least 95% identical to the sequence of SEQ ID NO: 208; or

Clause 146. The bifunctional molecule of any one of clauses 109-144, wherein the first moiety comprises a variable light chain (V_{L}) comprising framework regions LFR1, LFR2, LFR3, and LFR4 wherein LFR1 is at least 95% identical to the sequence of SEQ ID NO: 209, LFR2 is at least 95% identical to the sequence of SEQ ID NO: 210, LFR3 is at least 95% identical to the sequence of SEQ ID NO: 211; and LFR4 is at least 95% identical to the sequence of SEQ ID NO: 212.

Clause 147. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 213, LFR2 is at least 95% identical to the sequence of SEQ ID NO: 214, LFR3 is at least 95% identical to the sequence of SEQ ID NO: 215; and LFR4 is at least 95% identical to the sequence of SEQ ID NO: 216.

Clause 148. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 217, LFR2 is at least 95% identical to the sequence of SEQ ID NO: 218, LFR3 is at least 95% identical to the sequence of SEQ ID NO: 219; and LFR4 is at least 95% identical to the sequence of SEQ ID NO: 220.

Clause 149. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 221, LFR2 is at least 95% identical to the sequence of SEQ ID NO: 222, LFR3 is at least 95% identical to the sequence of SEQ ID NO: 223; and LFR4 is at least 95% identical to the sequence of SEQ ID NO: 224.

Clause 150. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 225, LFR2 is at least 95% identical to the sequence of SEQ ID NO: 226, LFR3 is at least 95% identical to the sequence of SEQ ID NO: 227; and LFR4 is at least 95% identical to the sequence of SEQ ID NO: 228.

Clause 151. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 229, LFR2 is at least 95% identical to the sequence of SEQ ID NO: 230, LFR3 is at least 95% identical to the sequence of SEQ ID NO: 231; and LFR4 is at least 95% identical to the sequence of SEQ ID NO: 232.

Clause 152. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 233, LFR2 is at least 95% identical to the sequence of SEQ ID NO: 234, LFR3 is at least 95% identical to the sequence of SEQ ID NO: 235; and LFR4 is at least 95% identical to the sequence of SEQ ID NO: 236.

Clause 153. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 237, LFR2 is at least 95% identical to the sequence of SEQ ID NO: 238, LFR3 is at least 95% identical to the sequence of SEQ ID NO: 239; and LFR4 is at least 95% identical to the sequence of SEQ ID NO: 240.

Clause 154. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 241, LFR2 is at least 95% identical to the sequence of SEQ ID NO: 242, LFR3 is at least 95% identical to the sequence of SEQ ID NO: 243; and LFR4 is at least 95% identical to the sequence of SEQ ID NO: 244.

Clause 155. The bifunctional molecule of any one of clauses 109-144, wherein the first moiety comprises a variable light chain (VL) comprising framework regions

LFR1, LFR2, LFR3, and LFR4 wherein LFR1 is at least 95% identical to the sequence of SEQ ID NO: 245, LFR2 is at least 95% identical to the sequence of SEQ ID NO: 246, LFR3 is at least 95% identical to the sequence of SEQ ID NO: 247; and LFR4 is at least 95% identical to the sequence of SEQ ID NO: 248.

Clause 156. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 249, LFR2 is at least 95% identical to the sequence of SEQ ID NO: 250, LFR3 is at least 95% identical to the sequence of SEQ ID NO: 251; and LFR4 is at least 95% identical to the sequence of SEQ ID NO: 252.

Clause 157. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 253, LFR2 is at least 95% identical to the sequence of SEQ ID NO: 254, LFR3 is at least 95% identical to the sequence of SEQ ID NO: 255; and LFR4 is at least 95% identical to the sequence of SEQ ID NO: 256.

Clause 158. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 257, LFR2 is at least 95% identical to the sequence of SEQ ID NO: 258, LFR3 is at least 95% identical to the sequence of SEQ ID NO: 259; and LFR4 is at least 95% identical to the sequence of SEQ ID NO: 260.

Clause 159. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 261, LFR2 is at least 95% identical to the sequence of SEQ ID NO: 262, LFR3 is at least 95% identical to the sequence of SEQ ID NO: 263; and LFR4 is at least 95% identical to the sequence of SEQ ID NO: 264.

Clause 160. The bifunctional molecule of any one of clauses 109-144, wherein the first moiety comprises a variable light chain (VL) comprising framework regions LFR1, LFR2, LFR3, and LFR4 wherein LFR1 is at least 95% identical to the sequence of SEQ ID NO: 265, LFR2 is at least 95% identical to the sequence of SEQ ID NO: 266, LFR3 is at least 95% identical to the sequence of SEQ ID NO: 267; and LFR4 is at least 95% identical to the sequence of SEQ ID NO: 268.

Clause 161. The bifunctional molecule of clause 145, wherein the first moiety comprises a variable heavy chain (VH) comprising framework regions HFR1, HFR2, HFR3, and HFR4 wherein HFR1 is at least 95% identical to the sequence of SEQ ID NO: 141, HFR2 is at least 95% identical to the sequence of SEQ ID NO: 142, HFR3 is at least 95% identical to the sequence of SEQ ID NO: 143; and HFR4 is at least 95% identical to the sequence of SEQ ID NO: 144.

Clause 162. The bifunctional molecule of clause 146, wherein the first moiety comprises a variable heavy chain (VH) comprising framework regions HFR1, HFR2, HFR3, and HFR4 wherein HFR1 is at least 95% identical to the sequence of SEQ ID NO: 145, HFR2 is at least 95% identical to the sequence of SEQ ID NO: 146, HFR3 is at least 95% identical to the sequence of SEQ ID NO: 147; and HFR4 is at least 95% identical to the sequence of SEQ ID NO: 148.

Clause 163. The bifunctional molecule of clause 147, wherein the first moiety comprises a variable heavy chain (VH) comprising framework regions HFR1, HFR2, HFR3, and HFR4 wherein HFR1 is at least 95% identical to the sequence of SEQ ID NO: 149, HFR2 is at least 95% identical to the sequence of SEQ ID NO: 150, HFR3 is at least 95% identical to the sequence of SEQ ID NO: 151; and HFR4 is at least 95% identical to the sequence of SEQ ID NO: 152.

Clause 164. The bifunctional molecule of clause 148, wherein the first moiety comprises a variable heavy chain (VH) comprising framework regions HFR1, HFR2, HFR3, and HFR4 wherein HFR1 is at least 95% identical to the sequence of SEQ ID NO: 153, HFR2 is at least 95% identical to the sequence of SEQ ID NO: 154, HFR3 is at least 95% identical to the sequence of SEQ ID NO: 155; and HFR4 is at least 95% identical to the sequence of SEQ ID NO: 156.

Clause 165. The bifunctional molecule of clause 149, wherein the first moiety comprises a variable heavy chain (VH) comprising framework regions HFR1, HFR2, HFR3, and HFR4 wherein HFR1 is at least 95% identical to the sequence of SEQ ID NO: 157, HFR2 is at least 95% identical to the sequence of SEQ ID NO: 158, HFR3 is at least 95% identical to the sequence of SEQ ID NO: 159; and HFR4 is at least 95% identical to the sequence of SEQ ID NO: 160.

Clause 166. The bifunctional molecule of clause 150, wherein the first moiety comprises a variable heavy chain (VH) comprising framework regions HFR1, HFR2, HFR3, and HFR4 wherein HFR1 is at least 95% identical to the sequence of SEQ ID NO: 161, HFR2 is at least 95% identical to the sequence of SEQ ID NO: 162, HFR3 is at least 95% identical to the sequence of SEQ ID NO: 163; and HFR4 is at least 95% identical to the sequence of SEQ ID NO: 164.

Clause 167. The bifunctional molecule of clause 151, wherein the first moiety comprises a variable heavy chain (VH) comprising framework regions HFR1, HFR2, HFR3, and HFR4 wherein HFR1 is at least 95% identical to the sequence of SEQ ID NO: 165, HFR2 is at least 95% identical to the sequence of SEQ ID NO: 166, HFR3 is at least 95% identical to the sequence of SEQ ID NO: 167; and HFR4 is at least 95% identical to the sequence of SEQ ID NO: 168.

Clause 168. The bifunctional molecule of clause 152, wherein the first moiety comprises a variable heavy chain (VH) comprising framework regions HFR1, HFR2, HFR3, and HFR4 wherein HFR1 is at least 95% identical to the sequence of SEQ ID NO: 169, HFR2 is at least 95% identical to the sequence of SEQ ID NO: 170, HFR3 is at least 95% identical to the sequence of SEQ ID NO: 171; and HFR4 is at least 95% identical to the sequence of SEQ ID NO: 172.

Clause 169. The bifunctional molecule of clause 153, wherein the first moiety comprises a variable heavy chain (VH) comprising framework regions HFR1, HFR2, HFR3, and HFR4 wherein HFR1 is at least 95% identical to the sequence of SEQ ID NO: 173, HFR2 is at least 95% identical to the sequence of SEQ ID NO: 174, HFR3 is at least 95% identical to the sequence of SEQ ID NO: 175; and HFR4 is at least 95% identical to the sequence of SEQ ID NO: 176.

Clause 170. The bifunctional molecule of clause 154, wherein the first moiety comprises a variable heavy chain (VH) comprising framework regions HFR1, HFR2, HFR3, and HFR4 wherein HFR1 is at least 95% identical to the sequence of SEQ ID NO: 177, HFR2 is at least 95% identical to the sequence of SEQ ID NO: 178, HFR3 is at least 95% identical to the sequence of SEQ ID NO: 179; and HFR4 is at least 95% identical to the sequence of SEQ ID NO: 180.

Clause 171. The bifunctional molecule of clause 155, wherein the first moiety comprises a variable heavy chain (VH) comprising framework regions HFR1, HFR2, HFR3, and HFR4 wherein HFR1 is at least 95% identical to the sequence of SEQ ID NO: 181, HFR2 is at least 95% identical to the sequence of SEQ ID NO: 182, HFR3 is at least 95% identical to the sequence of SEQ ID NO: 183; and HFR4 is at least 95% identical to the sequence of SEQ ID NO: 184.

Clause 172. The bifunctional molecule of clause 156, wherein the first moiety comprises a variable heavy chain (VH) comprising framework regions HFR1, HFR2, HFR3, and HFR4 wherein HFR1 is at least 95% identical to the sequence of SEQ ID NO: 185, HFR2 is at least 95% identical to the sequence of SEQ ID NO: 186, HFR3 is at least 95% identical to the sequence of SEQ ID NO: 187; and HFR4 is at least 95% identical to the sequence of SEQ ID NO: 188.

Clause 173. The bifunctional molecule of clause 157, wherein the first moiety comprises a variable heavy chain (VH) comprising framework regions HFR1, HFR2, HFR3, and HFR4 wherein HFR1 is at least 95% identical to the sequence of SEQ ID NO: 189, HFR2 is at least 95% identical to the sequence of SEQ ID NO: 190, HFR3 is at least 95% identical to the sequence of SEQ ID NO: 191; and HFR4 is at least 95% identical to the sequence of SEQ ID NO: 192.

Clause 174. The bifunctional molecule of clause 158, wherein the first moiety comprises a variable heavy chain (VH) comprising framework regions HFR1, HFR2, HFR3, and HFR4 wherein HFR1 is at least 95% identical to the sequence of SEQ ID NO: 193, HFR2 is at least 95% identical to the sequence of SEQ ID NO: 194, HFR3 is at least 95% identical to the sequence of SEQ ID NO: 195; and HFR4 is at least 95% identical to the sequence of SEQ ID NO: 196.

Clause 175. The bifunctional molecule of clause 159, wherein the first moiety comprises a variable heavy chain (VH) comprising framework regions HFR1, HFR2, HFR3, and HFR4 wherein HFR1 is at least 95% identical to the sequence of SEQ ID NO: 197, HFR2 is at least 95% identical to the sequence of SEQ ID NO: 198, HFR3 is at least 95% identical to the sequence of SEQ ID NO: 199; and HFR4 is at least 95% identical to the sequence of SEQ ID NO: 200.

Clause 176. The bifunctional molecule of clause 160, wherein the first moiety comprises a variable heavy chain (VH) comprising framework regions HFR1, HFR2, HFR3, and HFR4 wherein HFR1 is at least 95% identical to the sequence of SEQ ID NO: 201, HFR2 is at least 95% identical to the sequence of SEQ ID NO: 202, HFR3 is at least 95% identical to the sequence of SEQ ID NO: 203; and HFR4 is at least 95% identical to the sequence of SEQ ID NO: 204.

Clause 177. The bifunctional molecule of any one of clauses 109-176, wherein the first moiety comprises a variable light chain (VL) having an amino acid sequence that is at least 90% identical to an amino acid sequence selected from SEQ ID NOs: 17-32.

Clause 178. The bifunctional molecule of any one of clauses 109-177, wherein the first moiety comprises a variable heavy chain (VH) having an amino acid sequence that is at least 90% identical to an amino acid sequence selected from SEQ ID NOs: 1-16.

Clause 179. A method of degrading a soluble target molecule or a cell surface target molecule comprising:
(a) contacting the target molecule with a multi-specific antigen binding protein (ABP), wherein the ABP comprises a first antigen binding moiety that specifically binds to a cation-independent mannose 6 phosphate receptor (CI-M6PR) on the surface of a cell; and
(b) transporting the ABP, the target molecule, and the CI-M6PR to a lysosome within a cell, wherein the target molecule is degraded in the lysosome.

Clause 180. The method of clause 179, wherein the ABP further comprises a linked cargo moiety.

Clause 181. The method of clause 180, wherein the cargo moiety is a polypeptide that is fused to the ABP.

Clause 182. The method of clause 181, wherein the cargo moiety is conjugated to the ABP, optionally via a linker.

Clause 183. The method of any one of clauses 179-182, wherein the first antigen binding moiety binds to a domain of CI-M6PR selected from: domain 1, domain 4, domain 5, domain 6, domain 7, and domain 8.

Clause 184. The method of any one of clauses 179-182, wherein the first antigen binding moiety binds CI-M6PR with a dissociation equilibrium constant (K_{D}) of about 10 nM or less.

Clause 185. The method of any one of clauses 179-182, wherein the first antigen binding moiety binds CI-M6PR with a dissociation equilibrium constant (K_{D}) between about 1 nM and about 500 nM.

Clause 186. The method of any one of clauses 179-185, wherein the off rate (k_{off}) of the first antigen binding moiety for CI-M6PR is between 1×10⁻⁸ s⁻¹ and 0.1 s⁻¹.

Clause 187. The method of any one of clauses 179-186, wherein the binding of the first antigen binding moiety to CI-M6PR is pH dependent.

Clause 188. The method of any one of clauses 179-187, wherein the first antigen binding moiety is released from CI-M6PR at a pH of 7.4 or lower.

Clause 189. The method of any one of clauses 179-188, wherein the first antigen binding moiety is released from CI-M6PR at a pH of 6.0 or lower.

Clause 190. The method of any one of clauses 179-189, wherein the first antigen binding moiety is released from CI-M6PR at a pH of 5.5 or lower.

Clause 191. The method of any one of clauses 179-190, wherein the first antigen binding moiety is released from CI-M6PR at a pH of 5.0 or lower.

Clause 192. The method of any one of clauses 179-191, wherein the first antigen binding moiety comprises a light chain CDR3 (LCDR3) having the sequence of SEQ ID NO: 76 and a heavy chain CDR3 (HCDR3) having the sequence of SEQ ID NO: 35.

Clause 193. The method of any one of clauses 179-191, wherein the first antigen binding moiety comprises a light chain CDR3 having the sequence of SEQ ID NO: 78 and a heavy chain CDR3 having the amino acid sequence of SEQ ID NO: 38.

Clause 194. The method of any one of clauses 179-191, wherein the first antigen binding moiety comprises a light chain CDR3 having the sequence of SEQ ID NO: 81 and a heavy chain CDR3 having the sequence of SEQ ID NO: 41.

Clause 195. The method of any one of clauses 179-191, wherein the first antigen binding moiety comprises a light chain CDR3 having the sequence of SEQ ID NO: 84 and a heavy chain CDR3 having the sequence of SEQ ID NO: 44.

Clause 196. The method of any one of clauses 179-191, wherein the first antigen binding moiety comprises a light chain CDR3 having the sequence of SEQ ID NO: 85 and a heavy chain CDR3 having the sequence of SEQ ID NO: 46.

Clause 197. The method of any one of clauses 179-191, wherein the first antigen binding moiety comprises a light chain CDR3 having the sequence of SEQ ID NO: 88 and a heavy chain CDR3 having the sequence of SEQ ID NO: 49.

Clause 198. The method of any one of clauses 179-191, wherein the first antigen binding moiety comprises a light chain CDR3 having the sequence of SEQ ID NO: 91 and a heavy chain CDR3 having the sequence of SEQ ID NO: 52.

Clause 199. The method of any one of clauses 179-191, wherein the first antigen binding moiety comprises a light chain CDR3 having the sequence of SEQ ID NO: 94 and a heavy chain CDR3 having the sequence of SEQ ID NO: 54.

Clause 200. The method of any one of clauses 179-191, wherein the first antigen binding moiety comprises a light chain CDR3 having the sequence of SEQ ID NO: 96 and a heavy chain CDR3 having the sequence of SEQ ID NO: 55.

Clause 201. The method of any one of clauses 179-191, wherein the first antigen binding moiety comprises a light chain CDR3 having the sequence of SEQ ID NO: 99 and a heavy chain CDR3 having the sequence of SEQ ID NO: 58.

Clause 202. The method of any one of clauses 179-191, wherein the first antigen binding moiety comprises a light chain CDR3 having the sequence of SEQ ID NO: 101 and a heavy chain CDR3 having the sequence of SEQ ID NO: 60.

Clause 203. The method of any one of clauses 179-191, wherein the first antigen binding moiety comprises a light chain CDR3 having the sequence of SEQ ID NO: 104 and a heavy chain CDR3 having the sequence of SEQ ID NO: 63.

Clause 204. The method of any one of clauses 179-191, wherein the first antigen binding moiety comprises a light chain CDR3 having the sequence of SEQ ID NO: 107 and a heavy chain CDR3 having the sequence of SEQ ID NO: 66.

Clause 205. The method of any one of clauses 179-191, wherein the first antigen binding moiety comprises a light chain CDR3 having the sequence of SEQ ID NO: 110 and a heavy chain CDR3 having the sequence of SEQ ID NO: 69.

Clause 206. The method of any one of clauses 179-191, wherein the first antigen binding moiety comprises a light chain CDR3 having the sequence of SEQ ID NO: 113 and a heavy chain CDR3 having the sequence of SEQ ID NO: 72.

Clause 207. The method of any one of clauses 179-191, wherein the first antigen binding moiety comprises a light chain CDR3 having the sequence of SEQ ID NO: 116 and a heavy chain CDR3 having the sequence of SEQ ID NO: 73.

Clause 208. The method of clause 192, wherein the first antigen binding moiety further comprises a light chain CDR1 (LCDR1) having the sequence of SEQ ID NO: 74 and a heavy chain CDR1 (HCDR1) having the sequence of SEQ ID NO: 33; and a light chain CDR2 (LCDR2) having the sequence of SEQ ID NO: 75 and a heavy chain CDR2 (HCDR2) having the sequence of SEQ ID NO: 34.

Clause 209. The method of clause 193, wherein the first antigen binding moiety further comprises an LCDR1 having the sequence of SEQ ID NO: 77 and a heavy chain HCDR1 having the sequence of SEQ ID NO: 36; and an LCDR2 having the sequence of SEQ ID NO: 75 and a HCDR2 having the sequence of SEQ ID NO: 37.

Clause 210. The method of clause 194, wherein the first antigen binding moiety further comprises an LCDR1 having the sequence of SEQ ID NO: 79 and a heavy chain HCDR1 having the sequence of SEQ ID NO: 39; and an LCDR2 having the sequence of SEQ ID NO: 80 and a HCDR2 having the sequence of SEQ ID NO: 40.

Clause 211. The method of clause 195, wherein the first antigen binding moiety further comprises an LCDR1 having the sequence of SEQ ID NO: 82 and a heavy chain HCDR1 having the sequence of SEQ ID NO: 42; and an LCDR2 having the sequence of SEQ ID NO: 83 and a HCDR2 having the sequence of SEQ ID NO: 43.

Clause 212. The method of clause 196, wherein the first antigen binding moiety further comprises an LCDR1 having the sequence of SEQ ID NO: 79 and a heavy chain HCDR1 having the sequence of SEQ ID NO: 45; and an LCDR2 having the sequence of SEQ ID NO: 80 and a HCDR2 having the sequence of SEQ ID NO: 34.

Clause 213. The method of clause 197, wherein the first antigen binding moiety further comprises an LCDR1 having the sequence of SEQ ID NO: 86 and a heavy chain HCDR1 having the sequence of SEQ ID NO: 47; and an LCDR2 having the sequence of SEQ ID NO: 87 and a HCDR2 having the sequence of SEQ ID NO: 48.

Clause 214. The method of clause 198, wherein the first antigen binding moiety further comprises an LCDR1 having the sequence of SEQ ID NO: 89 and a heavy chain HCDR1 having the sequence of SEQ ID NO: 50; and an LCDR2 having the sequence of SEQ ID NO: 90 and a HCDR2 having the sequence of SEQ ID NO: 53.

Clause 215. The method of clause 199, wherein the first antigen binding moiety further comprises an LCDR1 having the sequence of SEQ ID NO: 92 and a heavy chain HCDR1 having the sequence of SEQ ID NO: 50; and an LCDR2 having the sequence of SEQ ID NO: 93 and a HCDR2 having the sequence of SEQ ID NO: 53.

Clause 216. The method of clause 200, wherein the first antigen binding moiety further comprises an LCDR1 having the sequence of SEQ ID NO: 95 and a heavy chain HCDR1 having the sequence of SEQ ID NO: 50; and an LCDR2 having the sequence of SEQ ID NO: 93 and a HCDR2 having the sequence of SEQ ID NO: 51.

Clause 217. The method of clause 201, wherein the first antigen binding moiety further comprises an LCDR1 having the sequence of SEQ ID NO: 97 and a heavy chain HCDR1 having the sequence of SEQ ID NO: 56; and an LCDR2 having the sequence of SEQ ID NO: 98 and a HCDR2 having the sequence of SEQ ID NO: 57.

Clause 218. The method of clause 202, wherein the first antigen binding moiety further comprises an LCDR1 having the sequence of SEQ ID NO: 100 and a heavy chain HCDR1 having the sequence of SEQ ID NO: 50; and an LCDR2 having the sequence of SEQ ID NO: 93 and a HCDR2 having the sequence of SEQ ID NO: 59.

Clause 219. The method of clause 203, wherein the first antigen binding moiety further comprises an LCDR1 having the sequence of SEQ ID NO: 102 and a heavy chain HCDR1 having the sequence of SEQ ID NO: 61; and an LCDR2 having the sequence of SEQ ID NO: 103 and a HCDR2 having the sequence of SEQ ID NO: 62.

Clause 220. The method of clause 204, wherein the first antigen binding moiety further comprises an LCDR1 having the sequence of SEQ ID NO: 105 and a heavy chain HCDR1 having the sequence of SEQ ID NO: 64; and an LCDR2 having the sequence of SEQ ID NO: 106 and a HCDR2 having the sequence of SEQ ID NO: 65.

Clause 221. The method of clause 205, wherein the first antigen binding moiety further comprises an LCDR1 having the sequence of SEQ ID NO: 108 and a heavy chain HCDR1 having the sequence of SEQ ID NO: 67; and an LCDR2 having the sequence of SEQ ID NO: 109 and a HCDR2 having the sequence of SEQ ID NO: 68.

Clause 222. The method of clause 206, wherein the first antigen binding moiety further comprises an LCDR1 having the sequence of SEQ ID NO: 111 and a heavy chain HCDR1 having the sequence of SEQ ID NO: 70; and an LCDR2 having the sequence of SEQ ID NO: 112 and a HCDR2 having the sequence of SEQ ID NO: 71.

Clause 223. The method of clause 207, wherein the first antigen binding moiety further comprises an LCDR1 having the sequence of SEQ ID NO: 114 and a heavy chain HCDR1 having the sequence of SEQ ID NO: 70; and an LCDR2 having the sequence of SEQ ID NO: 115 and a HCDR2 having the sequence of SEQ ID NO: 71.

Clause 224. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 205, LFR2 is at least 95% identical to the sequence of SEQ ID NO: 206, LFR3 is at least 95% identical to the sequence of SEQ ID NO: 207; and LFR4 is at least 95% identical to the sequence of SEQ ID NO: 208.

Clause 225. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 209, LFR2 is at least 95% identical to the sequence of SEQ ID NO: 210, LFR3 is at least 95% identical to the sequence of SEQ ID NO: 211; and LFR4 is at least 95% identical to the sequence of SEQ ID NO: 212.

Clause 226. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 213, LFR2 is at least 95% identical to the sequence of SEQ ID NO: 214, LFR3 is at least 95% identical to the sequence of SEQ ID NO: 215; and LFR4 is at least 95% identical to the sequence of SEQ ID NO: 216.

Clause 227. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 217, LFR2 is at least 95% identical to the sequence of SEQ ID NO: 218, LFR3 is at least 95% identical to the sequence of SEQ ID NO: 219; and LFR4 is at least 95% identical to the sequence of SEQ ID NO: 220.

Clause 228. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 221, LFR2 is at least 95% identical to the sequence of SEQ ID NO: 222, LFR3 is at least 95% identical to the sequence of SEQ ID NO: 223; and LFR4 is at least 95% identical to the sequence of SEQ ID NO: 224.

Clause 229. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 225, LFR2 is at least 95% identical to the sequence of SEQ ID NO: 226, LFR3 is at least 95% identical to the sequence of SEQ ID NO: 227; and LFR4 is at least 95% identical to the sequence of SEQ ID NO: 228.

Clause 230. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 229, LFR2 is at least 95% identical to the sequence of SEQ ID NO: 230, LFR3 is at least 95% identical to the sequence of SEQ ID NO: 231; and LFR4 is at least 95% identical to the sequence of SEQ ID NO: 232.

Clause 231. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 233, LFR2 is at least 95% identical to the sequence of SEQ ID NO: 234, LFR3 is at least 95% identical to the sequence of SEQ ID NO: 235; and LFR4 is at least 95% identical to the sequence of SEQ ID NO: 236.

Clause 232. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 237, LFR2 is at least 95% identical to the sequence of SEQ ID NO: 238, LFR3 is at least 95% identical to the sequence of SEQ ID NO: 239; and LFR4 is at least 95% identical to the sequence of SEQ ID NO: 240.

Clause 233. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 241, LFR2 is at least 95% identical to the sequence of SEQ ID NO: 242, LFR3 is at least 95% identical to the sequence of SEQ ID NO: 243; and LFR4 is at least 95% identical to the sequence of SEQ ID NO: 244.

Clause 234. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 245, LFR2 is at least 95% identical to the sequence of SEQ ID NO: 246, LFR3 is at least 95% identical to the sequence of SEQ ID NO: 247; and LFR4 is at least 95% identical to the sequence of SEQ ID NO: 248.

Clause 235. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 249, LFR2 is at least 95% identical to the sequence of SEQ ID NO: 250, LFR3 is at least 95% identical to the sequence of SEQ ID NO: 251; and LFR4 is at least 95% identical to the sequence of SEQ ID NO: 252.

Clause 236. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 253, LFR2 is at least 95% identical to the sequence of SEQ ID NO: 254, LFR3 is at least 95% identical to the sequence of SEQ ID NO: 255; and LFR4 is at least 95% identical to the sequence of SEQ ID NO: 256.

Clause 237. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 257, LFR2 is at least 95% identical to the sequence of SEQ ID NO: 258, LFR3 is at least 95% identical to the sequence of SEQ ID NO: 259; and LFR4 is at least 95% identical to the sequence of SEQ ID NO: 260.

Clause 238. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 261, LFR2 is at least 95% identical to the sequence of SEQ ID NO: 262, LFR3 is at least 95% identical to the sequence of SEQ ID NO: 263; and LFR4 is at least 95% identical to the sequence of SEQ ID NO: 264.

Clause 239. The method of any one of clauses 179-223, wherein the first antigen binding moiety comprises a variable light chain (VL) comprising framework regions LFR1, LFR2, LFR3, and LFR4 wherein LFR1 is at least 95% identical to the sequence of SEQ ID NO: 265, LFR2 is at least 95% identical to the sequence of SEQ ID NO: 266, LFR3 is at least 95% identical to the sequence of SEQ ID NO: 267; and LFR4 is at least 95% identical to the sequence of SEQ ID NO: 268.

Clause 240. The method of clause 224, wherein the first antigen binding moiety comprises a variable heavy chain (VH) comprising framework regions HFR1, HFR2, HFR3, and HFR4 wherein HFR1 is at least 95% identical to the sequence of SEQ ID NO: 141, HFR2 is at least 95% identical to the sequence of SEQ ID NO: 142, HFR3 is at least 95% identical to the sequence of SEQ ID NO: 143; and HFR4 is at least 95% identical to the sequence of SEQ ID NO: 144.

Clause 241. The method of clause 225, wherein the first antigen binding moiety comprises a variable heavy chain (VH) comprising framework regions HFR1, HFR2, HFR3, and HFR4 wherein HFR1 is at least 95% identical to the sequence of SEQ ID NO: 145, HFR2 is at least 95% identical to the sequence of SEQ ID NO: 146, HFR3 is at least 95% identical to the sequence of SEQ ID NO: 147; and HFR4 is at least 95% identical to the sequence of SEQ ID NO: 148.

Clause 242. The method of clause 226, wherein the first antigen binding moiety comprises a variable heavy chain (VH) comprising framework regions HFR1, HFR2, HFR3, and HFR4 wherein HFR1 is at least 95% identical to the sequence of SEQ ID NO: 149, HFR2 is at least 95% identical to the sequence of SEQ ID NO: 150, HFR3 is at least 95% identical to the sequence of SEQ ID NO: 151; and HFR4 is at least 95% identical to the sequence of SEQ ID NO: 152.

Clause 243. The method of clause 227, wherein the first antigen binding moiety comprises a variable heavy chain (VH) comprising framework regions HFR1, HFR2, HFR3, and HFR4 wherein HFR1 is at least 95% identical to the sequence of SEQ ID NO: 153, HFR2 is at least 95% identical to the sequence of SEQ ID NO: 154, HFR3 is at least 95% identical to the sequence of SEQ ID NO: 155; and HFR4 is at least 95% identical to the sequence of SEQ ID NO: 156.

Clause 244. The method of clause 228, wherein the first antigen binding moiety comprises a variable heavy chain (VH) comprising framework regions HFR1, HFR2, HFR3, and HFR4 wherein HFR1 is at least 95% identical to the sequence of SEQ ID NO: 157, HFR2 is at least 95% identical to the sequence of SEQ ID NO: 158, HFR3 is at least 95% identical to the sequence of SEQ ID NO: 159; and HFR4 is at least 95% identical to the sequence of SEQ ID NO: 160.

Clause 245. The method of clause 229, wherein the first antigen binding moiety comprises a variable heavy chain (VH) comprising framework regions HFR1, HFR2, HFR3, and HFR4 wherein HFR1 is at least 95% identical to the sequence of SEQ ID NO: 161, HFR2 is at least 95% identical to the sequence of SEQ ID NO: 162, HFR3 is at least 95% identical to the sequence of SEQ ID NO: 163; and HFR4 is at least 95% identical to the sequence of SEQ ID NO: 164.

Clause 246. The method of clause 230, wherein the first antigen binding moiety comprises a variable heavy chain (VH) comprising framework regions HFR1, HFR2, HFR3, and HFR4 wherein HFR1 is at least 95% identical to the sequence of SEQ ID NO: 165, HFR2 is at least 95% identical to the sequence of SEQ ID NO: 166, HFR3 is at least 95% identical to the sequence of SEQ ID NO: 167; and HFR4 is at least 95% identical to the sequence of SEQ ID NO: 168.

Clause 247. The method of clause 231, wherein the first antigen binding moiety comprises a variable heavy chain (VH) comprising framework regions HFR1, HFR2, HFR3, and HFR4 wherein HFR1 is at least 95% identical to the sequence of SEQ ID NO: 169, HFR2 is at least 95% identical to the sequence of SEQ ID NO: 170, HFR3 is at least 95% identical to the sequence of SEQ ID NO: 171; and HFR4 is at least 95% identical to the sequence of SEQ ID NO: 172.

Clause 248. The method of clause 232, wherein the first antigen binding moiety comprises a variable heavy chain (VH) comprising framework regions HFR1, HFR2, HFR3, and HFR4 wherein HFR1 is at least 95% identical to the sequence of SEQ ID NO: 173, HFR2 is at least 95% identical to the sequence of SEQ ID NO: 174, HFR3 is at least 95% identical to the sequence of SEQ ID NO: 175; and HFR4 is at least 95% identical to the sequence of SEQ ID NO: 176.

Clause 249. The method of clause 233, wherein the first antigen binding moiety comprises a variable heavy chain (VH) comprising framework regions HFR1, HFR2, HFR3, and HFR4 wherein HFR1 is at least 95% identical to the sequence of SEQ ID NO: 177, HFR2 is at least 95% identical to the sequence of SEQ ID NO: 178, HFR3 is at least 95% identical to the sequence of SEQ ID NO: 179; and HFR4 is at least 95% identical to the sequence of SEQ ID NO: 180.

Clause 250. The method of clause 234, wherein the first antigen binding moiety comprises a variable heavy chain (VH) comprising framework regions HFR1, HFR2, HFR3, and HFR4 wherein HFR1 is at least 95% identical to the sequence of SEQ ID NO: 181, HFR2 is at least 95% identical to the sequence of SEQ ID NO: 182, HFR3 is at least 95% identical to the sequence of SEQ ID NO: 183; and HFR4 is at least 95% identical to the sequence of SEQ ID NO: 184.

Clause 251. The method of clause 235, wherein the first antigen binding moiety comprises a variable heavy chain (VH) comprising framework regions HFR1, HFR2, HFR3, and HFR4 wherein HFR1 is at least 95% identical to the sequence of SEQ ID NO: 185, HFR2 is at least 95% identical to the sequence of SEQ ID NO: 186, HFR3 is at least 95% identical to the sequence of SEQ ID NO: 187; and HFR4 is at least 95% identical to the sequence of SEQ ID NO: 188.

Clause 252. The method of clause 236, wherein the first antigen binding moiety comprises a variable heavy chain (VH) comprising framework regions HFR1, HFR2, HFR3, and HFR4 wherein HFR1 is at least 95% identical to the sequence of SEQ ID NO: 189, HFR2 is at least 95% identical to the sequence of SEQ ID NO: 190, HFR3 is at least 95% identical to the sequence of SEQ ID NO: 191; and HFR4 is at least 95% identical to the sequence of SEQ ID NO: 192.

Clause 253. The method of clause 237, wherein the first antigen binding moiety comprises a variable heavy chain (VH) comprising framework regions HFR1, HFR2, HFR3, and HFR4 wherein HFR1 is at least 95% identical to the sequence of SEQ ID NO: 193, HFR2 is at least 95% identical to the sequence of SEQ ID NO: 194, HFR3 is at least 95% identical to the sequence of SEQ ID NO: 195; and HFR4 is at least 95% identical to the sequence of SEQ ID NO: 196.

Clause 254. The method of clause 238, wherein the first antigen binding moiety comprises a variable heavy chain (VH) comprising framework regions HFR1, HFR2, HFR3, and HFR4 wherein HFR1 is at least 95% identical to the sequence of SEQ ID NO: 197, HFR2 is at least 95% identical to the sequence of SEQ ID NO: 198, HFR3 is at least 95% identical to the sequence of SEQ ID NO: 199; and HFR4 is at least 95% identical to the sequence of SEQ ID NO: 200.

Clause 255 The method of clause 239, wherein the first antigen binding moiety comprises a variable heavy chain (VH) comprising framework regions HFR1, HFR2, HFR3, and HFR4 wherein HFR1 is at least 95% identical to the sequence of SEQ ID NO: 201, HFR2 is at least 95% identical to the sequence of SEQ ID NO: 202, HFR3 is at least 95% identical to the sequence of SEQ ID NO: 203; and HFR4 is at least 95% identical to the sequence of SEQ ID NO: 204.

Clause 256. The method of any one of clauses 179-255, wherein the first antigen binding moiety comprises a variable light chain (VL) having an amino acid sequence that is at least 90% identical to an amino acid sequence selected from SEQ ID NOs: 17-32.

Clause 257. The method of any one of clauses 179-256, wherein the first antigen binding moiety comprises a variable heavy chain (VH) having an amino acid sequence that is at least 90% identical to an amino acid sequence selected from SEQ ID NOs: 1-16.

Clause 258. The method of any one of clauses 179-257, wherein the first antigen binding moiety is an antibody fragment

Clause 259. The method of any one of clauses 179-257, further comprising
(c) transporting the ABP to the cell surface.

Clause 260. A method of internalizing a target molecule, the method comprising:
(a) contacting the target molecule with an antigen binding protein (ABP) of any one of clauses 1-108 or a bifunctional molecule of any one of clauses 109-178; and
(b) internalizing the ABP within a cell.

### 9. EQUIVALENTS

While the invention has been particularly shown and described with reference to a preferred embodiment and various alternate embodiments, it will be understood by persons skilled in the relevant art that various changes in form and details can be made therein without departing from the spirit and scope of the invention.

What is claimed is:

1. An antigen binding protein (ABP) comprising a first antigen binding moiety that specifically binds to an internalizing domain of cation-independent mannose 6 phosphate receptor (CI-M6PR) that facilitates intracellular transport of the ABP.

2. The ABP of claim 1, further comprising a target binding moiety that specifically binds to a target molecule.

3. The ABP of claim 2, wherein the target binding moiety is a second antigen binding moiety that specifically binds to the target molecule.

4. The ABP of claim 2, wherein the target binding moiety is conjugated to the first antigen binding moiety, optionally via a linker.

5. The ABP of any one of claims 2-4, wherein the target molecule is a soluble extracellular target molecule or a cell surface target molecule.

6. The ABP of any one of the preceding claims, further comprising a linked cargo moiety.

7. The ABP of claim 6, wherein the cargo moiety is a polypeptide that is fused to the first or second antigen binding moiety.

8. The ABP of claim 6, wherein the cargo moiety is conjugated to the first or second antigen binding moiety, optionally via a linker.

9. The ABP of claim 1 or 2, wherein the first antigen binding moiety binds to a domain of CI-M6PR selected from: domain 1, domain 4, domain 5, domain 6, domain 7, and domain 8.

10. The ABP of claim 1 or 2, wherein the first antigen binding moiety binds to domain 1 of CI-M6PR.

11. The ABP of claim 1 or 2, wherein the first antigen binding moiety binds to domain 4 of CI-M6PR.

12. The ABP of claim 1 or 2, wherein the first antigen binding moiety binds to domain 5 of CI-M6PR.

13. The ABP of claim 1 or 2, wherein the first antigen binding moiety binds to domain 6 of CI-M6PR.

14. The ABP of claim 1 or 2, wherein the first antigen binding moiety binds to domain 7 of CI-M6PR.

15. The ABP of claim 1 or 2, wherein the first antigen binding moiety binds to domain 8 of CI-M6PR.

16. The ABP of any one of the preceding claims, wherein the first antigen binding moiety binds the CI-M6PR with high affinity.

17. The ABP of claim 16, wherein the first antigen binding moiety binds CI-M6PR with a dissociation equilibrium constant (K_{D}) of about 10 nM or less.

18. The ABP of any one of the preceding claims, wherein the first antigen binding moiety binds CI-M6PR with a K_{D} between about 1 nM and about 500 nM.

19. The ABP of claim 18, wherein the first antigen binding moiety binds CI-M6PR with a K_{D} between about 10 nM and about 100 nM.

20. The ABP of claim 18, wherein the first antigen binding moiety binds CI-M6PR with a K_{D} between about 100 nM and about 200 nM.

21. The ABP of claim 18, wherein the first antigen binding moiety binds CI-M6PR with a K_{D} between about 200 nM and about 300 nM.

22. The ABP of claim 18, wherein the first antigen binding moiety binds CI-M6PR with a K_{D} between about 300 nM and about 400 nM.

23. The ABP of claim 18, wherein the first antigen binding moiety binds CI-M6PR with a K_{D} between about 400 nM and about 500 nM.

24. The ABP of any one of the preceding claims, wherein the off rate (K_{off}) of the first antigen binding moiety for CI-M6PR is between 1×10⁻⁸ s⁻¹ and 0.1 s⁻¹.

25. The ABP of any one of the preceding claims, wherein K_{off} of the first antigen binding moiety for CI-M6PR is between 1×10⁻⁶ s⁻¹ and 1×10⁻² s⁻¹.

26. The ABP of any one of the preceding claims, wherein K_{off} of the first antigen binding moiety for CI-M6PR is about 1×10⁻⁵ s⁻¹ or slower.

27. The ABP of any one of the preceding claims, wherein the binding of the first antigen binding moiety to CI-M6PR is pH dependent.

28. The ABP of any one of the preceding claims, wherein the first antigen binding moiety is released from CI-M6PR at a pH of 7.4 or lower.

29. The ABP of any one of the preceding claims, wherein the first antigen binding moiety is released from CI-M6PR at a pH of 6.5 or lower.

30. The ABP of any one of the preceding claims, wherein the first antigen binding moiety is released from CI-M6PR at a pH of 6.0 or lower.

31. The ABP of any one of the preceding claims, wherein the first antigen binding moiety is released from CI-M6PR at a pH of 5.5 or lower.

32. The ABP of any one of the preceding claims, wherein the first antigen binding moiety is released from CI-M6PR at a pH of 5.0 or lower.

33. The ABP of any one of the preceding claims, wherein the first antigen binding moiety comprises a light chain CDR3 (LCDR3) having the sequence of SEQ ID NO: 76 and a heavy chain CDR3 (HCDR3) having the sequence of SEQ ID NO: 35.

34. The ABP of any one of claims 1-32, wherein the first antigen binding moiety comprises a light chain CDR3 having the sequence of SEQ ID NO: 78 and a heavy chain CDR3 having the amino acid sequence of SEQ ID NO: 38.

35. The ABP of any one of claims 1-32, wherein the first antigen binding moiety comprises a light chain CDR3 having the sequence of SEQ ID NO: 81 and a heavy chain CDR3 having the sequence of SEQ ID NO: 41.

36. The ABP of any one of claims 1-32, wherein the first antigen binding moiety comprises a light chain CDR3 having the sequence of SEQ ID NO: 84 and a heavy chain CDR3 having the sequence of SEQ ID NO: 44.

37. The ABP of any one of claims 1-32, wherein the first antigen binding moiety comprises a light chain CDR3 having the sequence of SEQ ID NO: 85 and a heavy chain CDR3 having the sequence of SEQ ID NO: 46.

38. The ABP of any one of claims 1-32, wherein the first antigen binding moiety comprises a light chain CDR3 having the sequence of SEQ ID NO: 88 and a heavy chain CDR3 having the sequence of SEQ ID NO: 49.

39. The ABP of any one of claims 1-32, wherein the first antigen binding moiety comprises a light chain CDR3 having the sequence of SEQ ID NO: 91 and a heavy chain CDR3 having the sequence of SEQ ID NO: 52.

40. The ABP of any one of claims 1-32, wherein the first antigen binding moiety comprises a light chain CDR3 having the sequence of SEQ ID NO: 94 and a heavy chain CDR3 having the sequence of SEQ ID NO: 54.

41. The ABP of any one of claims 1-32, wherein the first antigen binding moiety comprises a light chain CDR3 having the sequence of SEQ ID NO: 96 and a heavy chain CDR3 having the sequence of SEQ ID NO: 55.

42. The ABP of any one of claims 1-32, wherein the first antigen binding moiety comprises a light chain CDR3 having the sequence of SEQ ID NO: 99 and a heavy chain CDR3 having the sequence of SEQ ID NO: 58.

43. The ABP of any one of claims 1-32, wherein the first antigen binding moiety comprises a light chain CDR3 having the sequence of SEQ ID NO: 101 and a heavy chain CDR3 having the sequence of SEQ ID NO: 60.

44. The ABP of any one of claims 1-32, wherein the first antigen binding moiety comprises a light chain CDR3 having the sequence of SEQ ID NO: 104 and a heavy chain CDR3 having the sequence of SEQ ID NO: 63.

45. The ABP of any one of claims 1-32, wherein the first antigen binding moiety comprises a light chain CDR3 having the sequence of SEQ ID NO: 107 and a heavy chain CDR3 having the sequence of SEQ ID NO: 66.

46. The ABP of any one of claims 1-32, wherein the first antigen binding moiety comprises a light chain CDR3 having the sequence of SEQ ID NO: 110 and a heavy chain CDR3 having the sequence of SEQ ID NO: 69.

47. The ABP of any one of claims 1-32, wherein the first antigen binding moiety comprises a light chain CDR3 having the sequence of SEQ ID NO: 113 and a heavy chain CDR3 having the sequence of SEQ ID NO: 72.

48. The ABP of any one of claims 1-32, wherein the first antigen binding moiety comprises a light chain CDR3 having the sequence of SEQ ID NO: 116 and a heavy chain CDR3 having the sequence of SEQ ID NO: 73.

49. The ABP of claim 33, wherein the first antigen binding moiety further comprises a light chain CDR1 (LCDR1) having the sequence of SEQ ID NO: 74 and a heavy chain CDR1 (HCDR1) having the sequence of SEQ ID NO: 33; and a light chain CDR2 (LCDR2) having the sequence of SEQ ID NO: 75 and a heavy chain CDR2 (HCDR2) having the sequence of SEQ ID NO: 34.

50. The ABP of claim 34, wherein the first antigen binding moiety further comprises an LCDR1 having the sequence of SEQ ID NO: 77 and a heavy chain HCDR1 having the sequence of SEQ ID NO: 36; and an LCDR2 having the sequence of SEQ ID NO: 75 and a HCDR2 having the sequence of SEQ ID NO: 37.

51. The ABP of claim 35, wherein the first antigen binding moiety further comprises an LCDR1 having the sequence of SEQ ID NO: 79 and a heavy chain HCDR1 having the sequence of SEQ ID NO: 39; and an LCDR2 having the sequence of SEQ ID NO: 80 and a HCDR2 having the sequence of SEQ ID NO: 40.

52. The ABP of claim 36, wherein the first antigen binding moiety further comprises an LCDR1 having the sequence of SEQ ID NO: 82 and a heavy chain HCDR1 having the sequence of SEQ ID NO: 42; and an LCDR2 having the sequence of SEQ ID NO: 83 and a HCDR2 having the sequence of SEQ ID NO: 43.

53. The ABP of claim 37, wherein the first antigen binding moiety further comprises an LCDR1 having the sequence of SEQ ID NO: 79 and a heavy chain HCDR1 having the sequence of SEQ ID NO: 45; and an LCDR2 having the sequence of SEQ ID NO: 80 and a HCDR2 having the sequence of SEQ ID NO: 34.

54. The ABP of claim 38, wherein the first antigen binding moiety further comprises an LCDR1 having the sequence of SEQ ID NO: 86 and a heavy chain HCDR1 having the sequence of SEQ ID NO: 47; and an LCDR2 having the sequence of SEQ ID NO: 87 and a HCDR2 having the sequence of SEQ ID NO: 48.

55. The ABP of claim 39, wherein the first antigen binding moiety further comprises an LCDR1 having the sequence of SEQ ID NO: 89 and a heavy chain HCDR1 having the sequence of SEQ ID NO: 50; and an LCDR2 having the sequence of SEQ ID NO: 90 and a HCDR2 having the sequence of SEQ ID NO: 53.

56. The ABP of claim 40, wherein the first antigen binding moiety further comprises an LCDR1 having the sequence of SEQ ID NO: 92 and a heavy chain HCDR1 having the sequence of SEQ ID NO: 50; and an LCDR2 having the sequence of SEQ ID NO: 93 and a HCDR2 having the sequence of SEQ ID NO: 53.

57. The ABP of claim 41, wherein the first antigen binding moiety further comprises an LCDR1 having the sequence of SEQ ID NO: 95 and a heavy chain HCDR1 having the sequence of SEQ ID NO: 50; and an LCDR2 having the sequence of SEQ ID NO: 93 and a HCDR2 having the sequence of SEQ ID NO: 51.

58. The ABP of claim 42, wherein the first antigen binding moiety further comprises an LCDR1 having the sequence of SEQ ID NO: 97 and a heavy chain HCDR1 having the sequence of SEQ ID NO: 56; and an LCDR2 having the sequence of SEQ ID NO: 98 and a HCDR2 having the sequence of SEQ ID NO: 57.

59. The ABP of claim 43, wherein the first antigen binding moiety further comprises an LCDR1 having the sequence of SEQ ID NO: 100 and a heavy chain HCDR1 having the sequence of SEQ ID NO: 50; and an LCDR2 having the sequence of SEQ ID NO: 93 and a HCDR2 having the sequence of SEQ ID NO: 59.

60. The ABP of claim 44, wherein the first antigen binding moiety further comprises an LCDR1 having the sequence of SEQ ID NO: 102 and a heavy chain HCDR1 having the sequence of SEQ ID NO: 61; and an LCDR2 having the sequence of SEQ ID NO: 103 and a HCDR2 having the sequence of SEQ ID NO: 62.

61. The ABP of claim 45, wherein the first antigen binding moiety further comprises an LCDR1 having the sequence of SEQ ID NO: 105 and a heavy chain HCDR1 having the sequence of SEQ ID NO: 64; and an LCDR2 having the sequence of SEQ ID NO: 106 and a HCDR2 having the sequence of SEQ ID NO: 65.

62. The ABP of claim 46, wherein the first antigen binding moiety further comprises an LCDR1 having the sequence of SEQ ID NO: 108 and a heavy chain HCDR1 having the sequence of SEQ ID NO: 67; and an LCDR2 having the sequence of SEQ ID NO: 109 and a HCDR2 having the sequence of SEQ ID NO: 68.

63. The ABP of claim 47, wherein the first antigen binding moiety further comprises an LCDR1 having the sequence of SEQ ID NO: 111 and a heavy chain HCDR1 having the sequence of SEQ ID NO: 70; and an LCDR2 having the sequence of SEQ ID NO: 112 and a HCDR2 having the sequence of SEQ ID NO: 71.

64. The ABP of claim 48, wherein the first antigen binding moiety further comprises an LCDR1 having the sequence of SEQ ID NO: 114 and a heavy chain HCDR1 having the sequence of SEQ ID NO: 70; and an LCDR2 having the sequence of SEQ ID NO: 115 and a HCDR2 having the sequence of SEQ ID NO: 71.

65. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 205, LFR2 is at least 95% identical to the sequence of SEQ ID NO: 206, LFR3 is at least 95% identical to the sequence of SEQ ID NO: 207; and LFR4 is at least 95% identical to the sequence of SEQ ID NO: 208.

66. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 209, LFR2 is at least 95% identical to the sequence of SEQ ID NO: 210, LFR3 is at least 95% identical to the sequence of SEQ ID NO: 211; and LFR4 is at least 95% identical to the sequence of SEQ ID NO: 212.

67. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 213, LFR2 is at least 95% identical to the sequence of SEQ ID NO: 214, LFR3 is at least 95% identical to the sequence of SEQ ID NO: 215; and LFR4 is at least 95% identical to the sequence of SEQ ID NO: 216.

68. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 217, LFR2 is at least 95% identical to the sequence of SEQ ID NO: 218, LFR3 is at least 95% identical to the sequence of SEQ ID NO: 219; and LFR4 is at least 95% identical to the sequence of SEQ ID NO: 220.

69. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 221, LFR2 is at least 95% identical to the sequence of SEQ ID NO: 222, LFR3 is at least 95% identical to the sequence of SEQ ID NO: 223; and LFR4 is at least 95% identical to the sequence of SEQ ID NO: 224.

70. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 225, LFR2 is at least 95% identical to the sequence of SEQ ID NO: 226, LFR3 is at least 95% identical to the sequence of SEQ ID NO: 227; and LFR4 is at least 95% identical to the sequence of SEQ ID NO: 228.

71. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 229, LFR2 is at least 95% identical to the sequence of SEQ ID NO: 230, LFR3 is at least 95% identical to the sequence of SEQ ID NO: 231; and LFR4 is at least 95% identical to the sequence of SEQ ID NO: 232.

72. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 233, LFR2 is at least 95% identical to the sequence of SEQ ID NO: 234, LFR3 is at least 95% identical to the sequence of SEQ ID NO: 235; and LFR4 is at least 95% identical to the sequence of SEQ ID NO: 236.

73. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 237, LFR2 is at least 95% identical to the sequence of SEQ ID NO: 238, LFR3 is at least 95% identical to the sequence of SEQ ID NO: 239; and LFR4 is at least 95% identical to the sequence of SEQ ID NO: 240.

74. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 241, LFR2 is at least 95% identical to the sequence of SEQ ID NO: 242, LFR3 is at least 95% identical to the sequence of SEQ ID NO: 243; and LFR4 is at least 95% identical to the sequence of SEQ ID NO: 244.

75. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 245, LFR2 is at least 95% identical to the sequence of SEQ ID NO: 246, LFR3 is at least 95% identical to the sequence of SEQ ID NO: 247; and LFR4 is at least 95% identical to the sequence of SEQ ID NO: 248.

76. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 249, LFR2 is at least 95% identical to the sequence of SEQ ID NO: 250, LFR3 is at least 95% identical to the sequence of SEQ ID NO: 251; and LFR4 is at least 95% identical to the sequence of SEQ ID NO: 252.

77. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 253, LFR2 is at least 95% identical to the sequence of SEQ ID NO: 254, LFR3 is at least 95% identical to the sequence of SEQ ID NO: 255; and LFR4 is at least 95% identical to the sequence of SEQ ID NO: 256.

78. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 257, LFR2 is at least 95% identical to the sequence of SEQ ID NO: 258, LFR3 is at least 95% identical to the sequence of SEQ ID NO: 259; and LFR4 is at least 95% identical to the sequence of SEQ ID NO: 260.

79. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 261, LFR2 is at least 95% identical to the sequence of SEQ ID NO: 262, LFR3 is at least 95% identical to the sequence of SEQ ID NO: 263; and LFR4 is at least 95% identical to the sequence of SEQ ID NO: 264.

80. The ABP of any one of the preceding claims, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 265, LFR2 is at least 95% identical to the sequence of SEQ ID NO: 266, LFR3 is at least 95% identical to the sequence of SEQ ID NO: 267; and LFR4 is at least 95% identical to the sequence of SEQ ID NO: 268.

81. The ABP of claim 65, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 141, HFR2 is at least 95% identical to the sequence of SEQ ID NO: 142, HFR3 is at least 95% identical to the sequence of SEQ ID NO: 143; and HFR4 is at least 95% identical to the sequence of SEQ ID NO: 144.

82. The ABP of claim 66, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 145, HFR2 is at least 95% identical to the sequence of SEQ ID NO: 146, HFR3 is at least 95% identical to the sequence of SEQ ID NO: 147; and HFR4 is at least 95% identical to the sequence of SEQ ID NO: 148.

83. The ABP of claim 67, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 149, HFR2 is at least 95% identical to the sequence of SEQ ID NO: 150, HFR3 is at least 95% identical to the sequence of SEQ ID NO: 151; and HFR4 is at least 95% identical to the sequence of SEQ ID NO: 152.

84. The ABP of claim 68, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 153, HFR2 is at least 95% identical to the sequence of SEQ ID NO: 154, HFR3 is at least 95% identical to the sequence of SEQ ID NO: 155; and HFR4 is at least 95% identical to the sequence of SEQ ID NO: 156.

85. The ABP of claim 69, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 157, HFR2 is at least 95% identical to the sequence of SEQ ID NO: 158, HFR3 is at least 95% identical to the sequence of SEQ ID NO: 159; and HFR4 is at least 95% identical to the sequence of SEQ ID NO: 160.

86. The ABP of claim 70, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 161, HFR2 is at least 95% identical to the sequence of SEQ ID NO: 162, HFR3 is at least 95% identical to the sequence of SEQ ID NO: 163; and HFR4 is at least 95% identical to the sequence of SEQ ID NO: 164.

87. The ABP of claim 71, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 165, HFR2 is at least 95% identical to the sequence of SEQ ID NO: 166, HFR3 is at least 95% identical to the sequence of SEQ ID NO: 167; and HFR4 is at least 95% identical to the sequence of SEQ ID NO: 168.

88. The ABP of claim 72, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 169, HFR2 is at least 95% identical to the sequence of SEQ ID NO: 170, HFR3 is at least 95% identical to the sequence of SEQ ID NO: 171; and HFR4 is at least 95% identical to the sequence of SEQ ID NO: 172.

89. The ABP of claim 73, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 173, HFR2 is at least 95% identical to the sequence of SEQ ID NO: 174, HFR3 is at least 95% identical to the sequence of SEQ ID NO: 175; and HFR4 is at least 95% identical to the sequence of SEQ ID NO: 176.

90. The ABP of claim 74, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 177, HFR2 is at least 95% identical to the sequence of SEQ ID NO: 178, HFR3 is at least 95% identical to the sequence of SEQ ID NO: 179; and HFR4 is at least 95% identical to the sequence of SEQ ID NO: 180.

91. The ABP of claim 75, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 181, HFR2 is at least 95% identical to the sequence of SEQ ID NO: 182, HFR3 is at least 95% identical to the sequence of SEQ ID NO: 183; and HFR4 is at least 95% identical to the sequence of SEQ ID NO: 184.

92. The ABP of claim 76, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 185, HFR2 is at least 95% identical to the sequence of SEQ ID NO: 186, HFR3 is at least 95% identical to the sequence of SEQ ID NO: 187; and HFR4 is at least 95% identical to the sequence of SEQ ID NO: 188.

93. The ABP of claim 77, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 189, HFR2 is at least 95% identical to the sequence of SEQ ID NO: 190, HFR3 is at least 95% identical to the sequence of SEQ ID NO: 191; and HFR4 is at least 95% identical to the sequence of SEQ ID NO: 192.

94. The ABP of claim 78, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 193, HFR2 is at least 95% identical to the sequence of SEQ ID NO: 194, HFR3 is at least 95% identical to the sequence of SEQ ID NO: 195; and HFR4 is at least 95% identical to the sequence of SEQ ID NO: 196.

95. The ABP of claim 79, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 197, HFR2 is at least 95% identical to the sequence of SEQ ID NO: 198, HFR3 is at least 95% identical to the sequence of SEQ ID NO: 199; and HFR4 is at least 95% identical to the sequence of SEQ ID NO: 200.

96. The ABP of claim 80, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 201, HFR2 is at least 95% identical to the sequence of SEQ ID NO: 202, HFR3 is at least 95% identical to the sequence of SEQ ID NO: 203; and HFR4 is at least 95% identical to the sequence of SEQ ID NO: 204.

97. The ABP of any of the preceding claims, wherein the first antigen binding moiety comprises a variable light chain (V_{L}) having an amino acid sequence that is at least 90% identical to an amino acid sequence selected from SEQ ID NOs: 17-32.

98. The ABP of any of the preceding claims, wherein the first antigen binding moiety comprises a variable heavy chain (V_{H}) having an amino acid sequence that is at least 90% identical to an amino acid sequence selected from SEQ ID NOs.: 1-16.

99. The ABP of any one of the preceding claims, wherein the first antigen binding moiety is an antibody fragment (e.g., single chain variable fragment (scFv) or antigen-binding fragment).

100. The ABP of any one of the preceding claims, wherein the ABP is transported to the lysosome.

101. The ABP of any one of the preceding claims, wherein the ABP is transported back to the cell surface following internalization.

102. The ABP of any one of claims 1-100, wherein the ABP is degraded in the cell.

103. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 82,
the LCDR2 has the sequence of SEQ ID NO: 83,
the LCDR3 has the sequence of SEQ ID NO: 84,
the HCDR 1 has the sequence of SEQ ID NO: 42,
the HCDR2 has the sequence of SEQ ID NO: 43, and
the HCDR3 has the sequence of SEQ ID NO: 44.

104. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 92,
the LCDR2 has the sequence of SEQ ID NO: 93,
the LCDR3 has the sequence of SEQ ID NO: 94,
the HCDR 1 has the sequence of SEQ ID NO: 50,
the HCDR2 has the sequence of SEQ ID NO: 53, and
the HCDR3 has the sequence of SEQ ID NO: 54.

105. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 97,
the LCDR2 has the sequence of SEQ ID NO: 98,
the LCDR3 has the sequence of SEQ ID NO: 99,
the HCDR1 has the sequence of SEQ ID NO: 56,
the HCDR2 has the sequence of SEQ ID NO: 57, and
the HCDR3 has the sequence of SEQ ID NO: 58.

106. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 100,
the LCDR2 has the sequence of SEQ ID NO: 93,
the LCDR3 has the sequence of SEQ ID NO: 101,
the HCDR 1 has the sequence of SEQ ID NO: 50,
the HCDR2 has the sequence of SEQ ID NO: 59, and
the HCDR3 has the sequence of SEQ ID NO: 60.

107. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 108,
the LCDR2 has the sequence of SEQ ID NO: 109,
the LCDR3 has the sequence of SEQ ID NO: 110,
the HCDR 1 has the sequence of SEQ ID NO: 67,
the HCDR2 has the sequence of SEQ ID NO: 68, and
the HCDR3 has the sequence of SEQ ID NO: 69.

108. An antigen binding protein (ABP) comprising a first antigen binding moiety that specifically binds to cation-independent mannose 6 phosphate receptor (CI-M6PR), comprising a light chain CDR1 (LCDR1), a light chain CDR2 (LCDR2), a light chain CDR3 (LCDR3), a heavy chain CDR1 (HCDR1), a heavy chain CDR2 (HCDR2), and a heavy chain CDR3 (HCDR3), wherein
the LCDR1 has the sequence of SEQ ID NO: 111,
the LCDR2 has the sequence of SEQ ID NO: 112,
the LCDR3 has the sequence of SEQ ID NO: 113,
the HCDR 1 has the sequence of SEQ ID NO: 70,
the HCDR2 has the sequence of SEQ ID NO: 71, and
the HCDR3 has the sequence of SEQ ID NO: 72.

109. A bifunctional molecule comprising:
a first moiety that specifically binds to cation-independent mannose 6 phosphate receptor (CI-M6PR), wherein the first moiety is an antibody or antibody fragment; and
a second moiety that specifically binds a cell surface target molecule or extracellular target molecule, wherein the second moiety is selected from an antibody, an antigen-binding fragment, a ligand, and a small molecule.

110. The bifunctional molecule of claim 109, wherein the bifunctional molecule is a polypeptide.

111. The bifunctional molecule of claim 109, wherein the first moiety and the second moiety are covalently attached via a linker.

112. The bifunctional molecule of claim 111, wherein the second moiety is a small molecule.

113. The bifunctional molecule of any one of claims 109-112, wherein the first binding moiety comprises a light chain CDR3 (LCDR3) having the sequence of SEQ ID NO: 76 and a heavy chain CDR3 (HCDR3) having the sequence of SEQ ID NO: 35.

114. The bifunctional molecule of any one of claims 109-112, wherein the first binding moiety comprises a light chain CDR3 having the sequence of SEQ ID NO: 78 and a heavy chain CDR3 having the amino acid sequence of SEQ ID NO: 38.

115. The bifunctional molecule of any one of claims 109-112, wherein the first binding moiety comprises a light chain CDR3 having the sequence of SEQ ID NO: 81 and a heavy chain CDR3 having the sequence of SEQ ID NO: 41.

116. The bifunctional molecule of any one of claims 109-112, wherein the first binding moiety comprises a light chain CDR3 having the sequence of SEQ ID NO: 84 and a heavy chain CDR3 having the sequence of SEQ ID NO: 44.

117. The bifunctional molecule of any one of claims 109-112, wherein the first binding moiety comprises a light chain CDR3 having the sequence of SEQ ID NO: 85 and a heavy chain CDR3 having the sequence of SEQ ID NO: 46.

118. The bifunctional molecule of any one of claims 109-112, wherein the first binding moiety comprises a light chain CDR3 having the sequence of SEQ ID NO: 88 and a heavy chain CDR3 having the sequence of SEQ ID NO: 49.

119. The bifunctional molecule of any one of claims 109-112, wherein the first binding moiety comprises a light chain CDR3 having the sequence of SEQ ID NO: 91 and a heavy chain CDR3 having the sequence of SEQ ID NO: 52.

120. The bifunctional molecule of any one of claims 109-112, wherein the first binding moiety comprises a light chain CDR3 having the sequence of SEQ ID NO: 94 and a heavy chain CDR3 having the sequence of SEQ ID NO: 54.

121. The bifunctional molecule of any one of claims 109-112, wherein the first binding moiety comprises a light chain CDR3 having the sequence of SEQ ID NO: 96 and a heavy chain CDR3 having the sequence of SEQ ID NO: 55.

122. The bifunctional molecule of any one of claims 109-112, wherein the first binding moiety comprises a light chain CDR3 having the sequence of SEQ ID NO: 99 and a heavy chain CDR3 having the sequence of SEQ ID NO: 58.

123. The bifunctional molecule of any one of claims 109-112, wherein the first binding moiety comprises a light chain CDR3 having the sequence of SEQ ID NO: 101 and a heavy chain CDR3 having the sequence of SEQ ID NO: 60.

124. The bifunctional molecule of any one of claims 109-112, wherein the first binding moiety comprises a light chain CDR3 having the sequence of SEQ ID NO: 104 and a heavy chain CDR3 having the sequence of SEQ ID NO: 63.

125. The bifunctional molecule of any one of claims 109-112, wherein the first binding moiety comprises a light chain CDR3 having the sequence of SEQ ID NO: 107 and a heavy chain CDR3 having the sequence of SEQ ID NO: 66.

126. The bifunctional molecule of any one of claims 109-112, wherein the first binding moiety comprises a light chain CDR3 having the sequence of SEQ ID NO: 110 and a heavy chain CDR3 having the sequence of SEQ ID NO: 69.

127. The bifunctional molecule of any one of claims 109-112, wherein the first binding moiety comprises a light chain CDR3 having the sequence of SEQ ID NO: 113 and a heavy chain CDR3 having the sequence of SEQ ID NO: 72.

128. The bifunctional molecule of any one of claims 109-112, wherein the first binding moiety comprises a light chain CDR3 having the sequence of SEQ ID NO: 116 and a heavy chain CDR3 having the sequence of SEQ ID NO: 73.

129. The bifunctional molecule of claim 113, wherein the first moiety further comprises a light chain CDR1 (LCDR1) having the sequence of SEQ ID NO: 74 and a heavy chain CDR1 (HCDR1) having the sequence of SEQ ID NO: 33; and a light chain CDR2 (LCDR2) having the sequence of SEQ ID NO: 75 and a heavy chain CDR2 (HCDR2) having the sequence of SEQ ID NO: 34.

130. The bifunctional molecule of claim 114, wherein the first moiety further comprises an LCDR1 having the sequence of SEQ ID NO: 77 and a heavy chain HCDR1 having the sequence of SEQ ID NO: 36; and an LCDR2 having the sequence of SEQ ID NO: 75 and a HCDR2 having the sequence of SEQ ID NO: 37.

131. The bifunctional molecule of claim 115, wherein the first moiety further comprises an LCDR1 having the sequence of SEQ ID NO: 79 and a heavy chain HCDR1 having the sequence of SEQ ID NO: 39; and an LCDR2 having the sequence of SEQ ID NO: 80 and a HCDR2 having the sequence of SEQ ID NO: 40.

132. The bifunctional molecule of claim 116, wherein the first moiety further comprises an LCDR1 having the sequence of SEQ ID NO: 82 and a heavy chain HCDR1 having the sequence of SEQ ID NO: 42; and an LCDR2 having the sequence of SEQ ID NO: 83 and a HCDR2 having the sequence of SEQ ID NO: 43.

133. The bifunctional molecule of claim 117, wherein the first moiety further comprises an LCDR1 having the sequence of SEQ ID NO: 79 and a heavy chain HCDR1 having the sequence of SEQ ID NO: 45; and an LCDR2 having the sequence of SEQ ID NO: 80 and a HCDR2 having the sequence of SEQ ID NO: 34.

134. The bifunctional molecule of claim 118, wherein the first moiety further comprises an LCDR1 having the sequence of SEQ ID NO: 86 and a heavy chain HCDR1 having the sequence of SEQ ID NO: 47; and an LCDR2 having the sequence of SEQ ID NO: 87 and a HCDR2 having the sequence of SEQ ID NO: 48.

135. The bifunctional molecule of claim 119, wherein the first moiety further comprises an LCDR1 having the sequence of SEQ ID NO: 89 and a heavy chain HCDR1 having the sequence of SEQ ID NO: 50; and an LCDR2 having the sequence of SEQ ID NO: 90 and a HCDR2 having the sequence of SEQ ID NO: 53.

136. The bifunctional molecule of claim 120, wherein the first moiety further comprises an LCDR1 having the sequence of SEQ ID NO: 92 and a heavy chain HCDR1 having the sequence of SEQ ID NO: 50; and an LCDR2 having the sequence of SEQ ID NO: 93 and a HCDR2 having the sequence of SEQ ID NO: 53.

137. The bifunctional molecule of claim 121, wherein the first moiety further comprises an LCDR1 having the sequence of SEQ ID NO: 95 and a heavy chain HCDR1 having the sequence of SEQ ID NO: 50; and an LCDR2 having the sequence of SEQ ID NO: 93 and a HCDR2 having the sequence of SEQ ID NO: 51.

138. The bifunctional molecule of claim 122, wherein the first moiety further comprises an LCDR1 having the sequence of SEQ ID NO: 97 and a heavy chain HCDR1 having the sequence of SEQ ID NO: 56; and an LCDR2 having the sequence of SEQ ID NO: 98 and a HCDR2 having the sequence of SEQ ID NO: 57.

139. The bifunctional molecule of claim 123, wherein the first moiety further comprises an LCDR1 having the sequence of SEQ ID NO: 100 and a heavy chain HCDR1 having the sequence of SEQ ID NO: 50; and an LCDR2 having the sequence of SEQ ID NO: 93 and a HCDR2 having the sequence of SEQ ID NO: 59.

140. The bifunctional molecule of claim 124, wherein the first moiety further comprises an LCDR1 having the sequence of SEQ ID NO: 102 and a heavy chain HCDR1 having the sequence of SEQ ID NO: 61; and an LCDR2 having the sequence of SEQ ID NO: 103 and a HCDR2 having the sequence of SEQ ID NO: 62.

141. The bifunctional molecule of claim 125, wherein the first moiety further comprises an LCDR1 having the sequence of SEQ ID NO: 105 and a heavy chain HCDR1 having the sequence of SEQ ID NO: 64; and an LCDR2 having the sequence of SEQ ID NO: 106 and a HCDR2 having the sequence of SEQ ID NO: 65.

142. The bifunctional molecule of claim 126, wherein the first moiety further comprises an LCDR1 having the sequence of SEQ ID NO: 108 and a heavy chain HCDR1 having the sequence of SEQ ID NO: 67; and an LCDR2 having the sequence of SEQ ID NO: 109 and a HCDR2 having the sequence of SEQ ID NO: 68.

143. The bifunctional molecule of claim 127, wherein the first moiety further comprises an LCDR1 having the sequence of SEQ ID NO: 111 and a heavy chain HCDR1 having the sequence of SEQ ID NO: 70; and an LCDR2 having the sequence of SEQ ID NO: 112 and a HCDR2 having the sequence of SEQ ID NO: 71.

144. The bifunctional molecule of claim 128, wherein the first moiety further comprises an LCDR1 having the sequence of SEQ ID NO: 114 and a heavy chain HCDR1 having the sequence of SEQ ID NO: 70; and an LCDR2 having the sequence of SEQ ID NO: 115 and a HCDR2 having the sequence of SEQ ID NO: 71.

145. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 205, LFR2 is at least 95% identical to the sequence of SEQ ID NO: 206, LFR3 is at least 95% identical to the sequence of SEQ ID NO: 207; and LFR4 is at least 95% identical to the sequence of SEQ ID NO: 208.

146. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 209, LFR2 is at least 95% identical to the sequence of SEQ ID NO: 210, LFR3 is at least 95% identical to the sequence of SEQ ID NO: 211; and LFR4 is at least 95% identical to the sequence of SEQ ID NO: 212.

147. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 213, LFR2 is at least 95% identical to the sequence of SEQ ID NO: 214, LFR3 is at least 95% identical to the sequence of SEQ ID NO: 215; and LFR4 is at least 95% identical to the sequence of SEQ ID NO: 216.

148. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 217, LFR2 is at least 95% identical to the sequence of SEQ ID NO: 218, LFR3 is at least 95% identical to the sequence of SEQ ID NO: 219; and LFR4 is at least 95% identical to the sequence of SEQ ID NO: 220.

149. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 221, LFR2 is at least 95% identical to the sequence of SEQ ID NO: 222, LFR3 is at least 95% identical to the sequence of SEQ ID NO: 223; and LFR4 is at least 95% identical to the sequence of SEQ ID NO: 224.

150. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 225, LFR2 is at least 95% identical to the sequence of SEQ ID NO: 226, LFR3 is at least 95% identical to the sequence of SEQ ID NO: 227; and LFR4 is at least 95% identical to the sequence of SEQ ID NO: 228.

151. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 229, LFR2 is at least 95% identical to the sequence of SEQ ID NO: 230, LFR3 is at least 95% identical to the sequence of SEQ ID NO: 231; and LFR4 is at least 95% identical to the sequence of SEQ ID NO: 232.

152. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 233, LFR2 is at least 95% identical to the sequence of SEQ ID NO: 234, LFR3 is at least 95% identical to the sequence of SEQ ID NO: 235; and LFR4 is at least 95% identical to the sequence of SEQ ID NO: 236.

153. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 237, LFR2 is at least 95% identical to the sequence of SEQ ID NO: 238, LFR3 is at least 95% identical to the sequence of SEQ ID NO: 239; and LFR4 is at least 95% identical to the sequence of SEQ ID NO: 240.

154. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 241, LFR2 is at least 95% identical to the sequence of SEQ ID NO: 242, LFR3 is at least 95% identical to the sequence of SEQ ID NO: 243; and LFR4 is at least 95% identical to the sequence of SEQ ID NO: 244.

155. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 245, LFR2 is at least 95% identical to the sequence of SEQ ID NO: 246, LFR3 is at least 95% identical to the sequence of SEQ ID NO: 247; and LFR4 is at least 95% identical to the sequence of SEQ ID NO: 248.

156. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 249, LFR2 is at least 95% identical to the sequence of SEQ ID NO: 250, LFR3 is at least 95% identical to the sequence of SEQ ID NO: 251; and LFR4 is at least 95% identical to the sequence of SEQ ID NO: 252.

157. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 253, LFR2 is at least 95% identical to the sequence of SEQ ID NO: 254, LFR3 is at least 95% identical to the sequence of SEQ ID NO: 255; and LFR4 is at least 95% identical to the sequence of SEQ ID NO: 256.

158. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 257, LFR2 is at least 95% identical to the sequence of SEQ ID NO: 258, LFR3 is at least 95% identical to the sequence of SEQ ID NO: 259; and LFR4 is at least 95% identical to the sequence of SEQ ID NO: 260.

159. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 261, LFR2 is at least 95% identical to the sequence of SEQ ID NO: 262, LFR3 is at least 95% identical to the sequence of SEQ ID NO: 263; and LFR4 is at least 95% identical to the sequence of SEQ ID NO: 264.

160. The bifunctional molecule of any one of claims 109-144, wherein the first moiety comprises a variable light chain (V_{L}) comprising framework regions LFR1, LFR2, LFR3, and LFR4 wherein LFR1 is at least 95% identical to the sequence of SEQ ID NO: 265, LFR2 is at least 95% identical to the sequence of SEQ ID NO: 266, LFR3 is at least 95% identical to the sequence of SEQ ID NO: 267; and LFR4 is at least 95% identical to the sequence of SEQ ID NO: 268.

161. The bifunctional molecule of claim 145, wherein the first moiety comprises a variable heavy chain (V_{H}) comprising framework regions HFR1, HFR2, HFR3, and HFR4 wherein HFR1 is at least 95% identical to the sequence of SEQ ID NO: 141, HFR2 is at least 95% identical to the sequence of SEQ ID NO: 142, HFR3 is at least 95% identical to the sequence of SEQ ID NO: 143; and HFR4 is at least 95% identical to the sequence of SEQ ID NO: 144.

162. The bifunctional molecule of claim 146, wherein the first moiety comprises a variable heavy chain (V_{H}) comprising framework regions HFR1, HFR2, HFR3, and HFR4 wherein HFR1 is at least 95% identical to the sequence of SEQ ID NO: 145, HFR2 is at least 95% identical to the sequence of SEQ ID NO: 146, HFR3 is at least 95% identical to the sequence of SEQ ID NO: 147; and HFR4 is at least 95% identical to the sequence of SEQ ID NO: 148.

163. The bifunctional molecule of claim 147, wherein the first moiety comprises a variable heavy chain (V_{H}) comprising framework regions HFR1, HFR2, HFR3, and HFR4 wherein HFR1 is at least 95% identical to the sequence of SEQ ID NO: 149, HFR2 is at least 95% identical to the sequence of SEQ ID NO: 150, HFR3 is at least 95% identical to the sequence of SEQ ID NO: 151; and HFR4 is at least 95% identical to the sequence of SEQ ID NO: 152.

164. The bifunctional molecule of claim 148, wherein the first moiety comprises a variable heavy chain (V_{H}) comprising framework regions HFR1, HFR2, HFR3, and HFR4 wherein HFR1 is at least 95% identical to the sequence of SEQ ID NO: 153, HFR2 is at least 95% identical to the sequence of SEQ ID NO: 154, HFR3 is at least 95% identical to the sequence of SEQ ID NO: 155; and HFR4 is at least 95% identical to the sequence of SEQ ID NO: 156.

165. The bifunctional molecule of claim 149, wherein the first moiety comprises a variable heavy chain (V_{H}) comprising framework regions HFR1, HFR2, HFR3, and HFR4 wherein HFR1 is at least 95% identical to the sequence of SEQ ID NO: 157, HFR2 is at least 95% identical to the sequence of SEQ ID NO: 158, HFR3 is at least 95% identical to the sequence of SEQ ID NO: 159; and HFR4 is at least 95% identical to the sequence of SEQ ID NO: 160.

166. The bifunctional molecule of claim 150, wherein the first moiety comprises a variable heavy chain (V_{H}) comprising framework regions HFR1, HFR2, HFR3, and HFR4 wherein HFR1 is at least 95% identical to the sequence of SEQ ID NO: 161, HFR2 is at least 95% identical to the sequence of SEQ ID NO: 162, HFR3 is at least 95% identical to the sequence of SEQ ID NO: 163; and HFR4 is at least 95% identical to the sequence of SEQ ID NO: 164.

167. The bifunctional molecule of claim 151, wherein the first moiety comprises a variable heavy chain (V_{H}) comprising framework regions HFR1, HFR2, HFR3, and HFR4 wherein HFR1 is at least 95% identical to the sequence of SEQ ID NO: 165, HFR2 is at least 95% identical to the sequence of SEQ ID NO: 166, HFR3 is at least 95% identical to the sequence of SEQ ID NO: 167; and HFR4 is at least 95% identical to the sequence of SEQ ID NO: 168.

168. The bifunctional molecule of claim 152, wherein the first moiety comprises a variable heavy chain (V_{H}) comprising framework regions HFR1, HFR2, HFR3, and HFR4 wherein HFR1 is at least 95% identical to the sequence of SEQ ID NO: 169, HFR2 is at least 95% identical to the sequence of SEQ ID NO: 170, HFR3 is at least 95% identical to the sequence of SEQ ID NO: 171; and HFR4 is at least 95% identical to the sequence of SEQ ID NO: 172.

169. The bifunctional molecule of claim 153, wherein the first moiety comprises a variable heavy chain (V_{H}) comprising framework regions HFR1, HFR2, HFR3, and HFR4 wherein HFR1 is at least 95% identical to the sequence of SEQ ID NO: 173, HFR2 is at least 95% identical to the sequence of SEQ ID NO: 174, HFR3 is at least 95% identical to the sequence of SEQ ID NO: 175; and HFR4 is at least 95% identical to the sequence of SEQ ID NO: 176.

170. The bifunctional molecule of claim 154, wherein the first moiety comprises a variable heavy chain (V_{H}) comprising framework regions HFR1, HFR2, HFR3, and HFR4 wherein HFR1 is at least 95% identical to the sequence of SEQ ID NO: 177, HFR2 is at least 95% identical to the sequence of SEQ ID NO: 178, HFR3 is at least 95% identical to the sequence of SEQ ID NO: 179; and HFR4 is at least 95% identical to the sequence of SEQ ID NO: 180.

171. The bifunctional molecule of claim 155, wherein the first moiety comprises a variable heavy chain (V_{H}) comprising framework regions HFR1, HFR2, HFR3, and HFR4 wherein HFR1 is at least 95% identical to the sequence of SEQ ID NO: 181, HFR2 is at least 95% identical to the sequence of SEQ ID NO: 182, HFR3 is at least 95% identical to the sequence of SEQ ID NO: 183; and HFR4 is at least 95% identical to the sequence of SEQ ID NO: 184.

172. The bifunctional molecule of claim 156, wherein the first moiety comprises a variable heavy chain (V_{H}) comprising framework regions HFR1, HFR2, HFR3, and HFR4 wherein HFR1 is at least 95% identical to the sequence of SEQ ID NO: 185, HFR2 is at least 95% identical to the sequence of SEQ ID NO: 186, HFR3 is at least 95% identical to the sequence of SEQ ID NO: 187; and HFR4 is at least 95% identical to the sequence of SEQ ID NO: 188.

173. The bifunctional molecule of claim 157, wherein the first moiety comprises a variable heavy chain (V_{H}) comprising framework regions HFR1, HFR2, HFR3, and HFR4 wherein HFR1 is at least 95% identical to the sequence of SEQ ID NO: 189, HFR2 is at least 95% identical to the sequence of SEQ ID NO: 190, HFR3 is at least 95% identical to the sequence of SEQ ID NO: 191; and HFR4 is at least 95% identical to the sequence of SEQ ID NO: 192.

174. The bifunctional molecule of claim 158, wherein the first moiety comprises a variable heavy chain (V_{H}) comprising framework regions HFR1, HFR2, HFR3, and HFR4 wherein HFR1 is at least 95% identical to the sequence of SEQ ID NO: 193, HFR2 is at least 95% identical to the sequence of SEQ ID NO: 194, HFR3 is at least 95% identical to the sequence of SEQ ID NO: 195; and HFR4 is at least 95% identical to the sequence of SEQ ID NO: 196.

175. The bifunctional molecule of claim 159, wherein the first moiety comprises a variable heavy chain (V_{H}) comprising framework regions HFR1, HFR2, HFR3, and HFR4 wherein HFR1 is at least 95% identical to the sequence of SEQ ID NO: 197, HFR2 is at least 95% identical to the sequence of SEQ ID NO: 198, HFR3 is at least 95% identical to the sequence of SEQ ID NO: 199; and HFR4 is at least 95% identical to the sequence of SEQ ID NO: 200.

176. The bifunctional molecule of claim 160, wherein the first moiety comprises a variable heavy chain (V_{H}) comprising framework regions HFR1, HFR2, HFR3, and HFR4 wherein HFR1 is at least 95% identical to the sequence of SEQ ID NO: 201, HFR2 is at least 95% identical to the sequence of SEQ ID NO: 202, HFR3 is at least 95% identical to the sequence of SEQ ID NO: 203; and HFR4 is at least 95% identical to the sequence of SEQ ID NO: 204.

177. The bifunctional molecule of any one of claims 109-176, wherein the first moiety comprises a variable light chain (V_{L}) having an amino acid sequence that is at least 90% identical to an amino acid sequence selected from SEQ ID NOs: 17-32.

178. The bifunctional molecule of any one of claims 109-177, wherein the first moiety comprises a variable heavy chain (V_{H}) having an amino acid sequence that is at least 90% identical to an amino acid sequence selected from SEQ ID NOs: 1-16.

179. A method of degrading a soluble target molecule or a cell surface target molecule comprising:
(a) contacting the target molecule with a multi-specific antigen binding protein (ABP), wherein the ABP comprises a first antigen binding moiety that specifically binds to a cation-independent mannose 6 phosphate receptor (CI-M6PR) on the surface of a cell; and
(b) transporting the ABP, the target molecule, and the CI-M6PR to a lysosome within a cell, wherein the target molecule is degraded in the lysosome.

180. The method of claim 179, wherein the ABP further comprises a linked cargo moiety.

181. The method of claim 180, wherein the cargo moiety is a polypeptide that is fused to the ABP.

182. The method of claim 181, wherein the cargo moiety is conjugated to the ABP, optionally via a linker.

183. The method of any one of claims 179-182, wherein the first antigen binding moiety binds to a domain of CI-M6PR selected from: domain 1, domain 4, domain 5, domain 6, domain 7, and domain 8.

184. The method of any one of claims 179-182, wherein the first antigen binding moiety binds CI-M6PR with a dissociation equilibrium constant (K_{D}) of about 10 nM or less.

185. The method of any one of claims 179-182, wherein the first antigen binding moiety binds CI-M6PR with a dissociation equilibrium constant (K_{D}) between about 1 nM and about 500 nM.

186. The method of any one of claims 179-185, wherein the off rate (k_{off}) of the first antigen binding moiety for CI-M6PR is between 1×10⁻⁸ s⁻¹ and 0.1 s⁻¹.

187. The method of any one of claims 179-186, wherein the binding of the first antigen binding moiety to CI-M6PR is pH dependent.

188. The method of any one of claims 179-187, wherein the first antigen binding moiety is released from CI-M6PR at a pH of 7.4 or lower.

189. The method of any one of claims 179-188, wherein the first antigen binding moiety is released from CI-M6PR at a pH of 6.0 or lower.

190. The method of any one of claims 179-189, wherein the first antigen binding moiety is released from CI-M6PR at a pH of 5.5 or lower.

191. The method of any one of claims 179-190, wherein the first antigen binding moiety is released from CI-M6PR at a pH of 5.0 or lower.

192. The method of any one of claims 179-191, wherein the first antigen binding moiety comprises a light chain CDR3 (LCDR3) having the sequence of SEQ ID NO: 76 and a heavy chain CDR3 (HCDR3) having the sequence of SEQ ID NO: 35.

193. The method of any one of claims 179-191, wherein the first antigen binding moiety comprises a light chain CDR3 having the sequence of SEQ ID NO: 78 and a heavy chain CDR3 having the amino acid sequence of SEQ ID NO: 38.

194. The method of any one of claims 179-191, wherein the first antigen binding moiety comprises a light chain CDR3 having the sequence of SEQ ID NO: 81 and a heavy chain CDR3 having the sequence of SEQ ID NO: 41.

195. The method of any one of claims 179-191, wherein the first antigen binding moiety comprises a light chain CDR3 having the sequence of SEQ ID NO: 84 and a heavy chain CDR3 having the sequence of SEQ ID NO: 44.

196. The method of any one of claims 179-191, wherein the first antigen binding moiety comprises a light chain CDR3 having the sequence of SEQ ID NO: 85 and a heavy chain CDR3 having the sequence of SEQ ID NO: 46.

197. The method of any one of claims 179-191, wherein the first antigen binding moiety comprises a light chain CDR3 having the sequence of SEQ ID NO: 88 and a heavy chain CDR3 having the sequence of SEQ ID NO: 49.

198. The method of any one of claims 179-191, wherein the first antigen binding moiety comprises a light chain CDR3 having the sequence of SEQ ID NO: 91 and a heavy chain CDR3 having the sequence of SEQ ID NO: 52.

199. The method of any one of claims 179-191, wherein the first antigen binding moiety comprises a light chain CDR3 having the sequence of SEQ ID NO: 94 and a heavy chain CDR3 having the sequence of SEQ ID NO: 54.

200. The method of any one of claims 179-191, wherein the first antigen binding moiety comprises a light chain CDR3 having the sequence of SEQ ID NO: 96 and a heavy chain CDR3 having the sequence of SEQ ID NO: 55.

201. The method of any one of claims 179-191, wherein the first antigen binding moiety comprises a light chain CDR3 having the sequence of SEQ ID NO: 99 and a heavy chain CDR3 having the sequence of SEQ ID NO: 58.

202. The method of any one of claims 179-191, wherein the first antigen binding moiety comprises a light chain CDR3 having the sequence of SEQ ID NO: 101 and a heavy chain CDR3 having the sequence of SEQ ID NO: 60.

203. The method of any one of claims 179-191, wherein the first antigen binding moiety comprises a light chain CDR3 having the sequence of SEQ ID NO: 104 and a heavy chain CDR3 having the sequence of SEQ ID NO: 63.

204. The method of any one of claims 179-191, wherein the first antigen binding moiety comprises a light chain CDR3 having the sequence of SEQ ID NO: 107 and a heavy chain CDR3 having the sequence of SEQ ID NO: 66.

205. The method of any one of claims 179-191, wherein the first antigen binding moiety comprises a light chain CDR3 having the sequence of SEQ ID NO: 110 and a heavy chain CDR3 having the sequence of SEQ ID NO: 69.

206. The method of any one of claims 179-191, wherein the first antigen binding moiety comprises a light chain CDR3 having the sequence of SEQ ID NO: 113 and a heavy chain CDR3 having the sequence of SEQ ID NO: 72.

207. The method of any one of claims 179-191, wherein the first antigen binding moiety comprises a light chain CDR3 having the sequence of SEQ ID NO: 116 and a heavy chain CDR3 having the sequence of SEQ ID NO: 73.

208. The method of claim 192, wherein the first antigen binding moiety further comprises a light chain CDR1 (LCDR1) having the sequence of SEQ ID NO: 74 and a heavy chain CDR1 (HCDR1) having the sequence of SEQ ID NO: 33; and a light chain CDR2 (LCDR2) having the sequence of SEQ ID NO: 75 and a heavy chain CDR2 (HCDR2) having the sequence of SEQ ID NO: 34.

209. The method of claim 193, wherein the first antigen binding moiety further comprises an LCDR1 having the sequence of SEQ ID NO: 77 and a heavy chain HCDR1 having the sequence of SEQ ID NO: 36; and an LCDR2 having the sequence of SEQ ID NO: 75 and a HCDR2 having the sequence of SEQ ID NO: 37.

210. The method of claim 194, wherein the first antigen binding moiety further comprises an LCDR1 having the sequence of SEQ ID NO: 79 and a heavy chain HCDR1 having the sequence of SEQ ID NO: 39; and an LCDR2 having the sequence of SEQ ID NO: 80 and a HCDR2 having the sequence of SEQ ID NO: 40.

211. The method of claim 195, wherein the first antigen binding moiety further comprises an LCDR1 having the sequence of SEQ ID NO: 82 and a heavy chain HCDR1 having the sequence of SEQ ID NO: 42; and an LCDR2 having the sequence of SEQ ID NO: 83 and a HCDR2 having the sequence of SEQ ID NO: 43.

212. The method of claim 196, wherein the first antigen binding moiety further comprises an LCDR1 having the sequence of SEQ ID NO: 79 and a heavy chain HCDR1 having the sequence of SEQ ID NO: 45; and an LCDR2 having the sequence of SEQ ID NO: 80 and a HCDR2 having the sequence of SEQ ID NO: 34.

213. The method of claim 197, wherein the first antigen binding moiety further comprises an LCDR1 having the sequence of SEQ ID NO: 86 and a heavy chain HCDR1 having the sequence of SEQ ID NO: 47; and an LCDR2 having the sequence of SEQ ID NO: 87 and a HCDR2 having the sequence of SEQ ID NO: 48.

214. The method of claim 198, wherein the first antigen binding moiety further comprises an LCDR1 having the sequence of SEQ ID NO: 89 and a heavy chain HCDR1 having the sequence of SEQ ID NO: 50; and an LCDR2 having the sequence of SEQ ID NO: 90 and a HCDR2 having the sequence of SEQ ID NO: 53.

215. The method of claim 199, wherein the first antigen binding moiety further comprises an LCDR1 having the sequence of SEQ ID NO: 92 and a heavy chain HCDR1 having the sequence of SEQ ID NO: 50; and an LCDR2 having the sequence of SEQ ID NO: 93 and a HCDR2 having the sequence of SEQ ID NO: 53.

216. The method of claim 200, wherein the first antigen binding moiety further comprises an LCDR1 having the sequence of SEQ ID NO: 95 and a heavy chain HCDR1 having the sequence of SEQ ID NO: 50; and an LCDR2 having the sequence of SEQ ID NO: 93 and a HCDR2 having the sequence of SEQ ID NO: 51.

217. The method of claim 201, wherein the first antigen binding moiety further comprises an LCDR1 having the sequence of SEQ ID NO: 97 and a heavy chain HCDR1 having the sequence of SEQ ID NO: 56; and an LCDR2 having the sequence of SEQ ID NO: 98 and a HCDR2 having the sequence of SEQ ID NO: 57.

218. The method of claim 202, wherein the first antigen binding moiety further comprises an LCDR1 having the sequence of SEQ ID NO: 100 and a heavy chain HCDR1 having the sequence of SEQ ID NO: 50; and an LCDR2 having the sequence of SEQ ID NO: 93 and a HCDR2 having the sequence of SEQ ID NO: 59.

219. The method of claim 203, wherein the first antigen binding moiety further comprises an LCDR1 having the sequence of SEQ ID NO: 102 and a heavy chain HCDR1 having the sequence of SEQ ID NO: 61; and an LCDR2 having the sequence of SEQ ID NO: 103 and a HCDR2 having the sequence of SEQ ID NO: 62.

220. The method of claim 204, wherein the first antigen binding moiety further comprises an LCDR1 having the sequence of SEQ ID NO: 105 and a heavy chain HCDR1 having the sequence of SEQ ID NO: 64; and an LCDR2 having the sequence of SEQ ID NO: 106 and a HCDR2 having the sequence of SEQ ID NO: 65.

221. The method of claim 205, wherein the first antigen binding moiety further comprises an LCDR1 having the sequence of SEQ ID NO: 108 and a heavy chain HCDR 1 having the sequence of SEQ ID NO: 67; and an LCDR2 having the sequence of SEQ ID NO: 109 and a HCDR2 having the sequence of SEQ ID NO: 68.

222. The method of claim 206, wherein the first antigen binding moiety further comprises an LCDR1 having the sequence of SEQ ID NO: 111 and a heavy chain HCDR1 having the sequence of SEQ ID NO: 70; and an LCDR2 having the sequence of SEQ ID NO: 112 and a HCDR2 having the sequence of SEQ ID NO: 71.

223. The method of claim 207, wherein the first antigen binding moiety further comprises an LCDR1 having the sequence of SEQ ID NO: 114 and a heavy chain HCDR 1 having the sequence of SEQ ID NO: 70; and an LCDR2 having the sequence of SEQ ID NO: 115 and a HCDR2 having the sequence of SEQ ID NO: 71.

224. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 205, LFR2 is at least 95% identical to the sequence of SEQ ID NO: 206, LFR3 is at least 95% identical to the sequence of SEQ ID NO: 207; and LFR4 is at least 95% identical to the sequence of SEQ ID NO: 208.

225. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 209, LFR2 is at least 95% identical to the sequence of SEQ ID NO: 210, LFR3 is at least 95% identical to the sequence of SEQ ID NO: 211; and LFR4 is at least 95% identical to the sequence of SEQ ID NO: 212.

226. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 213, LFR2 is at least 95% identical to the sequence of SEQ ID NO: 214, LFR3 is at least 95% identical to the sequence of SEQ ID NO: 215; and LFR4 is at least 95% identical to the sequence of SEQ ID NO: 216.

227. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 217, LFR2 is at least 95% identical to the sequence of SEQ ID NO: 218, LFR3 is at least 95% identical to the sequence of SEQ ID NO: 219; and LFR4 is at least 95% identical to the sequence of SEQ ID NO: 220.

228. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 221, LFR2 is at least 95% identical to the sequence of SEQ ID NO: 222, LFR3 is at least 95% identical to the sequence of SEQ ID NO: 223; and LFR4 is at least 95% identical to the sequence of SEQ ID NO: 224.

229. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 225, LFR2 is at least 95% identical to the sequence of SEQ ID NO: 226, LFR3 is at least 95% identical to the sequence of SEQ ID NO: 227; and LFR4 is at least 95% identical to the sequence of SEQ ID NO: 228.

230. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 229, LFR2 is at least 95% identical to the sequence of SEQ ID NO: 230, LFR3 is at least 95% identical to the sequence of SEQ ID NO: 231; and LFR4 is at least 95% identical to the sequence of SEQ ID NO: 232.

231. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 233, LFR2 is at least 95% identical to the sequence of SEQ ID NO: 234, LFR3 is at least 95% identical to the sequence of SEQ ID NO: 235; and LFR4 is at least 95% identical to the sequence of SEQ ID NO: 236.

232. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 237, LFR2 is at least 95% identical to the sequence of SEQ ID NO: 238, LFR3 is at least 95% identical to the sequence of SEQ ID NO: 239; and LFR4 is at least 95% identical to the sequence of SEQ ID NO: 240.

233. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 241, LFR2 is at least 95% identical to the sequence of SEQ ID NO: 242, LFR3 is at least 95% identical to the sequence of SEQ ID NO: 243; and LFR4 is at least 95% identical to the sequence of SEQ ID NO: 244.

234. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 245, LFR2 is at least 95% identical to the sequence of SEQ ID NO: 246, LFR3 is at least 95% identical to the sequence of SEQ ID NO: 247; and LFR4 is at least 95% identical to the sequence of SEQ ID NO: 248.

235. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 249, LFR2 is at least 95% identical to the sequence of SEQ ID NO: 250, LFR3 is at least 95% identical to the sequence of SEQ ID NO: 251; and LFR4 is at least 95% identical to the sequence of SEQ ID NO: 252.

236. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 253, LFR2 is at least 95% identical to the sequence of SEQ ID NO: 254, LFR3 is at least 95% identical to the sequence of SEQ ID NO: 255; and LFR4 is at least 95% identical to the sequence of SEQ ID NO: 256.

237. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 257, LFR2 is at least 95% identical to the sequence of SEQ ID NO: 258, LFR3 is at least 95% identical to the sequence of SEQ ID NO: 259; and LFR4 is at least 95% identical to the sequence of SEQ ID NO: 260.

238. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 261, LFR2 is at least 95% identical to the sequence of SEQ ID NO: 262, LFR3 is at least 95% identical to the sequence of SEQ ID NO: 263; and LFR4 is at least 95% identical to the sequence of SEQ ID NO: 264.

239. The method of any one of claims 179-223, wherein the first antigen binding moiety comprises a variable light chain (V_{L}) comprising framework regions LFR1, LFR2, LFR3, and LFR4 wherein LFR1 is at least 95% identical to the sequence of SEQ ID NO: 265, LFR2 is at least 95% identical to the sequence of SEQ ID NO: 266, LFR3 is at least 95% identical to the sequence of SEQ ID NO: 267; and LFR4 is at least 95% identical to the sequence of SEQ ID NO: 268.

240. The method of claim 224, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 141, HFR2 is at least 95% identical to the sequence of SEQ ID NO: 142, HFR3 is at least 95% identical to the sequence of SEQ ID NO: 143; and HFR4 is at least 95% identical to the sequence of SEQ ID NO: 144.

241. The method of claim 225, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 145, HFR2 is at least 95% identical to the sequence of SEQ ID NO: 146, HFR3 is at least 95% identical to the sequence of SEQ ID NO: 147; and HFR4 is at least 95% identical to the sequence of SEQ ID NO: 148.

242. The method of claim 226, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 149, HFR2 is at least 95% identical to the sequence of SEQ ID NO: 150, HFR3 is at least 95% identical to the sequence of SEQ ID NO: 151; and HFR4 is at least 95% identical to the sequence of SEQ ID NO: 152.

243. The method of claim 227, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 153, HFR2 is at least 95% identical to the sequence of SEQ ID NO: 154, HFR3 is at least 95% identical to the sequence of SEQ ID NO: 155; and HFR4 is at least 95% identical to the sequence of SEQ ID NO: 156.

244. The method of claim 228, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 157, HFR2 is at least 95% identical to the sequence of SEQ ID NO: 158, HFR3 is at least 95% identical to the sequence of SEQ ID NO: 159; and HFR4 is at least 95% identical to the sequence of SEQ ID NO: 160.

245. The method of claim 229, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 161, HFR2 is at least 95% identical to the sequence of SEQ ID NO: 162, HFR3 is at least 95% identical to the sequence of SEQ ID NO: 163; and HFR4 is at least 95% identical to the sequence of SEQ ID NO: 164.

246. The method of claim 230, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 165, HFR2 is at least 95% identical to the sequence of SEQ ID NO: 166, HFR3 is at least 95% identical to the sequence of SEQ ID NO: 167; and HFR4 is at least 95% identical to the sequence of SEQ ID NO: 168.

247. The method of claim 231, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 169, HFR2 is at least 95% identical to the sequence of SEQ ID NO: 170, HFR3 is at least 95% identical to the sequence of SEQ ID NO: 171; and HFR4 is at least 95% identical to the sequence of SEQ ID NO: 172.

248. The method of claim 232, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 173, HFR2 is at least 95% identical to the sequence of SEQ ID NO: 174, HFR3 is at least 95% identical to the sequence of SEQ ID NO: 175; and HFR4 is at least 95% identical to the sequence of SEQ ID NO: 176.

249. The method of claim 233, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 177, HFR2 is at least 95% identical to the sequence of SEQ ID NO: 178, HFR3 is at least 95% identical to the sequence of SEQ ID NO: 179; and HFR4 is at least 95% identical to the sequence of SEQ ID NO: 180.

250. The method of claim 234, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 181, HFR2 is at least 95% identical to the sequence of SEQ ID NO: 182, HFR3 is at least 95% identical to the sequence of SEQ ID NO: 183; and HFR4 is at least 95% identical to the sequence of SEQ ID NO: 184.

251. The method of claim 235, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 185, HFR2 is at least 95% identical to the sequence of SEQ ID NO: 186, HFR3 is at least 95% identical to the sequence of SEQ ID NO: 187; and HFR4 is at least 95% identical to the sequence of SEQ ID NO: 188.

252. The method of claim 236, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 189, HFR2 is at least 95% identical to the sequence of SEQ ID NO: 190, HFR3 is at least 95% identical to the sequence of SEQ ID NO: 191; and HFR4 is at least 95% identical to the sequence of SEQ ID NO: 192.

253. The method of claim 237, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 193, HFR2 is at least 95% identical to the sequence of SEQ ID NO: 194, HFR3 is at least 95% identical to the sequence of SEQ ID NO: 195; and HFR4 is at least 95% identical to the sequence of SEQ ID NO: 196.

254. The method of claim 238, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 197, HFR2 is at least 95% identical to the sequence of SEQ ID NO: 198, HFR3 is at least 95% identical to the sequence of SEQ ID NO: 199; and HFR4 is at least 95% identical to the sequence of SEQ ID NO: 200.

255. The method of claim 239, wherein the first antigen binding moiety comprises a variable heavy chain (V_{H}) comprising framework regions HFR1, HFR2, HFR3, and HFR4 wherein HFR1 is at least 95% identical to the sequence of SEQ ID NO: 201, HFR2 is at least 95% identical to the sequence of SEQ ID NO: 202, HFR3 is at least 95% identical to the sequence of SEQ ID NO: 203; and HFR4 is at least 95% identical to the sequence of SEQ ID NO: 204.

256. The method of any one of claims 179-255, wherein the first antigen binding moiety comprises a variable light chain (V_{L}) having an amino acid sequence that is at least 90% identical to an amino acid sequence selected from SEQ ID NOs: 17-32.

257. The method of any one of claims 179-256, wherein the first antigen binding moiety comprises a variable heavy chain (V_{H}) having an amino acid sequence that is at least 90% identical to an amino acid sequence selected from SEQ ID NOs: 1-16.

258. The method of any one of claims 179-257, wherein the first antigen binding moiety is an antibody fragment.

259. The method of any one of claims 179-257, further comprising
(c) transporting the ABP to the cell surface.

260. A method of internalizing a target molecule, the method comprising:
(a) contacting the target molecule with an antigen binding protein (ABP) of any one of claims 1-108 or a bifunctional molecule of any one of claims 109-178; and
(b) internalizing the ABP within a cell.
